(19) United States
(12) Patent Application Publication
(10) Pub. No.: US 2024/0190868 A1
(43) Pub. Date: Jun. 13, 2024

(54) SOS1 INHIBITORS

(71) Applicant: Acerand Therapeutics (USA) Limited, Carmel, IN (US)

(72) Inventors: Kuo-Long Yu, Zionsville, IN (US); Sanjeev Kumar, Waverly, IA (US); Bin Liu, Fishers, IN (US); Weitao Pan, Old Saybrook, CT (US)

(21) Appl. No.: 18/506,062

(22) Filed: Nov. 9, 2023

Related U.S. Application Data
(60) Provisional application No. 63/424,310, filed on Nov. 10, 2022.

(51) Int. Cl. C07D 471/04; C07D 471/14; C07D 498/14

(52) U.S. Cl. CPC C07D 471/04; C07D 471/14; C07D 498/14

(57) ABSTRACT

A compound of formula I:

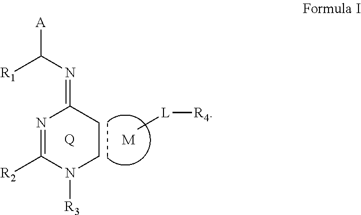

Each variable is defined herein. Also disclosed are a pharmaceutical composition containing a compound of formula I, a method of treating cancer using such a compound, and a method of inhibiting SOS1 with the compound.

### CROSS REFERENCES TO RELATED APPLICATIONS

This application claims the benefit of priority based on U.S. Application Ser. No. 63/424,310 filed on Nov. 10, 2022, the content and disclosure of which are incorporated herein by reference in their entirety.

### BACKGROUND

The RAS family of GTPases, including KRAS (V-Ki-ras2 Kirsten rat sarcoma viral oncogene homolog), NRAS (Neuroblastoma RAS viral oncogene homolog), and HRAS (Harvey murine sarcoma virus oncogene), are recognized as major oncogenes, occurring in up to 20 to 30% of human cancers.

Due to very high affinity of RAS proteins for GDP/GTP, the exchange from an RAS-GDP bound form (i.e., an inactive state) to an RAS-GTP bound form (i.e., an active state) is catalyzed by guanine nucleotide exchange factors (GEFs) such as Son of Sevenless 1 (SOS1) whereas the return to its inactive state (i.e., RAS-GDP bound form) is catalyzed by GTPase-activating proteins (GAPs). The activated RAS-GTP bound form interacts with a number of effectors to drive cell growth and differentiation.

In cancer cells, oncogenic activating mutations in RAS proteins, and/or the isoform SOS1, or inactivating mutations in GAPs result in constitutive activation of the RAS signaling pathway, which in turn leads to uncontrolled cancer cell proliferation and growth. Cancer patients with RAS mutations often have aggressive, metastatic disease with poor prognoses. Direct inhibition of RAS has proved extremely challenging. Alternative strategies to indirectly target the RAS signaling pathways have been explored, for example, inhibiting the enzymatic activity of GEFs such as SOS1 and or its interaction with RAS. These approaches should also lead to the attenuation of the RAS signaling pathway activity by preventing the formation of the active RAS-GTP bound form. Therefore, inhibition of SOS1 activity or its interaction with RAS is of therapeutic benefit. Indeed, certain benzylamino substituted pyridopyrimidione compounds and quinazoline compounds were developed as SOS1 inhibitors. See WO 2019/122129 and Hillig et al, Proc Natl Acad Sci USA, 2019, vol. 116(7), 2551-2560.

Nevertheless, there is still a large unmet need to develop SOS1 inhibitors for treating cancers and other diseases associated with or modulated by the SOS1 interaction with KRAS including cancers that harbor genetic alterations (mutations, fusions, translocations, amplification, and over-expression) in genes encoding ALK, AxL, BCR-ABL, c-Raf, c-Met, EGFR1-4, ErbB2, FGFR 1-4, Kras, NRas, HRas, NF1, NTRK, Ret, ROS, and other oncogenic signaling molecules.

### SUMMARY

The present invention is based on an unexpected discovery that certain compounds are effective SOS1 inhibitors, suitable for treating cancer and other diseases such as neurofibromatosis, Noonan syndrome (NS), cardiofaciocutaneous syndrome (CFC), and hereditary gingival fibromatosis type 1.

In one aspect, this invention relates to compounds of formula I:

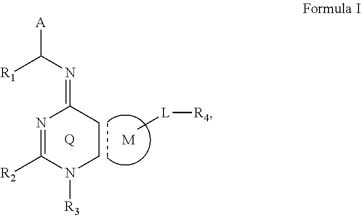

or a pharmaceutical acceptable salt, solvate, isomer, prodrug, or tautomer thereof, wherein:
A is C₃-C₆ cycloalkyl, 4-10 membered heterocycloalkyl, aryl, or 5-10 membered heteroaryl;
L is a single bond, —O—, —C(O)—, —C(O)O—, —O—CO—, —C(O)NR′—, —NR′—, —NR′CO—, —NR′SO₂—, —SO₂NR′—, —S—, —SO—, —S(O)₂—, C₁-C₃ alkylene, C₁-C₃ haloalkylene, C₂-C₃ alkenylene, or C₂-C₃ alkynylene, in which R′ is H, C₁-C₆ alkyl, C₁-C₆ haloalkyl, C₃-C₆ cycloalkyl, 3-6 membered heterocycloalkyl, 5-10 membered heteroaryl, or 6-10 membered aryl;
M is a C₅-C₁₀ cycloalkyl, 5-15 membered heterocyclyl, 6-10 membered aryl, or 5-10 membered heteroaryl;
Q indicates the left ring of formula I to which M is fused;
R₁ is CN, C₁-C₃ alkyl, C₂-C₃ alkenyl, or C₂-C₃ alkynyl;
R₂ is H, CN, halogen, C₁-C₃ alkyl, C₁-C₃ alkoxy, C₂-C₃ alkynyl, or C₃-C₆ cycloalkyl;
R₃ is C₁-C₆ alkyl, C₃-C₆ cycloalkyl, 4-6 membered heterocycloalkyl, 5-6 membered heteroaryl, or phenyl; alternatively, R₂ and R₃, together with the carbon atom to which R₂ bonds and the nitrogen atom to which R₃ bonds, form a 4-7 membered heterocycloalkyl;
R₄ is H, C₁-C₁₀ alkyl, C₁-C₁₀ alkylamino, C₁-C₁₀ alkylamido, C₂-C₁₀ alkylcarbonyl, C₁-C₁₀ alkylsulfonyl, C₁-C₁₀ alkysulfonamido, C₃-C₆ cycloalkyl, C₃-C₆ cycloalkylcarbonyl, C₃-C₆ cycloalkylamido, 3-10 membered heterocycloalkyl, 6-10 membered aryl, or 5-10 membered heteroaryl;
[symbol] is a single or double bond; and
each of alkyl, alkylene, alkenyl, alkynyl, alkoxy, haloalkylene, alkylamino, alkylamido, alkylcarbonyl, alkylsulfonyl, alkysulfonamido, alkynylene, cycloalkyl, cycloalkylcarbonyl, cycloalkylamido, heterocycloalkyl, heterocyclyl, phenyl, aryl, and heteroaryl is unsubstituted or substituted with one or more of deuterium, halogen, CN, oxo, C₁-C₆ alkyl, C₃-C₆ cycloalkyl, C₁-C₆ haloalkyl, C₂-C₆ alkenyl, C₂-C₆ alkynyl, —OR′, —C(O)R′, —C(O)OR′, —O—COR′, —C(O)NR′R″, —NR′R″, —SR′, —SOR′, —S(O)₂R′, and —POR′R″, in which each of R′ and R″, independently, is H, halogen, C₁-C₆ alkyl, C₁-C₆ haloalkyl, C₃-C₆ cycloalkyl, 3-6 membered heterocycloalkyl, 5-10 membered heteroaryl, or 6-10 membered aryl.

In another aspect, this invention relates to compounds of formula I.

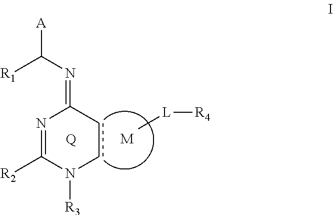

In this formula, A is C₃-C₆ cycloalkyl, 4-10 membered heterocycloalkyl, aryl (e.g., phenyl), or 5-10 (e.g., 5-7) membered heteroaryl;
L is a single bond, —O—, —C(O)—, —C(O)O—, —O—CO—, —C(O)NR′—, —NR′—, —NR′CO—, —NR′SO₂—, —SO₂NR′—, —S—, —SO—, —S(O)₂—, C₁-C₃ alkylene, C₁-C₃ haloalkylene, C₂-C₃ alkenyl, or C₂-C₃ alkynylene, in which R′ is H, C₁-C₆ alkyl, C₁-C₆ haloalkyl, C₃-C₆ cycloalkyl, 3-6 membered heterocycloalkyl, 5-10 membered heteroaryl, or 6 or 10 membered aryl (e.g., phenyl);
M is a C₅-C₁₀ cycloalkyl, 5-12 (e.g., 5-7) membered heterocycloalkyl, 6 or 10 membered aryl (e.g., phenyl), or 5-10 (e.g., 5-6) membered heteroaryl;
Q indicates the left ring as shown in Formula I to which M is fused;
R₁ is CN, C₁-C₃ alkyl, ethenyl, or ethynyl,
R₂ is H, CN, halogen, C₁-C₃ alkyl, C₁-C₃ alkoxy, ethenyl, or cyclopropyl; R₃ is C₁-C₆ alkyl, C₃-C₆ cycloalkyl, 4-6 membered heterocycloalkyl, 5-6 membered heteroaryl, or phenyl; alternatively, R₂ and R₃, together with the carbon atom to which R₂ bonds and the nitrogen atom to which R₃ bonds, form a 4-7 membered heterocycloalkyl;
R₄ is H, C₁-C₁₀ alkyl, C₃-C₆ cycloalkyl, 3-10 membered heterocycloalkyl, 6-10 membered aryl, or 5-10 membered heteroaryl;
[symbol] is a single or double bond; and
each of alkyl, cycloalkyl, heterocycloalkyl, phenyl, aryl, and heteroaryl is optionally substituted with one or more of deuterium, halogen, CN, oxo, C₁-C₆ alkyl, C₃-C₆ cycloalkyl, C₁-C₆ haloalkyl, C₂-C₆ alkenyl, C₂-C₆ alkynyl, —OR′, —C(O)R′, —C(O)OR′, —O—COR′, —C(O)NR′R″, —NR′R″, —SR′, —SOR′, —S(O)₂R′, or —POR′R″, in which each of R′ and R″, independently, is H, halogen, C₁-C₆ alkyl, C₁-C₆ haloalkyl, C₃-C₆ cycloalkyl, 3-6 membered heterocycloalkyl, 5-10 membered heteroaryl, or 6 or 10 membered aryl.

A subset of the compounds of Formula I is compounds of Formula II:

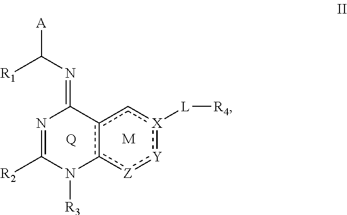

in which each of [symbol] is a single or double bond and the number of double bonds represented by [symbol] in formula II is two or three; X is C or N, and each of Y and Z, independently, is C═O, C—Rx, or N-Ry, provided that when X is N, then Y is C═O and Z is C—Rx; Rx is H, CN, halogen, C₁-C₃ alkyl, C₁-C₃ alkoxy, C₁-C₃ haloalkyl, cyclopropyl, four to six membered heterocycloalkoxy, or NR′R″; and Ry is absent, H, C₁-C₃ alkyl, or cyclopropyl. Variables A, L, and R₁ to R₄ are defined above.

Preferred compounds each have one of Formulas II-A to II-H shown below.

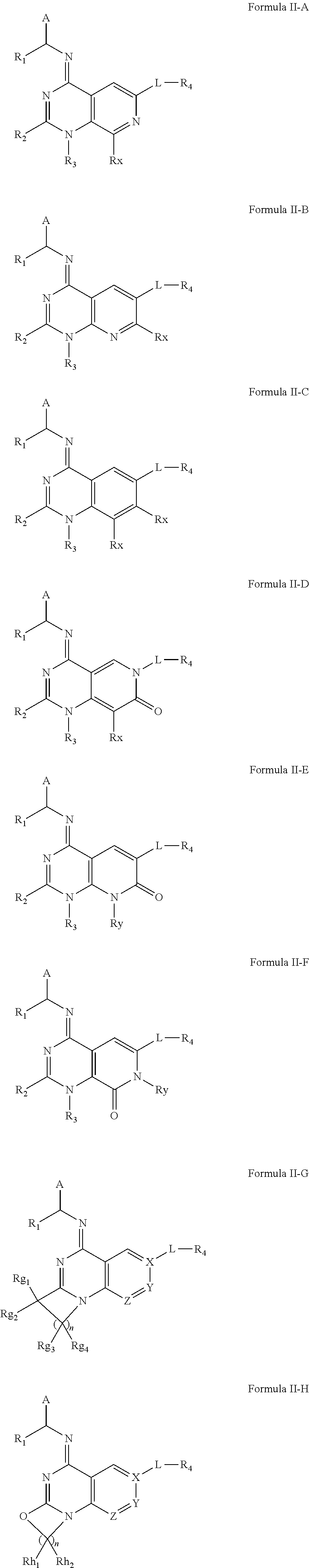

In Formulas II-G and II-H above, n is 2, 3, or 4, preferably 2; and each of Rg₁, Rg₂, Rg₃, Rg₄, Rh₁, and Rh₂, independently, is H, halogen, or methyl. All other variables are defined as above.

Among the compounds described above,

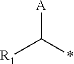

can be

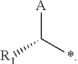

In another embodiment,

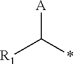

is

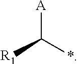

In preferred compounds, L is a single bond, —O—, —C(O)—, —NHC(O)—, —NHS(O)₂—, C₂-C₃ alkenylene, or C₂-C₃ alkynylene.

Another subset of compounds of this invention are compounds of Formula III:

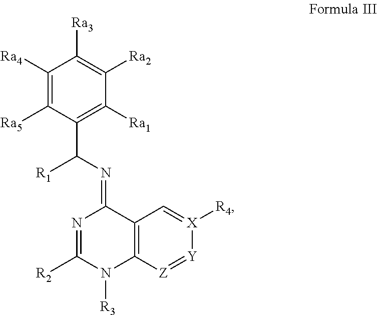

in which each of Ra₁, Ra₂, Ra₃, Ra₄, and Ra₅, independently, is H, halogen, amino, CN, C₁-C₆ alkyl, C₂-C₆ alkenyl, C₁-C₆ haloalkyl, C₃-C₆ cycloalkyl, or 4-6 membered heterocycloalkyl.

An additional subset of compounds of this invention are compounds of Formula IIIa or Formula IIIb:

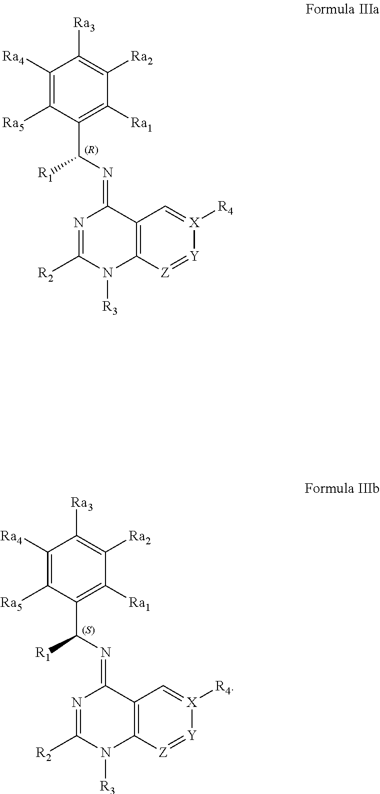

A further subset of the compounds of this invention are compounds of formula IV, V, or VI:

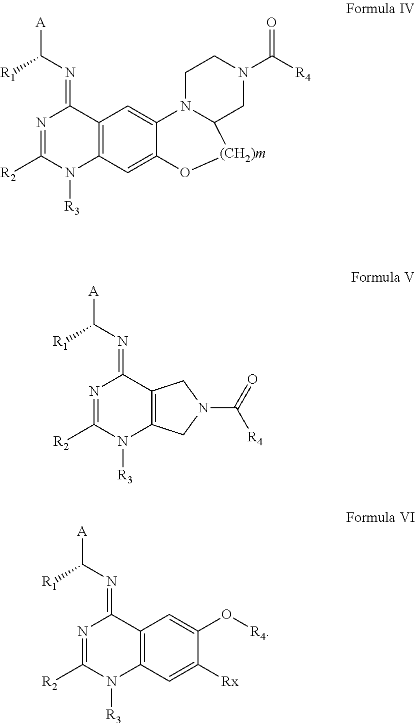

In formula IV above, m is 0, 1, 2, 3, or 4, preferably 1. All other variables are defined above.

In preferred compounds, A is phenyl, 2,3-dihydro-1H-indene, or benzofuran, each of which is unsubstituted or substituted with one or more groups selected from deuterium, halogen, NH₂, CN, C₁-C₄ alkyl, C₂-C₄ alkenyl, C₁-C₄ haloalkyl, and C₃-C₆ cycloalkyl.

In more preferred compounds, A is phenyl substituted with one or more groups selected from halogen (e.g. F, Cl, and Br), NH₂, CN, C₁-C₃ alkyl (e.g. CH₃), and C₁-C₃ haloalkyl (e.g. CF₃, CHF₂, and CF₂CH₃).

Among the compounds described above, preferred compounds have one or more of the following features:
(i) R₁ is methyl or ethynyl,
(ii) R₂ is H, C₁-C₃ alkyl (e.g., CH₃, CD₃), or cyclopropyl,
(iii) R₃ is C₁-C₄ alkyl (e.g., CH₃, CH(CH₃)₂, CD₃), C₃-C₆ cycloalkyl, or 4 to 6-membered heterocycloalkyl, preferably, C₁-C₄ alkyl (e.g., CH₃, CD₃) or cyclopropyl,
(iv) Rx is H, halogen, C₁-C₃ alkyl, cyclopropyl, C₁-C₃ alkoxy, NH₂, or C₁-C₃ alkylamino,
(v) Ry is H, C₁-C₃ alkyl, or cyclopropyl,
(vi) A is

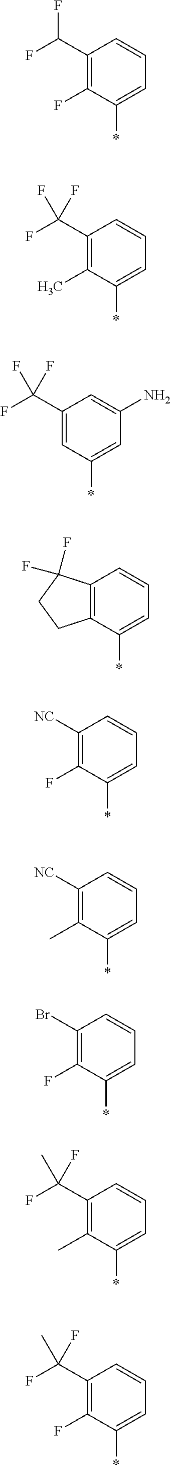

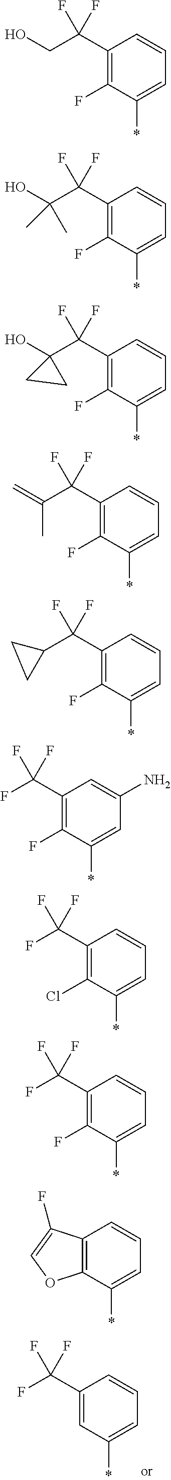

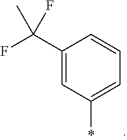 [SMILES: *c1cccc(C(C)(F)F)c1]

Preferably, A is

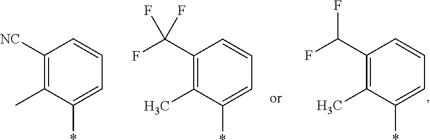

(vii) L is a single bond, and
(viii) R₄ is selected from the group consisting of:

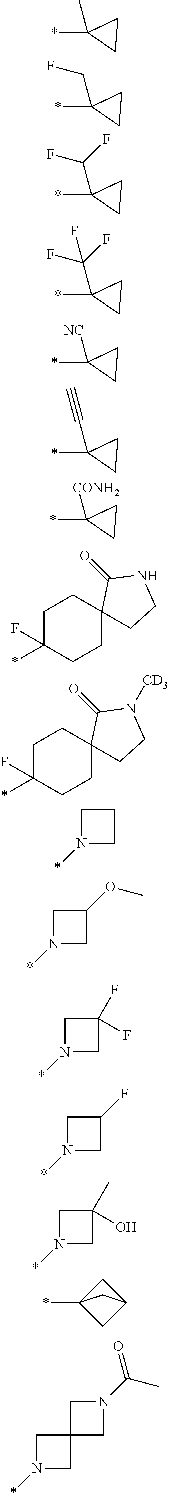

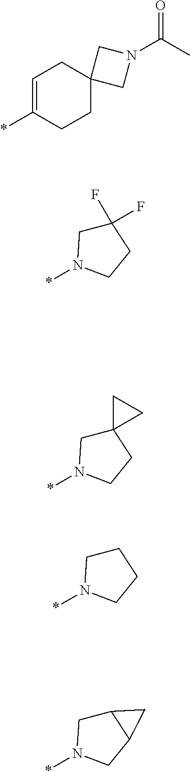 [SMILES: *C1=CCC2(CC1)CN(C(C)=O)C2 ; *N1CCC(F)(F)C1 ; *N1CCC2(CC2)C1 ; *N1CCCC1 ; *N1CC2CC2C1]

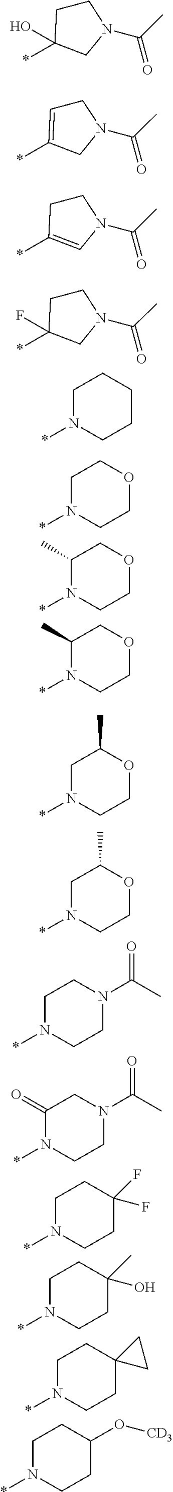

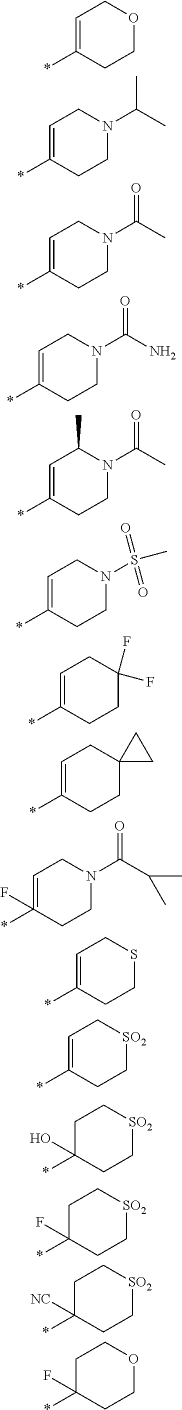

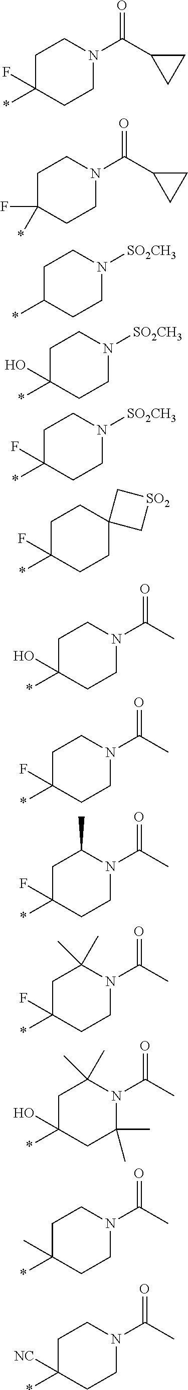

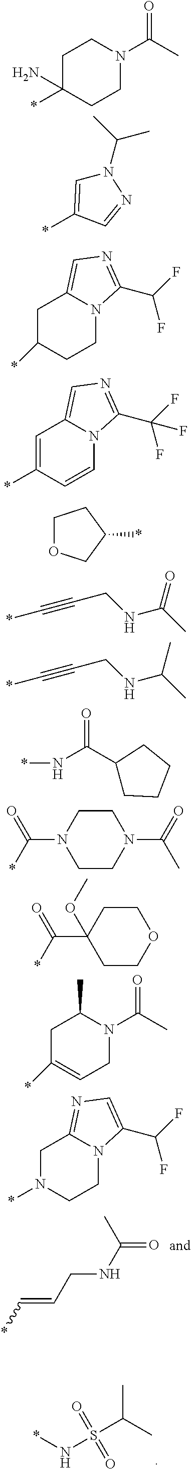

Preferably, R₄ is selected from the group consisting of

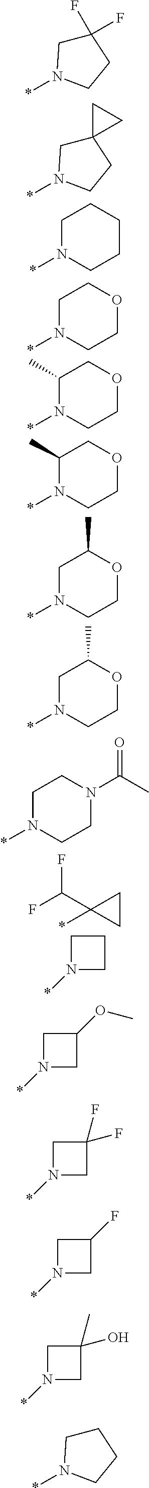

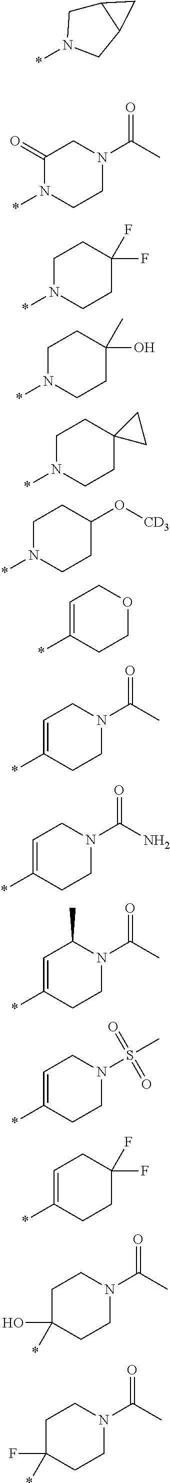

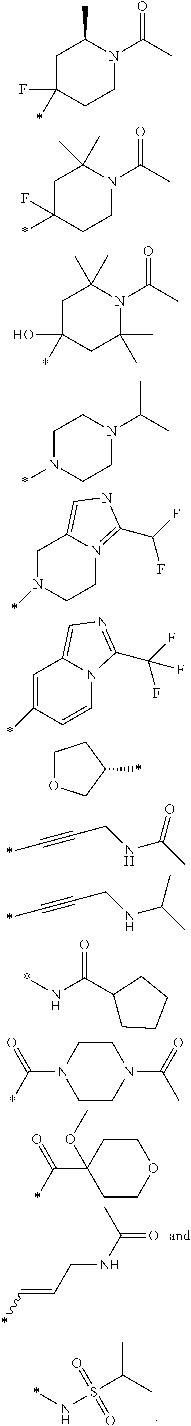

In preferred compounds of formula IV, V, and VI, m is 0, 1, or 2, and Rx is H or C₁-C₁₀ alkoxy. More preferably, R₁ is methyl or ethynyl (most preferably methyl); R₂ is H, C₁-C₃ alkyl (e.g., CH₃ and CD₃), or cyclopropyl; R₃ is C₁-C₄ alkyl (e.g., CH₃ and CD₃), C₃-C₆ cycloalkyl (e.g., cyclopropyl), or 4 to 6-membered heterocycloalkyl; R₄ is H, C₁-C₆ alkyl (e.g., CH₃ and CD₃), C₁-C₆ haloalkyl, C₃-C₆ cycloalkyl, or 4 to 6-membered heterocycloalkyl; A is

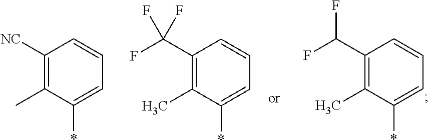

and m is 1 or 2.

In more preferred compounds of formula IV, R₁ is methyl; R₂ is H or methyl, R₃ is methyl, or R₂ and R₃, together with the carbon atom to which R₂ bonds and the nitrogen atom to which R₃ bonds, form a 5 or 6-membered heterocycloalkyl; R₄ is methyl or cyclopropyl; and m is 1 or 2.

Another aspect of this invention relates to a pharmaceutical composition containing any of the compounds described above and a pharmaceutically acceptable carrier thereof.

Also within the scope of this invention is a method of treating cancer including the step of administering to a subject in need thereof an effective amount of any of the compounds described above or a pharmaceutical composition containing such a compound.

Still within the scope of this invention is a method of inhibiting SOS1 by administering to a subject in need thereof an effective amount of any of the above-described compound or a pharmaceutical composition containing such a compound.

Table 1 below shows 113 exemplary compounds of the present invention.

TABLE 1

| | |
|---|---|
| 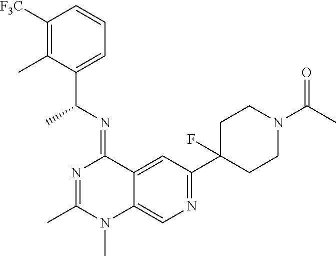 [SMILES: CC(=O)N1CCC(F)(c2cc3/c(=N/[C@H](C)c4cccc(C(F)(F)F)c4C)nc(C)n(C)c3cn2)CC1] | 1 |
| 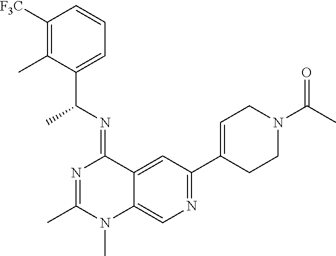 [SMILES: CC(=O)N1CC=C(c2cc3/c(=N/[C@H](C)c4cccc(C(F)(F)F)c4C)nc(C)n(C)c3cn2)CC1] | 2 |
| 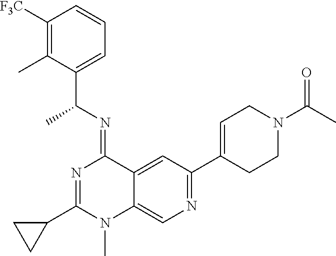 [SMILES: CC(=O)N1CC=C(c2cc3/c(=N/[C@H](C)c4cccc(C(F)(F)F)c4C)nc(C4CC4)n(C)c3cn2)CC1] | 3 |
| 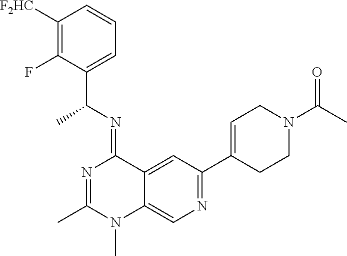 [SMILES: CC(=O)N1CC=C(c2cc3/c(=N/[C@H](C)c4cccc(C(F)F)c4F)nc(C)n(C)c3cn2)CC1] | 4 |
| 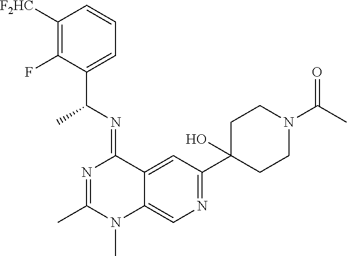 [SMILES: CC(=O)N1CCC(O)(c2cc3/c(=N/[C@H](C)c4cccc(C(F)F)c4F)nc(C)n(C)c3cn2)CC1] | 5 |
| 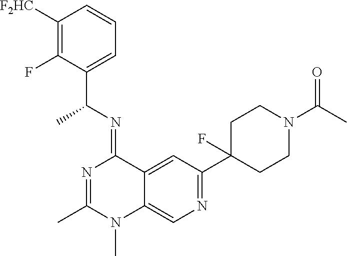 [SMILES: CC(=O)N1CCC(F)(c2cc3/c(=N/[C@H](C)c4cccc(C(F)F)c4F)nc(C)n(C)c3cn2)CC1] | 6 |
| 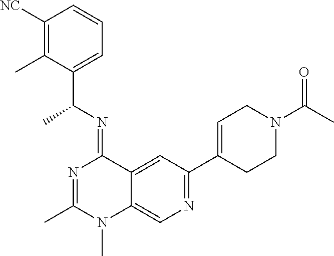 [SMILES: CC(=O)N1CC=C(c2cc3/c(=N/[C@H](C)c4cccc(C#N)c4C)nc(C)n(C)c3cn2)CC1] | 7 |
| 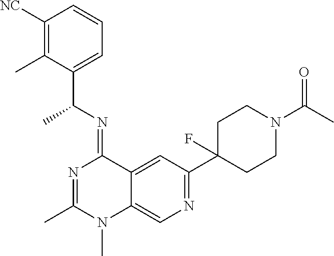 [SMILES: CC(=O)N1CCC(F)(c2cc3/c(=N/[C@H](C)c4cccc(C#N)c4C)nc(C)n(C)c3cn2)CC1] | 8 |
| 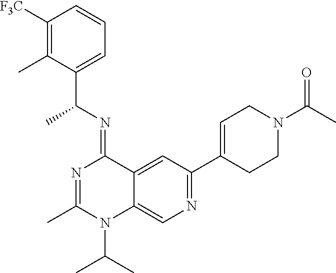 [SMILES: CC(=O)N1CC=C(c2cc3/c(=N/[C@H](C)c4cccc(C(F)(F)F)c4C)nc(C)n(C(C)C)c3cn2)CC1] | 9 |
| 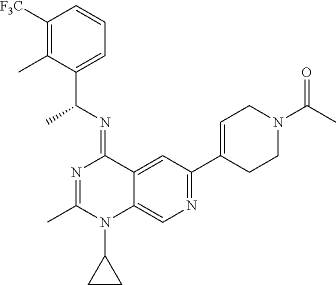 [SMILES: CC(=O)N1CC=C(c2cc3/c(=N/[C@H](C)c4cccc(C(F)(F)F)c4C)nc(C)n(C4CC4)c3cn2)CC1] | 10 |
| 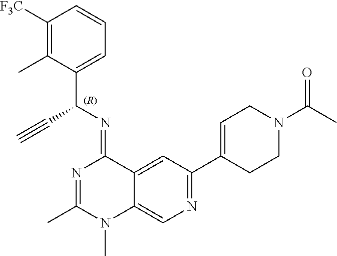 | 11 |
| 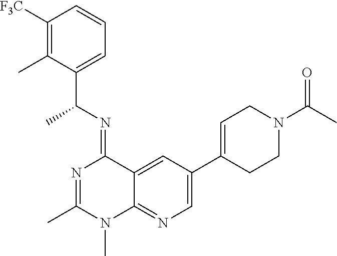 [SMILES: CC(=O)N1CC=C(c2cnc3c(c2)/c(=N/[C@H](C)c2cccc(C(F)(F)F)c2C)nc(C)n3C)CC1] | 12 |
| 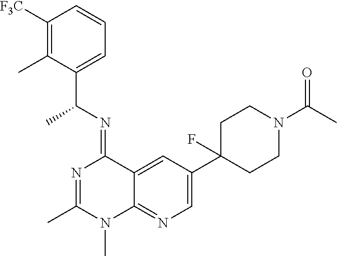 [SMILES: CC(=O)N1CCC(F)(c2cnc3c(c2)/c(=N/[C@H](C)c2cccc(C(F)(F)F)c2C)nc(C)n3C)CC1] | 13 |
| 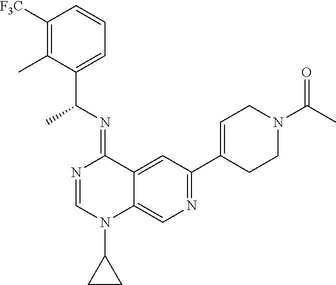 [SMILES: CC(=O)N1CC=C(c2cc3/c(=N/[C@H](C)c4cccc(C(F)(F)F)c4C)ncn(C4CC4)c3cn2)CC1] | 14 |
| 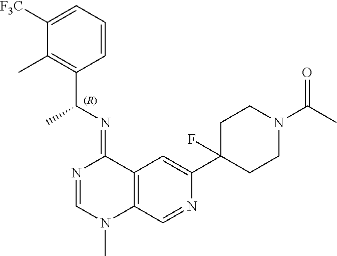 | 15 |
| 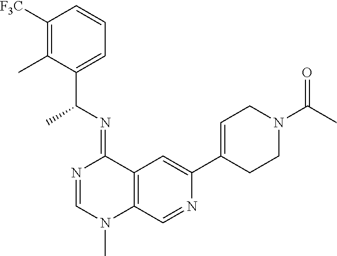 [SMILES: CC(=O)N1CC=C(c2cc3/c(=N/[C@H](C)c4cccc(C(F)(F)F)c4C)ncn(C)c3cn2)CC1] | 16 |
| 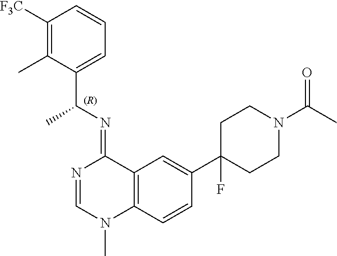 | 17 |
| 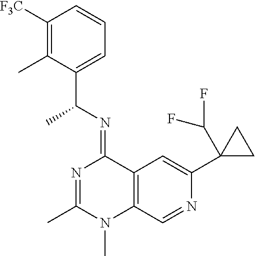 [SMILES: Cc1c([C@@H](C)/N=c2\nc(C)n(C)c3cnc(C4(C(F)F)CC4)cc23)cccc1C(F)(F)F] | 18 |
| 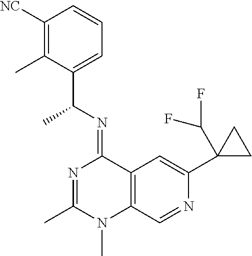 [SMILES: Cc1c(C#N)cccc1[C@@H](C)/N=c1\nc(C)n(C)c2cnc(C3(C(F)F)CC3)cc12] | 19 |
| 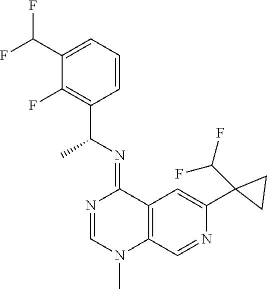 [SMILES: C[C@@H](/N=c1\ncn(C)c2cnc(C3(C(F)F)CC3)cc12)c1cccc(C(F)F)c1F] | 20 |
| 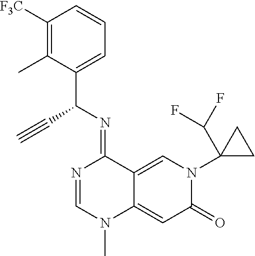 [SMILES: C#C[C@@H](/N=c1\ncn(C)c2cc(=O)n(C3(C(F)F)CC3)cc12)c1cccc(C(F)(F)F)c1C] | 21 |
| 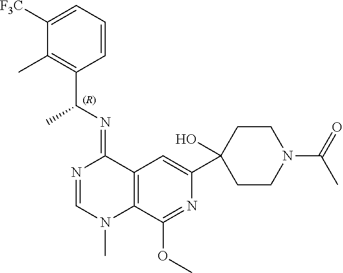 | 22 |
| 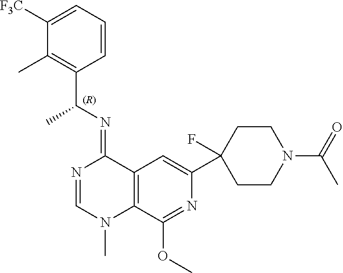 | 23 |
| 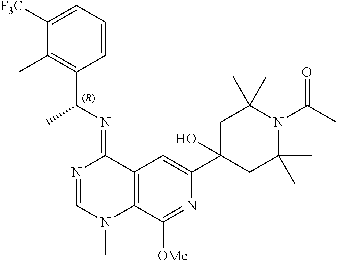 | 24 |
| 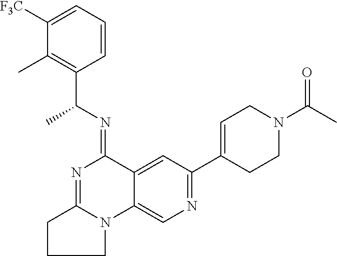 [SMILES: CC(=O)N1CC=C(c2cc3/c(=N/[C@H](C)c4cccc(C(F)(F)F)c4C)nc4n(c3cn2)CCC4)CC1] | 25 |
| 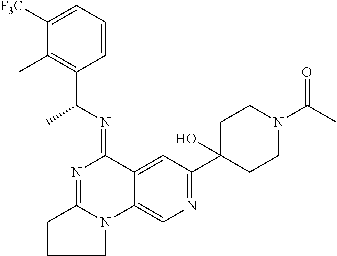 [SMILES: CC(=O)N1CCC(O)(c2cc3/c(=N/[C@H](C)c4cccc(C(F)(F)F)c4C)nc4n(c3cn2)CCC4)CC1] | 26 |
| 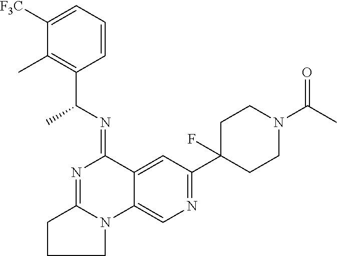 [SMILES: CC(=O)N1CCC(F)(c2cc3/c(=N/[C@H](C)c4cccc(C(F)(F)F)c4C)nc4n(c3cn2)CCC4)CC1] | 27 |
| 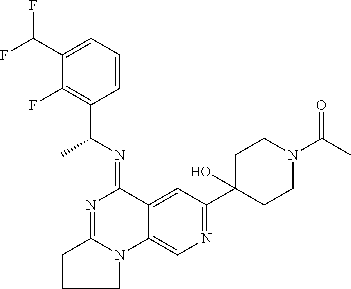 [SMILES: CC(=O)N1CCC(O)(c2cc3/c(=N/[C@H](C)c4cccc(C(F)F)c4F)nc4n(c3cn2)CCC4)CC1] | 28 |
| 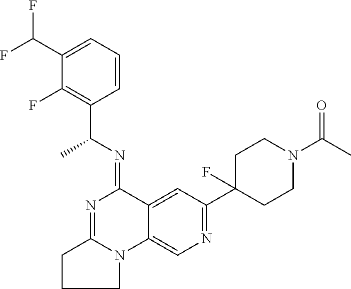 [SMILES: CC(=O)N1CCC(F)(c2cc3/c(=N/[C@H](C)c4cccc(C(F)F)c4F)nc4n(c3cn2)CCC4)CC1] | 29 |
| 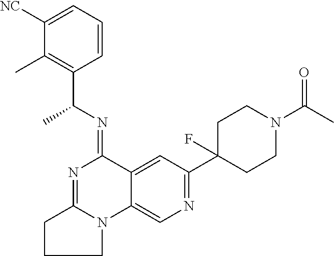 [SMILES: CC(=O)N1CCC(F)(c2cc3/c(=N/[C@H](C)c4cccc(C#N)c4C)nc4n(c3cn2)CCC4)CC1] | 30 |
| 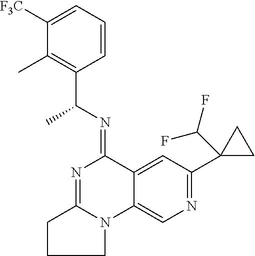 [SMILES: Cc1c([C@@H](C)/N=c2\nc3n(c4cnc(C5(C(F)F)CC5)cc24)CCC3)cccc1C(F)(F)F] | 31 |
| 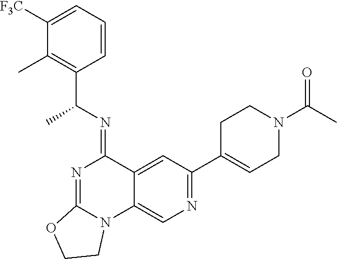 [SMILES: CC(=O)N1CC=C(c2cc3/c(=N/[C@H](C)c4cccc(C(F)(F)F)c4C)nc4n(c3cn2)CCO4)CC1] | 32 |
| 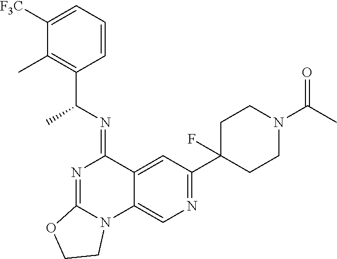 [SMILES: CC(=O)N1CCC(F)(c2cc3/c(=N/[C@H](C)c4cccc(C(F)(F)F)c4C)nc4n(c3cn2)CCO4)CC1] | 33 |
| 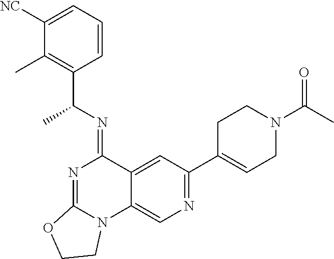 [SMILES: CC(=O)N1CC=C(c2cc3/c(=N/[C@H](C)c4cccc(C#N)c4C)nc4n(c3cn2)CCO4)CC1] | 34 |
| 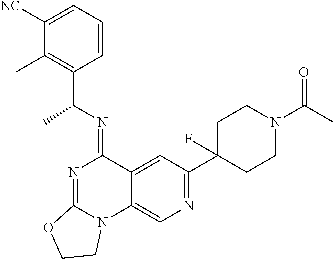 [SMILES: CC(=O)N1CCC(F)(c2cc3/c(=N/[C@H](C)c4cccc(C#N)c4C)nc4n(c3cn2)CCO4)CC1] | 35 |
| 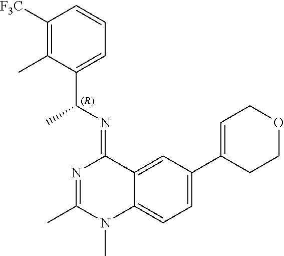 | 36 |
| 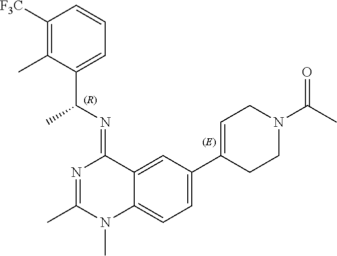 | 37 |
| 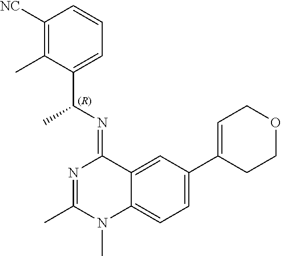 | 38 |
| 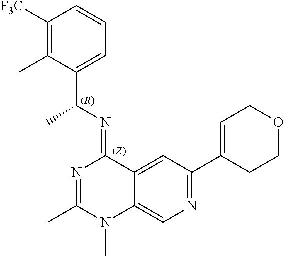 | 39 |
| 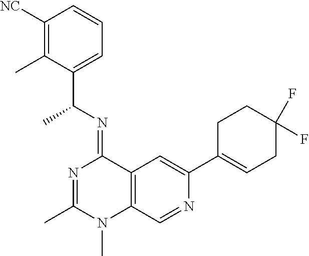 [SMILES: Cc1c(C#N)cccc1[C@@H](C)/N=c1\nc(C)n(C)c2cnc(C3=CCC(F)(F)CC3)cc12] | 40 |
| 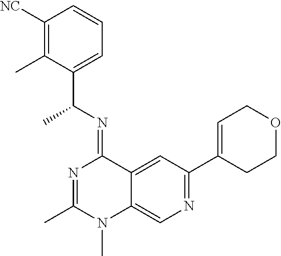 [SMILES: Cc1c(C#N)cccc1[C@@H](C)/N=c1\nc(C)n(C)c2cnc(C3=CCOCC3)cc12] | 41 |
| 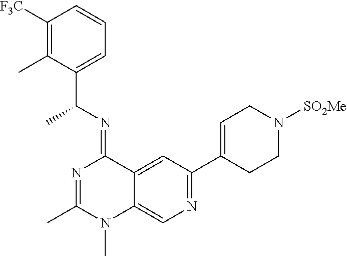 [SMILES: Cc1c([C@@H](C)/N=c2\nc(C)n(C)c3cnc(C4=CCN(S(C)(=O)=O)CC4)cc23)cccc1C(F)(F)F] | 42 |
| 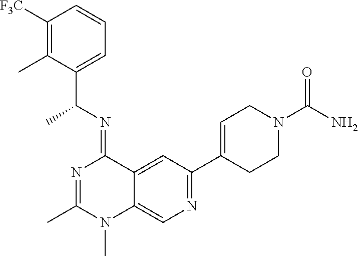 [SMILES: Cc1c([C@@H](C)/N=c2\nc(C)n(C)c3cnc(C4=CCN(C(N)=O)CC4)cc23)cccc1C(F)(F)F] | 43 |
| 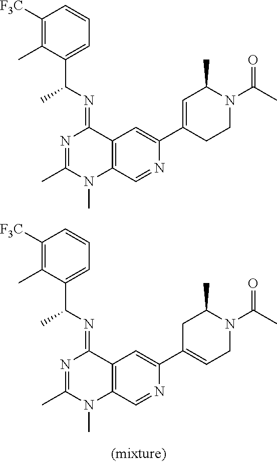 | 44 |
| 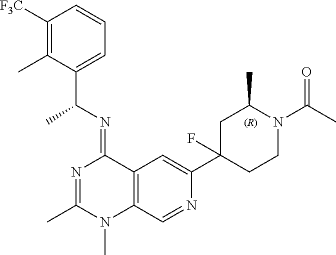 | 45 |
| 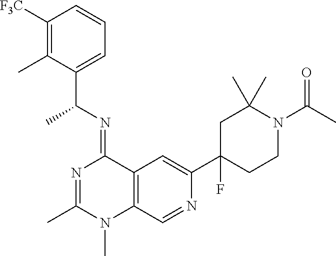 [SMILES: CC(=O)N1CCC(F)(c2cc3/c(=N/[C@H](C)c4cccc(C(F)(F)F)c4C)nc(C)n(C)c3cn2)CC1(C)C] | 46 |
| 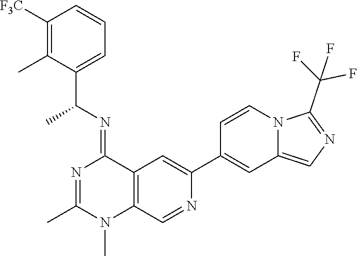 [SMILES: Cc1c([C@@H](C)/N=c2\nc(C)n(C)c3cnc(-c4ccn5c(C(F)(F)F)ncc5c4)cc23)cccc1C(F)(F)F] | 47 |
| 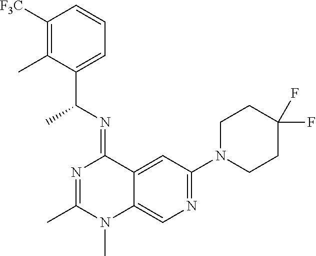 [SMILES: Cc1c([C@@H](C)/N=c2\nc(C)n(C)c3cnc(N4CCC(F)(F)CC4)cc23)cccc1C(F)(F)F] | 48 |
| 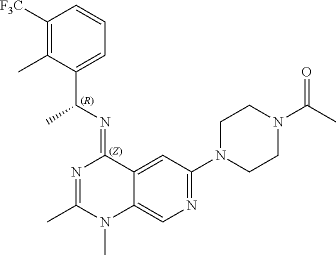 | 49 |
| 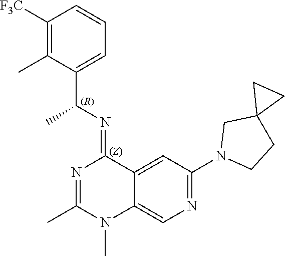 | 50 |
| 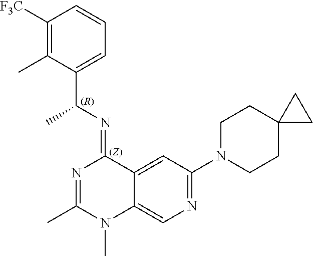 | 51 |
| 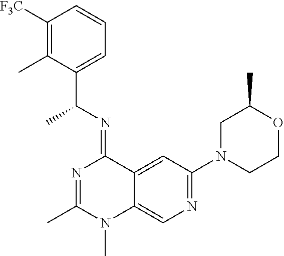 [SMILES: Cc1c([C@@H](C)/N=c2\nc(C)n(C)c3cnc(N4CCO[C@H](C)C4)cc23)cccc1C(F)(F)F] | 52 |
| 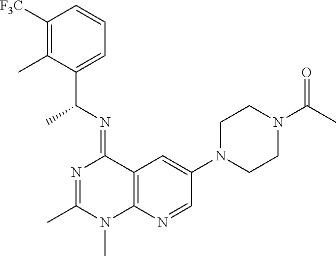 [SMILES: CC(=O)N1CCN(c2cnc3c(c2)/c(=N/[C@H](C)c2cccc(C(F)(F)F)c2C)nc(C)n3C)CC1] | 53 |
| 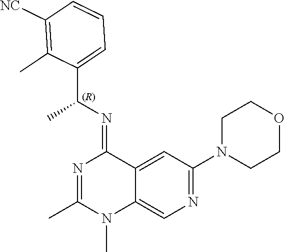 | 54 |
| 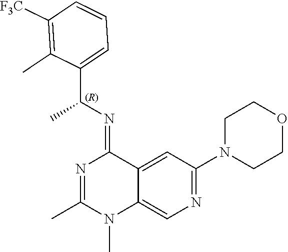 | 55 |
| 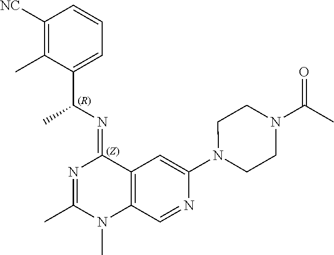 | 56 |
| 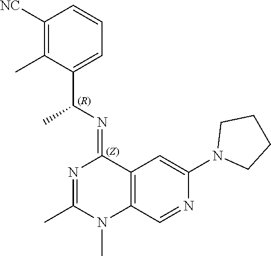 | 57 |
| 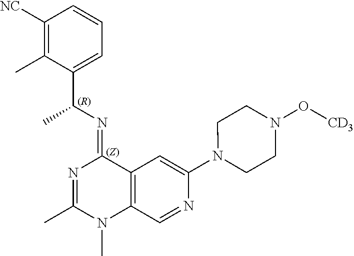 | 58 |
| 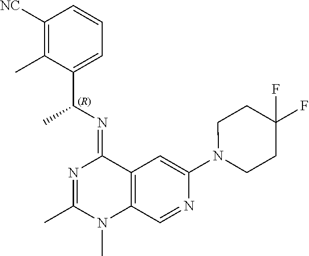 | 59 |
| 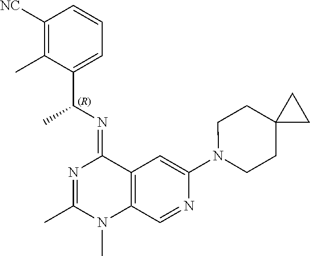 | 60 |
| 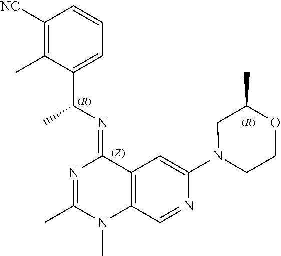 | 61 |
| 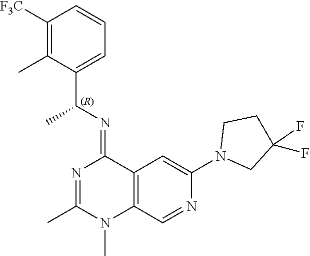 | 62 |
| 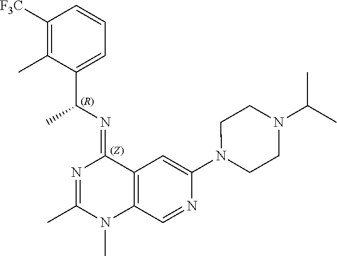 | 63 |
| 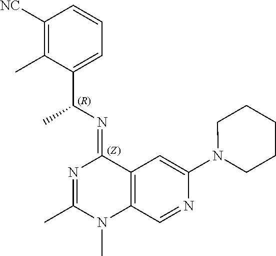 | 64 |
| 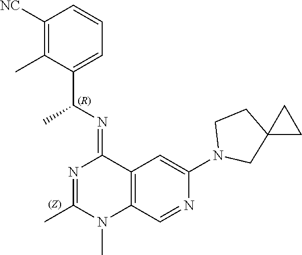 | 65 |
| 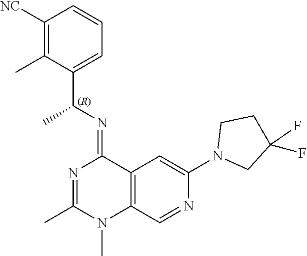 | 66 |
| 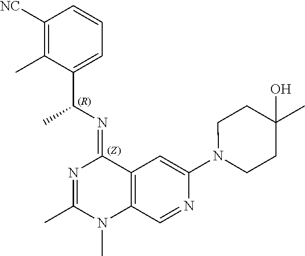 | 67 |
| 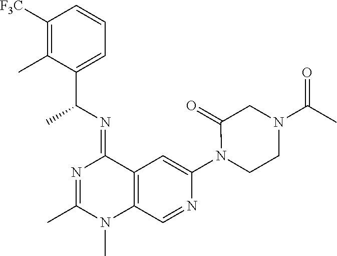 [SMILES: CC(=O)N1CCN(c2cc3/c(=N/[C@H](C)c4cccc(C(F)(F)F)c4C)nc(C)n(C)c3cn2)C(=O)C1] | 68 |
| 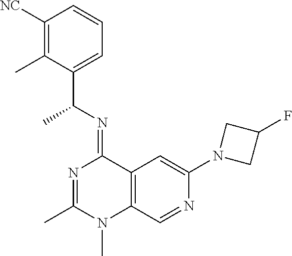 [SMILES: Cc1c(C#N)cccc1[C@@H](C)/N=c1\nc(C)n(C)c2cnc(N3CC(F)C3)cc12] | 69 |
| 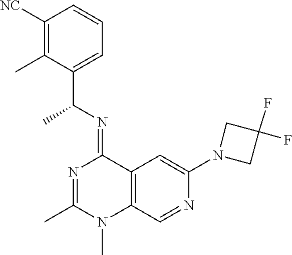 [SMILES: Cc1c(C#N)cccc1[C@@H](C)/N=c1\nc(C)n(C)c2cnc(N3CC(F)(F)C3)cc12] | 70 |
| 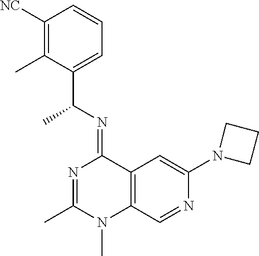 [SMILES: Cc1c(C#N)cccc1[C@@H](C)/N=c1\nc(C)n(C)c2cnc(N3CCC3)cc12] | 71 |
| 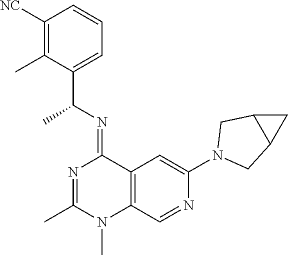 [SMILES: Cc1c(C#N)cccc1[C@@H](C)/N=c1\nc(C)n(C)c2cnc(N3CC4CC4C3)cc12] | 72 |
| 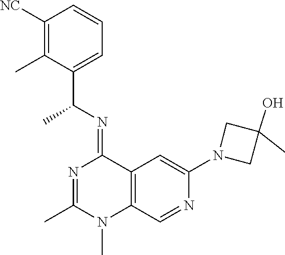 | 73 |
| 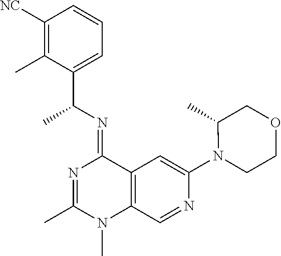 [SMILES: Cc1c(C#N)cccc1[C@@H](C)/N=c1\nc(C)n(C)c2cnc(N3CCOC[C@H]3C)cc12] | 74 |
| 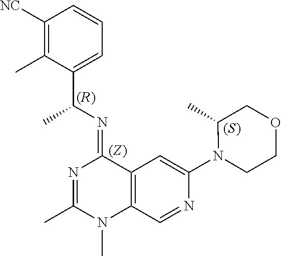 | 75 |
| 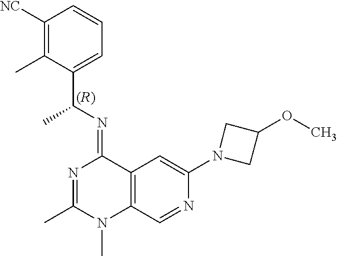 | 76 |
| 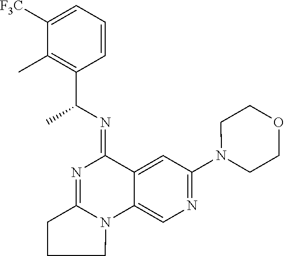 [SMILES: Cc1c([C@@H](C)/N=c2\nc3n(c4cnc(N5CCOCC5)cc24)CCC3)cccc1C(F)(F)F] | 77 |
| 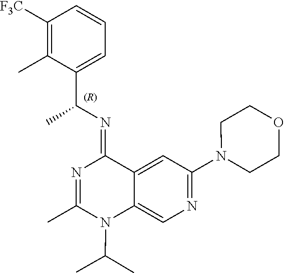 | 78 |
| 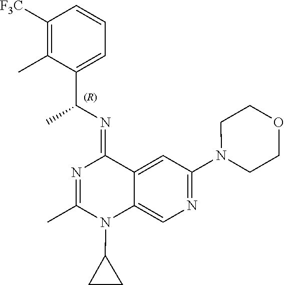 | 79 |
| 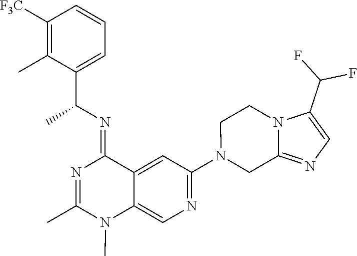 [SMILES: Cc1c([C@@H](C)/N=c2\nc(C)n(C)c3cnc(N4CCn5c(C(F)F)cnc5C4)cc23)cccc1C(F)(F)F] | 80 |
| 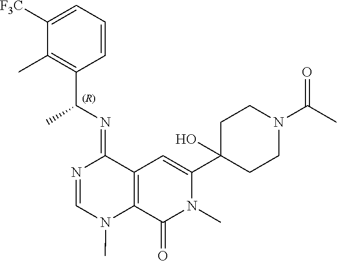 | 81 |
| 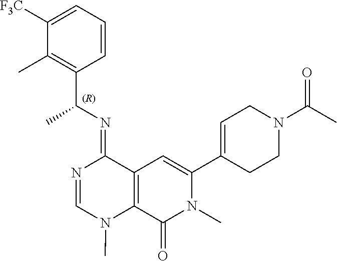 | 82 |
| 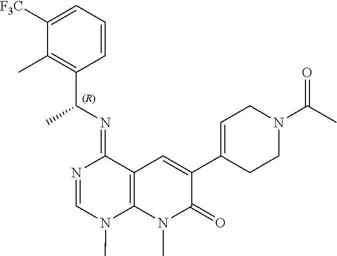 | 83 |
| 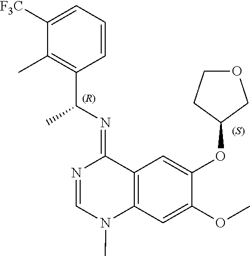 | 84 |
| 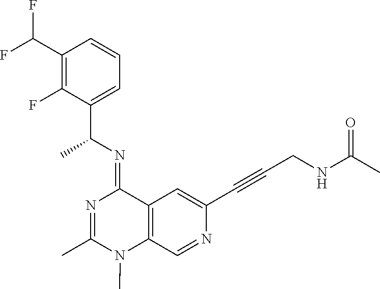 [SMILES: CC(=O)NCC#Cc1cc2/c(=N/[C@H](C)c3cccc(C(F)F)c3F)nc(C)n(C)c2cn1] | 85 |
| 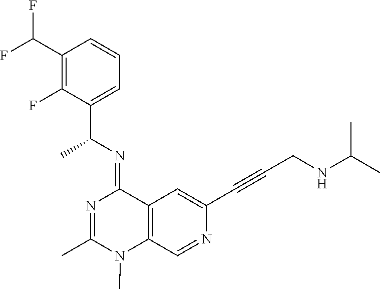 [SMILES: Cc1n/c(=N\[C@H](C)c2cccc(C(F)F)c2F)c2cc(C#CCNC(C)C)ncc2n1C] | 86 |
| 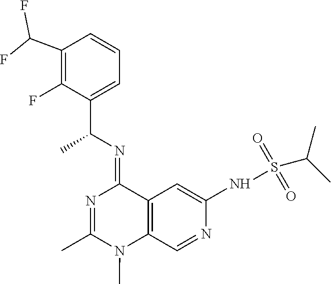 [SMILES: Cc1n/c(=N\[C@H](C)c2cccc(C(F)F)c2F)c2cc(NS(=O)(=O)C(C)C)ncc2n1C] | 87 |
| 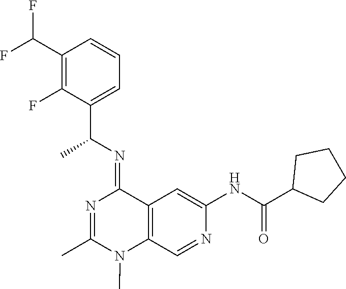 [SMILES: Cc1n/c(=N\[C@H](C)c2cccc(C(F)F)c2F)c2cc(NC(=O)C3CCCC3)ncc2n1C] | 88 |
| 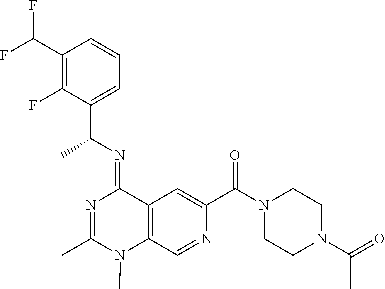 [SMILES: CC(=O)N1CCN(C(=O)c2cc3/c(=N/[C@H](C)c4cccc(C(F)F)c4F)nc(C)n(C)c3cn2)CC1] | 89 |
| 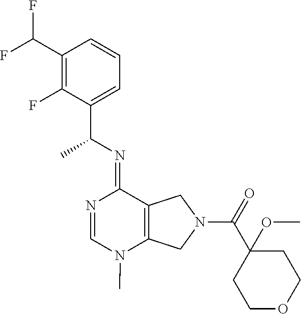 [SMILES: COC1(C(=O)N2Cc3c(n(C)cn/c3=N\[C@H](C)c3cccc(C(F)F)c3F)C2)CCOCC1] | 90 |
| 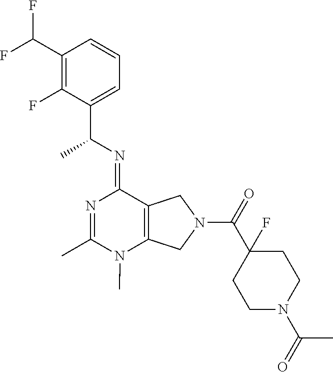 [SMILES: CC(=O)N1CCC(F)(C(=O)N2Cc3c(n(C)c(C)n/c3=N\[C@H](C)c3cccc(C(F)F)c3F)C2)CC1] | 91 |
| 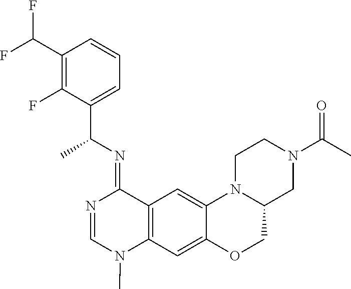 [SMILES: CC(=O)N1CCN2c3cc4/c(=N/[C@H](C)c5cccc(C(F)F)c5F)ncn(C)c4cc3OC[C@H]2C1] | 92 |
| 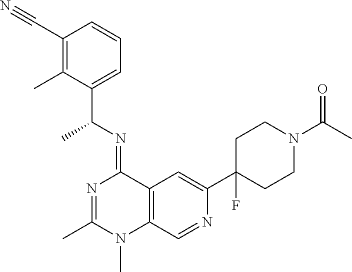 [SMILES: CC(=O)N1CCC(F)(c2cc3/c(=N/[C@H](C)c4cccc(C#N)c4C)nc(C)n(C)c3cn2)CC1] | 93 |
| 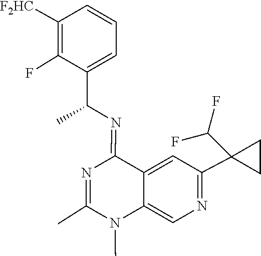 [SMILES: Cc1n/c(=N\[C@H](C)c2cccc(C(F)F)c2F)c2cc(C3(C(F)F)CC3)ncc2n1C] | 94 |
| 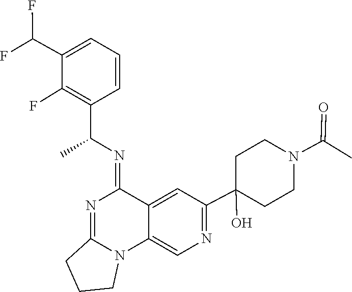 [SMILES: CC(=O)N1CCC(O)(c2cc3/c(=N/[C@H](C)c4cccc(C(F)F)c4F)nc4n(c3cn2)CCC4)CC1] | 95 |
| 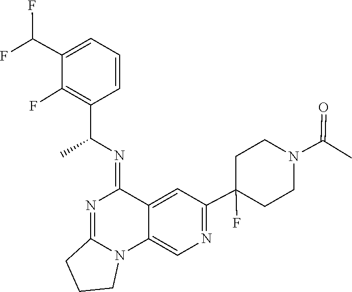 [SMILES: CC(=O)N1CCC(F)(c2cc3/c(=N/[C@H](C)c4cccc(C(F)F)c4F)nc4n(c3cn2)CCC4)CC1] | 96 |
| 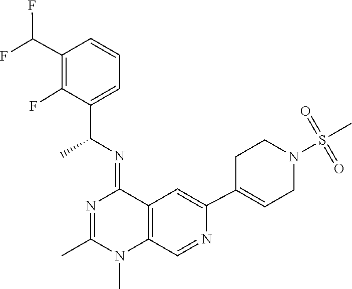 [SMILES: Cc1n/c(=N\[C@H](C)c2cccc(C(F)F)c2F)c2cc(C3=CCN(S(C)(=O)=O)CC3)ncc2n1C] | 97 |
| 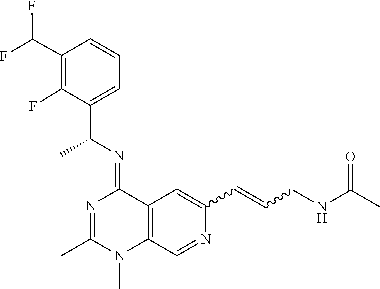 [SMILES: CC(=O)NCC=Cc1cc2/c(=N/[C@H](C)c3cccc(C(F)F)c3F)nc(C)n(C)c2cn1] | 98 |
| 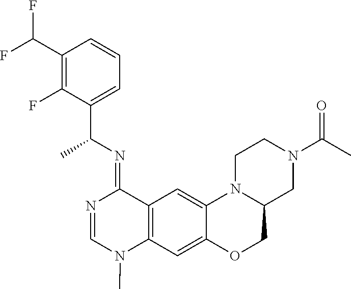 [SMILES: CC(=O)N1CCN2c3cc4/c(=N/[C@H](C)c5cccc(C(F)F)c5F)ncn(C)c4cc3OC[C@@H]2C1] | 99 |
| 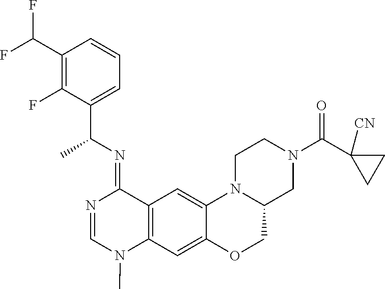 [SMILES: C[C@@H](/N=c1\ncn(C)c2cc3c(cc12)N1CCN(C(=O)C2(C#N)CC2)C[C@@H]1CO3)c1cccc(C(F)F)c1F] | 100 |
| 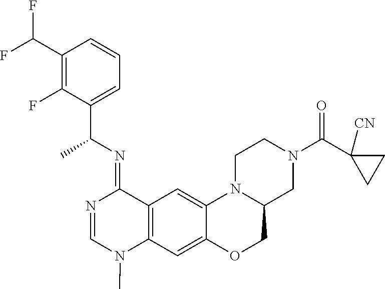 [SMILES: C[C@@H](/N=c1\ncn(C)c2cc3c(cc12)N1CCN(C(=O)C2(C#N)CC2)C[C@H]1CO3)c1cccc(C(F)F)c1F] | 101 |
| 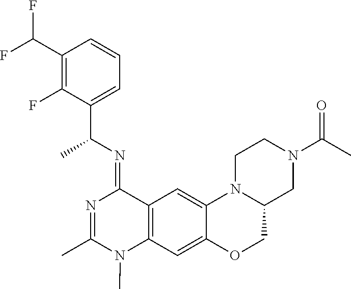 [SMILES: CC(=O)N1CCN2c3cc4/c(=N/[C@H](C)c5cccc(C(F)F)c5F)nc(C)n(C)c4cc3OC[C@H]2C1] | 102 |
| 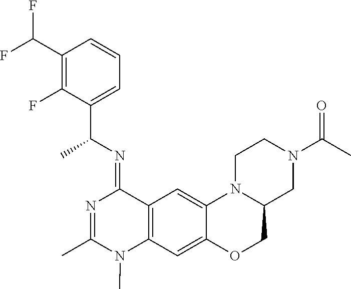 [SMILES: CC(=O)N1CCN2c3cc4/c(=N/[C@H](C)c5cccc(C(F)F)c5F)nc(C)n(C)c4cc3OC[C@@H]2C1] | 103 |
| 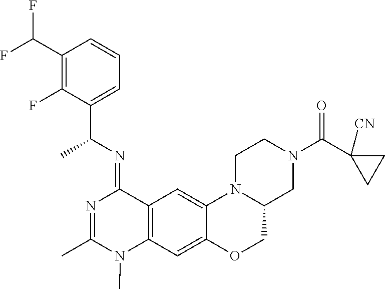 [SMILES: Cc1n/c(=N\[C@H](C)c2cccc(C(F)F)c2F)c2cc3c(cc2n1C)OC[C@H]1CN(C(=O)C2(C#N)CC2)CCN31] | 104 |
| 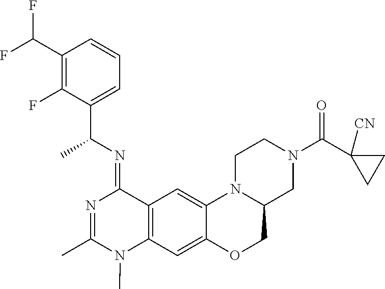 [SMILES: Cc1n/c(=N\[C@H](C)c2cccc(C(F)F)c2F)c2cc3c(cc2n1C)OC[C@@H]1CN(C(=O)C2(C#N)CC2)CCN31] | 105 |
| 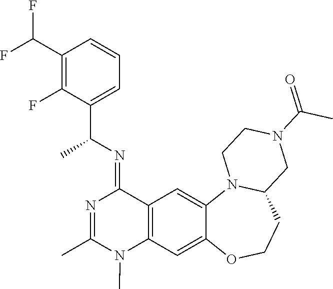 [SMILES: CC(=O)N1CCN2c3cc4/c(=N/[C@H](C)c5cccc(C(F)F)c5F)nc(C)n(C)c4cc3OCC[C@H]2C1] | 106 |
| 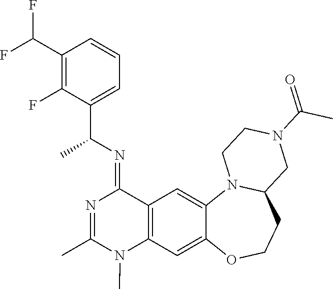 [SMILES: CC(=O)N1CCN2c3cc4/c(=N/[C@H](C)c5cccc(C(F)F)c5F)nc(C)n(C)c4cc3OCC[C@@H]2C1] | 107 |
| 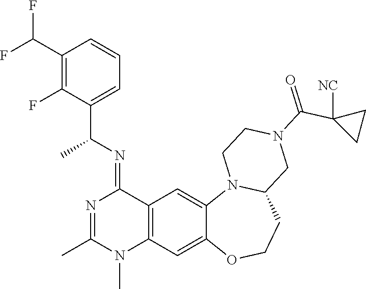 [SMILES: Cc1n/c(=N\[C@H](C)c2cccc(C(F)F)c2F)c2cc3c(cc2n1C)OCC[C@H]1CN(C(=O)C2(C#N)CC2)CCN31] | 108 |
| 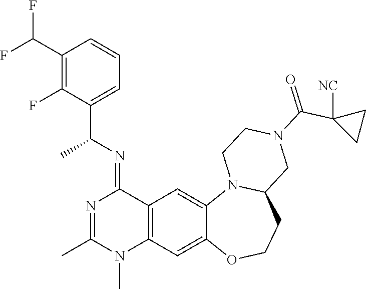 [SMILES: Cc1n/c(=N\[C@H](C)c2cccc(C(F)F)c2F)c2cc3c(cc2n1C)OCC[C@@H]1CN(C(=O)C2(C#N)CC2)CCN31] | 109 |
| 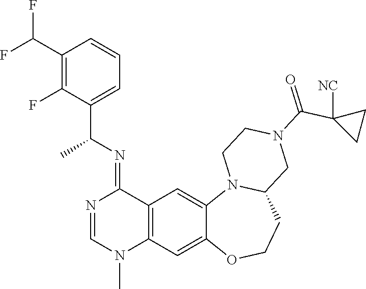 [SMILES: C[C@@H](/N=c1\ncn(C)c2cc3c(cc12)N1CCN(C(=O)C2(C#N)CC2)C[C@@H]1CCO3)c1cccc(C(F)F)c1F] | 110 |
| 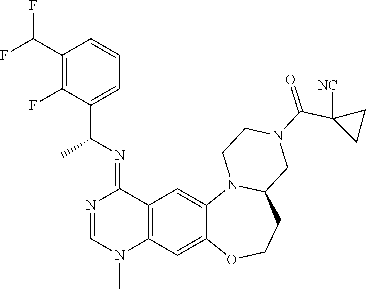 [SMILES: C[C@@H](/N=c1\ncn(C)c2cc3c(cc12)N1CCN(C(=O)C2(C#N)CC2)C[C@H]1CCO3)c1cccc(C(F)F)c1F] | 111 |
| 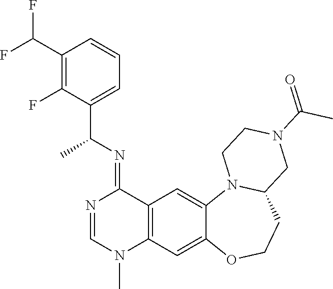 [SMILES: CC(=O)N1CCN2c3cc4/c(=N/[C@H](C)c5cccc(C(F)F)c5F)ncn(C)c4cc3OCC[C@H]2C1] | 112 |
| 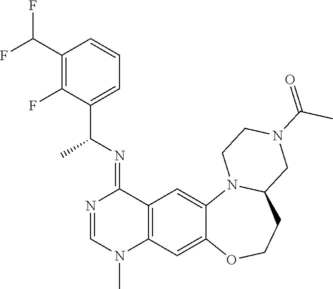 [SMILES: CC(=O)N1CCN2c3cc4/c(=N/[C@H](C)c5cccc(C(F)F)c5F)ncn(C)c4cc3OCC[C@@H]2C1] | 113 |

Preferred compounds include Compounds: 1, 4, 6, 8, 13, 15, 18, 27, 29, 30, 31, 33, 35, 45, 55, 82, 84, and 92-97. More preferably, the compound is one of Compounds 1, 6, 8, 13, 18, 27, 29, 30, 31, and 33, 92, 93 and 96.

The term “halogen” herein refers to a fluoro, chloro, bromo, or iodo radical. A particular halogen is a fluoro radical.

The term “alkyl” refers to a straight or branched hydrocarbon group, containing 1-20 carbon atoms (e.g., C₁₋₁₀, C₁₋₆, C₁₋₄, and C₁₋₃) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl. The term “alkylene” refers to a straight or branched hydrocarbon group, containing 1-20 carbon atoms (e.g., C₁₋₁₀, C₁₋₆, C₁₋₄, and C₁₋₃) and two monovalent radical centers derived by the removal of two hydrogen atoms from the same or two different carbon atoms of a parent alkane. The term “haloalkylene” refers to alkylene substituted with one or more halogens (fluoro, chloro, bromo, or iodo).

The term “haloalkyl” refers to alkyl substituted with one or more halogens (fluoro, chloro, bromo, or iodo). Examples include fluoromethyl, difluoromethyl, trifluoromethyl, fluoroethyl (e.g., 1-fluoroetyl and 2-fluoroethyl), difluoroethyl (e.g., 1,1-, 1,2-, and 2,2-difluoroethyl), and trifluoroethyl (e.g., 2,2,2-trifluoroethyl).

The term “alkoxy” refers to an —O-alkyl group. Examples are methoxy, ethoxy, propoxy, and isopropoxy. Alkoxy also includes haloalkoxy, namely, alkoxy substituted with one or more halogens, e.g., —O—CH₂Cl and —O—CHClCH₂Cl.

The term “alkylamino” refers to —NR′-alkyl, in which R′ is H, C₁-C₁₀ alkyl, C₃-C₁₀ cycloalkyl, C₃-C₁₀ heterocycloalkyl, aryl, or heteroaryl.

The term “alkylamido” refers to —NR′—C(O)-alkyl, in which R′ is H, C₁-C₁₀ alkyl, C₃-C₁₀ cycloalkyl, C₃-C₁₀ heterocycloalkyl, aryl, or heteroaryl.

The term “alkylcarbonyl” refers to —C(O)-alkyl.

The term “alkylsulfonyl” refers to —S(O)₂-alkyl.

The term “alkysulfonamido” refers to —NR′—S(O)₂-alkyl, in which R′ is H, C₁-C₁₀ alkyl, C₃-C₁₀ cycloalkyl, C₃-C₁₀ heterocycloalkyl, aryl, or heteroaryl.

The term “cycloalkyl” refers to a nonaromatic, saturated or unsaturated monocyclic, bicyclic, tricyclic, or tetracyclic hydrocarbon group containing 3 to 12 carbons (e.g., C₃₋₁₀, C₃₋₈, C₄₋₇, and C₃₋₆). Cycloalkyl also includes fused, bridged, and spiro ring systems. Examples are cyclopropyl, cyclobutyl, cyclopentyl, cyclopentenyl, cyclohexyl, cyclohexenyl, cycloheptyl, cyclooctyl, bicyclo[1.1.1]pentyl, bicyclo[2.1.1]hexyl, bicyclo[2.1.1]heptyl, bicyclo[2.2.2]octanyl, and decahydronaphthalene.

A bicyclic hydrocarbon group or ring system includes a fused bicyclic ring, a bridged bicyclic ring, or a spiro bicyclic ring. A fused bicyclic ring has two rings (e.g., hydrocarbon or heterohydrocarbon) sharing two common atoms (i.e., bridgehead atoms) that are connected to each other. In a bridged bicyclic ring, the two bridgehead atoms are not connected directly but separated by at least one atom. A spiro bicyclic ring refers to a ring system having two rings sharing only one common atom.

The term “cycloalkylamido” refers to —NR′-cycloalkyl, in which R′ is H, C₁-C₁₀ alkyl, C₃-C₁₀ cycloalkyl, C₃-C₁₀ heterocycloalkyl, aryl, or heteroaryl.

The term “heterocycloalkyl” refers to a nonaromatic, saturated or unsaturated, 3-8 membered monocyclic, 8-12 membered bicyclic, or 11-15 membered tricyclic ring system having one or more heteroatoms (e.g., O, N, P, and S). The term also includes fused, bridged, and spiro ring systems. Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, 2-azaspiro[3.3]heptanyl, quinuclidinyl, pyridinyl-2(1H)-one, 3-azabicyclo[3.1.0]hexanyl, 3,6-dihydro-2H-pyranyl, 5-azaspiro[2.4]heptanyl, 3,6-dihydro-2H-pyran 6-azaspiro[2.5]octanyl, 5,6,7,8-tetrahydroimidazo[1,2-a]pyrazinyl, 1,2,3,6-tetrahydropyridinyl, 1,2,3,4,4a,5-hexahydrobenzo[b]pyrazino[1,2-d][1,4]oxazinyl, 2,3,4,4a,5,6-hexahydro-1H-benzo[b]pyrazino[1,2-d][1,4]oxazepinyl and 8-azabicyclo[3.2.1]octanyl. The term “heterocycloalkoxy” refers to an —O-heterocycloalkyl group.

The term “heterocyclyl” refers to heterocycloalkyl, heteroaryl, benzene fused heterocycloalkyl or heteroaryl, or cycloalkyl fused heterocycloalkyl or heteroaryl. Heterocyclyl includes 3-8 membered monocyclic, 8-12 membered bicyclic, or 11-20 (e.g., 11-15) membered tricyclic ring system.

The term “alkenyl” refers to a straight or branched, monovalent, unsaturated aliphatic chain having 2 to 20 carbon atoms (e.g., C₂₋₄, C₂₋₆, and C₂₋₁₀) and one or more carbon-carbon double bonds. Examples are ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, and 2-pentenyl. The term “alkenylene” refers to a straight or branched, bivalent, unsaturated aliphatic chain having 2 to 20 carbon atoms (e.g., C₂₋₄, C₂₋₆, and C₂₋₁₀) and one or more carbon-carbon double bonds.

The term “alkynyl” refers to a straight or branched aliphatic chain having 2 to 20 carbon atoms (e.g., C₂₋₄, C₂₋₆, and C₂₋₁₀) and one or more carbon-carbon triple bonds. Examples are ethynyl, 2-propynyl, 2-butynyl, 3-methylbutynyl, and 1-pentynyl. The term “alkynylene” refers to a straight or branched, bivalent, unsaturated aliphatic chain having 2 to 20 carbon atoms (e.g., C₂₋₄, C₂₋₆, and C₂₋₁₀) and one or more carbon-carbon triple bonds.

The term “aryl” refers a 6-carbon monocyclic, 10-carbon bicyclic, 14-carbon tricyclic aromatic ring system wherein each ring can have one or more (e.g., 1 to 10, 1 to 5, and 1 to 3) substituents. Examples include phenyl, biphenyl, 1- or 2-naphthyl, 1,2-dihydronaphthyl, 1,2,3,4-tetrahydronaphthyl, indenyl, and indanyl.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, quinolinyl, indolyl, thiazolyl, and benzothiazolyl.

Alkyl, alkylene, haloalkylene, alkoxyl, cycloalkyl, heterocycloalkyl, heterocyclyl, heterocycloalkoxy, alkenyl, alkynyl, aryl, and heteroaryl mentioned herein include both substituted and unsubstituted moieties. Examples of a substituent include deuterium, halogen (e.g., F, Cl, and Br), amino, hydroxy, alkyl and haloalkyl (e.g., methyl, fluoromethyl, difluoromethyl, trifluoromethyl, difluoroethyl, and 1,1-difluoro-2-hydroxylethan-1-yl), alkenyl and haloalkenyl (e.g., ethylenyl and 3,3-difluoro-2-methylpropen-3-yl), cycloalkyl (e.g., cyclopropyl and cyclobutyl), cycloheteroalkyl (e.g., tetrahydrofuranyl), —CN, —CONR₇R₈, —NR₇R₈, —NR₇COR₈, —NR₇SO₂R₈, —N₇COOR₈, —COR₇, —COOR₇, —SR₇, —SONR₇R₈, and —OR₉, wherein R₇, R₈, and R₉ are on each occurrence independently selected from the group consisting of hydrogens, C₁₋₆ alkyl, C₂₋₄ alkenyl, and C₂₋₄ alkynyl optionally substituted with 1-3 halogens. All substituents can be further substituted.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

The depiction of an asterisk (*) in a chemical formula represents the point of attachment of the group to the corresponding parent formula.

The present invention also features use of one or more of the above-described compounds for treating cancer or for the manufacture of a medicament for treating cancer.

The term “treating” or “treatment” refers to administering one or more of the compounds to a subject with the purpose to confer a therapeutic effect, e.g., to slow, interrupt, arrest, control, or stop of the progression of an existing disorder and/or symptoms thereof, but does not necessarily indicate a total elimination of all symptoms. “An effective amount” refers to the amount of a compound that is required to confer the therapeutic effect. Effective doses will vary, as recognized by those skilled in the art, depending on the types of symptoms treated, route of administration, excipient usage, and the possibility of co-usage with other therapeutic treatment.

The cancer is caused by KRAS mutation, SOS1 oncogenic mutation, or oncogenic mutation/overexpression of receptor tyrosine kinases such as EGFR, FGFR, etc. and selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcoma. Preferably, the cancer is pancreatic, non-small cell lung cancer, cholangiocarcinoma, or colorectal cancer.

The term “subject” refers to an animal such as a mammal including a human. A human is a preferred subject.

A compound of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition., Academic Press, 2020).

This invention further features treating diseases by inhibiting SOS1 activity, defined as a RASopathy. The disease is selected from the group consisting of Neurofibromatosis type 1 (NF1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome), and Hereditary gingival fibromatosis.

To practice the method of the present invention, a composition or a kit containing one or more of the above-described compounds can be administered alone or co-administered with at least one other pharmacologically active substance simultaneously, concurrently, sequentially, successively, alternately, or separately. Simultaneous administration, also referring to as concomitant administration, includes administration at substantially the same time. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g., by oral, enteral, parenteral, nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

The term “parenteral” as used herein refers to subcutaneous, intracutaneous, intravenous, intraperitoneal, intramuscular, intraarticular, intraarterial, intrasynovial, intrasternal, intrathecal, intralesional, or intracranial injection, as well as any suitable infusion technique.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents.

A composition having one or more of the above-described compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound.

Examples include colloidal silicon oxide, magnesium stearate, cellulose, sodium lauryl sulfate, and D&C Yellow #10.

The details of one or more embodiments of the invention are set forth in the description below. Other features, objects, and advantages of the invention will be apparent from the description and from the claims.

### DETAILED DESCRIPTION

The present invention is based on a surprising discovery that the compounds of formula I are effective in inhibiting SOS1 activity and treating cancer.

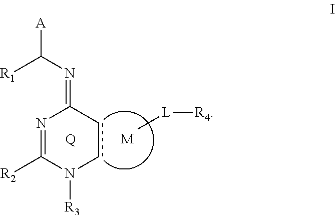

Variables R₁-R₄, A, L, ring Q, and ring M are defined above. Described in detail below are compounds of formula I, as well as their syntheses and their use in treating cancer and/or inhibiting SOS1.

Subsets of the compounds of Formula I include compounds of formulas II, II-A, II-B, II-C, II-D, II-E, II-F, II-G, II-H, III, IV, V, and VI. See supra.

In these subsets, ring M is a phenyl or a five-fifteen membered heteroaryl or heterocycloalkyl ring. The number of the double bonds in ring M is 1, 2 or 3.

The compounds of formula I include all cis-trans isomers, enantiomers, diastereomers, and mixtures thereof in any ratio.

The compounds of any of the above formulas can be prepared by synthetic methods well known in the art. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

The compounds thus prepared can be purified following conventional methods such as crystallization, distillation/vacuum distillation, flash chromatography over silica, and preparative liquid chromatography.

Efficacy of the compounds of this invention can be initially determined using in vitro homogeneous time-resolved fluorescence (HTRF) based protein-protein interaction assay, pERK potency assay, or 3D cell proliferation assay, all described below. The selected compounds can be further tested to verify their efficacy, e.g., by administering it to an animal. Based on the results, an appropriate dosage range and administration route can be determined.

A compound of this invention is preferably formulated into a pharmaceutical composition containing a pharmaceutical carrier. The pharmaceutical composition is then given to a subject in need thereof to inhibit SOS thus treating cancer.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

Set forth below are examples illustrating preparation and efficacy evaluation of compounds of this invention.

The abbreviations as used herein are provided in Table 2 below with their definitions, which are used conventionally in the art.

TABLE 2

| Abbr. | Name | Abbr. | Name |
|---|---|---|---|
| Ac | acetyl | AcCl | acetyl chloride |
| AcOH | acetic acid | ACN | acetonitrile |
| Ac₂O | acetic anhydride | BINAP | 2,2′-bis(diphenylphosphino)- |
| | | | 1,1′-binaphthyl |
| n-Bu | n-butyl | t-Bu | t-butyl |
| t-BuOH | t-butyl alcohol | t-BuOK | potassium t-butoxide |
| t-BuONa | Sodium t-butoxide | CH(OEt)₃ | triethyl orthoformate |
| DAST | diethylaminosulfur | DBU | 1,8-Diazabicyclo[5.4.0]undec-7- |
| | trifluoride | | ene |
| DCE | 1,2-dichloroethane | DCM | dichloromethane |
| DEA | diethylamine | DIBAL-H | Diisobutylaluminium hydride |
| DIEA | N,N-diisopropylethylamine | DIPEA | N,N-diisopropylethylamine |
| DMA | Dimethylacetamide | Dmap | 4-(dimethylamino)-pyridine |
| DMF | dimethylformamide | DMSO | dimethyl sulfoxide |
| EDCI or | 1-Ethyl-3-(3- | Et | ethyl |
| EDC | dimethylamino- | | |
| | propyl)carbodiimide | | |
| EtOAc, EA | ethyl acetate | EtOH | ethanol |
| GSD | geometric standard | HATU | 1[bis(dimethylamino)methylene]- |
| | deviation | | 1H-1,2,3-triazolo[4,5-b] |
| | | | pyridinium 3-oxide |
| | | | hexafluorophosphate |
| HCCP | phosphonitrilic chloride | HOBt | Hydroxybenzotriazole |
| | trimer | | |
| HPLC | High-Performance Liquid | IPA or | isopropyl alcohol |
| | Chromatography | i-PrOH | |
| KOCN | Potassium cyanate | LCMS | Liquid Chromatography-Mass |
| | | | Spectrometry |
| LDA | lithium diisopropylamide | LiHMDS or | lithium hexamethyldisilylamide |
| | | LHMDS | |
| m-CPBA | meta-chloroperoxybenzoic | Me | methyl |
| | acid | | |
| MeCN | acetonitrile | MeI | iodomethane |
| MeOH | methanol | Mn(TMHD)₃ | Tris(2,2,6,6-tetramethyl-3,5- |
| | | | heptanedionato)manganese(III) |
| MS | mass spectrometry | MsCl | methanesulfonyl chloride |
| MTBE | methyl tert-butyl ether | NBS | N-bromosuccinimide |
| NMR | nuclear magnetic resonance | Pd₂(dba)₃ | tris(dibenzylideneacetone) |
| | | | dipalladium (0) |
| Pd(dppf)Cl₂ | [1,1′-bis(diphenylphos- | Pd(OAc)₂ | palladium (II) acetate |
| | phino)ferrocene]dichloropal | | |
| | -ladium (II) | | |
| PE | petroleum ether | Ph | phenyl |
| PhSiH₃ | phenylsilane | Q-Phos | pentaphenyl(di-tert- |
| | | | butylphosphino)ferrocene |
| RuPhos Pd | (2-dicyclohexylphosphino- | TBS | tris buffered saline |
| G3 | 2′,6′-diisopropoxy-1,1′- | | |
| | biphenyl)[2-(2′-amino-1,1′- | | |
| | biphenyl)]palladium(II) | | |
| | methanesulfonate | | |
| TEA/Et₃N | triethylamine | TFA | trifluoroacetic acid |
| TFAA | Trifluoroacetic anhydride | THF | tetrahydrofuran |
| TLC | thin layer chromatography | TMSA | (trimethylsilyl)acetylene |
| TsOH | p-toluenesulfonic acid | X-PHOS | 2-dicyclohexylphosphino- |
| | | | 2′,4′,6′-triisopropylbiphenyl |
| PMBNH₂ | 4-Methoxylbenzylamine | | |

### General Synthetic Procedures

Provided below are exemplary processes for preparing compounds of formula I including compounds of formulas II-A, II-B, II-C, II-D, II-E, II-F, II-Gi, II-H, III, IV, V or VI. These processes for preparing these compounds and useful intermediates provide further features of the invention and are illustrated in the following procedures.

Each compound illustrated below was obtained as a free compound (e.g., a free base), a salt (e.g., a formate and a trifluoroacetate), or any combination thereof.

Generally, compounds of formulas II-A to II-D and VI can be synthesized using the procedures illustrated in Schemes I to III, and Scheme IX to X.

Compounds of formulas II-E and II-F can be synthesized using the procedure illustrated in Schemes VII and VI respectively.

Compounds of formulas II-G and II-H can be synthesized using the procedure illustrated in Schemes IV and V respectively.

Compounds of formulas IV and V can be synthesized using the procedure illustrated in Schemes XII and XI respectively.

Compounds of formulas VI can be synthesized using the procedure illustrated in Scheme VIII.

As shown in Scheme I, amide I-2 is prepared by reacting acid I-1 with ammonium hydroxide in the presence of amide coupling reagents, such as HOBt/EDCI or HATU. The fluorine atom is displaced by R₃NH₂, followed by ring closure with dehydrating agents, such as acyl chloride R₂COCl, (or triethyl orthoformate when R₂ is H) to yield fused bicyclic intermediate I-4. Alternatively, the cyclization is accomplished by treating the resulting acylated I-3 with NaOMe. Subsequently, I-4 is converted to a thione I-5 using Lawesson's reagent or P₂S₅. The resulting thione reacts with methyl iodide to generate salt I-6 having a methylthio group, which then reacts with an amine I-7 (prepared according to procedure described in WO2019/201848, WO2021/074227, WO2022/058344, WO201922129, WO2021/092115, WO2020/180768) to produce intermediate I-8. Finally, R₄ is introduced to yield the final product of formula I-9, using a method as exemplified in Scheme II, including, but not limited to, Suzuki, Buchwald-Hartwig, Stille, and Negishi coupling reactions. As showing in Scheme II, Ar represents the heteroaryl ring system of I-9.

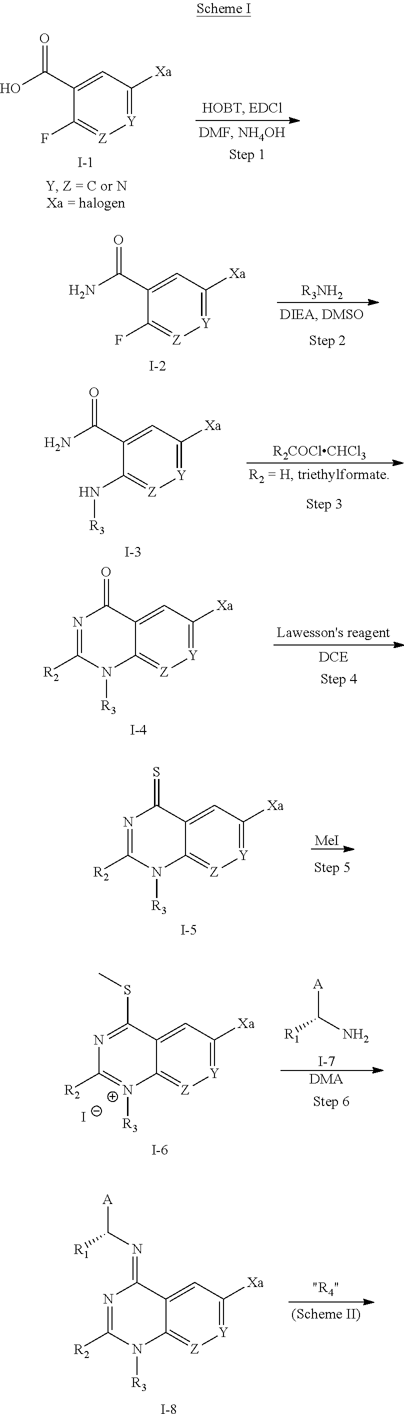

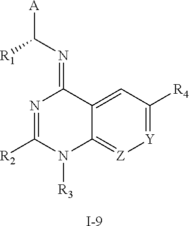

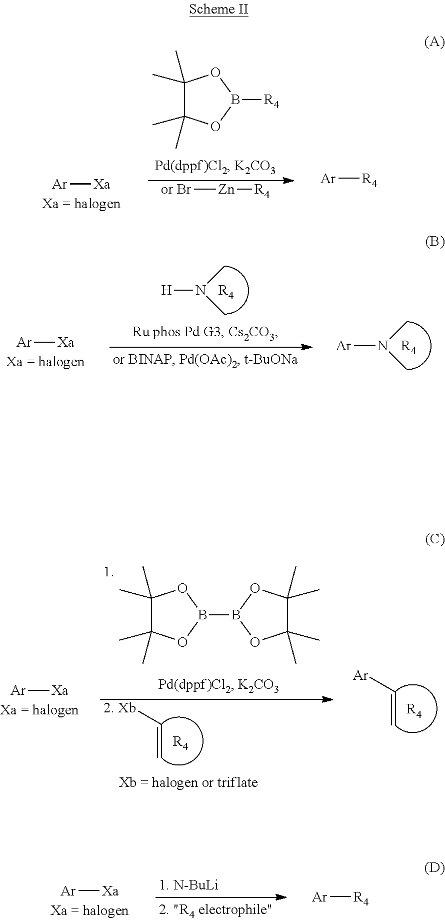

Alternatively, when R₂ is H and R₃ is methyl, compounds of formula III-2 can be prepared from III-1 obtained according to the methods described in WO2019/201848, WO2021/074227, WO2022/058344, WO201922129, WO2021/092115, WO2020/180768 and CN114539245. As illustrated in Scheme III below, treating intermediate III-1 with methyl iodide and Cs₂CO₃ or trimethyloxonium tetrafluoroborate yields product of formula III-2.

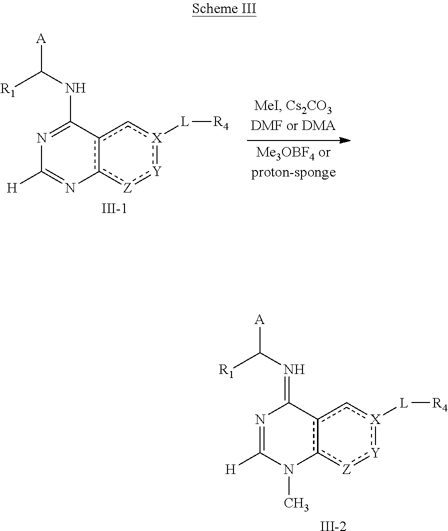

For compounds of formula II-G in which R₂ and R₃ are linked together via a linker, the synthesis is depicted in Scheme IV. Firstly, pyridine derivative IV-1 is brominated using NBS to yield IV-2. The amination of ester IV-2 yields amide IV-3. The amino group on the pyridine ring of IV-3 is acylated to obtain IV-4, which is treated with a base (e.g., t-BuOK) to afford IV-5. Subsequently, IV-5 reacts with Lawesson's reagent and the resulting thione IV-6 is treated with MeI to afford iodide salt IV-7. Coupling of IV-7 with amine I-7 yields imine IV-8. Introducing “R₄” substituent yields compound IV-9 as exemplary compounds of formula II-G.

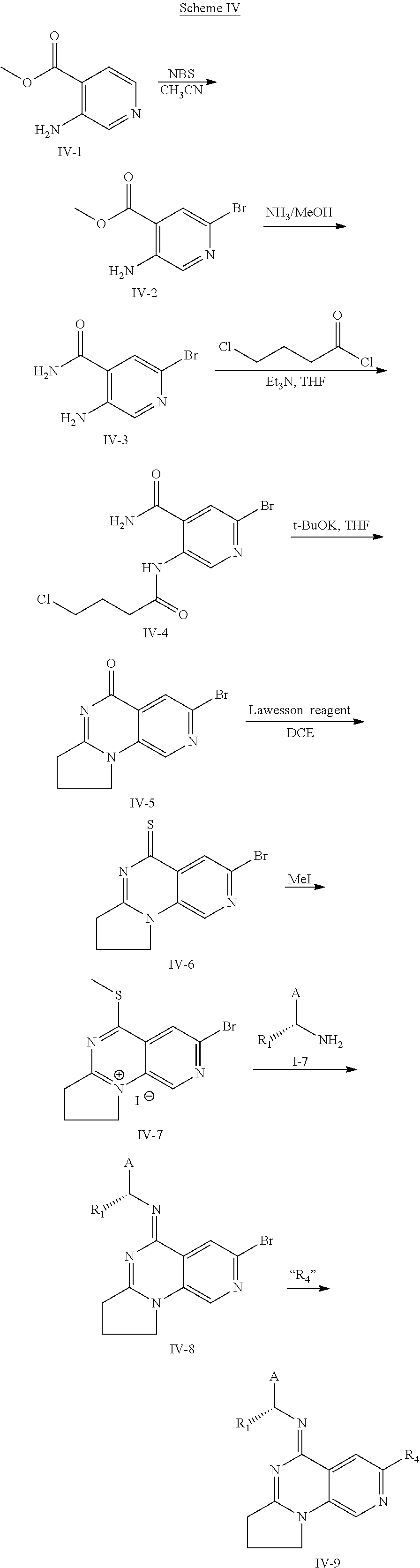

Scheme V below illustrates the syntheses of compounds of formula II-H. Pyridine derivative V-1 reacts with CS₂ in the presence of DBU, followed by S-ethylation with ethyl iodide gives ethyl thioether V-2. Chloride V-3 is obtained by chlorinating intermediate V-2 using thionyl chloride. Intermediate V-3 is coupled with amine I-7 to afford V-4, followed by oxidation with m-CPBA to yield sulfone V-5. Displacement of the resulting sulfone with 2-((tert-butyldimethylsilyl)oxy)-ethan-1-ol in the presence of NaH followed by in-situ silyl deprotection yields alcohol V-6, which is subsequently converted to chloride V-7 with thionyl chloride. V-7 is converted to the tricyclic ring V-8 by using a base such as K₂CO₃. Finally, introduction of R₄ into V-8 produces compound V-9 as exemplary compounds of formula II-H.

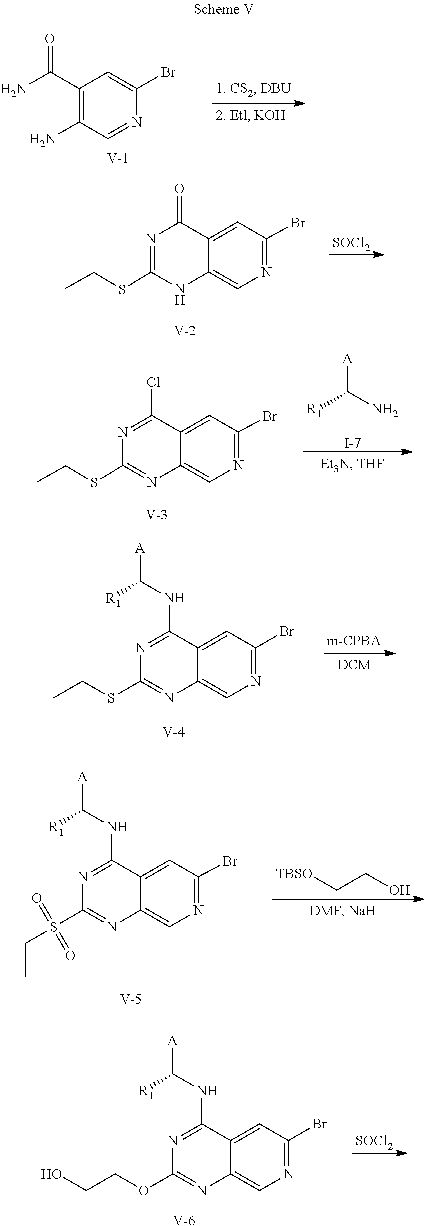

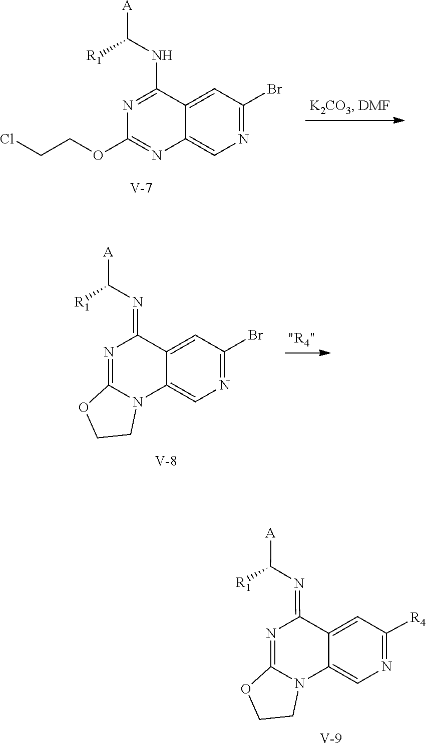

Compounds of formula II-F are prepared following Scheme VI, pyrido[3,4-d]pyrimidine derivative VI-1 is coupled with amine I-7 (R₁(CH)ANH₂) to yield VI-2. The methoxy ether in VI-2 is hydrolyzed with HBr in a suitable solvent (e.g., acetic acid) to yield VI-3. VI-3 is treated with alkyl halide RyI (e.g., methyl iodide) in the presence of a base (e.g., Cs₂CO₃) to give intermediate VI-4. R₄ is introduced to yield intermediate VI-5, using a method exemplified in Scheme II, including, but not limited to, Suzuki, Buchwald-Hartwig, Stille, and Negishi coupling reactions. Treating intermediate VI-5 with alkyl halide (e.g., methyl iodide) in the presence of a base (e.g., Cs₂CO₃) in a suitable solvent (e.g, DMF) yield products of formula VI-6.

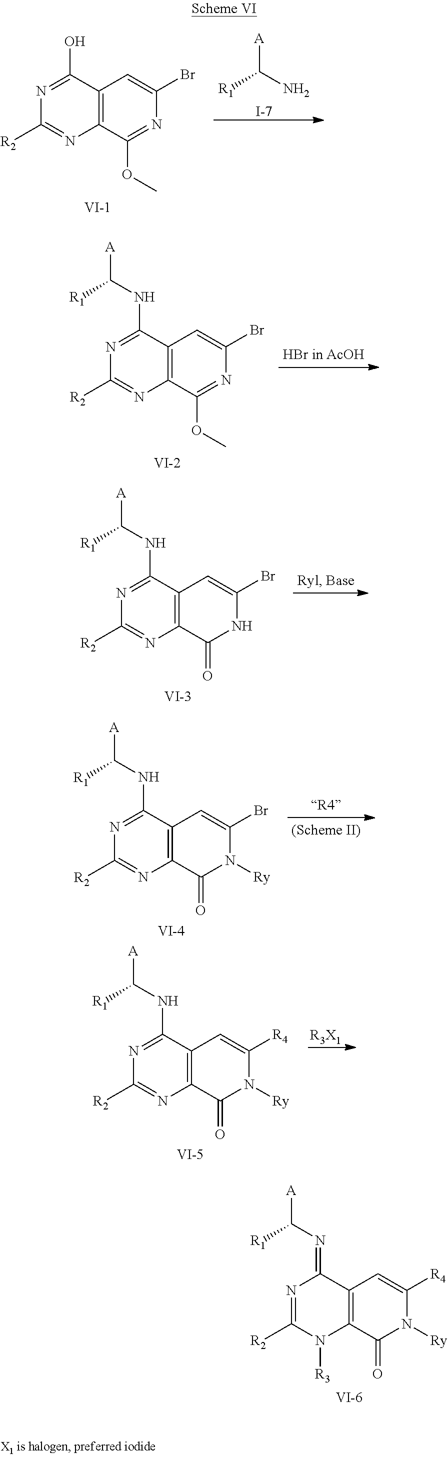

Compounds of formula II-E are prepared following Scheme VII. 4-Hydroxy-pyrimidine derivative VII-1 is converted to triflate VII-2 using Tf₂O in the presence of a base (e.g., pyridine) in a suitable solvent (e.g., DCM). Triflate VII-2 undergoes displacement with amine I-7 in the presence of a base (e.g., DIEA) and a suitable solvent (e.g., dioxane) to yield VII-3. R₄ group in VII-3 is introduced following scheme II via, e.g., Suzuki, Buchwald-Hartwig, Stille, and Negishi coupling reactions. Treating intermediate VII-3 with alkyl halide (e.g., methyl iodide) in a suitable solvent (e.g., DMF or DMA) yields the product of formula VII-4.

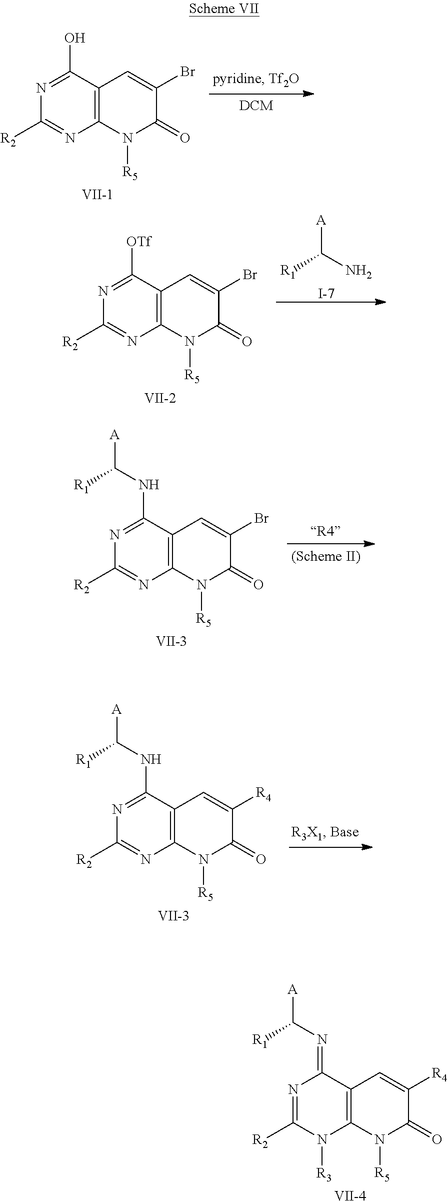

Compounds of formula II-C can also be prepared following Scheme VIII, 2-amino-4,5-dimethoxybenzoic acid VIII-1 is treated with triphosgene to yield VIII-2. VIII-2 is alkylated with alkyl halide (e.g., methyl iodide) in the presence of base (e.g., NaH) in a suitable solvent (e.g., DMF) to yield VIII-3. VIII-3 on treatment with ammonium hydroxide in a suitable solvent (e.g., THF) yields VIII-4. VIII-4 is converted to pyrimidone derivative VIII-5 on treatment with R₂CH(OEt)₃ in the presence of suitable acid (e.g., acetic acid) in a solvent (e.g., ethanol). VIII-5 is selectively demethylated using L-methionine and methanesulfonic acid to yield VIII-6. The hydroxyl group in VIII-6 is alkylated with alkyl tosylate (e.g., (3R)— tetrahydrofuran-3-yl 4-methylbenzenesulfonate) in the presence of base (e.g., potassium carbonate) in a suitable solvent (e.g., DMF) to yield ether VIII-7. VIII-7 is converted to thione VIII-8, which reacts with methyl iodide to generate salt VIII-9. Salt VIII-9 reacts with an amine I-7 to yield the final product of formula VIII-10.

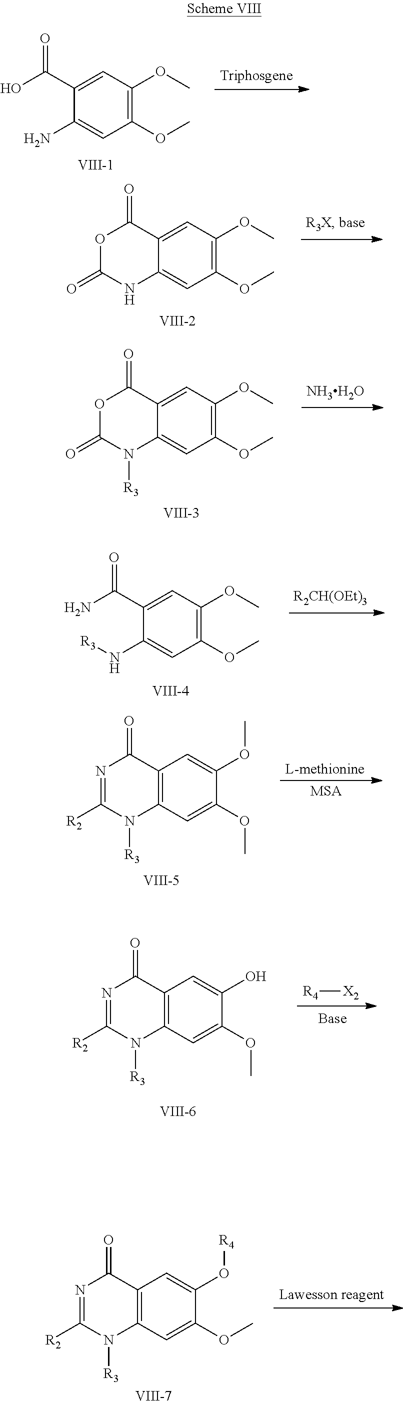

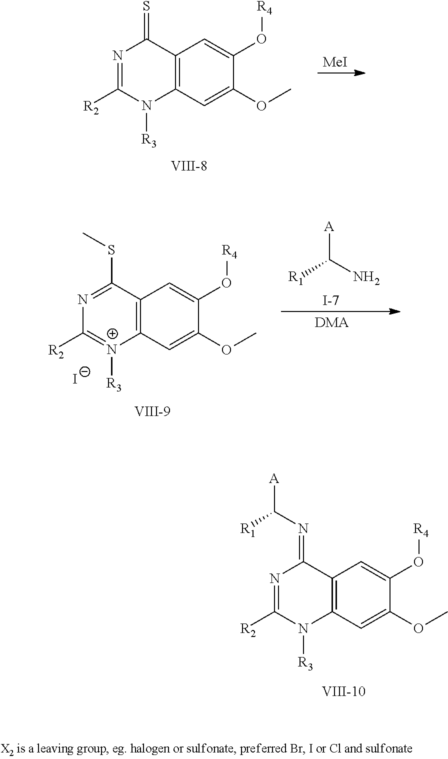

Compounds of formula II-C can also be prepared following Scheme IX, intermediate I-8 undergoes palladium (e.g., Pd(PPh₃)₂Cl₂) and CuI catalyzed coupling with alkyne derivatives in the presence of a base (e.g., TEA) in a suitable solvent (e.g., DMSO) to yield compounds of formula IX-1. Alternatively, intermediate I-8 can undergo palladium (Pd(dppf)Cl₂) catalyzed coupling with alkene boronic esters in the presence of a base (e.g., K₂CO₃) in a suitable solvent (e.g., dioxane and water) to yield compounds of formula IX-2.

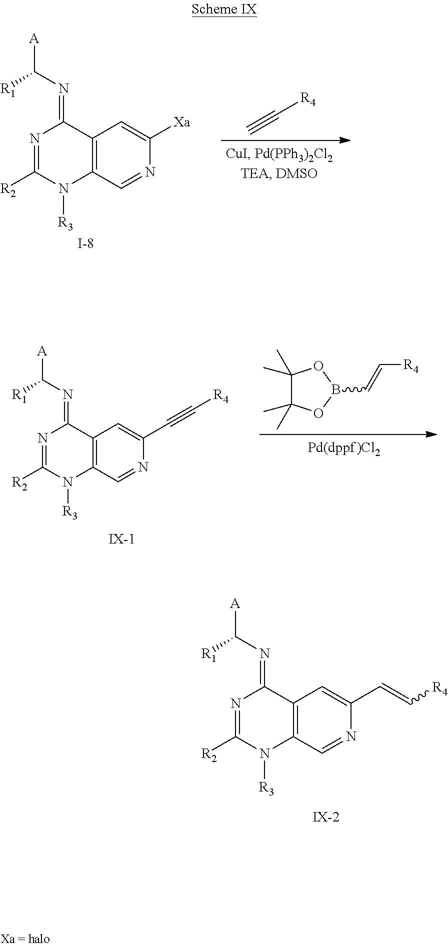

Compounds of formula II-B are prepared following Scheme X below. Intermediate I-8 is treated with an alkyl sulfonamide (e.g., propane-2-sulfonamide) in a CuI-catalyzed N-heteroarylation coupling reaction with a base (e.g., K₂CO₃) and a ligand (N,N′-dimethylethyl-enediamine) in a suitable solvent (e.g., DMF) to yield compounds of formula X-1. Alternatively, applying a nucleophilic displacement reaction, intermediate I-8 is converted to amide X-2 using palladium (e.g., Ruphos and Ruphos Pd G3) catalyzed coupling with an alkyl amide, such as cyclopentanecarboxamide, in the presence of a base (e.g., Cs₂CO₃). Further, intermediate I-8 is converted to amide X-3 under palladium catalyzed carbonylation in the presence of an amine, such as piperidines or piperazines.

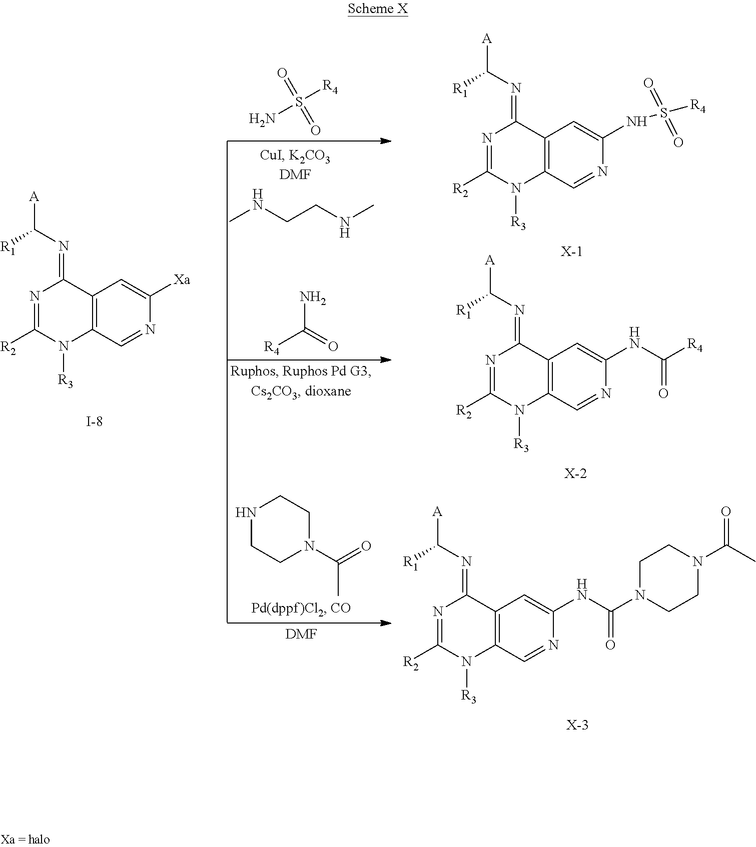

Compounds of formula V are prepared following Scheme XI. Chloride atom in tert-butyl 2,4-dichloro-5,7-dihydro-6H-pyrrolo[3,4-d]pyrimidine-6-carboxylate XI-1 is displaced with amine I-7 in the presence of a base (e.g., DIEA) in a suitable solvent (e.g., DMA) to give intermediate XI-2. Intermediate XI-2 undergoes palladium (Pd/C) catalyzed de-chlorination in the presence of a suitable base (e.g., TEA) in a suitable solvent (e.g., MeOH) to yield XI-3 (R₂=H). Alternatively, 2-chloro pyrimidine derivative XI-2 can be alkylated by palladium (e.g., Pd(dppf)Cl₂) catalyzed Suzuki coupling with trialkylboroxine (e.g., trimethylboroxine) in the presence of a base (e.g., K₂CO₃) in a suitable solvent (e.g., dioxane and water) to yield intermediate XI-3 (R₂=Me). Boc group in XI-3 is deprotected with a suitable acid (e.g., HCl in EtOAc) to yield XI-4. XI-4 is converted to amide XI-5 using carboxylic acid derivative R₄COOH (e.g., 4-methoxytetrahydro-2H-pyran-4-carboxylic acid) in the presence of a suitable coupling agent (e.g., T3P) and a base (e.g., DIEA) in a suitable solvent (e.g., MeCN) to yield amide derivative XI-5. Treating intermediate XI-5 with alkyl halide (e.g., methyl iodide) in a suitable solvent (e.g., DMF) yields the product of formula XI-6.

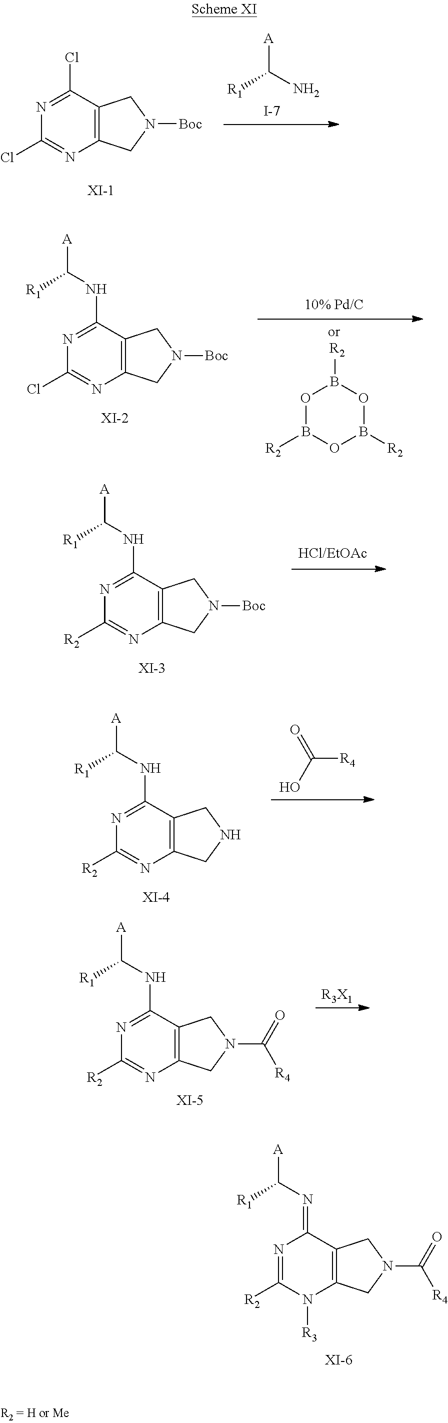

Compounds of formula IV are prepared following Scheme XII. For the synthesis of compounds of formula XII-2, the starting material XII-1 can be prepared using literature procedures (e.g., J. Med. Chem. 2022, 65 (23), 15856-15877 and 2022, 65 (19), 13158-13171; WO2023001229; WO2023001229 and WO2022135610). Compound XII-1 is treated with MeI in appropriate solvent, such as DMF, to produce compounds of formula XII-2.

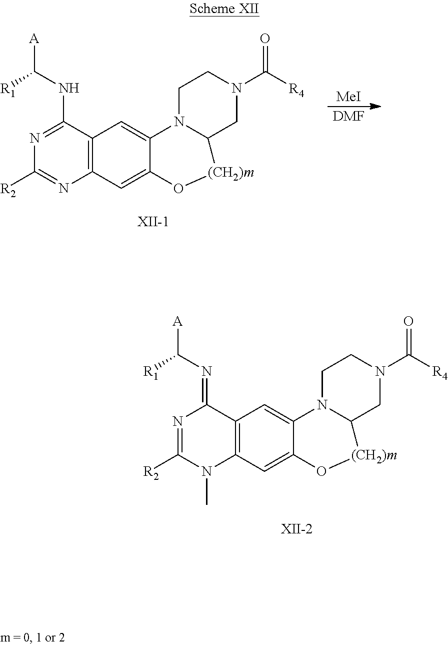

### EXAMPLES

### Synthesis of (R,Z)-6-bromo-1,2-dimethyl-N-(1-(2-methyl-3-(trifluoromethyl)phenyl)ethyl)-quinazolin-4(1H)-imine

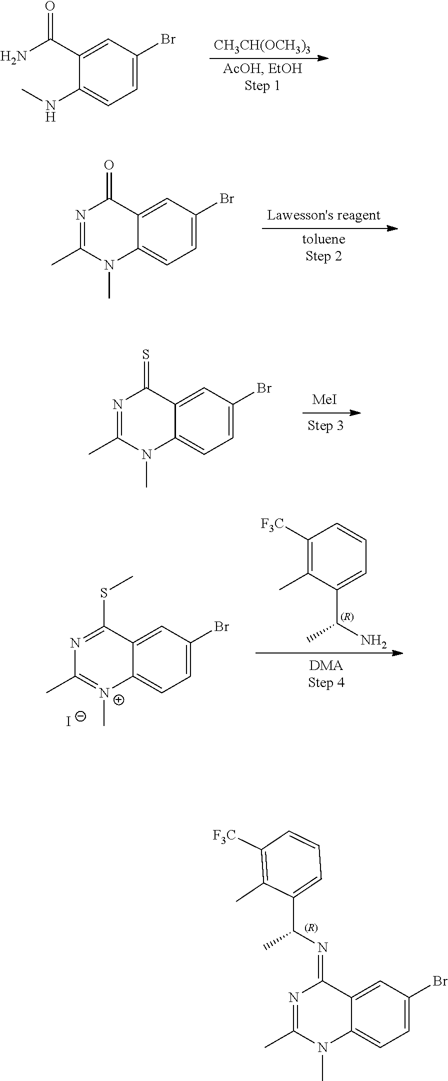

### Step 1. Synthesis of 6-bromo-1,2-dimethylquinazolin-4(1H)-one

To a solution of 5-bromo-2-(methylamino)benzamide (18 g, 78.6 mmol) in EtOH (300 mL) was added 1,1,1-triethoxyethane (216 mL, 1.18 mol) and AcOH (67 mL, 1.18 mol) at 25° C. The mixture was stirred at 120° C. for 12 hours, cooled to room temperature, and filtered. The filter cake was washed with EtOAc, 100 mL) and dried under vacuum to give the title compound (17 g, 67.2 mmol, 85.5% yield) as a grey solid.

LCMS m/e: 253, 255 (MH⁺).

### Step 2. Synthesis of 6-bromo-1,2-dimethylquinazoline-4(1H)-thione

To a solution of 6-bromo-1,2-dimethylquinazolin-4(1H)-one (5 g, 19.8 mmol) in toluene (40 mL) was added Lawesson's reagent (7.99 g, 19.8 mmol). The mixture was stirred at 120° C. for 1 hr. The reaction mixture was cooled to room temperature, poured into water (200 mL), and then extracted with DCM (100 mL×3). The combined organic layers were washed with brine (150 mL), dried over anhydrous Na₂SO₄, filtered, and concentrated to dryness. The residue was purified by flash column chromatography (silica gel, 0-10% MeOH in DCM) to give the title compound (3 g, 11.1 mmol, 56.4% yield) as a yellow solid.

LCMS m/e: 269, 271 (MH⁺).

### Step 3. Synthesis of 6-bromo-1,2-dimethyl-4-(methylthio)quinazolin-1-ium iodide

A solution of 6-bromo-1,2-dimethylquinazoline-4(1H)-thione (100 mg, 0.392 mmol) in CH₃I (5 mL) was stirred at 25° C. for 3 hrs. The reaction mixture was concentrated to give the title compound (100 mg, 0.351 mmol, 89.5% yield) as a yellow solid.

LCMS m/e: 284, 286 (M).

### Step 4. Synthesis of (R,Z)-6-bromo-1,2-dimethyl-N-(1-(2-methyl-3-(trifluoromethyl)phenyl)-ethyl)quinazolin-4(1H)-imine

(R)-1-(2-Methyl-3-(trifluoromethyl)phenyl)ethan-1-amine (850 mg, 4.21 mmol) was mixed with 6-bromo-1,2-dimethyl-4-(methylthio)quinazolin-1-ium iodide (1 g, 3.51 mmol) in DMA (5 mL). The reaction mixture was stirred at 25° C. for 2 hrs, poured into water (100 mL), and extracted with EtOAc (100 mL×3). The combined organic layers were washed with brine (150 mL×3), dried over anhydrous Na₂SO₄, filtered, and concentrated to dryness. The residue was purified by a reverse phase column chromatography (0-33%, CH₃CN in H₂O) to give the title compound (500 mg, 1.14 mmol, 32.5% yield) as a brown solid.

LCMS m/e: 438 (MH⁺).

Following a similar procedure, (R,Z)-3-(1-((6-bromo-1,2-dimethylquinazolin-4(1H)-ylidene)amino)ethyl)-2-methylbenzonitrile was prepared. See its structure below.

LCMS m/e: 395, 397 (MH⁺).

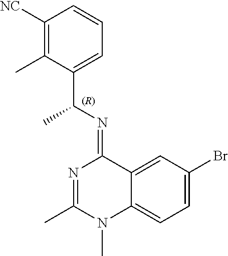

### Synthesis of (R,Z)-6-bromo-2-cyclopropyl-1-methyl-N-(1-(2-methyl-3-(trifluoromethyl)-phenyl)ethyl)pyrido[3,4-d]pyrimidin-4(1H)-imine

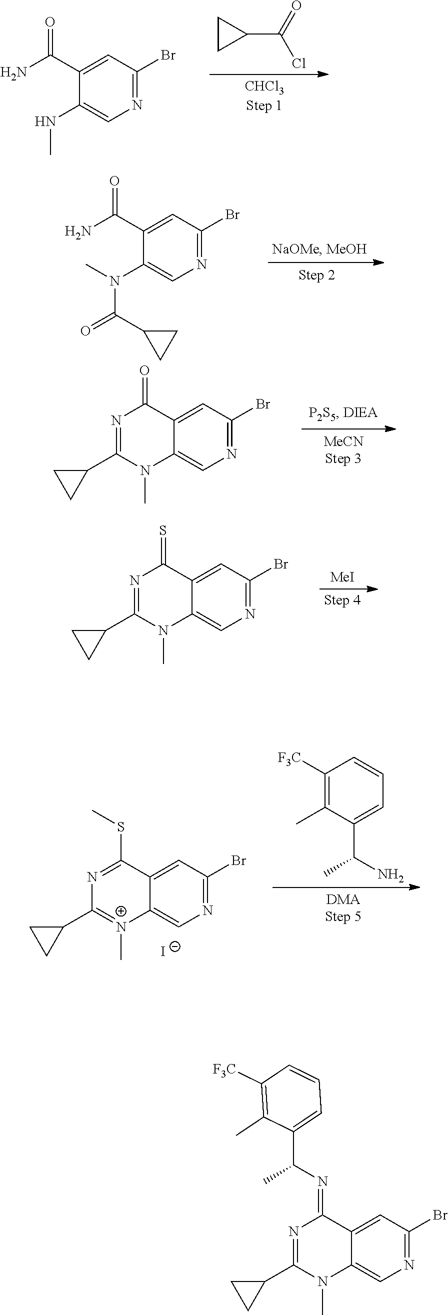

### Step 1. Synthesis of 2-bromo-5-(N-methylcyclopropanecarboxamido)isonicotinamide

To a solution of 2-bromo-5-(methylamino)isonicotinamide (2 g, 8.69 mmol) in CHCl₃ (40 mL) was added cyclopropanecarbonyl chloride (2.73 g, 26.1 mmol) and DIEA (3.37 g, 26.1 mmol). The mixture was stirred at 50° C. for 2 hrs. The resulting mixture was cooled to room temperature, concentrated, basified with saturated NaHCO₃ solution to pH 8, and extracted with EtOAc (100 mL×3). The combined organic layers were dried over anhydrous Na₂SO₄, filtered, and concentrated to dryness. The residue was purified by flash column chromatography on silica gel (gradient, PE to PE:EtOAc=3:1) to give the title compound (2.21 g, 7.38 mmol, 84.9% yield) as a white solid.

LC-MS m/e: 298.1, 300.1 (MH⁺).

### Step 2. Synthesis of 6-bromo-2-cyclopropyl-1-methylpyrido[3,4-d]pyrimidin-4(1H)-one

To a solution of 2-bromo-5-(N-methylcyclopropanecarboxamido)isonicotinamide (745 mg, 2.5 mmol) in MeOH (25 mL) was added NaOMe (540 mg, 10 mmol) and stirred at room temperature for 2 hrs. The mixture was diluted with brine (20 mL) and extracted with EtOAc (60 mL×3). The combined organic layers were dried over anhydrous Na₂SO₄, filtered, and concentrated to dryness. The residue was purified by flash column chromatography on silica gel (gradient, PE to PE:EtOAc=2:1) to give the title compound (400 mg, 1.43 mmol, 57.2% yield) as a white solid.

LC-MS m/e: 280.1, 282.1 (MH⁺).

### Step 3. Synthesis of 6-bromo-2-cyclopropyl-1-methylpyrido[3,4-d]pyrimidine-4(1H)-thione

To a solution of 6-bromo-2-cyclopropyl-1-methylpyrido[3,4-d]pyrimidin-4(1H)-one (400 mg, 1.43 mmol) in MeCN (25 mL) was added DIEA (738 mg, 5.72 mmol) and P₂S₅ (636 mg, 2.86 mmol). The mixture was stirred at 70° C. for 2 hrs under N₂ atmosphere. The mixture was cooled to room temperature, quenched with saturated NaHCO₃ solution (50 mL), and extracted with EtOAc (60 mL×3). The combined organic layers were dried over anhydrous Na₂SO₄, filtered, and concentrated to dryness. The residue was purified by flash column chromatography on silica gel (gradient, PE to PE:EtOAc=3:1) to give the title compound (300 mg, 1.01 mmol, 70.6% yield) as a yellow solid.

LC-MS m/e: 296.2, 298.2 (MH⁺).

### Step 4. Synthesis of 6-bromo-2-cyclopropyl-1-methyl-4-(methylthio)pyrido[3,4-d]pyrimidin-1-ium iodide

A solution of 6-bromo-2-cyclopropyl-1-methylpyrido[3,4-d]pyrimidine-4(1H)-thione (300 mg, 1.01 mmol) in MeI (10 mL) was stirred at room temperature for 1 hr. The mixture was concentrated to give the title compound (300 mg, 0.96 mmol, 96.4% yield) as a brown solid.

LC-MS m/e: 310.2, 312.2 (MH⁺).

### Step 5. Synthesis of (R,Z)-6-bromo-2-cyclopropyl-1-methyl-N-(1-(2-methyl-3-(trifluoromethyl)-phenyl)ethyl)pyrido[3,4-d]pyrimidin-4(1H)-imine

To a solution of 6-bromo-2-cyclopropyl-1-methyl-4-(methylthio)pyrido[3,4-d]pyrimidin-1-ium iodide (300 mg, 0.96 mmol) in DMA (20 mL) was added (R)-1-(2-methyl-3-(trifluoro-methyl)phenyl)ethan-1-amine (234.1 mg, 1.15 mmol) and stirred at room temperature for 1 hr. Then the mixture was poured into water (25 mL) and extracted with EtOAc (50 mL×3). The combined organic layers were washed with brine (50 mL), dried over anhydrous Na₂SO₄, filtered, and concentrated to dryness. The residue was purified by flash column chromatography on silica gel (gradient, DCM to DCM:CH₃OH=15:1) to give the title compound (200 mg, 0.43 mmol, 44.8% yield) as a brown solid.

LC-MS m/e: 465.3, 467.3 (MH⁺).

### Synthesis of (R, Z)-6-chloro-1,2-dimethyl-N-(1-(2-methyl-3-(trifluoromethyl)phenyl)ethyl)-pyrido[3,4-d]pyrimidin-4(1H)-imine

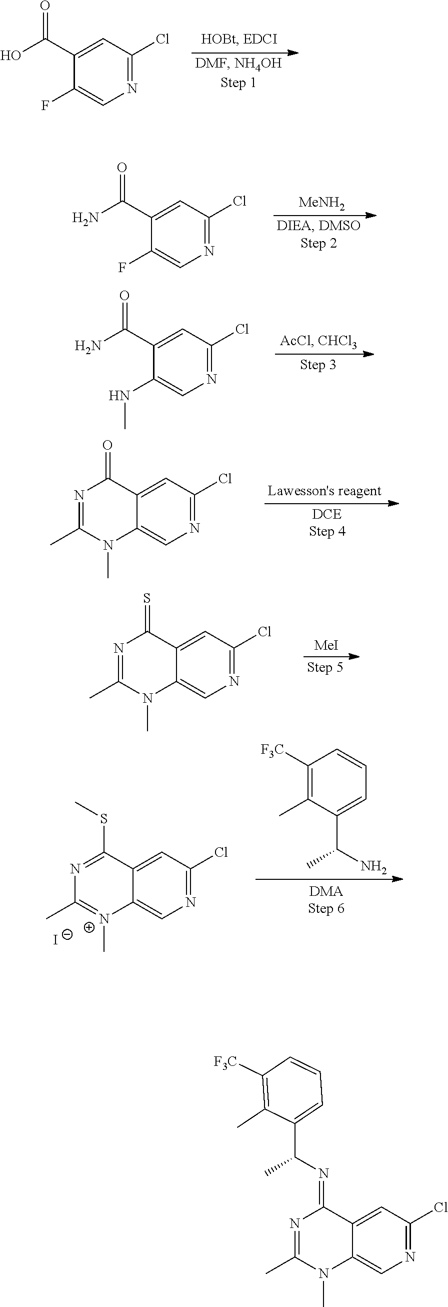

### Step 1. Synthesis of 2-chloro-5-fluoroisonicotinamide

To a solution of 2-chloro-5-fluoroisonicotinic acid (200 g, 1.14 mmol) and HOBt (185 g, 1.37 mmol) in DMF (1800 mL) was added EDCI (262 g, 1.37 mol) at 0° C. The mixture was stirred at room temperature for 2 hrs. A NH₄OH solution (244 g, 25%) was added to the mixture at 0° C. and stirred for another 1 hr. The resulting mixture was diluted with water (1 L) and extracted with EtOAc (1.6 L×3). The combined organic layers were washed with brine (1 L), dried over anhydrous Na₂SO₄, filtered, and concentrated to dryness. The residue was triturated with a mixture of PE (150 mL) and DCM (50 mL) to give the title compound (134 g, 766 mmol, 67.4% yield) as a white solid.

LC-MS m/e: 175.3, 177.3 (MH⁺).

### Step 2. Synthesis of 2-chloro-5-(methylamino)isonicotinamide

A mixture of 2-chloro-5-fluoroisonicotinamide (134 g, 766 mmol), methylamine hydrochloride (104 g, 1.54 mol) and DIEA (507 mL, 2.92 mol) in DMSO (900 mL) was stirred at 95° C. in a sealed tube overnight. The mixture was cooled to room temperature, diluted with water (400 mL), and extracted with EtOAc (800 mL×3). The combined organic layers were washed with brine (800 mL), dried over anhydrous Na₂SO₄, filtered, and concentrated to give the title compound (110 g, 594 mmol, 77.3% yield) as a yellow solid. The crude product was used directly in the next step without further purification.

LC-MS m/e: 186.1, 188.1 (MH⁺).

### Step 3. Synthesis of 6-chloro-1,2-dimethylpyrido[3,4-d]pyrimidin-4(1H)-one

A solution of 2-chloro-5-(methylamino)isonicotinamide (72 g, 388 mmol) and acetyl chloride (55.6 mL, 776 mmol) in CHCl₃ (1500 mL) was stirred at 80° C. overnight. The mixture was cooled to room temperature and concentrated. The residue was basified with saturated NaHCO₃ solution (600 mL) and extracted with EtOAc (500 mL×2). The combined organic layers were dried over anhydrous Na₂SO₄, filtered, and concentrated to give the title compound (56 g, 267 mmol, 68.7% yield) as a yellow solid.

LC-MS m/e: 210.1, 212.1 (MH⁺).

### Step 4. Synthesis of 6-chloro-1,2-dimethylpyrido[3,4-d]pyrimidine-4(1H)-thione

A mixture of 6-chloro-1,2-dimethylpyrido[3,4-d]pyrimidin-4(1H)-one (42 g, 200 mmol) and Lawesson's reagent (49 g, 120 mmol) in dichloroethane (500 mL) was stirred at 80° C. overnight. The mixture was cooled to room temperature and concentrated. The residue was diluted with HCl solution (2000 mL, 1M) and washed with EtOAc (800 mL). The aqueous layer was adjusted to pH 7 with NaHCO₃ and extracted with DCM (1000 mL×2). The combined organic layers were dried over anhydrous Na₂SO₄, filtered, and concentrated to give the title compound (23 g, 102 mmol, 51% yield) as a brown solid. The crude product was used directly in the next step without further purification.

LC-MS m/e: 226.1, 228.1 (MH⁺).

### Step 5. Synthesis of (6-chloro-1-methyl-4-(methylthio)pyrido[3,4-d]pyrimidin-1-ium-2-yl)methane iodide

6-Chloro-1,2-dimethylpyrido[3,4-d]pyrimidine-4(1H)-thione (18 g, 79.6 mmol) in iodomethane (50 mL) was stirred at room temperature for 4 hrs. The mixture was concentrated to give the title compound (19.1 g, 79.6 mmol, 100% yield) as a red solid. The crude product was used directly in the next step without further purification.

LC-MS m/e: 240.1, 242.1 (M⁺).

### Step 6. Synthesis of (R, Z)-6-chloro-1,2-dimethyl-N-(1-(2-methyl-3-(trifluoromethyl)phenyl)-ethyl)pyrido[3,4-d]pyrimidin-4(1H)-imine

To a solution of (6-chloro-1-methyl-4-(methylthio)pyrido[3,4-d]pyrimidin-1-ium-2-yl)methane iodide (19.1 g, 79.6 mmol) in DMA (100 mL) was added (R)-1-(2-methyl-3-(trifluoromethyl)phenyl)ethan-1-amine (16.2 g, 79 mmol). The mixture was stirred at room temperature for 15 mins. The resulting mixture was diluted with water (300 mL) and extracted with DCM (500 mL×2). The combined organic layers were dried over anhydrous Na₂SO₄, filtered, concentrated, and purified by neutral Al₂O₃ column chromatography (gradient, PE to PE:DCM:MeOH=16:3:1) to give the title compound (11.9 g, 30.2 mmol, 38% yield) as a brown solid.

LC-MS m/e: 395.1, 397.1 (MH⁺).

### Synthesis of (R,Z)-6-chloro-N-(1-(3-(difluoromethyl)-2-fluorophenyl)ethyl)-1,2-dimethylpyrido[3,4-d]pyrimidin-4(1H)-imine

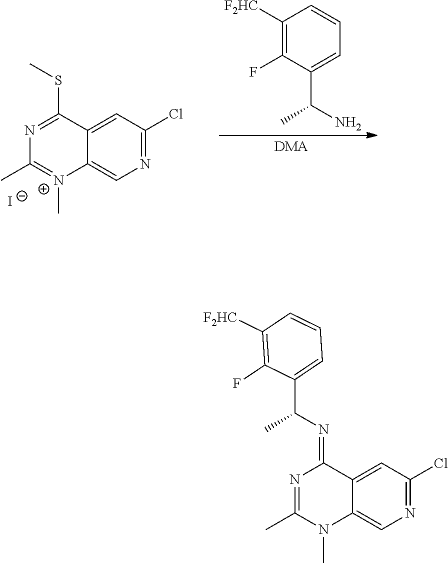

To a solution of (6-chloro-1-methyl-4-(methylthio)pyrido[3,4-d]pyrimidin-1-ium-2-yl)methanide (2 g, 8.27 mmol) in DMA (15 mL) was added (R)-1-(3-(difluoromethyl)-2-fluorophenyl)ethan-1-amine (1.88 g, 9.93 mmol) and stirred at room temperature for 1 hr. The reaction mixture was poured into water (100 mL) and extracted with DCM (150 mL×3). The combined organic layers were washed with brine (100 mL×3), dried over anhydrous Na₂SO₄, filtered and concentrated to dryness. The residue was purified by neutral Al₂O₃ column chromatography (gradient, DCM to DCM:MeOH=20:1) to give the title compound (1.6 g, 4.2 mmol, 50.8% yield) as a yellow solid. LC-MS m/e: 381.4 (MH⁺).

Applying a similar procedure described above, the following intermediates were prepared (Table 3 below from left to right):
(R,Z)-6-chloro-1-isopropyl-2-methyl-N-(1-(2-methyl-3-(trifluoromethyl)phenyl)ethyl)-pyrido[3,4-d]pyrimidin-4(1H)-imine,
(R,Z)-3-(1-((6-chloro-1,2-dimethylpyrido[3,4-d]pyrimidin-4(1H)-ylidene)amino)ethyl)-2-methylbenzonitrile, and
(R,Z)-6-chloro-1-cyclopropyl-2-methyl-N-(1-(2-methyl-3-(trifluoromethyl)phenyl)ethyl)-pyrido[3,4-d]pyrimidin-4(1H)-imine.

TABLE 3

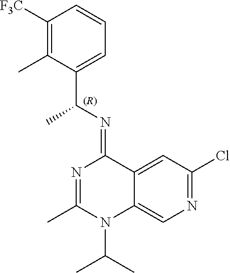

LC-MS m/e (MH⁺)
423.5, 425.5

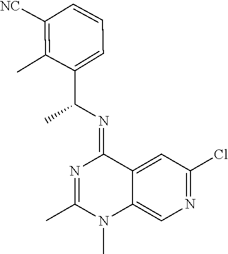 [SMILES: Cc1c(C#N)cccc1[C@@H](C)/N=c1\nc(C)n(C)c2cnc(Cl)cc12]

LC-MS m/e (MH⁺)
352.3, 354.3

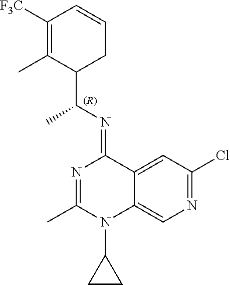

LC-MS m/e (MH⁺)
421.2, 423.2

### Synthesis of (R,Z)-6-chloro-1,2-dimethyl-N-(1-(2-methyl-3-(trifluoromethyl)phenyl)prop-2-yn-1-yl)pyrido[3,4-d]pyrimidin-4(1H)-imine

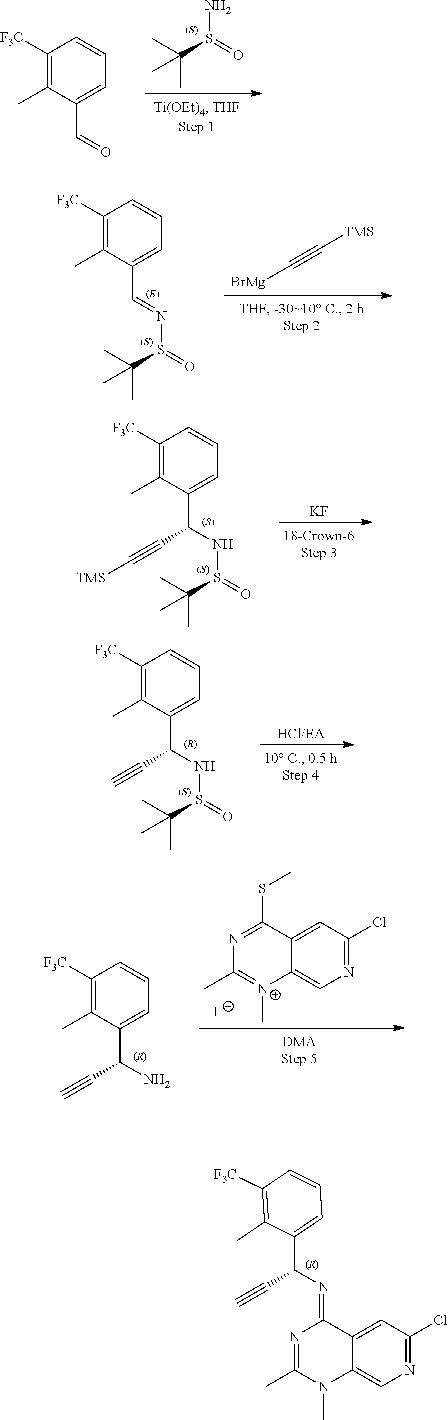

### Step 1. Synthesis of (S,E)-2-methyl-N-(2-methyl-3-(trifluoromethyl)benzylidene)propane-2-sulfinamide

To a solution of 2-methyl-3-(trifluoromethyl)benzaldehyde (23 g, 122 mmol) and (S)-(−)-2-methyl-2-propanesulfinamide (22.2 g, 183 mmol) in THF (300 mL) and Ti(OEt)₄ (77 mL, 0.37 mol) was added at 15° C. under N₂. The reaction mixture was stirred at 80° C. for 12 hours. The mixture was diluted with EtOAc (1 L) and water (50 mL), then filtered. The organic layer was washed with brine (200 mL), dried over anhydrous Na₂SO₄, and filtered. The filtrate was concentrated under reduced pressure to give the title compound (32.9 g, 113 mmol, 92% yield) as a white solid. The residue was used in the next step without further purification.

¹H NMR (400 MHz, CDCl₃) δ 8.96 (s, 1H), 8.11 (d, J=7.8 Hz, 1H), 7.76 (d, J=7.8 Hz, 1H), 7.38 (t, J=7.8 Hz, 1H), 2.68 (d, J=1.0 Hz, 3H), 1.27 (s, 9H).

LC-MS m/e: 292 (MH⁺).

### Step 2. Synthesis of (S)-2-methyl-N—((S)-1-(2-methyl-3-(trifluoromethyl)phenyl)-3-(trimethylsilyl)prop-2-yn-1-yl)propane-2-sulfinamide

To a solution of (S,E)-2-methyl-N-(2-methyl-3-(trifluoromethyl)benzylidene)propane-2-sulfinamide (5 g, 17.2 mmol) in THF (100 mL) was added trimethylsilylethynyl magnesium bromide in THF (1 M, 51.5 mL, 51.5 mmol) at −30° C. under N₂. The reaction mixture was gradually warmed up to 10° C. and stirred for 1 hour, followed by addition of saturated NH₄Cl aqueous solution (10 mL) and brine (50 mL) and then extraction with EtOAc (100 mL×2). The combined extracts were dried over anhydrous Na₂SO₄, filtered. The filtrate was concentrated under reduced pressure and purified by flash chromatography (gradient, PE/EtOAc=10/1 to 5/1) to give the title compound (6.3 g, 16.2 mmol, 94% yield) as a colorless oil.

¹H NMR (400 MHz, DMSO-d₆) δ 7.92 (d, J=7.7 Hz, 1H), 7.65 (d, J=7.7 Hz, 1H), 7.44 (t, J=7.8 Hz, 1H), 6.27 (d, J=7.6 Hz, 1H), 5.49 (d, J=7.6 Hz, 1H), 2.45 (s, 3H), 1.11 (s, 9H), 0.16 (s, 9H).

### Step 3. Synthesis of (S)-2-methyl-N—((R)-1-(2-methyl-3-(trifluoromethyl)phenyl)prop-2-yn-1-yl)propane-2-sulfinamide

To a solution of (S)-2-methyl-N—((S)-1-(2-methyl-3-(trifluoromethyl)phenyl)-3-(trimethylsilyl)prop-2-yn-1-yl)propane-2-sulfinamide (6.69 g, 17.2 mmol) in THF (166 mL) and H₂O (3.4 mL) at 0° C. was added a solution of 18-crown-6 (5 g, 18.9 mmol) and potassium fluoride (0.443 mL, 18.9 mmol) in a mixture of THF (61.75 mL) and H₂O (1.25 mL). The reaction mixture was stirred at 0° C. for 2 hrs and then concentrated under reduced pressure. The resulting crude product was purified by flash chromatography (gradient, EtOAc/PE=1/10 to 1/2) to give the title compound (5.3 g, 16.7 mmol, 97% yield) as a colorless oil.

¹H NMR (400 MHz, DMSO-d₆) δ 7.94 (d, J=7.8 Hz, 1H), 7.66 (d, J=7.7 Hz, 1H), 7.45 (t, J=7.8 Hz, 1H), 6.22 (d, J=7.8 Hz, 1H), 5.45 (dd, J=7.7, 2.4 Hz, 1H), 3.60 (d, J=2.4 Hz, 1H), 2.45 (s, 3H), 1.08 (d, J=23.6 Hz, 9H).

### Step 4. Synthesis of (R)-1-(2-methyl-3-(trifluoromethyl)phenyl)prop-2-yn-1-amine

To a solution of (S)-2-methyl-N—((R)-1-(2-methyl-3-(trifluoromethyl)phenyl)prop-2-yn-1-yl)propane-2-sulfinamide (5.3 g, 16.7 mmol) in EtOAc (40 mL) in an ice bath was slowly added a solution of HCl in EtOAc (2 N, 20.9 mL, 41.8 mmol) and stirred at 10° C. for 0.5 hrs.

The reaction mixture was concentrated under reduced pressure. The residue was diluted with MTBE (30 mL), stirred at 25° C. for 0.5 hrs, and then filtered. The solid collected was washed with MTBE (20 mL) and dried in vacuum to provide the title hydrochloride of compound (3.5 g, 16.4 mmol, 98.3% yield) as a white solid.

¹H NMR (400 MHz, DMSO-d₆) δ 9.12 (s, 3H), 7.99 (d, J=7.8 Hz, 1H), 7.78 (d, J=7.8 Hz, 1H), 7.56 (t, J=7.9 Hz, 1H), 5.65 (d, J=2.3 Hz, 1H), 3.96 (d, J=2.4 Hz, 1H), 2.50 (s, 3 H).

### Step 4

(R)-1-(2-methyl-3-(trifluoromethyl)phenyl)prop-2-yn-1-amine hydrochloride was neutralized using either one of the procedures below to provide the corresponding freebase. a) Solid K₂CO₃ (2 eq.) was added to the HCl salt in DMA. After stirring for 10 min, the solution was filtered. The filtrate was collected to give a solution of the compound as a free base. b) The HCl salt was added to a saturated NaHCO₃ aqueous solution. EA was added to extract the free base. The organic layers were combined, dried over MgSO₄, and evaporated to afford the compound as the free base.

### Step 5. Synthesis of (R,Z)-6-chloro-1,2-dimethyl-N-(1-(2-methyl-3-(trifluoromethyl)phenyl)prop-2-yn-1-yl)pyrido[3,4-d]pyrimidin-4(1H)-imine

(R)-1-(2-Methyl-3-(trifluoromethyl)phenyl)prop-2-yn-1-amine (350 mg, 1.642 mmol) was mixed and stirred with 6-chloro-1,2-dimethyl-4-(methylthio)-1,2-dihydropyrido[3,4-d]pyrimidine (397 mg, 1.649 mmol) in DMA (5 mL) at 25° C. for 2 hrs. The reaction mixture was poured into ice water (50 mL) and extracted with DCM (50 mL×4). The combined organic layers were washed with brine (100 mL×3), dried over anhydrous Na₂SO₄, filtered, and concentrated to dryness. The residue was purified by neutral Al₂O₃ gel (gradient, PE to PE:DCM:MeOH=16:3:1) to give the title compound (200 mg, 0.494 mmol, 30.1% yield) as a yellow solid.

LCMS m/e: 405.8, 407.8 (MH⁺).

### Synthesis of (R,Z)-6-bromo-1,2-dimethyl-N-(1-(2-methyl-3-(trifluoromethyl)phenyl)-ethyl)pyrido[2,3-d]pyrimidin-4(1H)-imine

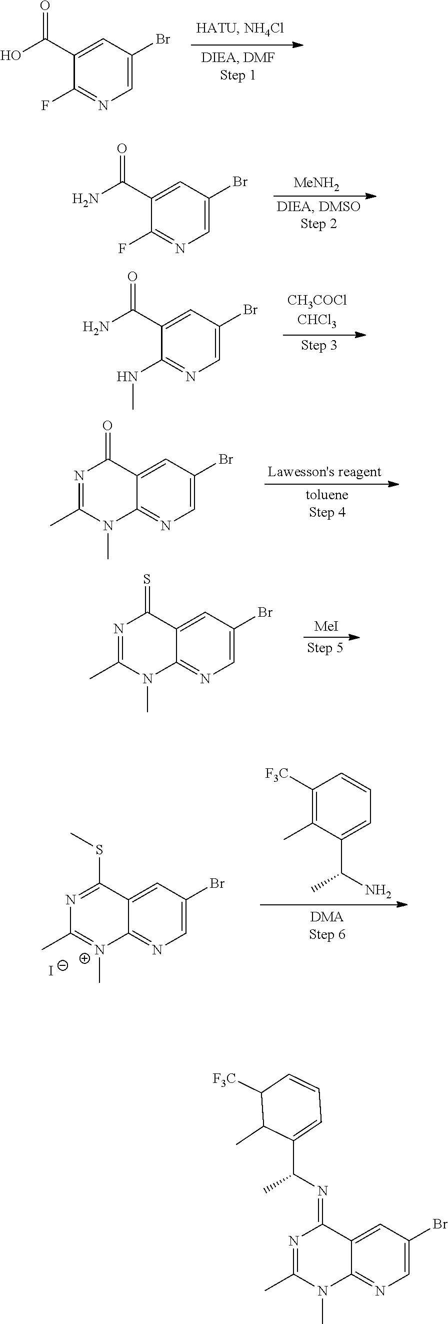

### Step 1. Synthesis of 5-bromo-2-fluoronicotinamide

To a solution of 5-bromo-2-fluoronicotinic acid (35 g, 159 mmol) and HATU (90.7 g, 239 mmol) in DMF (500 mL) was added NH₄Cl (25.5 g, 477 mmol) and DIEA (131 mL, 795 mmol) and the mixture was stirred at room temperature overnight. The resulting solution was poured into water (1.5 L) and extracted with EtOAc (1.5 L×3). The combined organic layers were washed with brine (1.5 L×3), dried over anhydrous Na₂SO₄, and filtered. The filtrate was concentrated in vacuum and triturated with a mixture of PE (150 mL) and DCM (50 mL). The solid was collected and dried in vacuum to give the title compound (27 g, 123 mmol, 77.5% yield) as yellow solid.

LC-MS m/e: 219, 221 (MH⁺).

### Step 2. Synthesis of 5-bromo-2-(methylamino)nicotinamide

To a solution of 5-bromo-2-fluoronicotinamide (27 g, 123 mmol) and methylamine hydrochloride (16.7 g, 247 mmol) in DMSO (200 mL) was added DIEA (61.1 mL, 370 mmol) and stirred at 90° C. in a sealed tube overnight. The resulting solution was cooled to room temperature, poured into water (1 L) and extracted with EtOAc (1 L×3). The combined organic layers were washed with brine (1.5 L×3), dried over anhydrous Na₂SO₄, and filtered. The filtrate was concentrated in vacuum and subsequently triturated with a mixture of PE (150 mL) and DCM (50 mL). The solid was collected and dried in vacuum to give the title compound (27 g, 117 mmol, 95.4% yield) as yellow solid.

LC-MS m/e: 229, 231 (MH⁺).

### Step 3. Synthesis of 6-bromo-1,2-dimethylpyrido[2,3-d]pyrimidin-4(1H)-one

To a solution of 5-bromo-2-(methylamino)nicotinamide (10 g, 43.5 mmol) in CHCl₃ (50 mL) was added CH₃COCl (34.1 g, 435 mmol) and stirred at 70° C. overnight. The reaction mixture was cooled to room temperature, distilled off the excess CH₃COCl, quenched with saturated NaHCO₃ solution (200 mL), and extracted with DCM/MeOH (10/1 in v/v) (100 mL×3). The combined organic phases were dried over anhydrous Na₂SO₄, filtered, and concentrated in vacuum to give the title compound (1.7 g, 6.69 mmol, 15.4% yield) as yellow solid.

LC-MS m/e: 254, 256 (MH⁺).

### Step 4. Synthesis of 6-bromo-1,2-dimethylpyrido[2,3-d]pyrimidine-4(1H)-thione

To a solution of 6-bromo-1,2-dimethylpyrido[2,3-d]pyrimidin-4(1H)-one (1.7 g, 6.7 mmol) in toluene (50 mL) was added Lawesson's reagent (1.62 g, 4 mmol). The reaction mixture was stirred at 80° C. overnight. The resulting solution was cooled to room temperature, poured into water (200 mL), and extracted with EtOAc (200 mL×3). The combined organic layers were washed with brine (200 mL), dried over anhydrous Na₂SO₄, and filtered. The filtrate was concentrated in vacuum and purified by neutral Al₂O₃ gel (gradient, PE to PE:DCM:MeOH=16:3:1) to give the title compound (1 g, 3.7 mmol, 55.3% yield) as red solid.

LC-MS m/e: 270, 272 (MH⁺).

### Step 5. Synthesis of (6-bromo-1-methyl-4-(methylthio)pyrido[2,3-d]pyrimidin-1-ium-2-yl)-methane iodide

A solution of 6-bromo-1,2-dimethylpyrido[2,3-d]pyrimidine-4(1H)-thione (1 g, 3.7 mmol) in CH₃I (10 mL) was stirred at room temperature for 2 hrs. The reaction solution was concentrated in vacuum to give crude title compound (1 g, 3.49 mmol, 94.4% yield) as a red solid, which was used directly in the next step without further purification.

LC-MS m/e: 284, 286 (M).

### Step 6. Synthesis of (R,Z)-6-bromo-1,2-dimethyl-N-(1-(2-methyl-3-(trifluoromethyl)phenyl)-ethyl)pyrido[2,3-d]pyrimidin-4(1H)-imine

To a solution of (6-bromo-1-methyl-4-(methylthio)pyrido[2,3-d]pyrimidin-1-ium-2-yl)-methane iodide (1 g, 3.49 mmol) in DMA (5 mL) was added (R)-1-(2-methyl-3-(trifluoro-methyl)phenyl)ethan-1-amine (852 mg, 4.19 mmol) and stirred at room temperature for 1 hr. The resulting mixture was poured into water (100 mL) and extracted with EtOAc (100 mL×2). The combined organic layers were washed with brine (100 mL), dried over anhydrous Na₂SO₄, and filtered. The filtrate was concentrated in vacuum and purified by neutral Al₂O₃ gel (gradient, DCM to DCM:MeOH=10:1) to give the title compound (860 mg, 1.96 mmol, 56% yield) as yellow solid.

LC-MS m/e: 441.4, 443.4 (MH⁺).

### Synthesis of (R,Z)-6-bromo-1-cyclopropyl-N-(1-(2-methyl-3-(trifluoromethyl)phenyl)-ethyl)pyrido[3,4-d]pyrimidin-4(1H)-imine

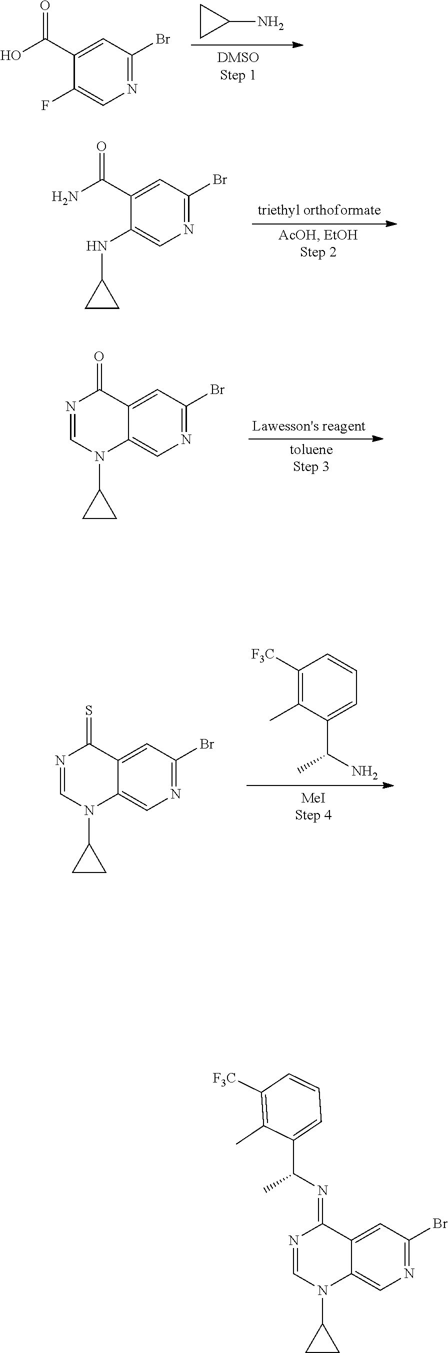

### Step 1. Synthesis of 2-bromo-5-(cyclopropylamino)pyridine-4-carboxamide

To a solution of 2-bromo-5-fluoropyridine-4-carboxamide (1.9 g, 8.68 mmol) in DMSO (10 mL) was added cyclopropanamine (1.2 mL, 17.4 mmol) and DIEA (4.5 mL, 26 mmol) and stirred at 90° C. in a sealed tube overnight. The mixture was cooled to room temperature, diluted with water (100 mL), and extracted with EtOAc (100 mL×3). The combined organic layers were washed with brine (100 mL), dried over anhydrous Na₂SO₄, filtered, and concentrated to give the title compound (1.85 g, 7.22 mmol, 83.3% yield) as a yellow solid.

LC-MS m/e: 256, 258 (MH⁺).

### Step 2. Synthesis of 6-bromo-1-cyclopropylpyrido[3,4-d]pyrimidin-4(1H)-one

To 2-bromo-5-(cyclopropylamino)pyridine-4-carboxamide (1.4 g, 5.47 mmol) in EtOH (60 mL) was added triethyl orthoformate (13.7 mL, 82 mmol) and AcOH (4.7 mL, 82 mmol) and stirred at 120° C. for 16 hrs. The reaction mixture was cooled to room temperature and concentrated to dryness. The residue was purified by silica gel column chromatography (gradient, PE to PE:EtOAc=3:1) to give the title compound (800 mg, 3 mmol, 55% yield) as a yellow solid.

LC-MS m/e: 266, 268 (MH⁺).

### Step 3. Synthesis of 6-bromo-1-cyclopropylpyrido[3,4-d]pyrimidine-4(1H)-thione

6-Bromo-1-cyclopropylpyrido[3,4-d]pyrimidin-4(1H)-one (700 mg, 2.6 mmol) was mixed with Lawesson's reagent (851 mg, 2.1 mmol) in 1,2-dichlorethane (40 mL) and stirred at 80° C. overnight. The reaction was cooled to room temperature, concentrated. The residue was dissolved in 1N HCl solution (200 mL) and washed with EtOAc (200 mL). Then the aqueous layer was treated with NaHCO₃ to ˜pH 7 and extracted with DCM (300 mL×2). The combined organic layers were dried over anhydrous Na₂SO₄ and filtered. The filtrate was concentrated to give the tittle compound (400 mg, 1.42 mmol, 53.9% yield) as a brown solid.

LC-MS m/e: 282, 284 (MH⁺).

### Step 4. Synthesis of (R,Z)-6-bromo-1-cyclopropyl-N-(1-(2-methyl-3-(trifluoromethyl)phenyl)-ethyl)pyrido[3,4-d]pyrimidin-4(1H)-imine

A solution of 6-bromo-1-cyclopropylpyrido[3,4-d]pyrimidine-4(1H)-thione (370 mg, 1.3 mmol) in CH₃I (4 mL) was stirred at room temperature for 4 hrs and then (1R)-1-[2-methyl-3-(trifluoromethyl)phenyl]ethan-1-amine (799 mg, 3.9 mmol) was added. The resulting mixture was stirred at room temperature for 3 hrs. The mixture was concentrated and purified by neutral Al₂O₃ column chromatography (gradient, PE to PE:DCM:MeOH=45:5:1) to give the tittle compound (500 mg, 1.11 mmol, 84.5% yield) as a brown solid.

LC-MS m/e: 451.1, 453.1 (MH⁺).

### Examples 1-13

### Synthesis of (R,Z)-1-(4-(1,2-dimethyl-4-((1-(2-methyl-3-(trifluoromethyl)phenyl)ethyl)-imino)-1,4-dihydropyrido[3,4-d]pyrimidin-6-yl)-4-fluoropiperidin-1-yl)ethan-1-one (Compound 1) and (R,Z)-1-(4-(1,2-dimethyl-4-((1-(2-methyl-3-(trifluoromethyl)phenyl)ethyl)imino)-1,4-dihydropyrido[3,4-d]pyrimidin-6-yl)-3,6-dihydropyridin-1(2H)-yl)ethan-1-one (Compound 2)

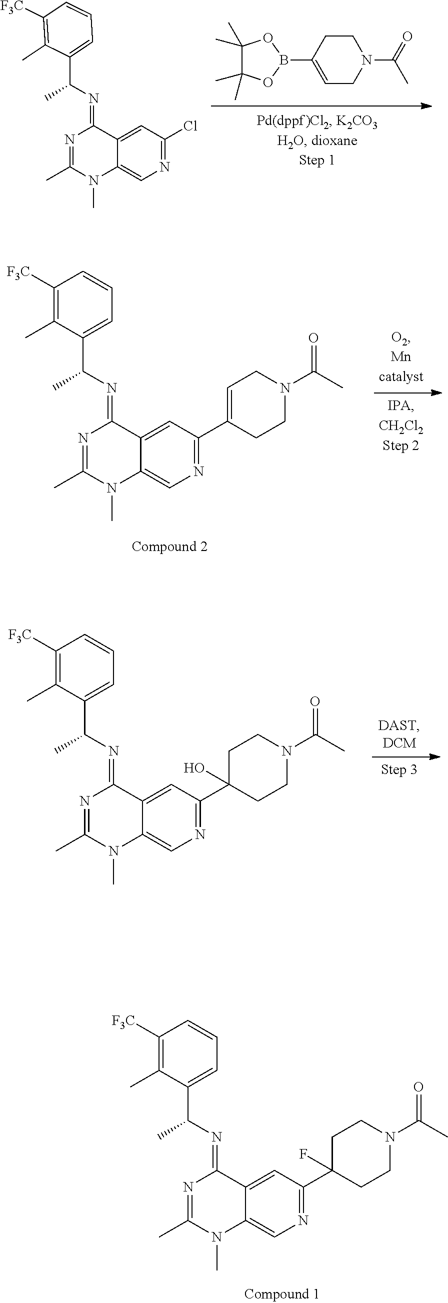

### Step 1. Synthesis of (R,Z)-1-(4-(1,2-dimethyl-4-((1-(2-methyl-3-(trifluoromethyl)phenyl)ethyl)imino)-1,4-dihydropyrido[3,4-d]pyrimidin-6-yl)-3,6-dihydropyridin-1(2H)-yl)ethan-1-one (Compound 2)

A mixture of (R,Z)-6-chloro-1,2-dimethyl-N-(1-(2-methyl-3-(trifluoromethyl)phenyl)-ethyl)pyrido[3,4-d]pyrimidin-4(1H)-imine (11.9 g, 30.2 mmol), 1-(4-(4,4,5,5-tetramethyl-1,3,2-dioxaborolan-2-yl)-3,6-dihydropyridin-1(2H)-yl)ethan-1-one (11.4 g, 45 mmol), Pd(dppf)Cl₂ (1.1 g, 1.5 mmol) and K₂CO₃ (12.6 g, 90.6 mmol) in dioxane (200 mL) and water (40 mL) was stirred at 100° C. for 4 hrs. The reaction mixture was cooled to room temperature, diluted with water (200 mL) and extracted with EtOAc (500 mL×2). The combined organic layers were dried over anhydrous Na₂SO₄, filtered, concentrated, and purified by Al₂O₃ column chromatography (100% PE to PE:DCM:MeOH=40:4:1) to afford the title compound (12 g, 24.8 mmol, 82.7% yield) as a yellow solid.

¹HNMR (400 MHz, DMSO-d₆) δ 8.91 (d, J=4.5 Hz, 1H), 8.25 (s, 1H), 8.18 (d, J=7.9 Hz, 1H), 7.95 (d, J=7.7 Hz, 1H), 7.53 (d, J=7.7 Hz, 1H), 7.37 (t, J=7.7 Hz, 1H), 6.77 (s, 1H), 5.79 (q, J=6.5 Hz, 1H), 4.23 (s, 1H), 4.18 (s, 1H), 3.74-3.66 (m, 4H), 2.68 (s, 1H), 2.58 (s, 1H), 2.53 (s, 3H), 2.48 (s, 3H), 2.07 (d, J=15.5 Hz, 3H), 1.44 (d, J=6.5 Hz, 3H).

LC-MS m/e: 484.7 (MH⁺).

### Step 2. Synthesis of (R,Z)-1-(4-(1,2-dimethyl-4-((1-(2-methyl-3-(trifluoromethyl)phenyl)-ethyl)imino)-1,4-dihydropyrido[3,4-d]pyrimidin-6-yl)-4-hydroxypiperidin-1-yl)ethan-1-one

To a solution of (R,Z)-1-(4-(1,2-dimethyl-4-((1-(2-methyl-3-(trifluoromethyl)phenyl)-ethyl)imino)-1,4-dihydropyrido[3,4-d]pyrimidin-6-yl)-3,6-dihydropyridin-1(2H)-yl)ethan-1-one (5.3 g, 10.9 mmol) and Mn(TMHD)₃ (2 g, 3.31 mmol) in isopropyl alcohol (180 mL) and DCM (20 mL) was added phenylsilane (1.8 g, 16.7 mmol) at 0° C. under O₂ and stirred for 4 hrs. The reaction mixture was quenched with a saturated Na₂S₂O₃ solution (40 mL), diluted with water (150 mL), and extracted with EtOAc (300 mL×2). The combined organic layers were dried over anhydrous Na₂SO₄ and filtered. The filtrate was concentrated under reduced pressure and purified by neutral Al₂O₃ column chromatography (gradient, 100% PE to PE:DCM:MeOH=25:4:1) to afford the title compound (2.4 g, 4.8 mmol, 44% yield) as a yellow solid.

LCMS m/e: 502.3 (MH⁺).

### Step 3. Synthesis of (R,Z)-1-(4-(1,2-dimethyl-4-((1-(2-methyl-3-(trifluoromethyl)phenyl)ethyl)imino)-1,4-dihydropyrido[3,4-d]pyrimidin-6-yl)-4-fluoropiperidin-1-yl)ethan-1-one ((Compound 1)

To a solution of (R,Z)-1-(4-(1,2-dimethyl-4-((1-(2-methyl-3-(trifluoromethyl)phenyl)-ethyl)imino)-1,4-dihydropyrido[3,4-d]pyrimidin-6-yl)-4-hydroxypiperidin-1-yl)ethan-1-one (1.1 g, 2.2 mmol) in DCM (40 mL) was added DAST (1.5 mL, 11 mmol) at −40° C. and stirred at −20° C. for 3 hrs. The reaction mixture was quenched with saturated NaHCO₃ solution (40 mL) and extracted with DCM (50 mL×2). The combined organic layers were dried over Na₂SO₄, filtered, concentrated, and purified by preparative-HPLC (0%-40% MeCN in H2O with 0.1% formic acid) to afford a formate salt of the title compound (340 mg).

¹H NMR (400 MHz, DMSO-d₆) δ 8.84 (s, 1H), 8.20 (s, 1H), 8.18 (s, 1H), 7.93 (d, J=7.9 Hz, 1H), 7.52 (d, J=7.8 Hz, 1H), 7.37 (t, J=7.6 Hz, 1H), 5.80-5.73 (m, 1H), 4.43 (d, J=12.7 Hz, 1H), 3.88 (d, J=12.1 Hz, 1H), 3.62 (s, 3H), 2.90 (t, J=12.0 Hz, 2H), 2.50 (s, 3H), 2.45 (s, 3H), 2.34-2.11 (m, 2H), 2.07 (s, 3H), 2.04-1.89 (m, 2H), 1.40 (d, J=6.3 Hz, 3H). LCMS m/e: 504.3 (MH⁺).

### Synthesis of (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-3,6-dihydropyridin-1(2H)-yl)ethan-1-one (compound 4), (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-4-hydroxypiperidin-1-yl)ethan-1-one (Compound 5), and (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-4-fluoropiperidin-1-yl)ethan-1-one (Compound 6)

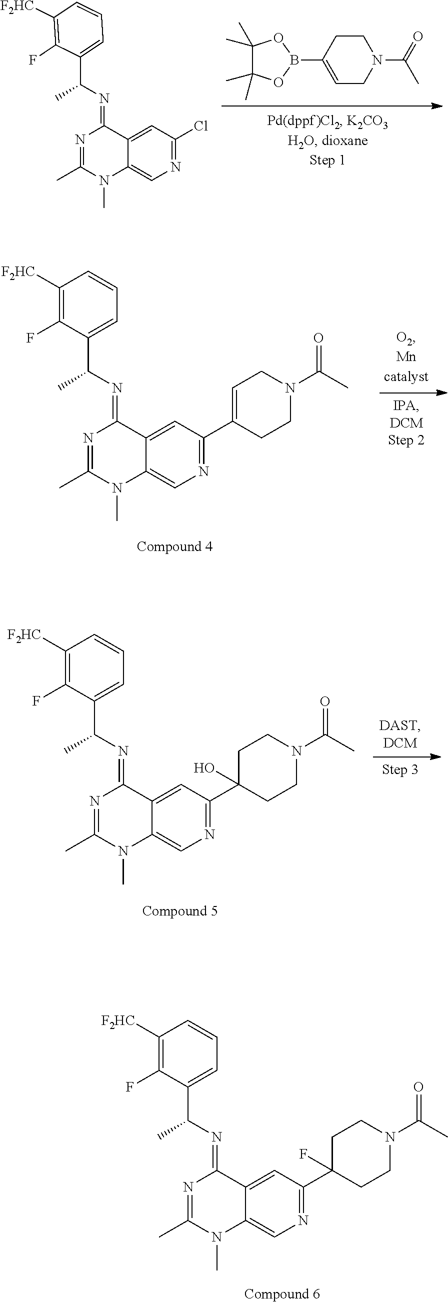 [SMILES: Cc1n/c(=N\[C@H](C)c2cccc(C(F)F)c2F)c2cc(Cl)ncc2n1C ; CC(=O)N1CC=C(B2OC(C)(C)C(C)(C)O2)CC1 ; CC(=O)N1CC=C(c2cc3/c(=N/[C@H](C)c4cccc(C(F)F)c4F)nc(C)n(C)c3cn2)CC1 ; CC(=O)N1CCC(O)(c2cc3/c(=N/[C@H](C)c4cccc(C(F)F)c4F)nc(C)n(C)c3cn2)CC1 ; CC(=O)N1CCC(F)(c2cc3/c(=N/[C@H](C)c4cccc(C(F)F)c4F)nc(C)n(C)c3cn2)CC1]

### Step 1. Synthesis of (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-3,6-dihydropyridin-1(2H)-yl)ethan-1-one (Compound 4)

To a solution of (R,Z)-6-chloro-N-(1-(3-(difluoromethyl)-2-fluorophenyl)ethyl)-1,2-dimethylpyrido[3,4-d]pyrimidin-4(1H)-imine (1.5 g, 3.94 mmol) in 1,4-dioxane (50 mL) and H₂O (10 mL) were added K₂CO₃ (1.63 g, 11.8 mmol), 1-[4-(4,4,5,5-tetramethyl-1,3,2-dioxaborolan-2-yl)-1,2,3,6-tetrahydropyridin-1-yl]ethan-1-one (1.48 g, 5.91 mmol) and Pd(dppf)Cl₂ (0.58 g, 0.788 mmol) and stirred at 100° C. under N₂ for 5 hrs. The reaction mixture was cooled to room temperature, poured into water (20 mL) and extracted with EtOAc (50 mL×3). The combined organic layers were washed with brine (50 mL), dried over anhydrous Na₂SO₄, filtered and concentrated to dryness. The residue was purified by silica gel column chromatography (gradient, DCM to DCM:MeOH=10:1) to give the title compound (1.3 g) as a yellow solid.

¹HNMR (400 MHz, DMSO-d₆) δ 9.02 (d, J=5.5 Hz, 1H), 8.30 (d, J=7.5 Hz, 1H), 7.94 (t, J=7.1 Hz, 1H), 7.50 (t, J=6.9 Hz, 1H), 7.37-7.07 (m, 2H), 6.82 (s, 1H), 5.85 (q, J=6.6 Hz, 1H), 4.29-4.19 (m, 2H), 3.74 (s, 3H), 3.72-3.65 (m, 2H), 2.74-2.60 (m, 2H), 2.53 (s, 3H), 2.08 (d, J=15.1 Hz, 3H), 1.50 (d, J=6.6 Hz, 3H). LC-MS m/e: 470.2 (MH⁺)

### Step 2. Synthesis of (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-4-hydroxypiperidin-1-yl)ethan-1-one (Compound 5)

To a solution of (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-3,6-dihydropyridin-1(2H)-yl)ethan-1-one (300 mg, 0.639 mmol) and Mn(THMD)₃ (193 mg, 0.319 mmol) in i-PrOH (18 mL) and DCM (2 mL) was added PhSiH₃ (0.119 mL, 0.958 mmol) at 0° C. under O₂ and stirred at 0° C. for 5 hrs.

The reaction mixture was poured into water (20 mL) and extracted with DCM (30×3). The combined organic layers were washed with brine (30 mL), dried over anhydrous Na₂SO₄, filtered and concentrated to dryness. The residue was purified by neutral Al₂O₃ column chromatography (gradient, 100% PE to PE:DCM:MeOH=25:4:1) to give the title compound (150 mg, 0.308 mmol, 48.2% yield) as yellow solid.

¹HNMR (400 MHz, DMSO-d₆) δ 8.85 (s, 1H), 8.44 (s, 1H), 8.23 (s, 1H), 7.91 (t, J=7.2 Hz, 1H), 7.49 (t, J=6.9 Hz, 1H), 7.37-7.05 (m, 2H), 5.83 (q, J=6.6 Hz, 1H), 4.32 (d, J=11.7 Hz, 1H), 3.75-3.69 (m, 1H), 3.66 (s, 3H), 3.44 (t, J=13.0 Hz, 1H), 2.93 (t, J=12.6 Hz, 1H), 2.48 (s, 3H), 2.18-2.08 m, 1H), 2.04 (s, 3H), 2.02-1.93 (m, 1H), 1.63-1.50 (m, 2H), 1.44 (d, J=6.7 Hz, 3H). LC-MS m/e: 488 (MH⁺).

### Step 3. Synthesis of (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-4-fluoropiperidin-1-yl)ethan-1-one (Compound 6)

To a solution of (R,Z)-1-(4-(4-((1-(3-(difluoromethyl)-2-fluorophenyl)ethyl)imino)-1,2-dimethyl-1,4-dihydropyrido[3,4-d]pyrimidin-6-yl)-4-hydroxypiperidin-1-yl)ethan-1-one (150 mg, 0.308 mmol) in DCM (10 mL) was added DAST (0.203 mL, 1.54 mmol) dropwise at −30° C. and stirred at −30° C. for 1 hr. The reaction was quenched with saturated NaHCO₃ solution (20 mL) and extracted with DCM (20 mL×2). The combined organic layers were dried over Na₂SO₄, filtered, concentrated, and purified by reverse phase column (SilaSep™ C18 silica flash cartridge, 0%-40% MeCN in H₂O with 0.1% FA) to give the title compound (11.1 mg, 0.023 mmol, 7.37% yield) as a yellow solid.

¹HNMR (400 MHz, DMSO-d₆) δ 8.81 (s, 1H), 8.17 (s, 1H), 7.92 (t, J=6.9 Hz, 1H), 7.46 (d, J=6.8 Hz, 1H), 7.37-7.05 (m, 2H), 5.79 (d, J=6.7 Hz, 1H), 4.48-4.39 (m, 1H), 3.93-3.85 (m, 1H), 3.61 (s, 3H), 2.95-2.86 (m, 1H), 2.44 (s, 3H), 2.36-2.12 (m, 2H), 2.08 (s, 3H), 2.03-1.87 (m, 3H), 1.39 (d, J=6.6 Hz, 3H). LC-MS m/e: 490 (MH⁺).

### Examples 3 and 7-14

Applying a similar synthetic procedure described above, Compounds 3 and 7-14 of the invention were prepared.

Compound 3: ¹H NMR (400 MHz, DMSO-d₆) δ 9.02 (d, J=5.2 Hz, 1H), 8.31 (s, 1H), 7.80 (d, J=7.8 Hz, 1H), 7.53 (d, J=7.7 Hz, 1H), 7.33 (t, J=7.8 Hz, 1H), 6.81 (d, J=3.5 Hz, 1H), 5.62 (q, J=6.7 Hz, 1H), 4.28-4.22 (m, 1H), 4.20-4.17 (m, 1H), 3.89 (s, 3H), 3.72-3.65 (m, 2H), 2.74-2.66 (m, 1H), 2.64-2.54 (m, 1H), 2.49 (s, 3H), 2.32-2.23 (m, 1H), 2.08 (d, J=15.6 Hz, 3H), 1.47 (d, J=6.6 Hz, 3H), 1.27-1.16 (m, 1H), 1.16-1.08 (m, 1H), 1.06-0.94 (m, 1H), 0.79-0.65 (m, 1H); LC-MS m/e: 510.4 (MH⁺).

Formate salt of Compound 7: ¹HNMR (400 MHz, DMSO-d₆) δ 1H NMR (400 MHz, DMSO) δ 8.88 (s, 1H), 8.21-8.20 (m, 1H), 8.16-8.06 (m, 1H), 7.96 (d, J=7.8 Hz, 1H), 7.61 (d, J=7.6 Hz, 1H), 7.37 (t, J=7.7 Hz, 1H), 6.75 (d, J=8.4 Hz, 1H), 5.70 (q, J=6.4 Hz, 1H), 4.20 (d, J=22.2 Hz, 2H), 3.72-3.62 (m, 5H), 2.72-2.64 (m, 1H), 2.61 (s, 3H), 2.59-2.55 (m, 1H), 2.47 (s, 3H), 2.07 (d, J=15.8 Hz, 3H), 1.41 (d, J=6.3 Hz, 3H); LCMS m/e: 441.6 (MH⁺).

Compound 8: ¹HNMR (400 MHz, DMSO-d₆) δ 8.94 (s, 1H), 8.24 (s, 1H), 7.94 (d, J=7.8 Hz, 1H), 7.62 (d, J=7.6 Hz, 1H), 7.39 (t, J=7.7 Hz, 1H), 5.70 (q, J=6.4 Hz, 1H), 4.44 (d, J=12.4 Hz, 1H), 3.88 (d, J=14.0 Hz, 1H), 3.68 (s, 3H), 3.40 (t, J=12.9 Hz, 1H), 2.90 (t, J=12.9 Hz, 1H), 2.62 (s, 3H), 2.51 (s, 3H), 2.37-2.12 (m, 2H), 2.08 (s, 3H), 2.02-1.90 (m, 2H), 1.43 (d, J=6.5 Hz, 3H); LCMS m/e: 461.2 (MH⁺).

Compound 9: ¹H NMR (400 MHz, DMSO-d₆) δ 8.96 (s, 1H), 8.13 (d, J=8.1 Hz, 1H), 7.96 (d, J=8.0 Hz, 1H), 7.51 (d, J=7.9 Hz, 1H), 7.36 (t, J=7.7 Hz, 1H), 6.72 (d, J=11.4 Hz, 1H), 5.81-5.70 (m, 1H), 4.89-4.73 (m, 1H), 4.20 (d, J=22.9 Hz, 2H), 3.66 (dd, J=11.6, 5.9 Hz, 2H), 3.26-3.20 (m, 3H), 2.63 (d, J=40.8 Hz, 2H), 2.51 (s, 3H), 2.07 (d, J=15.9 Hz, 3H), 1.59 (t, J=7.5 Hz, 6H), 1.39 (d, J=6.5 Hz, 3H); LCMS m/e: 512 (MH⁺).

Formate salt of Compound 10: ¹H NMR (400 MHz, DMSO-d₆) δ 9.02 (s, 1H), 8.19 (s, 1H), 8.05 (d, J=6.3 Hz, 1H), 7.97 (d, J=7.8 Hz, 1H), 7.52 (d, J=7.8 Hz, 1H), 7.37 (t, J=7.8 Hz, 1H), 6.78-6.69 (m, 1H), 5.79 (q, J=6.5 Hz, 1H), 4.20 (d, J=22.9 Hz, 2H), 3.71-3.64 (m, 3H), 3.25-3.21 (s, 3H), 2.71-2.56 (m, 2H), 2.53 (s, 3H), 2.07 (d, J=15.9 Hz, 3H), 1.39 (d, J=6.5 Hz, 3H), 1.30 (d, J=6.5 Hz, 2H), 1.01-0.89 (m, 2H); LCMS m/e: 510 (MH⁺).

Compound 11: ¹HNMR (400 MHz, DMSO-d₆) δ 8.85 (s, 1H), 8.02 (dd, J=7.6, 4.5 Hz, 2H), 7.60 (d, J=7.7 Hz, 1H), 7.41 (t, J=7.8 Hz, 1H), 6.74 (d, J=12.4 Hz, 1H), 6.51 (d, J=2.4 Hz, 1H), 4.19 (d, J=22.3 Hz, 2H), 3.74-3.60 (m, 5H), 3.20-3.17 (m, 1H), 2.69-2.59 (m, 2H), 2.53 (s, 3H), 2.47 (s, 3H), 2.06 (d, J=15.5 Hz, 3H); LCMS m/e: 494.6 (MH⁺).

Compound 12: ¹H NMR (400 MHz, DMSO-d₆) δ 8.73 (s, 1H), 8.48 (d, J=5.8 Hz, 1H), 7.97 (d, J=8.0 Hz, 1H), 7.51 (d, J=7.6 Hz, 1H), 7.35 (t, J=7.7 Hz, 1H), 6.35 (s, 1H), 5.79 (q, J=6.5 Hz, 1H), 4.16 (d, J=17.5 Hz, 2H), 3.70-3.62 (m, 5H), 2.70-2.56 (m, 2H), 2.52 (s, 3H), 2.46 (s, 3H), 2.07 (d, J=15.7 Hz, 3H), 1.39 (d, J=6.6 Hz, 3H); LCMS m/e: 484 (MH⁺).

Trifluoroacetate salt of Compound 13: ¹H NMR (400 MHz, DMSO-d₆) δ 10.76 (d, J=17.5 Hz, 1H), 9.24 (s, 2H), 7.83 (d, J=7.7 Hz, 1H), 7.63 (d, J=7.7 Hz, 1H), 7.44 (t, J=7.8 Hz, 1H), 6.00-5.86 (m, 1H), 4.56 (d, J=11.7 Hz, 1H), 3.99 (s, 3H), 3.94 (s, 1H), 3.44 (t, J=12.3 Hz, 1H), 2.91 (t, J=13.8 Hz, 1H), 2.77 (s, 3H), 2.61 (s, 3H), 2.52 (s, 1H), 2.25 (ddd, J=32.6, 23.4, 15.8 Hz, 3H), 2.10 (s, 3H), 1.67 (d, J=6.9 Hz, 3H); LCMS m/e: 504 (MH⁺).

Compound 14: ¹HNMR (400 MHz, DMSO-d₆) δ 9.02 (d, J=3.9 Hz, 1H), 8.19 (s, 1H), 8.14-8.09 (m, 2H), 8.02 (d, J=7.7 Hz, 1H), 7.54 (d, J=7.6 Hz, 1H), 7.39 (t, J=7.8 Hz, 1H), 6.74 (d, J=11.2 Hz, 1H), 5.80 (q, J=6.5 Hz, 1H), 4.23 (d, J=2.4 Hz, 1H), 4.17 (d, J=2.5 Hz, 1H), 3.65 (s, 1H), 3.35-3.31 (m, 1H), 2.67 (s, 1H), 2.58 (s, 1H), 2.47 (s, 3H), 2.07 (d, J=15.0 Hz, 3H), 1.39 (d, J=6.6 Hz, 3H), 1.14 (d, J=5.9 Hz, 2H), 1.04 (d, J=2.9 Hz, 2H); LCMS m/e: 496.2 (MH⁺).

### Examples 15-17

### Synthesis of (R,Z)-1-(4-fluoro-4-(1-methyl-4-((1-(2-methyl-3-(trifluoromethyl)phenyl)-ethyl)imino)-1,4-dihydropyrido[3,4-d]pyrimidin-6-yl)piperidin-1-yl)ethan-1-one hydrochloride (Compound 15)

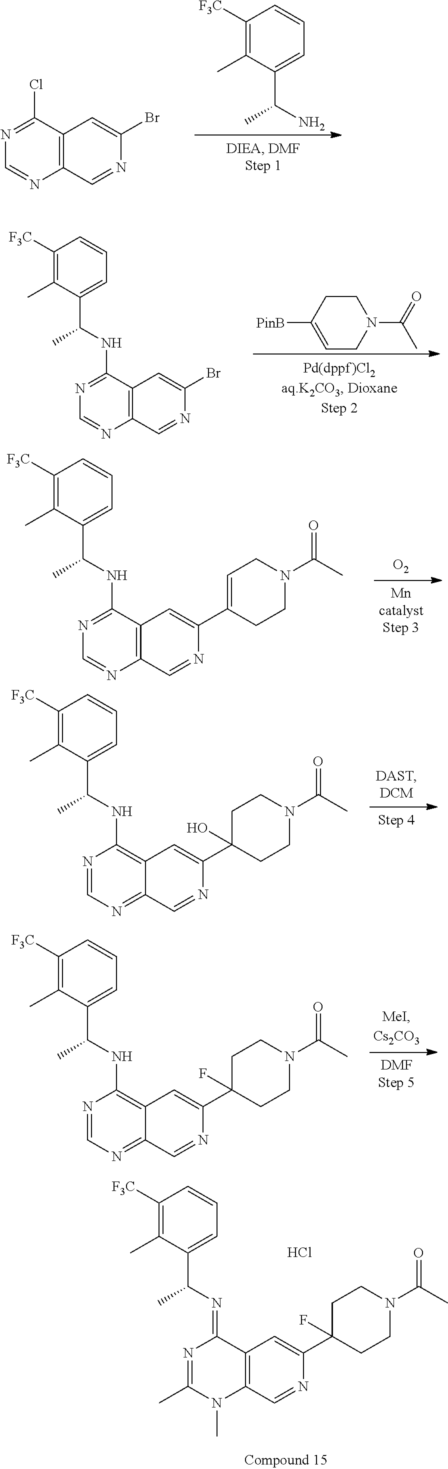

### Step 1. Synthesis of (R)-6-bromo-N-(1-(2-methyl-3-(trifluoromethyl)phenyl)ethyl)pyrido[3,4-d]pyrimidin-4-amine

To a solution of (R)—N-methyl-1-(2-methyl-3-(trifluoromethyl)phenyl)ethan-1-amine hydrochloride (594 mg, 2.34 mmol) in DMF (5 mL) was added DIEA (1.41 mL, 8.51 mmol) at 25° C. The mixture was stirred at 25° C. for 10 mins and 6-bromo-4-chloropyrido[3,4-d]pyrimidine (520 mg, 2.13 mmol) was added at 25° C. After stirring at 25° C. for 1 hr, the reaction mixture was poured into ice-water (60 mL) and extracted with EtOAc (60 mL×3). The combined organic layers were washed with brine (60 mL×3), dried over anhydrous Na₂SO₄, filtered, and concentrated to dryness. The residue was purified by flash column (gradient, PE to PE:EtOAc=2:1) to afford the title compound (880 mg, 2.07 mmol, 97.3% yield) as a pale-yellow solid.

LC-MS m/e: 427 (MH⁺).

### Step 2. Synthesis of (R)-1-(4-(4-((1-(2-methyl-3-(trifluoromethyl)phenyl)ethyl)amino)pyrido-[3,4-d]pyrimidin-6-yl)-3,6-dihydropyridin-1(2H)-yl)ethan-1-one

A mixture of (R)-6-bromo-N-(1-(2-methyl-3-(trifluoromethyl)phenyl)ethyl)pyrido[3,4-d]pyrimidin-4-amine (140 mg, 0.34 mmol), 1-(4-(4,4,5,5-tetramethyl-1,3,2-dioxaborolan-2-yl)-3,6-dihydropyridin-1(2H)-yl)ethan-1-one (103 mg, 0.409 mmol), Pd(dppf)Cl₂ (25 mg, 0.034 mmol) and K₂CO₃ (118 mg, 0.851 mmol) in dioxane (3 mL) and water (0.4 mL) was stirred at 100° C. under N₂ for 2.5 hrs. The reaction mixture was cooled to room temperature, diluted with water (20 mL), and extracted with EtOAc (50 mL×2). The combined organic layers were dried over anhydrous Na₂SO₄, filtered, concentrated, and purified by neutral Al₂O₃ gel (gradient, PE to PE:DCM:MeOH=40:4:1) to afford the title compound (80 mg, 0.176 mmol, 51.6% yield) as a yellow solid.

LC-MS m/e: 455 (MH⁺).

### Step 3. Synthesis of (R)-1-(4-hydroxy-4-(4-((1-(2-methyl-3-(trifluoromethyl)phenyl)ethyl)-amino)pyrido[3,4-d]pyrimidin-6-yl)piperidin-1-yl)ethan-1-one

To a solution of (R)-1-(4-(4-((1-(2-methyl-3-(trifluoromethyl)phenyl)ethyl)amino)-pyrido[3,4-d]pyrimidin-6-yl)-3,6-dihydropyridin-1(2H)-yl)ethan-1-one (0.9 g, 1.98 mmol) and Mn(TMHD)₃ (0.36 g, 0.59 mmol) in isopropyl alcohol (50 mL) and DCM (10 mL) was added phenylsilane (0.32 g, 2.97 mmol) at 0° C. under O₂ and stirred at room temperature for 1 hrs. The mixture was diluted with DCM (100 mL). The organic layer was washed with H₂O (10 mL) and brine (50 mL), dried over anhydrous Na₂SO₄, filtered, concentrated, and purified by flash column chromatography on silica gel (gradient, DCM to DCM:MeOH=15:1) to afford the title compound (0.9 g, 1.9 mmol, 96.2% yield) as a yellow solid.

¹H NMR (400 MHz, DMSO-d₆) δ 9.11 (d, J=7.1 Hz, 1H), 9.01 (s, 1H), 8.64 (s, 1H), 8.50 (s, 1H), 7.81 (d, J=7.8 Hz, 1H), 7.57 (d, J=7.9 Hz, 1H), 7.38 (t, J=7.8 Hz, 1H), 5.84-5.71 (m, 1H), 5.52 (s, 1H), 4.34 (d, J=11.9 Hz, 1H), 3.76 (d, J=12.8 Hz, 1H), 3.48 (t, J=12.7 Hz, 1H), 2.99 (t, J=12.5 Hz, 1H), 2.55 (s, 3H), 2.24-2.12 (m, 1H), 2.06 (d, J=10.2 Hz, 4H), 1.67 (t, J=15.0 Hz, 2H), 1.58 (d, J=6.8 Hz, 3H).

LC-MS m/e: 474(MH⁺).

### Step 4. Synthesis of (R)-1-(4-fluoro-4-(4-((1-(2-methyl-3-(trifluoromethyl)phenyl)ethyl)-amino)pyrido[3,4-d]pyrimidin-6-yl)piperidin-1-yl)ethan-1-one

To a solution (R)-1-(4-hydroxy-4-(4-((1-(2-methyl-3-(trifluoromethyl)phenyl)-ethyl)amino)pyrido[3,4-d]pyrimidin-6-yl)piperidin-1-yl)ethan-1-one (0.7 g, 1.48 mmol) in DCM (20 mL) was added DAST (0.36 g, 2.2 mmol) at −40° C. under N₂ and stirred for 30 mins. The reaction mixture was stirred at 0° C. for 2 hrs under N₂ and diluted with DCM (100 mL). The reaction was quenched with saturated NaHCO₃ solution (20 mL), washed with brine (50 mL), dried over anhydrous Na₂SO₄, filtered, concentrated, and purified by flash column chromatography on silica gel (gradient, DCM to DCM:MeOH=30:1) to afford the title compound (0.7 g, 1.47 mmol, 99.6% yield) as a yellow solid.

¹H NMR (400 MHz, DMSO-d₆) δ 9.10 (d, J=6.8 Hz, 1H), 9.05 (s, 1H), 8.61 (s, 1H), 8.54 (s, 1H), 7.79 (d, J=7.8 Hz, 1H), 7.57 (d, J=7.5 Hz, 1H), 7.38 (t, J=7.8 Hz, 1H), 5.76 (d, J=6.3 Hz, 1H), 4.46 (d, J=11.9 Hz, 1H), 3.90 (d, J=13.5 Hz, 1H), 3.45 (d, J=13.0 Hz, 1H), 2.94 (t, J=12.7 Hz, 1H), 2.55 (s, 3H), 2.42-2.17 (m, 2H), 2.09 (s, 3H), 2.02 (d, J=12.1 Hz, 2H), 1.58 (d, J=6.7 Hz, 3H).

LC-MS m/e: 476 (MH⁺).

### Step 5. Synthesis of (R,Z)-1-(4-fluoro-4-(1-methyl-4-((1-(2-methyl-3-(trifluoromethyl)phenyl)-ethyl)imino)-1,4-dihydropyrido[3,4-d]pyrimidin-6-yl)piperidin-1-yl)ethan-1-one hydrochloride (Compound 15)

To a solution of (R)-1-(4-fluoro-4-(4-((1-(2-methyl-3-(trifluoromethyl)phenyl)ethyl)-amino)pyrido[3,4-d]pyrimin-6-yl)piperidin-1-yl)ethan-1-one (1 g, 2.1 mmol) in DMF (15 mL) was added Cs₂CO₃ (2.05 g, 6.3 mmol), the mixture was stirred at rt. for 20 mins under N₂, Iodomethane (0.36 g, 2.52 mmol) was added at 0° C. and stirred at room temperature for 3 hrs. The mixture was diluted with EtOAc (100 mL), washed with saturated NH₄Cl solution (50 mL) and brine (50 mL), dried over anhydrous Na₂SO₄, filtered, concentrated under reduced pressure, and purified by flash column chromatography on silica gel (gradient, DCM to DCM:MeOH=10:1) to afford the crude compound, which was dissolved in EtOAc (10 mL) and a HCl solution (1 mL, 2 M in EtOAc) at 0° C. The resulting suspension was filtered. The cake was dried under vacuum to give the title compound (0.58 g, 1.1 mmol, 52.3% yield) as a yellow solid.

¹H NMR (400 MHz, DMSO-d₆) δ 11.07 (d, J=6.4 Hz, 1H), 9.44 (s, 1H), 9.04 (s, 1H), 8.94 (s, 1H), 7.84 (d, J=7.6 Hz, 1H), 7.64 (d, J=8.0 Hz, 1H), 7.46 (t, J=8.0 Hz, 1H), 6.01-5.84 (m, 1H), 4.48 (d, J=12.0 Hz, 1H), 4.07 (s, 3H), 3.93 (d, J=14.0 Hz, 1H), 3.46 (d, J=13.2 Hz, 1H), 2.95 (t, J=12.8 Hz, 1H), 2.55 (s, 3H), 2.39-2.14 (m, 2H), 2.09 (s, 3H), 2.04 (s, 2H), 1.69 (d, J=6.8 Hz, 3H).

LC-MS m/e: 490 (MH⁺).

Applying a similar procedure as described above, the following compounds (Compound 16 and 17) were prepared.

Compound 16: ¹HNMR (400 MHz, DMSO-d₆) δ 8.76 (s, 1H), 8.16-7.97 (m, 3H), 7.54 (d, J=7.6 Hz, 1H), 7.39 (t, J=7.8 Hz, 1H), 6.75 (d, J=9.9 Hz, 1H), 5.80-5.71 (m, 1H), 4.21 (d, J=22.3 Hz, 2H), 3.72-3.65 (m, 2H), 3.65 (s, 3H), 2.73-2.53 (m, 2H), 2.47 (s, 3H), 2.07 (d, J=15.7 Hz, 3H), 1.40 (d, J=6.5 Hz, 3H); LC-MS m/e: 470.4 (MH⁺).

Compound 17: ¹H NMR (400 MHz, DMSO-d₆) δ 8.27 (s, 1H), 7.98 (d, J=7.8 Hz, 2H), 7.69 (s, 1H), 7.51 (d, J=7.5 Hz, 1H), 7.38 (t, J=7.8 Hz, 2H), 5.81-5.68 (m, 1H), 4.48 (d, J=11.5 Hz, 1H), 3.87 (d, J=10.6 Hz, 1H), 3.54 (s, 3H), 3.42-3.36 (m, 1H), 2.91-2.81 (m, 1H), 2.47 (s, 3H), 2.08 (s, 3H), 2.07-1.84 (m, 4H), 1.37 (d, J=6.4 Hz, 3H); LC-MS m/e: 489.3 (MH⁺).

### Examples 18 and 19

### Synthesis of (R,Z)-6-(1-(difluoromethyl)cyclopropyl)-1,2-dimethyl-N-(1-(2-methyl-3-(trifluoromethyl)phenyl)ethyl)pyrido[3,4-d]pyrimidin-4(1H)-imine (Compound 18)

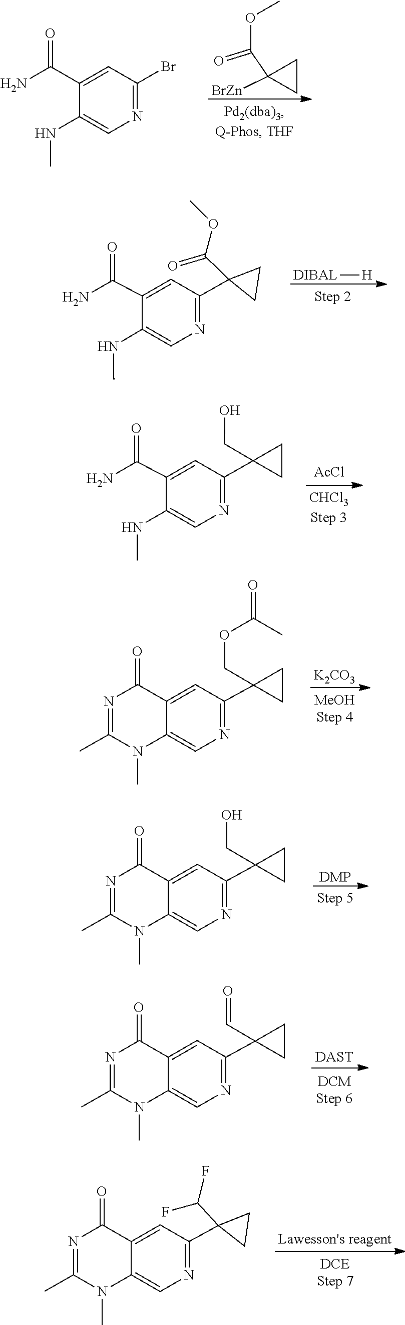

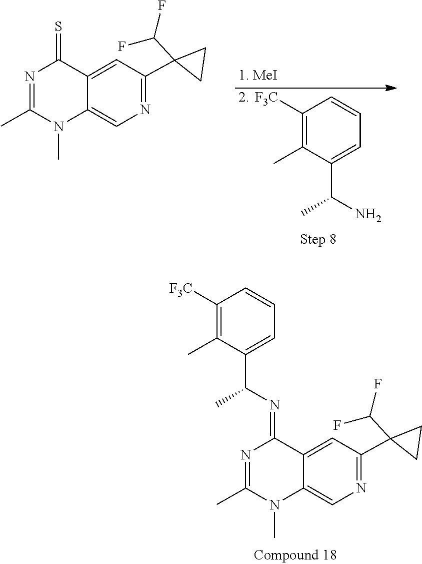 [SMILES: Cc1nc(=S)c2cc(C3(C(F)F)CC3)ncc2n1C ; "2." ; Cc1c([C@@H](C)N)cccc1C(F)(F)F ; Cc1c([C@@H](C)/N=c2\nc(C)n(C)c3cnc(C4(C(F)F)CC4)cc23)cccc1C(F)(F)F]

### Step 1. Synthesis of methyl 1-(4-carbamoyl-5-(methylamino)pyridin-2-yl)cyclopropane-1-carboxylate

To a solution of 2-bromo-5-(methylamino)isonicotinamide (5 g, 21.7 mmol), Pd₂(dba)₃ (1.99 g, 2.17 mmol) and Q-Phos (3.07 g, 4.34 mmol) in THF (50 mL) was added (1-(methoxy-carbonyl)cyclopropyl)zinc(II) bromide (145 mL, 109 mmol, 0.75 M in THF). The mixture was stirred at 25° C. for 2 hrs, diluted with water (150 mL) and extracted with EtOAc (200 mL×2). The combined organic layers were washed with brine (200 mL), dried over anhydrous Na₂SO₄, filtered, and concentrated to dryness. The residue was purified by silica gel column chromatography (gradient, PE to PE:EtOAc=3:1) to afford the tittle compound (4 g, 16.1 mmol, 74% yield) as a red solid.

LC-MS m/e: 250.3 (MH⁺).

### Step 2. Synthesis of 2-(1-(hydroxymethyl)cyclopropyl)-5-(methylamino)isonicotinamide

To a solution of methyl 1-(4-carbamoyl-5-(methylamino)pyridin-2-yl)cyclopropane-1-carboxylate (4 g, 16.1 mmol) in DCM (50 mL) was added DIBAL-H (53.7 mL, 80.6 mmol, 1.5M in hexane) at −50° C. and stirred at −30° C. for 0.5 hrs. The reaction mixture was quenched with water (100 mL) and extracted with EtOAc (200 mL×2). The combined organic layers were washed with brine (200 mL), dried over anhydrous Na₂SO₄, filtered, and concentrated to dryness. The residue was purified by silica gel column chromatography (gradient, DCM to DCM:MeOH=10:1) to afford the tittle compound (2.2 g, 9.95 mmol, 61.8% yield) as a yellow solid.

LC-MS m/e: 222.2 (MH⁺).

### Step 3. Synthesis of (1-(1,2-dimethyl-4-oxo-1,4-dihydropyrido[3,4-d]pyrimidin-6-yl)cyclopropyl)methyl acetate

To a solution of 2-(1-(hydroxymethyl)cyclopropyl)-5-(methylamino)isonicotinamide (2.2 g, 9.95 mmol) in CHCl₃ (50 mL) was added acetyl chloride (3.5 mL, 49.7 mmol) and stirred at 80° C. for 15 hrs. The reaction mixture was cooled to room temperature, concentrated, basified with saturated NaHCO₃ solution (100 mL), and extracted with DCM (200 mL×2). The combined organic layers were washed with brine (200 mL), dried over anhydrous Na₂SO₄, filtered, and concentrated to dryness. The residue was purified by neutral Al₂O₃ column chromatography (gradient, PE to PE:DCM:MeOH=20:3:1) to afford the tittle compound (1.7 g, 5.92 mmol, 59.5% yield) as a yellow solid.

LC-MS m/e: 288.1 (MH⁺).

### Step 4. Synthesis of 6-(1-(hydroxymethyl)cyclopropyl)-1,2-dimethylpyrido[3,4-d]pyrimidin-4(1H)-one

To a solution of (1-(1,2-dimethyl-4-oxo-1,4-dihydropyrido[3,4-d]pyrimidin-6-yl)cyclopropyl)methyl acetate (1.7 g, 5.92 mmol) in MeOH (15 mL) was added K₂CO₃ (2.12 g, 15.3 mmol) and stirred at 25° C. for 1.5 hrs. The reaction mixture was adjusted to ˜pH 7 with AcOH, concentrated, and purified by neutral Al₂O₃ column chromatography (gradient, PE to PE:DCM:MeOH=16:3:1) to give the tittle compound (1.3 g, 5.31 mmol, 89.6% yield) as a yellow solid.

LC-MS m/e: 246.1 (MH⁺).

### Step 5. Synthesis of 1-(1,2-dimethyl-4-oxo-1,4-dihydropyrido[3,4-d]pyrimidin-6-yl)cyclopropane-1-carbaldehyde

To a solution of 6-(1-(hydroxymethyl)cyclopropyl)-1,2-dimethylpyrido[3,4-d]pyrimidin-4(1H)-one (1.3 g, 5.31 mmol) in DCM (40 mL) was added Dess-Martin periodinane (4.5 g, 10.62 mmol) at 0° C. and stirred at 0° C. for 2 hrs. The mixture was directly purified by neutral Al₂O₃ column chromatography (gradient, PE to PE:DCM:MeOH=16:4:1) to afford the tittle compound (600 mg, 2.5 mmol, 46.5% yield) as yellow solid.

LC-MS m/e: 244.2 (MH⁺).

### Step 6. Synthesis of 6-(1-(difluoromethyl)cyclopropyl)-1,2-dimethylpyrido[3,4-d]pyrimidin-4(1H)-one

To a solution of 1-(1,2-dimethyl-4-oxo-1,4-dihydropyrido[3,4-d]pyrimidin-6-yl)-cyclopropane-1-carbaldehyde (600 mg, 2.5 mmol) in DCM (20 mL) was added DAST (1.7 mL, 12.3 mmol) at 0° C. and stirred at 0° C. for 2 hrs. The mixture was quenched with saturated NaHCO₃ solution (40 mL) and extracted with DCM (50 mL×2). The combined organic layers were washed with brine (100 mL), dried over anhydrous Na₂SO₄, and filtered. The filtrate was concentrated in vacuum and purified by neutral Al₂O₃ column chromatography (gradient, PE to PE:DCM:MeOH=16:4:1) to afford the title compound (150 mg, 0.565 mmol, 22.9% yield) as yellow solid.

LC-MS m/e: 266 (MH⁺).

### Step 7. Synthesis of 6-(1-(difluoromethyl)cyclopropyl)-1,2-dimethylpyrido[3,4-d]pyrimidine-4(1H)-thione

A mixture of 6-(1-(difluoromethyl)cyclopropyl)-1,2-dimethylpyrido[3,4-d]pyrimidin-4(1H)-one (150 mg, 0.565 mmol) and Lawesson's reagent (228 mg, 0.57 mmol) in dichloroethane (5 mL) was stirred at 85° C. for 3 hrs. The reaction mixture was cooled to room temperature and concentrated. The residue was dissolved in 1N HCl solution (50 mL) and washed with EtOAc (50 mL). Then the aqueous layer was treated with NaHCO₃ to ˜pH 7 and extracted with DCM (50 mL×2). The combined organic layers were dried over anhydrous Na₂SO₄, filtered, and concentrated to give the tittle compound (100 mg, 0.356 mmol, 62.7% yield) as a brown solid. The crude product was used directly in the next step without further purification.

LC-MS m/e: 282.1 (MH⁺).

### Step 8. Synthesis of (R,Z)-6-(1-(difluoromethyl)cyclopropyl)-1,2-dimethyl-N-(1-(2-methyl-3-(trifluoromethyl)phenyl)ethyl)pyrido[3,4-d]pyrimidin-4(1H)-imine (Compound 18)

To a solution of 6-(1-(difluoromethyl)cyclopropyl)-1,2-dimethylpyrido[3,4-d]pyrimidine-4(1H)-thione (50 mg, 0.178 mmol) in MeI (2 mL) was added (R)-1-(2-methyl-3-(trifluoro-methyl)phenyl)ethan-1-amine (181 mg, 0.892 mmol). The mixture was stirred at room temperature for 10 mins. Then the mixture was concentrated and purified by pre-HPLC to afford the tittle compound (30 mg, 0.067 mmol, 37.6%) as a yellow solid.

¹HNMR (400 MHz, DMSO-d₆) δ 9.07 (s, 1H), 8.27 (s, 1H), 7.88 (d, J=7.5 Hz, 1H), 7.56 (d, J=7.7 Hz, 1H), 7.40 (t, J=8.0 Hz, 1H), 6.44 (t, J=55.7 Hz, 1H), 5.81 (q, J=6.8 Hz, 1H), 3.75 (s, 3H), 2.58-2.52 (m, 6H), 1.50 (s, 3H), 1.34-1.28 (m, 4H).

LCMS m/e: 451.2 (MH⁺).

Following a similar procedure as described above, compound 19 was prepared.

¹HNMR (400 MHz, DMSO-d₆) δ 8.93 (s, 1H), 8.19 (s, 1H), 7.91 (d, J=8.1 Hz, 1H), 7.62 (d, J=7.6 Hz, 1H), 7.38 (t, J=7.7 Hz, 1H), 6.40 (t, J=56.1 Hz, 1H), 5.71 (q, J=6.8 Hz, 1H), 3.69 (s, 3H), 2.62 (s, 3H), 2.50 (s, 3H), 1.44 (d, J=6.7 Hz, 3H), 1.33-1.33 (m, 4H).

LCMS m/e: 408.3 (MH⁺).

### Example 20

### Synthesis of (R,Z)—N-(1-(3-(difluoromethyl)-2-fluorophenyl)ethyl)-6-(1-(difluoromethyl)-cyclopropyl)-1-methylpyrido[3,4-d]pyrimidin-4(1H)-imine (Compound 20)

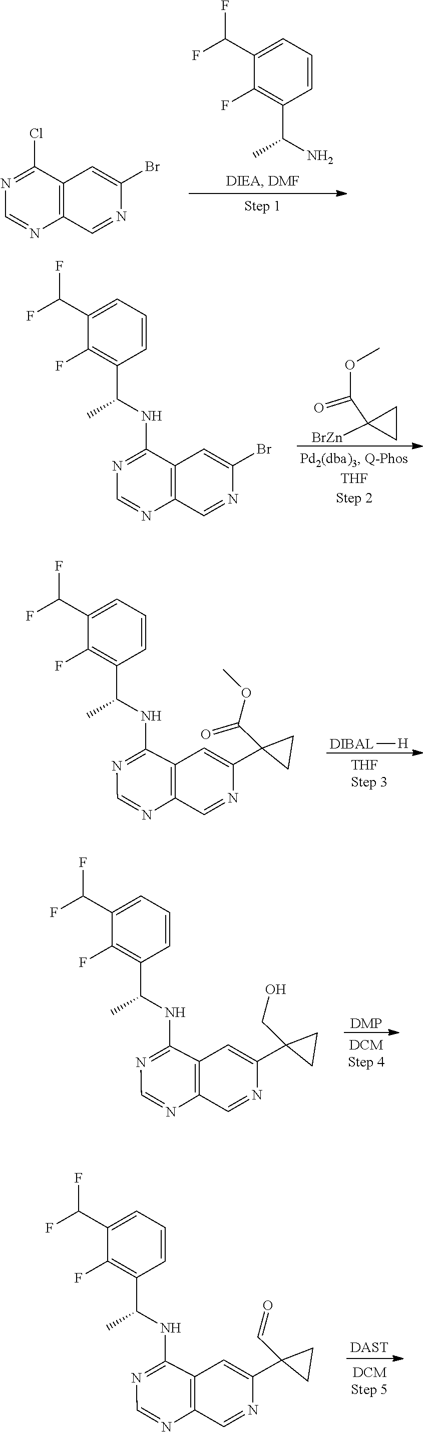

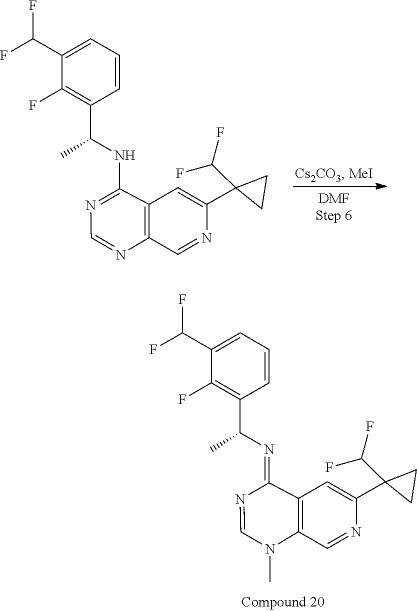

### Step 1. Synthesis of (R)-6-bromo-N-(1-(3-(difluoromethyl)-2-fluorophenyl)ethyl)pyrido[3,4-d]pyrimidin-4-amine

To a solution of 6-bromo-4-chloropyrido[3,4-d]pyrimidine (1.5 g, 6.14 mmol) in DMF (10 mL) were added DIEA (3.17 g, 24.542 mmol and (R)-1-(3-(difluoromethyl)-2-fluoro-phenyl)ethan-1-amine (1.28 g, 6.75 mmol). After stirring the reaction mixture at 25° C. for 2 hrs, the reaction mixture was poured into water (100 mL) and extracted with EtOAc (100 mL×3). The combined organic layers were washed with brine (100 mL×3), dried over anhydrous Na₂SO₄, filtered, and concentrated to dryness. The residue was purified by column chromatography (gradient, PE to PE:EtOAc=1:1) to afford the title compound (2.2 g, 5.54 mmol, 90.3% yield) as a white solid.

LC-MS m/e: 397.2, 399.2 (MH⁺).

### Step 2. Synthesis of methyl (R)-1-(4-((1-(3-(difluoromethyl)-2-fluorophenyl)ethyl)-amino)pyrido[3,4-d]pyrimidin-6-yl)cyclopropane-1-carboxylate

To a solution of 6-bromo-N-[(1R)-1-[3-(difluoromethyl)-2-fluorophenyl]ethyl]pyrido-[3,4-d]pyrimidin-4-amine (1 g, 2.52 mmol) in THF (10 mL) were added Pd₂(dba)₃ (230 mg, 0.252 mmol), Q-Phos (360 mg, 0.504 mmol) and (1-(methoxycarbonyl)cyclopropyl)zinc(II) bromide (31.5 mL, 0.75 M in THF). The mixture was stirred at 25° C. for 2 hrs, poured into saturated NH₄Cl solution (50 mL) and extracted with EtOAc (100 mL×3). The combined organic layers were washed with brine (100 mL), dried over anhydrous Na₂SO₄, filtered, and concentrated to dryness. The residue was purified by column chromatography (gradient, PE to PE:EtOAc=1:1) to afford the title compound (632 mg, 1.52 mmol, 60.3% yield) as a yellow solid.

LC-MS m/e: 417.3 (MH⁺).

### Step 3. Synthesis of (R)-(1-(4-((1-(3-(difluoromethyl)-2-fluorophenyl)ethyl)amino)pyrido[3,4-d]pyrimidin-6-yl)cyclopropyl)methanol

To a solution of methyl (R)-1-(4-((1-(3-(difluoromethyl)-2-fluorophenyl)ethyl)amino)-pyrido[3,4-d]pyrimidin-6-yl)cyclopropane-1-carboxylate (632 mg, 1.52 mmol) in DCM (10 mL) was added DIBAL-H (3 mL, 4.5 mmol, 1.5 M in hexane) at −20° C. and stirred at −20° C. for 1 h. The reaction mixture was quenched with saturated NH₄Cl solution (100 mL) and extracted with EtOAc (100 mL×3). The combined organic layers were washed with brine (100 mL), dried over anhydrous Na₂SO₄, filtered, and concentrated to dryness. The residue was purified by column chromatography (gradient, DCM to DCM:MeOH=30:1) to afford the title compound (300 mg, 0.772 mmol, 50.8% yield) as a yellow solid.

LC-MS m/e: 389.1 (MH⁺)

### Step 4. Synthesis of (R)-1-(4-((1-(3-(difluoromethyl)-2-fluorophenyl)ethyl)amino)pyrido[3,4-d]pyrimidin-6-yl)cyclopropane-1-carbaldehyde

To a solution of (R)-(1-(4-((1-(3-(difluoromethyl)-2-fluorophenyl)ethyl)amino)-pyrido[3,4-d]pyrimidin-6-yl)cyclopropyl)methanol (300 mg, 0.772 mmol) in DCM (10 mL) was added Dess-Martin periodinane (682 mg, 1.54 mmol) at 0° C. After stirring at 0° C. for 2 hrs, the reaction mixture was poured into saturated NaHCO₃ solution (50 mL) and extracted with DCM (100 mL×3). The combined organic layers were washed with brine (100 mL), dried over anhydrous Na₂SO₄, filtered, and concentrated to dryness. The residue was purified by column chromatography (gradient, DCM to DCM:MeOH=30:1) to afford the title compound (150 mg, 0.388 mmol, 50.3% yield) as a yellow solid.

LC-MS m/e: 387.1 (MH⁺).

### Step 5. Synthesis of (R)—N-(1-(3-(difluoromethyl)-2-fluorophenyl)ethyl)-6-(1-(difluoromethyl)cyclopropyl)pyrido[3,4-d]pyrimidin-4-amine

To a solution of (R)-1-(4-((1-(3-(difluoromethyl)-2-fluorophenyl)ethyl)amino)pyrido[3,4-d]pyrimidin-6-yl)cyclopropane-1-carbaldehyde in DCM (20 mL) was added DAST (125 mg, 0.777 mmol) at −40° C. under N₂. The reaction mixture was stirred at −40° C. for 30 mins and then at 0° C. for 2 hrs under N₂. The resulting mixture was diluted with DCM (100 mL), washed with saturated NaHCO₃ solution (20 mL) and brine (20 mL). The organic layer was dried over anhydrous Na₂SO₄, filtered, concentrated under reduced pressure, and purified by flash chromatography on silica gel (gradient, DCM to DCM:MeOH=30:1) to afford the title compound (125 mg, 0.306 mmol, 78.5% yield) as a yellow solid.

LC-MS m/e: 409 (MH⁺).

### Step 6. Synthesis of (R,Z)—N-(1-(3-(difluoromethyl)-2-fluorophenyl)ethyl)-6-(1-(difluoro-methyl)cyclopropyl)-1-methylpyrido[3,4-d]pyrimidin-4(1H)-imine (Compound 20)

To a solution of (R)—N-(1-(3-(difluoromethyl)-2-fluorophenyl)ethyl)-6-(1-(difluoro-methyl)cyclopropyl)pyrido[3,4-d]pyrimidin-4-amine (150 mg, 0.37 mmol) in DMF (10 mL) was added Cs₂CO₃ (362 mg, 1.1 mmol). The mixture was stirred at room temperature for 20 min under N₂. MeI (62.5 mg, 0.44 mmol) was added at 0° C. and stirred at room temperature for 3 hrs. The mixture was diluted with EtOAc (50 mL), washed with saturated NH₄Cl solution (25 mL) and brine (25 mL×3), dried over anhydrous Na₂SO₄, filtered, and concentrated to dryness. The residue was purified by prep-HPLC (SilaSep™ C18 silica flash cartridge, 20%-95% MeCN in H₂O with 0.1% TFA) to afford trifluoroacetate salt of the title compound (58.8 mg) as a yellow solid.

¹H NMR (400 MHz, CD₃OD) δ 9.35 (s, 1H), 8.81 (s, 1H), 8.57 (s, 1H), 7.71 (t, J=7.3 Hz, 1H), 7.56 (t, J=7.0 Hz, 1H), 7.32 (t, J=7.7 Hz, 1H), 6.99 (t, J=54.7 Hz, 1H), 6.48 (t, J=56.7 Hz, 1H), 6.07 (q, J=6.9 Hz, 1H), 4.14 (s, 3H), 1.81 (d, J=7.0 Hz, 3H), 1.45 (s, 4H).

LC-MS m/e: 423.6 (MH⁺).

### Example 21

### Synthesis of. (R,Z)-6-(1-(difluoromethyl)cyclopropyl)-1-methyl-4-((1-(2-methyl-3-(tri-fluoromethyl)phenyl)prop-2-yn-1-yl)imino)-4,6-dihydropyrido[4,3-d]pyrimidin-7(1H)-one (Compound 21)

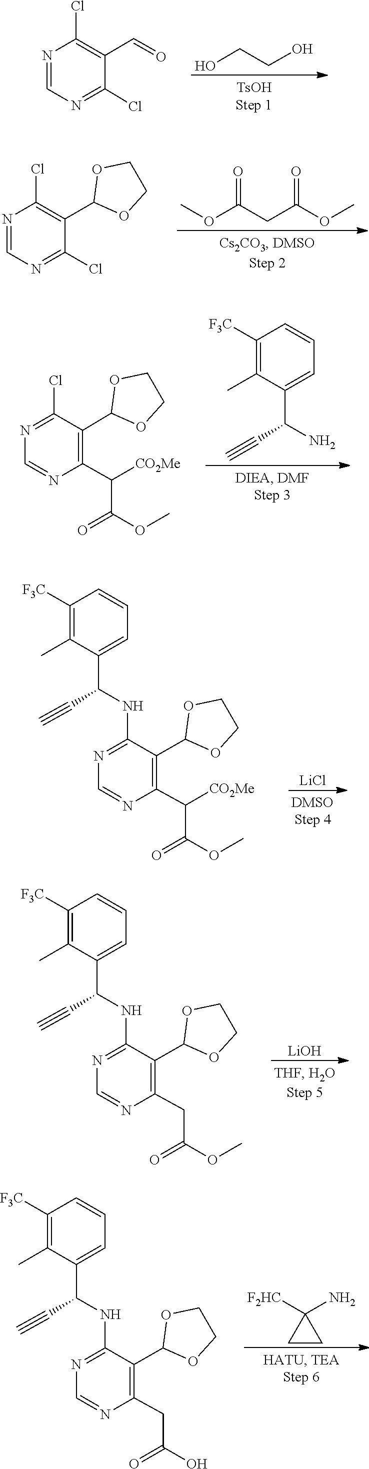

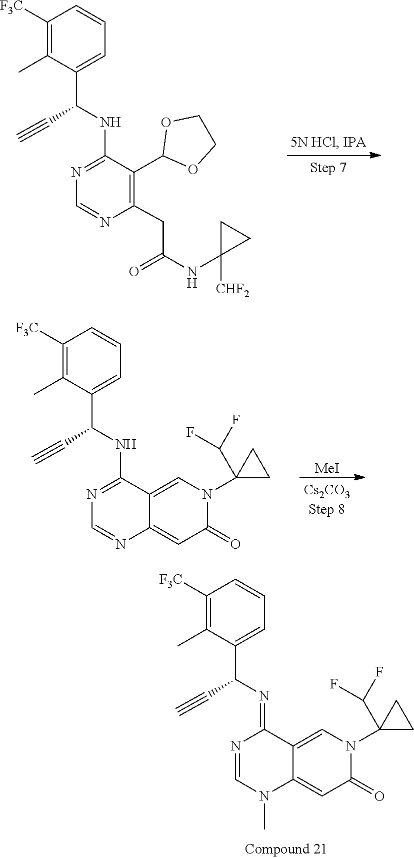

### Step 1. Synthesis of 4,6-dichloro-5-(1,3-dioxolan-2-yl)pyrimidine

To a solution of 4,6-dichloropyrimidine-5-carbaldehyde (2 g, 11.3 mmol) in toluene (50 mL) was added ethylene glycol (701 mg, 11.3 mmol) and TsOH (389 mg, 2.26 mmol). The mixture was stirred at 120° C. for 2 hrs, cooled to room temperature, diluted with water (100 mL) and extracted with EtOAc (100 mL×3). The combined organic extracts were washed with brine (100 mL), dried over anhydrous Na₂SO₄, and filtered. The filtrate was concentrated and purified by silica gel column chromatography (gradient, PE:EtOAc=10:1 to PE:EtOAc=5:1) to afford the title compound (1.8 g, 8.14 mmol, 72.1% yield) as a white solid.

LCMS m/e: 223.4 (MH⁺).

### Step 2. Synthesis of dimethyl 2-(6-chloro-5-(1,3-dioxolan-2-yl)pyrimidin-4-yl)malonate

To a solution of 4,6-dichloro-5-(1,3-dioxolan-2-yl)pyrimidine (1.8 g, 8.14 mmol) in DMSO (25 mL) were added dimethyl malonate (2.15 g, 16.3 mmol) and Cs₂CO₃ (5.3 g, 16.3 mmol). The reaction mixture was stirred at 80° C. for 2 hrs, cooled to room temperature, diluted with water (100 mL), and extracted with EtOAc (100 mL×3). The combined organic layers were washed with brine (100 mL), dried over anhydrous Na₂SO₄, and filtered. The filtrate was concentrated and purified by silica gel column chromatography (gradient, PE:EtOAc from 5:1 to 3:1) to afford the title compound (2 g, 6.31 mmol, 77.6% yield) as a white solid.

LCMS m/e:317.7 (MH⁺).

### Step 3. Synthesis of dimethyl (R)-2-(5-(1,3-dioxolan-2-yl)-6-((1-(2-methyl-3-(trifluoromethyl)-phenyl)prop-2-yn-1-yl)amino)pyrimidin-4-yl)malonate

To a solution of dimethyl 2-(6-chloro-5-(1,3-dioxolan-2-yl)pyrimidin-4-yl)malonate (793 mg, 2.51 mmol) in DMSO (15 mL) were added (R)-1-(2-methyl-3-(trifluoromethyl)-phenyl)prop-2-yn-1-amine (500 mg, 2.09 mmol) and DIEA (809 mg, 6.28 mmol). The mixture was stirred at 100° C. for 2 hrs, cooled to room temperature, diluted with water (50 mL), and extracted with EtOAc (50 mL×3). The combined organic layers were washed with brine (50 mL), dried over anhydrous Na₂SO₄, and filtered. The filtrate was concentrated and purified by silica gel column chromatography (gradient, PE:EtOAc=5:1 to PE:EtOAc=2:1) to afford the title compound (400 mg, 0.81 mmol, 38.8% yield) as a white solid.

LCMS m/e: 494.4 (MH⁺).

### Step 4. Synthesis of methyl (R)-2-(5-(1,3-dioxolan-2-yl)-6-((1-(2-methyl-3-(trifluoromethyl)-phenyl)prop-2-yn-1-yl)amino)pyrimidin-4-yl)acetate

To a solution of dimethyl (R)-2-(5-(1,3-dioxolan-2-yl)-6-((1-(2-methyl-3-(tri-fluoromethyl)phenyl)prop-2-yn-1-yl)amino)pyrimidin-4-yl)malonate (1 g, 2.03 mmol) in DMSO (25 mL) was added LiCl (429 mg, 10.13 mmol). The mixture was stirred at 120° C. for 2 hrs, cooled to room temperature, diluted with water (50 mL), and extracted with EtOAc (50 mL×3). The combined organic layers were washed with brine (100 mL), dried over anhydrous Na₂SO₄, and filtered. The filtrate was concentrated and purified by silica gel column chromatography (gradient, PE:EtOAc=5:1 to PE:EtOAc=2:1) to give the title compound (450 mg, 1.03 mmol, 50.9% yield) as a white solid.

LCMS m/e: 436.4 (MH⁺).

### Step 5. Synthesis of (R)-2-(5-(1,3-dioxolan-2-yl)-6-((1-(2-methyl-3-(trifluoromethyl)-phenyl)prop-2-yn-1-yl)amino)pyrimidin-4-yl)acetic acid

To a solution of methyl (R)-2-(5-(1,3-dioxolan-2-yl)-6-((1-(2-methyl-3-(trifluoromethyl)-phenyl)prop-2-yn-1-yl)amino)pyrimidin-4-yl)acetate (450 mg, 1.03 mmol) in t-BuOH (10 mL) was added a LiOH solution (5 M, 0.41 mL) and stirred at room temperature for 0.5 hrs. The reaction mixture was concentrated under reduced pressure to give the title compound (400 mg, 0.95 mmol, 92.2% yield) as a white solid.

LCMS m/e: 422.4 (MH⁺).

### Step 6. Synthesis of (R)-2-(5-(1,3-dioxolan-2-yl)-6-((1-(2-methyl-3-(trifluoromethyl)phenyl)-prop-2-yn-1-yl)amino)pyrimidin-4-yl)-N-(1-(difluoromethyl)cyclopropyl)acetamide

To a solution of (R)-2-(5-(1,3-dioxolan-2-yl)-6-((1-(2-methyl-3-(trifluoromethyl)phenyl)-prop-2-yn-1-yl)amino)pyrimidin-4-yl)acetic acid (400 mg, 0.95 mmol) in DMF (20 mL) was added 1-(difluoromethyl)cyclopropan-1-amine (204 mg, 1.9 mmol), HATU (722 mg, 1.9 mmol) and DIEA (368 mg, 2.85 mmol). After stirred at room temperature for 0.5 hrs, the reaction mixture was diluted with water (50 mL) and extracted with EtOAc (50 mL×3). The combined organic layers were washed with brine (50 mL), dried over anhydrous Na₂SO₄, and filtered. The filtrate was concentrated and purified by silica gel column chromatography (gradient, PE:EtOAc=3:1 to PE:EtOAc=1:1) to afford the title compound (250 mg, 0.49 mmol, 51.6% yield) as a white solid.

LCMS m/e: 511.5 (MH⁺).

### Step 7. Synthesis of (R)-6-(1-(difluoromethyl)cyclopropyl)-4-((1-(2-methyl-3-(trifluoromethyl)-phenyl)prop-2-yn-1-yl)amino)pyrido[4,3-d]pyrimidin-7(6H)-one

To a solution of (R)-2-(5-(1,3-dioxolan-2-yl)-6-((1-(2-methyl-3-(trifluoromethyl)phenyl)-prop-2-yn-1-yl)amino)pyrimidin-4-yl)-N-(1-(difluoromethyl)cyclopropyl)acetamide (250 mg, 0.49 mmol) in i-PrOH (25 mL) was added HCl solution (5 M, 2 mL). The reaction mixture was stirred at 100° C. for 2 hrs, cooled to room temperature, basified with saturated NaHCO₃ solution (60 mL), and extracted with EtOAc (60 mL×3). The combined organic layers were washed with brine (50 mL), dried over anhydrous Na₂SO₄, and filtered. The filtrate was concentrated and purified by prep-HPLC to afford the title compound (73 mg, 0.16 mmol, 33.2% yield) as a white solid.

¹H NMR (400 MHz, CD₃OD) δ 9.22 (s, 1H), 8.26 (s, 1H), 8.05 (d, J=7.9 Hz, 1H), 7.68 (d, J=7.7 Hz, 1H), 7.43 (t, J=7.8 Hz, 1H), 6.74 (d, J=2.3 Hz, 1H), 6.45 (s, 1H), 6.23 (t, J=57.5 Hz, 1H), 3.08 (d, J=2.4 Hz, 1H), 2.50 (s, 3H), 1.64-1.50 (m, 2H), 1.47-1.35 (m, 2H).

LCMS m/e: 449.2 (MH⁺).

### Step 8. Synthesis of (R,Z)-6-(1-(difluoromethyl)cyclopropyl)-1-methyl-4-((1-(2-methyl-3-(trifluoromethyl)phenyl)prop-2-yn-1-yl)imino)-4,6-dihydropyrido[4,3-d]pyrimidin-7(1H)-one (Compound 21)

To a solution of (R)-6-(1-(difluoromethyl)cyclopropyl)-4-((1-(2-methyl-3-(trifluoro-methyl)phenyl)prop-2-yn-1-yl)amino)pyrido[4,3-d]pyrimidin-7(6H)-one (35 mg, 0.078 mmol) in DMF (5 mL) was added Cs₂CO₃ (51 mg, 0.16 mmol) and CH₃I (11 mg, 0.078 mmol). The mixture was stirred at 60° C. for 2 hrs, diluted with water (60 mL), and then extracted with EtOAc (60 mL×3). The combined organic layers were washed with brine (60 mL), dried over anhydrous Na₂SO₄, and filtered. The filtrate was concentrated under reduced pressure and purified by prep-HPLC to afford the title compound (31 mg, 0.067 mmol, 85.9% yield) as a white solid.

¹H NMR (400 MHz, DMSO-d₆) δ 8.19 (s, 1H), 7.97 (d, J=7.7 Hz, 1H), 7.88 (s, 1H), 7.61 (d, J=7.6 Hz, 1H), 7.42 (t, J=7.8 Hz, 1H), 6.47 (d, J=2.4 Hz, 1H), 6.18 (t, J=56.3 Hz, 1H), 6.01 (s, 1H), 3.35 (s, 3H), 3.22 (d, J=2.4 Hz, 1H), 2.48 (s, 3H), 1.49-1.37 (m, 2H), 1.37-1.20 (m, 2H).

LCMS m/e: 463.2 (MH⁺).

### Examples 22 and 23

### Synthesis of (R,Z)-1-(4-hydroxy-4-(8-methoxy-1-methyl-4-((1-(2-methyl-3-(trifluoro-methyl)phenyl)ethyl)imino)-1,4-dihydropyrido[3,4-d]pyrimidin-6-yl)piperidin-1-yl)ethan-1-one (Compound 22) and (R,Z)-1-(4-fluoro-4-(8-methoxy-1-methyl-4-((1-(2-methyl-3-(trifluoromethyl)phenyl)ethyl)imino)-1,4-dihydropyrido[3,4-d]pyrimidin-6-yl)piperidin-1-yl)ethan-1-one (Compound 23)

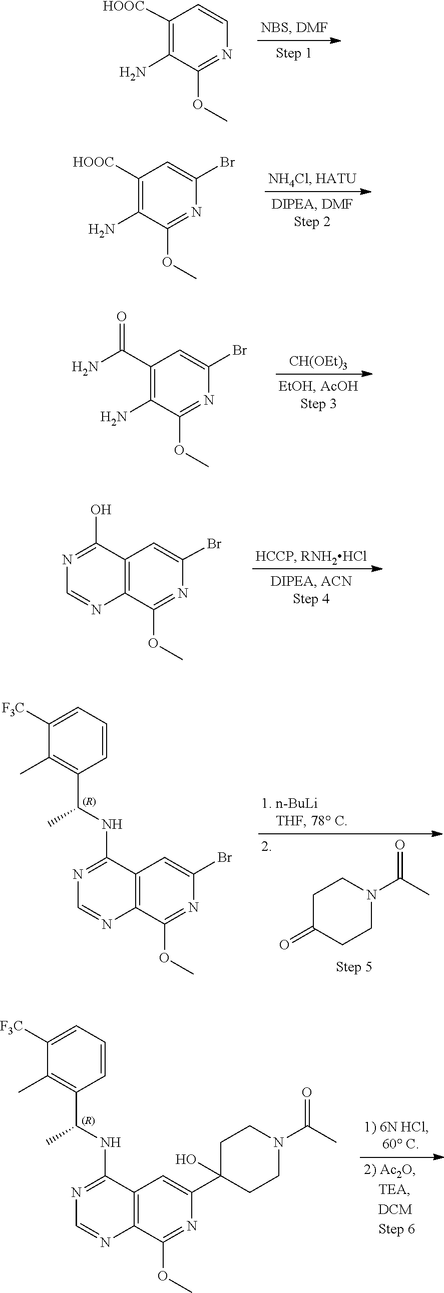

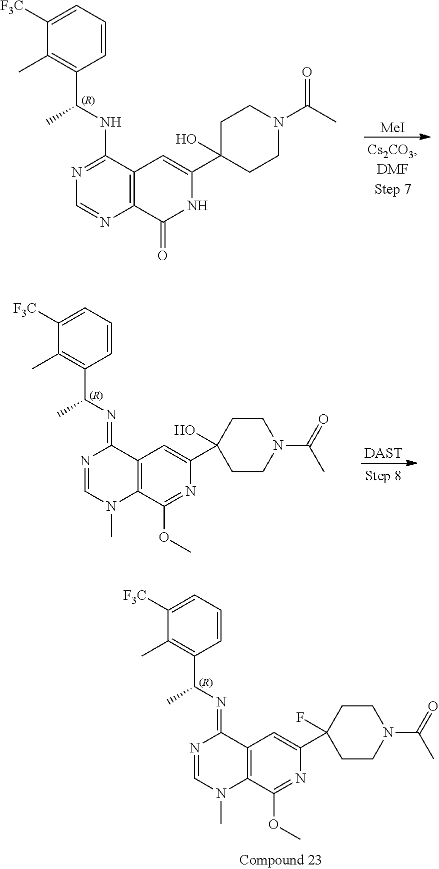

### Step 1. Synthesis of 3-amino-6-bromo-2-methoxyisonicotinic Acid

To a solution of 3-amino-2-methoxyisonicotinic acid (5 g, 27.3 mmol) in DMF (30 mL) was added NBS (5.11 g, 28.7 mmol) in portions. The mixture was stirred for 1 hr at 20° C., diluted with water (40 mL) and extracted with EtOAc (100 mL×2). The combined organic layers were washed with brine, dried over anhydrous Na₂SO₄, filtered, and concentrated to dryness. The residue was purified by flash column chromatography on silica gel (gradient, PE to PE:EtOAc=3:1) to afford the title compound (4.2 g, 17 mmol, 62.1% yield) as a brown solid. LC-MS m/e: 247, 249 (MH⁺).

### Step 2. Synthesis of 3-amino-6-bromo-2-methoxyisonicotinamide

To a solution of 3-amino-6-bromo-2-methoxyisonicotinic acid (4.2 g, 17 mmol) in DMF (20 mL) was added DIPEA (4.39 g, 34 mmol), NH₄Cl (1.36 g, 25.5 mmol) and HATU (7.75 g, 20.4 mmol) and stirred for 1 hr at 20° C. The reaction mixture was diluted with water (40 mL) and extracted with EtOAc (100 mL×2). The combined organic layers were washed with brine (100 mL), dried over anhydrous Na₂SO₄, filtered, and concentrated to dryness. The residue was purified by flash column chromatography on silica gel (gradient, PE to PE:EtOAc=1:1) to afford the title compound (4 g, 16.2 mmol, 95.6% yield) as a yellow solid.

LC-MS m/e: 246, 248 (MH⁺).

### Step 3. Synthesis of 6-bromo-8-methoxypyrido[3,4-d]pyrimidin-4-ol

To a solution of 3-amino-6-bromo-2-methoxyisonicotinamide (4 g, 16.2 mmol) in EtOH (80 mL) was added AcOH (13.9 mL, 243 mmol) and CH(OEt)₃ (36 g, 243 mmol) and stirred at 120° C. for 3 hrs. The resulting mixture was cooled to room temperature, concentrated under reduced pressure and the residue was triturated with EtOAc (50 mL) to afford the title compound (3.4 g, 13.2 mmol, 81.6% yield) as a yellow solid.

LC-MS m/e: 256, 258 (MH⁺).

### Step 4. Synthesis of (R)-6-bromo-8-methoxy-N-(1-(2-methyl-3-(trifluoromethyl)phenyl)-ethyl)pyrido[3,4-d]pyrimidin-4-amine

To a solution of 6-bromo-8-methoxypyrido[3,4-d]pyrimidin-4-ol (3.4 g, 13.2 mmol) in MeCN (30 mL) was added DIPEA (6.85 g, 53.1 mmol) and HCCP (5.08 g, 14.6 mmol). The mixture was stirred at 25° C. for 1 hr, followed by the addition of (1R)-1-[2-methyl-3-(trifluoro-methyl)phenyl]ethan-1-amine hydrochloride (3.46 g, 14.6 mmol). After stirring for 1 hr at 25° C., the mixture was diluted with water (30 mL) and extracted with EtOAc (50 mL×2). The combined organic layers were washed with brine (50 mL), dried over anhydrous Na₂SO₄, filtered, and concentrated to dryness. The residue was purified by flash column chromatography on silica gel (gradient, DCM to DCM:MeOH=30:1) to afford the title compound (4.5 g, 10.1 mmol, 76.8% yield) as a yellow solid.

LC-MS m/e: 441, 443 (MH⁺).

### Step 5. Synthesis of (R)-1-(4-hydroxy-4-(8-methoxy-4-((1-(2-methyl-3-(trifluoromethyl)-phenyl)ethyl)amino)pyrido[3,4-d]pyrimidin-6-yl)piperidin-1-yl)ethan-1-one

To a solution of (R)-6-bromo-8-methoxy-N-(1-(2-methyl-3-(trifluoromethyl)phenyl)-ethyl)pyrido[3,4-d]pyrimidin-4-amine (600 mg, 1.36 mmol) in THF (20 mL) was added dropwise n-BuLi (3.39 mL, 5.43 mmol, 1.6 M in Hexane) at −65° C. under N₂ and then stirred for 0.5 hrs. Subsequently, 1-acetylpiperidin-4-one (0.168 mL, 1.36 mmol) was added to the mixture and stirred at −65° C. for another 0.5 hrs. The reaction mixture was quenched with water (10 mL) and extracted with EtOAc (30 mL×2). The combined organic layers were washed with brine (50 mL), dried over anhydrous Na₂SO₄, filtered, and concentrated to dryness. The residue was purified by flash chromatography on silica gel (gradient, DCM to DCM:MeOH=20:1) to afford the title compound (100 mg, 0.2 mmol, 14.6% yield) as a yellow solid.

LC-MS m/e: 504 (MH⁺).

### Step 6. Synthesis of (R)-6-(1-acetyl-4-hydroxypiperidin-4-yl)-4-((1-(2-methyl-3-(trifluoromethyl)phenyl)ethyl)amino)pyrido[3,4-d]pyrimidin-8(7H)-one

A mixture of (R)-1-(4-hydroxy-4-(8-methoxy-4-((1-(2-methyl-3-(trifluoro-methyl)phen-yl)ethyl)amino)pyrido[3,4-d]pyrimidin-6-yl)piperidin-1-yl)ethan-1-one (100 mg, 0.199 mmol) and 6N HCl (10 mL, 60 mmol) was stirred at 60° C. for 18 hrs. The reaction mixture was cooled to 0° C., basified with an aqueous NaOH solution (10%) to pH 8, and extracted with EtOAc (20 mL×2). The combined organic layers were washed with brine, dried over anhydrous Na₂SO₄, filtered, and concentrated to dryness. The residue was dissolved in DCM (1 mL) followed by the addition of Et₃N (60 mg, 0.596 mmol) and AC₂O (0.047 mL, 0.497 mmol). The mixture obtained was stirred at 20° C. for 2 hrs, diluted with water (10 mL) and extracted with DCM (20 mL×2). The combined organic layers were washed with brine (10 mL), dried over anhydrous Na₂SO₄, filtered, and concentrated to dryness. The residue was purified by prep-HPLC (SilaSep™ C18 silica flash cartridge, 0%-40% MeCN in H₂O with 0.1% formic acid) to afford the title compound (30 mg, 0.061 mmol, 20.5% yield) as an off-white solid.

¹H NMR (400 MHz, CD₃OD) δ 8.42 (d, J=1.3 Hz, 1H), 7.66 (d, J=8.0 Hz, 1H), 7.54 (d, J=7.7 Hz, 1H), 7.30 (t, J=7.8 Hz, 1H), 7.02 (s, 1H), 5.77 (m, 1H), 4.59-4.49 (m, 1H), 3.89 (m, 1H), 3.60 (m, 1H), 3.10 (m, 1H), 2.57 (s, 3H), 2.15 (d, J=2.1 Hz, 3H), 2.10-1.99 (m, 2H), 1.92 (m, 2H), 1.61 (d, J=7.0 Hz, 3H).

LC-MS m/e: 490 (MH⁺).

### Step 7. Synthesis of (R,Z)-1-(4-hydroxy-4-(8-methoxy-1-methyl-4-((1-(2-methyl-3-(trifluoromethyl)phenyl)ethyl)imino)-1,4-dihydropyrido[3,4-d]pyrimidin-6-yl)piperidin-1-yl)ethan-1-one (Compound 22)

To a solution of (R)-6-(1-acetyl-4-hydroxypiperidin-4-yl)-4-((1-(2-methyl-3-(trifluoro-methyl)phenyl)ethyl)amino)pyrido[3,4-d]pyrimidin-8(7H)-one (20 mg, 0.041 mmol) in DMF (0.5 mL) was added Cs₂CO₃ (33 mg, 0.102 mmol). The mixture was stirred at 20° C. for 0.5 hrs. Iodomethane (15 mg, 0.102 mmol) in DMF (0.1 mL) was then added dropwise. The resulting mixture was stirred at 20° C. for 2 hrs, diluted with water (10 mL), and extracted with EtOAc (10 mL×2). The combined organic layers were washed with brine (10 mL×3), dried over anhydrous Na₂SO₄, and filtered. The filtrate was concentrated under reduced pressure and purified by prep-HPLC (SilaSep™ C18 silica flash cartridge, 0%-40% MeCN in H₂O with 0.1% formic acid) to afford the title compound (10.5 mg, 0.02 mmol, 49.5% yield) as an off-white solid.

¹H NMR (400 MHz, DMSO-d₆) δ 8.23 (s, 1H), 8.02 (s, 1H), 7.99 (s, 1H), 7.96 (d, J=7.8 Hz, 1H), 7.53 (d, J=7.5 Hz, 1H), 7.39 (t, J=7.8 Hz, 1H), 5.73 (q, J=6.5 Hz, 1H), 4.36-4.27 (m, 1H), 3.94 (s, 3H), 3.77 (s, 3H), 3.75-3.68 (m, 1H), 3.46-3.36 (m, 1H), 2.97-2.86 (m, 1H), 2.46 (s, 3H), 2.15-2.06 (m, 1H), 2.03 (s, 3H), 2.01-1.93 (m, 1H), 1.61-1.48 (m, 2H), 1.38 (d, J=6.6 Hz, 3H).

LC-MS m/e: 518 (MH⁺).

### Step 8. Synthesis of (R,Z)-1-(4-fluoro-4-(8-methoxy-1-methyl-4-((1-(2-methyl-3-(trifluoromethyl)phenyl)ethyl)imino)-1,4-dihydropyrido[3,4-d]pyrimidin-6-yl)piperidin-1-yl)ethan-1-one (Compound 23)

To a solution of (R,Z)-1-(4-hydroxy-4-(8-methoxy-1-methyl-4-((1-(2-methyl-3-(tri-fluoromethyl)phenyl)ethyl)imino)-1,4-dihydropyrido[3,4-d]pyrimidin-6-yl)piperidin-1-yl)ethan-1-one (40 mg, 0.077 mmol) in DCM (1 mL) was added dropwise DAST (31 mg, 0.193 mmol). The mixture was stirred at 5° C. for 0.5 hrs, diluted with DCM (30 mL) and quenched with saturated NaHCO₃ solution (20 mL). The organic layer was washed with brine (10 mL), dried over anhydrous Na₂SO₄, filtered, and concentrated to dryness. The residue was purified by prep-HPLC (SilaSep™ C18 silica flash cartridge, 0%-40% MeCN in H₂O with 0.1% NH₄HCO₃) to afford the title compound (4.7 mg, 0.009 mmol, 11.7% yield) as a white solid.

¹HNMR (400 MHz, DMSO-d₆) δ 7.95 (m, 2H), 7.83 (s, 1H), 7.53 (d, J=7.6 Hz, 1H), 7.39 (t, J=7.8 Hz, 1H), 5.70 (q, J=6.5 Hz, 1H), 4.46-4.38 (m, 1H), 3.95 (s, 3H), 3.91-3.83 (m, 1H), 3.76 (s, 3H), 3.43-3.40 (m, 1H), 2.94-2.84 (m, 1H), 2.46 (s, 3H), 2.34-2.09 (m, 2H), 2.07 (s, 3H), 2.03-1.83 (m, 2H), 1.36 (d, J=6.6 Hz, 3H).

LC-MS m/e: 520 (MH⁺).

### Example 24

### Synthesis of (R,Z)-1-(4-hydroxy-4-(8-methoxy-1-methyl-4-((1-(2-methyl-3-(trifluoromethyl)phenyl)ethyl)imino)-1,4-dihydropyrido[3,4-d]pyrimidin-6-yl)-2,2,6,6-tetramethylpiperidin-1-yl)ethan-1-one (Compound 24)

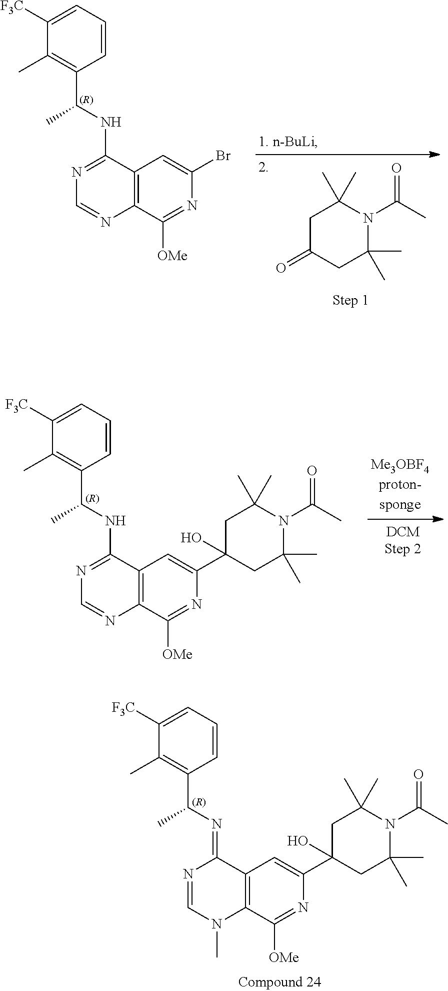

### Step 1. Synthesis of (R)-1-(4-hydroxy-4-(8-methoxy-4-((1-(2-methyl-3-(trifluoromethyl)phenyl)-ethyl)amino)pyrido[3,4-d]pyrimidin-6-yl)-2,2,6,6-tetramethylpiperidin-1-yl)ethan-1-one

To a solution of (R)-6-bromo-8-methoxy-N-(1-(2-methyl-3-(trifluoromethyl)phenyl)-ethyl)pyrido[3,4-d]pyrimidin-4-amine (500 mg, 1.13 mmol) in THF (20 mL) was added n-BuLi (2.26 mL, 5.66 mmol, 2.5 M in Hexane) dropwise at −70° C., the mixture was stirred at −70° C. for 0.5 hrs and then 1-acetyl-2,2,6,6-tetramethylpiperidin-4-one (447 mg, 2.26 mmol) was added. The resulting mixture was stirred at −70° C. for 0.5 hrs, quenched with water (10 mL) and extracted with EtOAc (30 mL×2). The combined organic layers were washed with brine (10 mL), dried over anhydrous Na₂SO₄, filtered, and concentrated to dryness. The residue was purified by flash chromatography (gradient, PE to PE:EtOAc=1:1) and prep-HPLC (SilaSep™ C18 silica flash cartridge, 0%-40% MeCN in H₂O with 0.1% FA) to afford the title compound (8 mg, 0.014 mmol, 1.26% yield) as a yellow solid.

¹H NMR (400 MHz, DMSO-d₆) δ 8.83 (s, 1H), 8.43 (s, 1H), 8.18 (s, 1H), 7.79 (d, J=7.8 Hz, 1H), 7.57 (d, J=7.7 Hz, 1H), 7.38 (t, J=7.8 Hz, 1H), 5.76 (p, J=6.8 Hz, 1H), 5.30 (s, 1H), 4.01 (s, 3H), 2.89-2.72 (m, 2H), 2.54 (s, 3H), 2.26-2.20 (m, 2H), 2.11 (s, 3H), 1.68-1.59 (m, 9H), 1.42 (s, 6H).

LC-MS m/e: 560 (MH⁺).

### Step 2. Synthesis of (R,Z)-1-(4-hydroxy-4-(8-methoxy-1-methyl-4-((1-(2-methyl-3-(trifluoromethyl)phenyl)ethyl)imino)-1,4-dihydropyrido[3,4-d]pyrimidin-6-yl)-2,2,6,6-tetramethylpiperidin-1-yl)ethan-1-one (Compound 24)

To a solution of (R)-1-(4-hydroxy-4-(8-methoxy-4-((1-(2-methyl-3-(trifluoromethyl)-phenyl)ethyl)amino)pyrido[3,4-d]pyrimidin-6-yl)-2,2,6,6-tetramethylpiperidin-1-yl)ethan-1-one (25 mg, 0.045 mmol) in DCM (0.6 mL) was added proton-sponge (9.6 mg, 0.045 mmol) and trimethyloxonium tetrafluoroborate (6.6 mg, 0.045 mmol). The mixture was stirred at 20° C. for 0.5 hrs, diluted with water (3 mL) and extracted with DCM (10 mL×2). The combined organic layers were washed with brine and dried over anhydrous Na₂SO₄, filtered and concentrated to dryness. The residue was purified by prep-HPLC (SilaSep™ C18 silica flash cartridge, 0%-40% MeCN in H₂O with 0.1% FA) to afford a formate salt of the title compound (7 mg) as a white solid.

¹H NMR (400 MHz, DMSO-d₆) δ 8.37 (s, 1H), 8.16 (s, 1H), 7.91 (d, J=7.7 Hz, 1H), 7.58 (d, J=7.8 Hz, 1H), 7.42 (t, J=7.8 Hz, 1H), 5.80 (q, J=6.6 Hz, 1H), 5.48 (s, 1H), 4.02 (s, 3H), 3.93 (s, 3H), 2.69 (dd, J=24.9, 15.0 Hz, 2H), 2.51 (s, 3H), 2.17-2.07 (m, 5H), 1.57 (d, J=1.4 Hz, 6H), 1.51 (d, J=6.5 Hz, 3H), 1.41 (d, J=2.6 Hz, 6H).

LC-MS m/e: 574 (MH⁺).

### Examples 25-30

### Synthesis of (R,Z)-1-(4-(5-((1-(2-methyl-3-(trifluoromethyl)phenyl)ethyl)imino)-5,7,8,9-tetrahydropyrido[4,3-e]pyrrolo[1,2-a]pyrimidin-3-yl)-3,6-dihydropyridin-1(2H)-yl)ethan-1-one (Compound 25), (R,Z)-1-(4-hydroxy-4-(5-((1-(2-methyl-3-(trifluoromethyl)phenyl)ethyl)-imino)-5,7,8,9-tetrahydropyrido[4,3-e]pyrrolo[1,2-a]pyrimidin-3-yl)piperidin-1-yl)ethan-1-one (Compound 26) and (R,Z)-1-(4-fluoro-4-(5-((1-(2-methyl-3-(trifluoromethyl)phenyl)ethyl)imino)-5,7,8,9-tetrahydropyrido[4,3-e]pyrrolo[1,2-a]pyrimidin-3-yl)piperidin-1-yl)ethan-1-one (Compound 27)

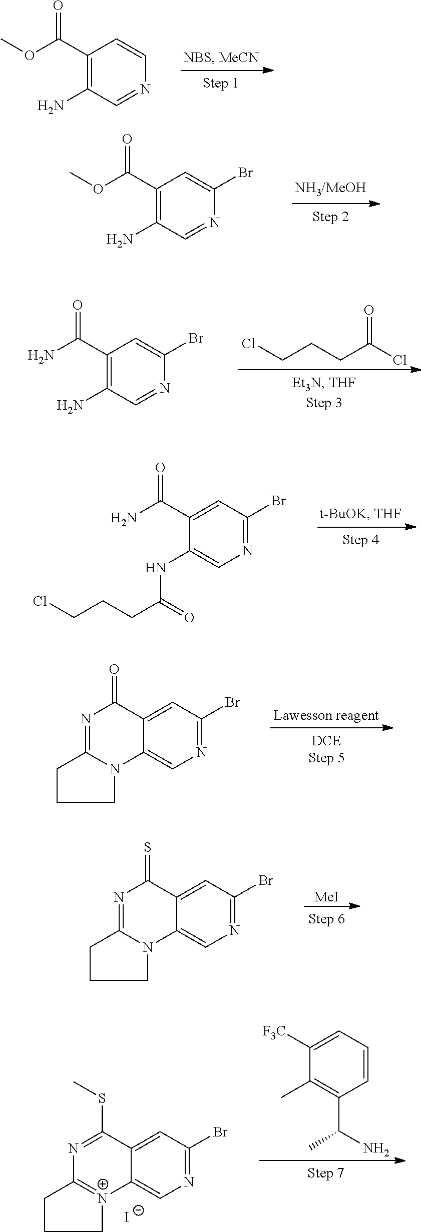

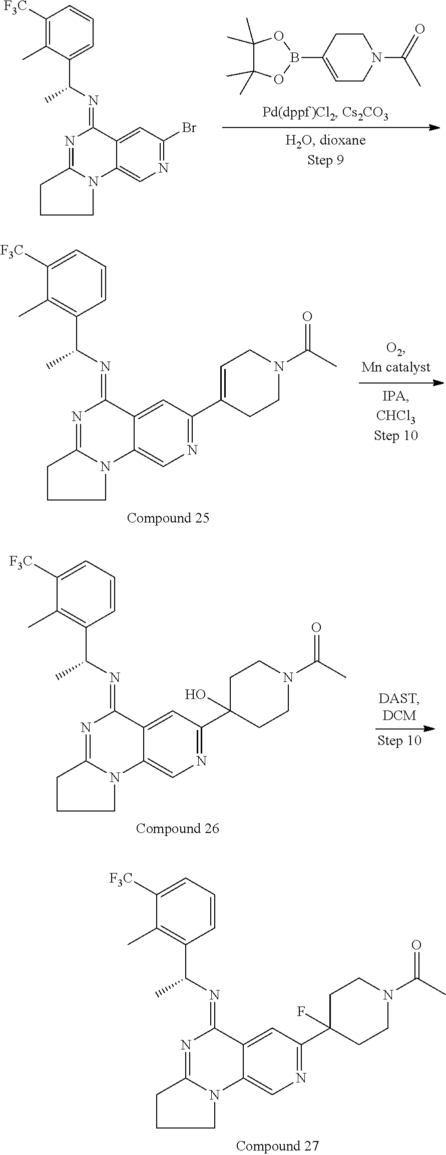

### Step 1. Synthesis of methyl 5-amino-2-bromoisonicotinate

To a solution of methyl 3-aminoisonicotinate (20 g, 132 mmol) in DMF (300 mL) was added NBS (24.6 g, 138 mmol) and stirred at 20° C. for 3 hrs. The mixture was quenched with water (500 mL) and filtered. The filter cake was washed with EtOAc (300 mL) and dried under vacuum to afford the title compound (22 g, 95.2 mmol, 72.4% yield) as a yellow solid.

¹H NMR (400 MHz, DMSO-d6) δ 8.06 (s, 1H), 7.60 (s, 1H), 6.80 (s, 2H), 3.84 (s, 3H).

### Step 2. Synthesis of 5-amino-2-bromoisonicotinamide

A mixture of methyl 5-amino-2-bromoisonicotinate (22 g, 95.2 mmol) and NH₃ solution (204 mL, 1.43 mol, 7 M in MeOH) was stirred at 20° C. for 18 hrs. The mixture was concentrated to dryness and the residue was titrated with DCM (100 mL) to afford the title compound (14 g, 64.8 mmol, 68.1% yield) as a yellow solid.

LC-MS m/e: 216, 218 (MH⁺).

### Step 3. Synthesis of 2-bromo-5-(4-chlorobutanamido)isonicotinamide

To a solution of 5-amino-2-bromoisonicotinamide (4.8 g, 22.2 mmol) and Et₃N (6.18 mL, 44.4 mmol) in THF (100 mL) was added 4-chlorobutanoyl chloride (3.76 mL, 33.3 mmol) at 0° C. and stirred at room temperature overnight. The resulting solution was poured into water (100 mL) and extracted with EtOAc (100 mL×2). The combined organic layers were washed with brine (100 mL), dried over Na₂SO₄, and filtered. The filtrate was concentrated under vacuum and purified by silica gel column chromatography (gradient: DCM to DCM:MeOH=50:1) to afford the title compound (5.5 g, 17.2 mmol, 77.2% yield) as yellow solid.

LC-MS m/e: 320.6, 322.6 (MH⁺).

### Step 4. Synthesis of 3-bromo-8,9-dihydropyrido[4,3-e]pyrrolo[1,2-a]pyrimidin-5(7H)-one

Potassium tert-butoxide (3.15 g, 28.1 mmol) was slowly added to a solution of 2-bromo-5-(4-chlorobutanamido)isonicotinamide (4.5 g, 14 mmol) in THF (70 mL) and the resulting mixture was stirred at room temperature overnight. The resulting reaction mixture was poured into ice water (250 mL) and extracted with EtOAc (250 mL×2). The combined organic layers were washed with brine (100 mL), dried over Na₂SO₄, and filtered. The filtrate was concentrated in vacuum and purified by silica gel column chromatography (gradient: DCM to DCM:MeOH=33:1) to afford the title compound (1.5 g, 5.64 mmol, 40.2% yield) as orange solid.

LC-MS m/e: 266, 268 (MH⁺).

### Step 5. Synthesis of 3-bromo-8,9-dihydropyrido[4,3-e]pyrrolo[1,2-a]pyrimidine-5(7H)-thione

To a solution of 3-bromo-8,9-dihydropyrido[4,3-e]pyrrolo[1,2-a]pyrimidin-5(7H)-one (1.5 g, 5.64 mmol) in toluene (20 mL) was added Lawesson's reagent (2.28 g, 5.64 mmol) and stirred at 110° C. for 2 hrs under N₂. The resulting solution was cooled to room temperature, poured into water (100 mL) and extracted with EtOAc (200 mL×2). The combined organic layers were washed with brine (100 mL), dried over Na₂SO₄, and filtered. The filtrate was concentrated under vacuum and purified by neutral Al₂O₃ column chromatography (gradient: PE to PE:DCM=3:1) to afford the title compound (300 mg, 0.513 mmol, 71% yield) as red solid.

LC-MS m/e: 282, 284 (MH⁺).

### Step 6. Synthesis of 3-bromo-5-(methylthio)-8,9-dihydro-7H-pyrido[4,3-e]pyrrolo[1,2-a]pyrimidin-10-ium-1-ide

A solution of 3-bromo-8,9-dihydropyrido[4,3-e]pyrrolo[1,2-a]pyrimidine-5(7H)-thione (170 mg, 0.602 mmol) in CH₃I (10 mL) was stirred at room temperature for 1 hr. The reaction mixture was concentrated under vacuum to give the title compound (170 mg, 0.57 mmol, 94.6% yield) as red solid.

LC-MS m/e: 296, 298 (MH⁺).

### Step 7. Synthesis of (R,Z)-3-bromo-N-(1-(2-methyl-3-(trifluoromethyl)phenyl)ethyl)-8,9-dihydropyrido[4,3-e]pyrrolo[1,2-a]pyrimidin-5(7H)-imine

To a solution of 3-bromo-5-(methylthio)-8,9-dihydro-7H-pyrido[4,3-e]pyrrolo[1,2-a]pyrimidin-10-ium-1-ide (150 mg, 0.503 mmol) in DMA (5 mL) was added (R)-1-(2-methyl-3-(trifluoromethyl)phenyl)ethan-1-amine (123 mg, 0.604 mmol) and stirred at room temperature for 1 hr. The resulting reaction mixture was poured into water (50 mL) and extracted with EtOAc (50 mL×2). The combined organic layers were washed with brine (50 mL), dried over Na₂SO₄, and filtered. The filtrate was concentrated under vacuum and purified by silica gel column chromatography (gradient: DCM to DCM:MeOH=50:1) to afford the title compound (170 mg, 0.377 mmol, 74.9% yield) as yellow solid.

LC-MS m/e: 451.3, 453.3 (MH⁺).

### Step 8. Synthesis of (R,Z)-1-(4-(5-((1-(2-methyl-3-(trifluoromethyl)phenyl)ethyl)imino)-5,7,8,9-tetrahydropyrido[4,3-e]pyrrolo[1,2-a]pyrimidin-3-yl)-3,6-dihydropyridin-1(2H)-yl)ethan-1-one (Compound 25)

To a solution of (R,Z)-3-bromo-N-(1-(2-methyl-3-(trifluoromethyl)phenyl)ethyl)-8,9-di-hydropyrido[4,3-e]pyrrolo[1,2-a]pyrimidin-5(7H)-imine (90 mg, 0.199 mmol) and 1-[4-(4,4,5,5-tetramethyl-1,3,2-dioxaborolan-2-yl)-1,2,3,6-tetrahydropyridin-1-yl]ethan-1-one (100 mg, 0.398 mmol) in dioxane (4 mL) and H₂O (2 mL) was added Cs₂CO₃ (195 mg, 0.597 mmol) and Pd(dppf)Cl₂ (146 mg, 0.199 mmol) and stirred at 100° C. under N₂ for 2 hrs. The resulting solution was cooled to room temperature, poured into water (50 mL) and extracted with EtOAc (50 mL×2). The combined organic layers were washed with brine (50 mL), dried over Na₂SO₄, and filtered. The filtrate was concentrated under vacuum and purified by neutral Al₂O₃ column chromatography (gradient, PE to PE:DCM:MeOH=16:4:1) to afford the crude product, which was purified by prep-HPLC (SilaSep™ C18 silica flash cartridge, 0%-40% MeCN in H₂O with 0.1% FA) to afford a formate salt of the title compound (37.7 mg) as light-yellow solid

¹H NMR (400 MHz, CDCl₃) δ 8.91 (s, 1H), 8.54 (d, J=15.3 Hz, 1H), 8.43 (s, 1H), 8.02 (d, J=7.9 Hz, 1H), 7.49 (d, J=7.7 Hz, 1H), 7.29 (d, J=7.9 Hz, 1H), 6.78 (d, J=30.5 Hz, 1H), 5.83 (q, J=6.6 Hz, 1H), 4.33 (s, 1H), 4.22 (s, 1H), 4.20-4.09 (m, 2H), 3.84 (t, J=5.6 Hz, 1H), 3.69 (t, J=5.6 Hz, 1H), 3.11-2.89 (m, 2H), 2.75 (d, J=27.2 Hz, 2H), 2.57 (s, 3H), 2.41-2.35 (m, 2H), 2.17 (d, J=12.6 Hz, 3H), 1.62-1.46 (m, 3H).

LC-MS m/e: 496 (MH⁺).

### Step 9. Synthesis of (R,Z)-1-(4-hydroxy-4-(5-((1-(2-methyl-3-(trifluoromethyl)phenyl)ethyl)-imino)-5,7,8,9-tetrahydropyrido[4,3-e]pyrrolo[1,2-a]pyrimidin-3-yl)piperidin-1-yl)ethan-1-one (Compound 26)

To a solution of (R,Z)-1-(4-(5-((1-(2-methyl-3-(trifluoromethyl)phenyl)ethyl)imino)-5,7,8,9-tetrahydropyrido[4,3-e]pyrrolo[1,2-a]pyrimidin-3-yl)-3,6-dihydropyridin-1(2H)-yl)ethan-1-one (460 mg, 0.928 mmol) and Mn(dpm)₃ (337 mg, 0.557 mmol) in DCM (18 mL) and i-PrOH (2 mL) was added PhSiH₃ (201 mg, 1.86 mmol) at 0° C. under O₂. After stirring for 1.5 hrs at 0° C., the reaction mixture was poured into water (100 mL) and extracted with DCM (100 mL×2). The combined organic layers were washed with brine (100 mL), dried over Na₂SO₄, and filtered. The filtrate was concentrated in vacuum and purified by neutral Al₂O₃ column chromatography (gradient, PE to PE:DCM:MeOH=16:4:1) to afford the title compound (320 mg, 0.623 mmol, 67.1% yield) as a light-yellow solid.

¹H NMR (400 MHz, CDCl₃) δ 9.65 (d, J=16.6 Hz, 1H), 8.86 (d, J=4.8 Hz, 1H), 8.10-7.96 (m, 1H), 7.53 (d, J=7.5 Hz, 1H), 7.34-7.27 (m, 1H), 6.02-5.89 (m, 1H), 4.66-4.44 (m, 3H), 3.69 (t, J=7.9 Hz, 2H), 3.43-3.10 (m, 4H), 2.63 (s, 3H), 2.60-2.54 (m, 2H), 2.26-1.95 (m, 7H), 1.84 (d, J=7.0 Hz, 3H).

LC-MS m/e: 514 (MH⁺).

### Step 10. Synthesis of (R,Z)-1-(4-fluoro-4-(5-((1-(2-methyl-3-(trifluoromethyl)phenyl)ethyl)-imino)-5,7,8,9-tetrahydropyrido[4,3-e]pyrrolo[1,2-a]pyrimidin-3-yl)piperidin-1-yl)ethan-1-one (Compound 27)

To a solution of (R,Z)-1-(4-hydroxy-4-(5-((1-(2-methyl-3-(trifluoromethyl)phenyl)ethyl)-imino)-5,7,8,9-tetrahydropyrido[4,3-e]pyrrolo[1,2-a]pyrimidin-3-yl)piperidin-1-yl)ethan-1-one (260 mg, 0.506 mmol) in DCM (10 mL) was added DAST (0.334 mL, 2.53 mmol) at −20° C. and stirred under N₂ for 2 hrs. The resulting solution was poured into a cold saturated NaHCO₃ solution (50 mL) and extracted with DCM (50 mL×2). The combined organic layers were washed with brine (50 mL), dried over Na₂SO₄, and filtered. The filtrate was concentrated under vacuum and purified by prep-HPLC to afford the title compound (92.7 mg, 0.181 mmol, 35.7% yield) as a light-yellow solid. The compound was converted to its formate salt.

¹H NMR (400 MHz, CDCl₃) δ 9.23 (s, 1H), 8.79 (s, 1H), 8.45 (s, 1H), 8.00 (d, J=7.7 Hz, 1H), 7.52 (d, J=7.0 Hz, 1H), 7.32-7.28 (m, 1H), 5.93-5.85 (m, 1H), 4.64 (d, J=12.1 Hz, 1H), 4.56-4.41 (m, 2H), 3.83 (d, J=13.8 Hz, 1H), 3.56 (t, J=12.9 Hz, 1H), 3.37-3.11 (m, 2H), 3.03 (t, J=12.9 Hz, 1H), 2.60 (s, 3H), 2.56-2.47 (m, 2H), 2.34-2.16 (m, 2H), 2.14 (d, J=1.8 Hz, 3H), 2.11-1.99 (m, 2H), 1.75 (d, J=6.7 Hz, 3H).

LC-MS m/e: 516 (MH⁺).

Following a similar procedure, the following three compounds (Compound 28-30) were prepared.

Compound 28: ¹H NMR (400 MHz, CDCl₃) δ 9.63 (d, J=8.8 Hz, 1H), 8.84 (d, J=3.2 Hz, 1H), 8.54 (s, 1H), 7.96 (t, J=7.0 Hz, 1H), 7.53-7.47 (m, 1H), 7.26-7.22 (m, 1H), 6.89 (t, J=55.0 Hz, 1H), 6.11-6.08 (m, 1H), 4.62-4.49 (m, 3H), 3.68 (d, J=6.8 Hz, 2H), 3.42-3.26 (m, 2H), 3.13 (t, J=12.2 Hz, 1H), 2.62-2.55 (m, 2H), 2.25-1.99 (m, 5H), 1.85 (d, J=7.2 Hz, 3H), 1.77-1.61 (m, 2H); LC-MS m/e: 500.2 (MH⁺).

Formate salt of Compound 29: ¹H NMR (400 MHz, CDCl₃) δ 8.98 (s, 1H), 8.65 (s, 1H), 8.44 (s, 1H), 7.96 (t, J=7.4 Hz, 1H), 7.46 (t, J=6.8 Hz, 1H), 7.25-7.19 (m, 1H), 6.90 (t, J=55.1 Hz, 1H), 5.98 (p, J=6.8 Hz, 1H), 4.66 (d, J=10.3 Hz, 1H), 4.44-4.23 (m, 2H), 3.83 (d, J=11.2 Hz, 1H), 3.57 (t, J=13.1 Hz, 1H), 3.29-3.09 (m, 2H), 3.09-2.99 (m, 1H), 2.60-2.42 (m, 2H), 2.38-2.18 (m, 2H), 2.15 (s, 3H), 2.13-2.07 (m, 1H), 2.06-1.98 (m, 1H), 1.70 (d, J=6.8 Hz, 3H); LC-MS m/e: 502 (MH⁺).

Formate salt of Compound 30: ¹H NMR (400 MHz, CDCl₃) δ 9.16 (s, 1H), 8.76 (s, 1H), 8.43 (d, J=3.2 Hz, 1H), 8.07 (d, J=7.6 Hz, 1H), 7.50 (d, J=7.6 Hz, 1H), 7.33-7.29 (m, 1H), 5.88-5.76 (m, 1H), 4.64 (d, J=8.4 Hz, 2H), 4.49-4.45 (m, 2H), 3.85-3.81 (m, 1H), 3.59-3.53 (m, 1H), 3.33-3.21 (m, 2H), 3.03 (t, J=12.8 Hz, 1H), 2.71 (s, 3H), 2.57-2.51 (m, 2H), 2.31-2.18 (m, 1H), 2.15 (d, J=2.0 Hz, 3H), 2.12-2.01 (m, 2H), 1.72 (d, J=6.8 Hz, 3H); LC-MS m/e: 473.1 (MH⁺).

### Example 31

### Synthesis of (R,Z)-3-(1-(difluoromethyl)cyclopropyl)-N-(1-(2-methyl-3-(trifluoromethyl)phenyl)ethyl)-8,9-dihydropyrido[4,3-e]pyrrolo[1,2-a]pyrimidin-5(7H)-imine (Compound 31)

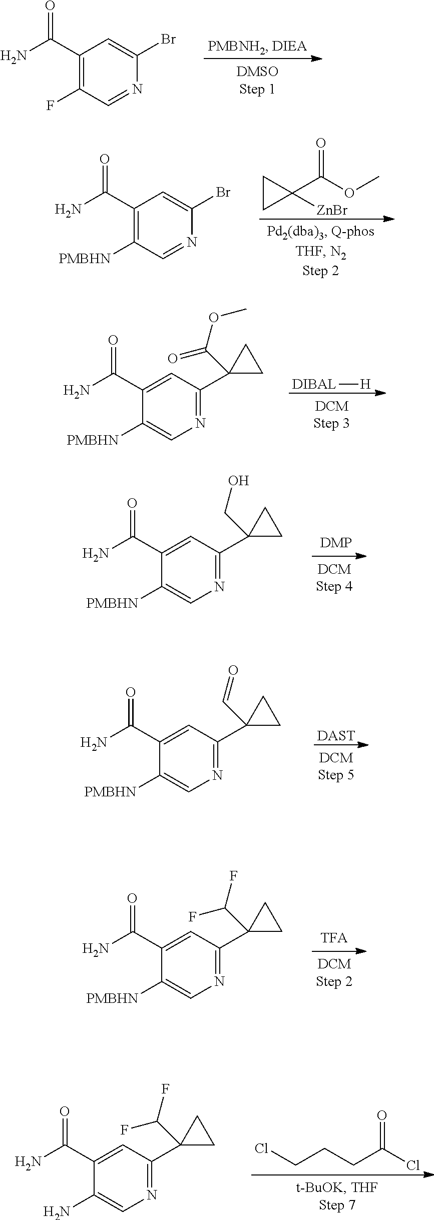

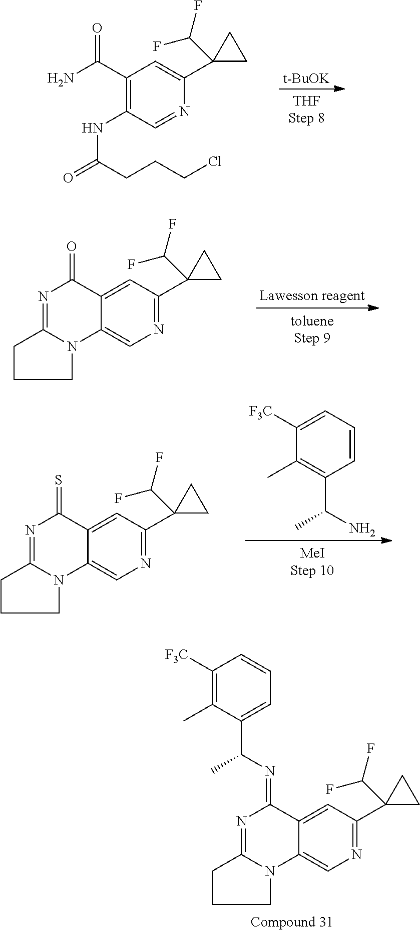

### Step 1. Synthesis of 2-bromo-5-((4-methoxybenzyl)amino)isonicotinamide

To a solution of 2-bromo-5-fluoroisonicotinamide (3.2 g, 14.6 mmol) and PMBNH₂ (3 g, 21.9 mmol) in DMSO (30 mL) was added DIEA (5.1 mL, 29.3 mmol). The reaction mixture was stirred at 90° C. overnight. The resulting mixture was cooled to room temperature, poured into water (150 mL), and extracted with EtOAc (150 mL×3). The combined organic layers were washed with brine (150 mL×3), dried over anhydrous Na₂SO₄, and filtered. The filtrate was concentrated to dryness. The residue was triturated with a mixture of PE (150 mL) and DCM (50 mL). The solid obtained was dried under vacuum to afford the title compound (3.79 g, 11.32 mmol, 77.5% yield) as yellow solid.

LC-MS m/e: 336, 338 (MH⁺).

### Step 2. Synthesis of methyl 1-(4-carbamoyl-5-((4-methoxybenzyl)amino)pyridin-2-yl)cyclopropane-1-carboxylate

To a solution of 2-bromo-5-((4-methoxybenzyl)amino)isonicotinamide (3.2 g, 9.52 mmol), Q-Phos (1.35 g, 1.9 mmol), and Pd₂(dba)₃ (872 mg, 0.952 mmol) in THF (120 mL) was added (1-(methoxycarbonyl)cyclopropyl)zinc(II) bromide (102 mL, 76.5 mmol, 0.75 M in THF) at room temperature and stirred for 5 hrs. The mixture was quenched with water (200 mL) and extracted with EtOAc (400 mL×2). The combined organic layers were dried over anhydrous Na₂SO₄ and filtered. The filtrate was concentrated under reduced pressure and purified by silica gel chromatography (gradient, PE to PE:EtOAc=2:1) to afford the tittle compound (2 g, 5.63 mmol, 59.2% yield) as a red solid.

LC-MS m/e: 356.0 (MH⁺).

### Step 3. Synthesis of 2-(1-(hydroxymethyl)cyclopropyl)-5-((4-methoxybenzyl)amino)-isonicotinamide

To a solution of methyl 1-(4-carbamoyl-5-((4-methoxybenzyl)amino)pyridin-2-yl)-cyclopropane-1-carboxylate (2 g, 5.63 mmol) in THF (50 mL) was added DIBAL-H (22.5 mL, 22.5 mmol, 1 M in hexane) at 0° C. The mixture was stirred at room temperature for 2 hrs, quenched with water (50 mL), and extracted with EtOAc (100 mL×2). The combined organic layers were dried over anhydrous Na₂SO₄ and filtered. The filtrate was concentrated under reduced pressure and purified by silica gel chromatography (gradient, DCM to DCM:MeOH=20:1) to afford the title compound (1.3 g, 3.98 mmol, 70.6% yield) as a brown solid.

LC-MS m/e: 328 (MH⁺).

### Step 4. Synthesis of 2-(1-formylcyclopropyl)-5-((4-methoxybenzyl)amino)isonicotinamide

To a solution of 2-(1-(hydroxymethyl)cyclopropyl)-5-((4-methoxybenzyl)amino)-iso-nicotinamide (1.3 g, 3.98 mmol) in DCM (50 mL) was added Dess-Martin periodinane (4.22 g, 9.95 mmol) at 0° C. and stirred at room temperature for 2 hrs. The mixture was quenched with saturated NaHCO₃ solution (50 mL), and the aqueous layer was extracted with DCM (50 mL×2). The combined organic layers were dried over anhydrous Na₂SO₄ and filtered. The filtrate was concentrated under reduced pressure and purified by silica gel chromatography (gradient, DCM to DCM:MeOH=20:1) to afford the title compound (600 mg, 1.85 mmol, 46.4% yield) as a yellow solid.

LC-MS m/e: 326 (MH⁺).

### Step 5. Synthesis of 2-(1-(difluoromethyl)cyclopropyl)-5-((4-methoxybenzyl)amino)-isonicotinamide

To a solution of 2-(1-formylcyclopropyl)-5-((4-methoxybenzyl)amino)isonicotinamide (600 mg, 1.85 mmol) in DCM (20 mL) was added DAST (1.49 g, 9.25 mmol) at −10° C. and stirred at 0° C. for 2 hrs. The mixture was quenched with saturated NaHCO₃ solution (50 mL) and extracted with DCM (50 mL×2). The combined organic layers were dried over anhydrous Na₂SO₄ and filtered. The filtrate was concentrated under reduced pressure and purified by silica gel chromatography (gradient, DCM to DCM:MeOH=20:1) to afford the title compound (300 mg, 0.865 mmol, 46.7% yield) as a yellow solid.

LC-MS m/e: 348 (MH⁺).

### Step 6. Synthesis of 5-amino-2-(1-(difluoromethyl)cyclopropyl)isonicotinamide

To a solution of 2-(1-(difluoromethyl)cyclopropyl)-5-((4-methoxybenzyl)amino)-isonicotinamide (300 mg, 0.865 mmol) in DCM (4 mL) was added TFA (2 mL) at 0° C. and stirred at room temperature for 2 hrs. The mixture was concentrated to afford the crude tittle compound (196 mg, 0.865 mmol, 100% yield) as a brown solid. The crude product was used directly in the next step without further purification.

LC-MS m/e: 228 (MH⁺).

### Step 7. Synthesis of 5-(4-chlorobutanamido)-2-(1-(difluoromethyl)cyclopropyl)-isonicotinamide

To a solution of 5-amino-2-(1-(difluoromethyl)cyclopropyl)isonicotinamide (196 mg, 0.865 mmol) and TEA (0.73 mL, 5.3 mmol) in THF (10 mL) was added 4-chlorobutanoyl chloride (0.3 mL, 2.68 mmol) at 0° C. The reaction mixture was stirred at room temperature for 2 hrs. The mixture was diluted with water (50 mL) and extracted with DCM (100 mL×2). The combined organic layers were dried over anhydrous Na₂SO₄ and filtered. The filtrate was concentrated under reduced pressure and purified by neutral Al₂O₃ column chromatography (gradient, PE to PE:DCM:MeOH=40:2:1) to afford the title compound (128 mg, 0.388 mmol, 44.8% yield) as a yellow solid.

LC-MS m/e: 332 (MH⁺).

### Step 8. Synthesis of 3-(1-(difluoromethyl)cyclopropyl)-8,9-dihydropyrido[4,3-e]pyrrolo[1,2-a]pyrimidin-5(7H)-one

To a solution of 5-(4-chlorobutanamido)-2-(1-(difluoromethyl)cyclopropyl)-isonicotin-amide (200 mg, 0.604 mmol) in THF (10 mL) was added potassium tert-butoxide (1.2 mL, 1.2 mmol, 1 M in THF) and stirred at room temperature for 1 hr. The mixture was concentrated and purified by neutral Al₂O₃ column chromatography (gradient, PE to PE:DCM:MeOH=16:3:1) to afford the title compound (70 mg, 0.252 mmol, 41.7% yield) as a yellow solid.

LC-MS m/e: 278 (MH⁺).

### Step 9. Synthesis of 3-(1-(difluoromethyl)cyclopropyl)-8,9-dihydropyrido[4,3-e]pyrrolo[1,2-a]pyrimidine-5(7H)-thione

A solution of 3-(1-(difluoromethyl)cyclopropyl)-8,9-dihydropyrido[4,3-e]pyrrolo[1,2-a]pyrimidin-5(7H)-one (70 mg, 0.252 mmol) and Lawesson's reagent (102 mg, 0.252 mmol) in toluene (5 mL) was stirred at 80° C. overnight. The reaction was cooled to room temperature and concentrated to dryness. The residue was dissolved in 1N HCl solution (20 mL) and washed with EtOAc (50 mL). Then the aqueous layer was treated with NaHCO₃ to ˜pH 7 and extracted with DCM (40 mL×3). The combined organic layers were dried over anhydrous Na₂SO₄, filtered, and concentrated to afford the title compound (70 mg, 0.239 mmol, 94.5% yield) as a brown solid.

LC-MS m/e: 294 (MH⁺).

### Step 10. Synthesis of (R,Z)-3-(1-(difluoromethyl)cyclopropyl)-N-(1-(2-methyl-3-(trifluoromethyl)phenyl)ethyl)-8,9-dihydropyrido[4,3-e]pyrrolo[1,2-a]pyrimidin-5(7H)-imine (Compound 31)

To a solution of 3-(1-(difluoromethyl)cyclopropyl)-8,9-dihydropyrido[4,3-e]pyrrolo[1,2-a]pyrimidine-5(7H)-thione (70 mg, 0.239 mmol) in iodomethane (2 mL) was added (R)-1-(2-methyl-3-(trifluoromethyl)phenyl)ethan-1-amine (242 mg, 1.19 mmol) at room temperature. The reaction was stirred at room temperature for 10 mins, concentrated, and purified by pre-HPLC to afford the title compound (16 mg, 0.035 mmol, 14.5% yield) as a yellow solid. The compound was converted to its formate salt.

¹HNMR (400 MHz, DMSO-d₆) δ 8.55 (s, 1H), 8.19 (s, 1H), 8.10 (s, 1H), 8.01 (d, J=7.8 Hz, 1H), 7.52 (d, J=7.7 Hz, 1H), 7.37 (d, J=7.9 Hz, 1H), 6.34 (t, J=56.0 Hz, 1H), 5.73 (q, J=6.6 Hz, 1H), 4.08 (m, 2H), 3.00-2.78 (m, 2H), 2.50 (s, 3H), 2.26-2.12 (m, 2H), 1.37 (d, J=6.6 Hz, 3H), 1.24 (s, 4H). LC-MS m/e: 463 (MH⁺).

### Examples 32-42

### Synthesis of. (R,Z)-1-(4-(5-((1-(2-methyl-3-(trifluoromethyl)phenyl)ethyl)imino)-8,9-dihydro-5H-oxazolo[3,2-a]pyrido[4,3-e]pyrimidin-3-yl)-3,6-dihydropyridin-1(2H)-yl)ethan-1-one (Compound 32) and

### (R,Z)-1-(4-fluoro-4-(5-((1-(2-methyl-3-(trifluoromethyl)phenyl)ethyl)imino)-8,9-dihydro-5H-oxazolo[3,2-a]pyrido[4,3-e]pyrimidin-3-yl)piperidin-1-yl)ethan-1-one (Compound 33)

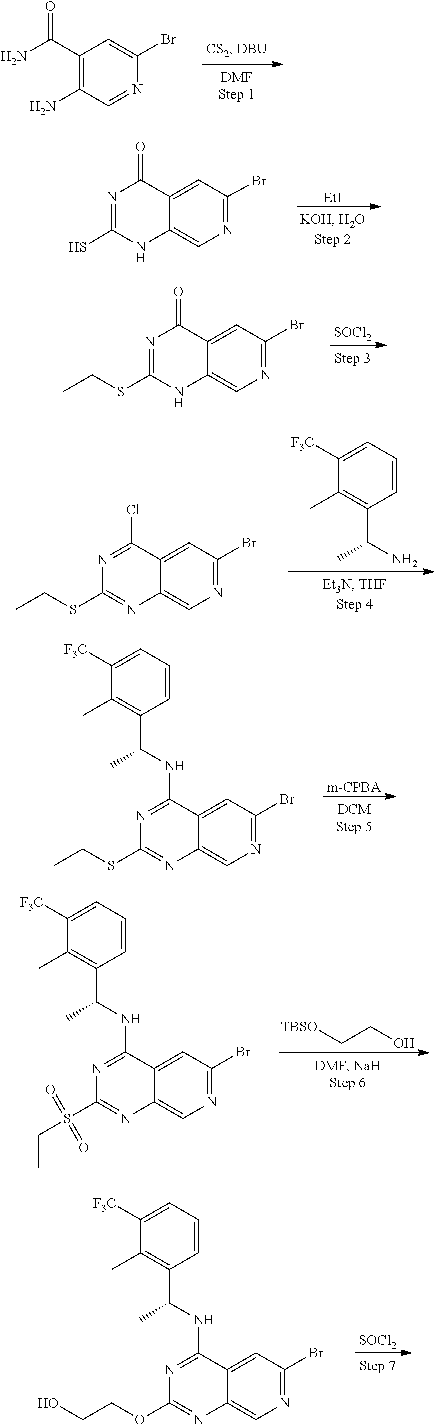

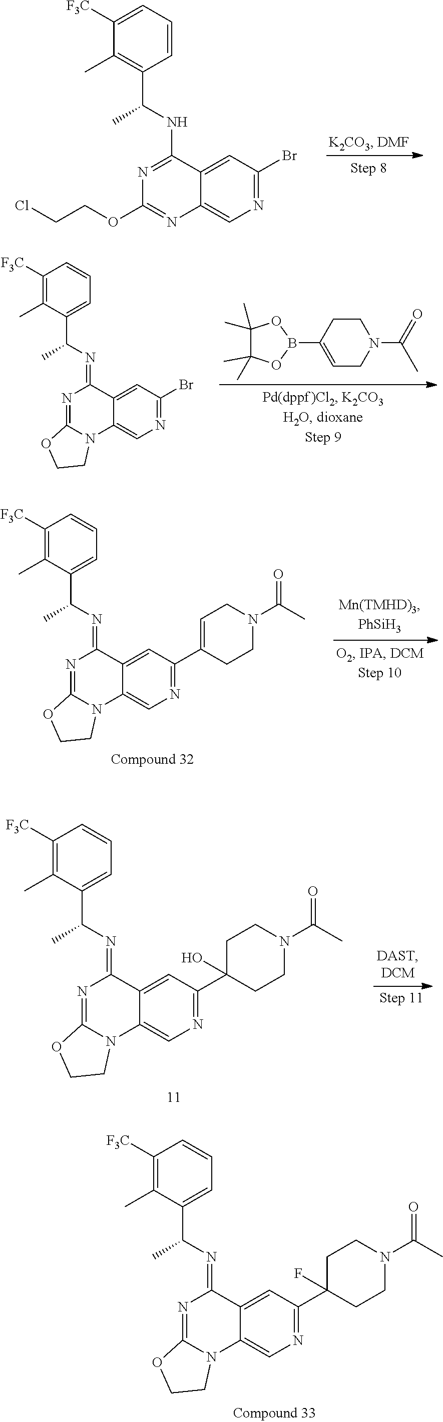

### Step 1. Synthesis of 6-bromo-2-mercaptopyrido[3,4-d]pyrimidin-4(1H)-one

To a solution of 5-amino-2-bromoisonicotinamide (3 g, 13.89 mmol) in DMF (50 mL) was added CS₂ (5.3 g, 69.4 mmol) and DBU (17.4 g, 69.4 mmol). The mixture was stirred at 60° C. for 12 hrs. The reaction mixture was cooled to room temperature, purified by reverse phase column chromatography (gradient, 100% H₂O to H₂O:ACN=60%) to afford the title compound (3 g, 11.62 mmol, 83.7% yield) as a brown solid.

LCMS m/e: 258.1, 260.1 (MH⁺).

### Step 2. Synthesis of 6-bromo-2-(ethylthio)pyrido[3,4-d]pyrimidin-4(1H)-one

To 6-bromo-2-mercaptopyrido[3,4-d]pyrimidin-4(1H)-one (1.5 g, 5.81 mmol) in EtOH (55 mL) was added KOH (2 M, 5.2 mL) and EtI (1.8 g, 11.62 mmol). After stirring at room temperature for 2 hrs, the reaction mixture was diluted with water (100 mL) and extracted with EtOAc (100 mL×3). The combined organic layers were washed with brine (100 mL), dried over Na₂SO₄, and filtered. The filtrate was concentrated under reduced pressure to afford the title compound (1.5 g, 5.2 mmol, 88.2% yield) as a white solid.

LCMS m/e: 286.1, 288.1 (MH⁺).

### Step 3. Synthesis of 6-bromo-4-chloro-2-(ethylthio)pyrido[3,4-d]pyrimidine

A mixture of 6-bromo-2-(ethylthio)pyrido[3,4-d]pyrimidin-4(1H)-one (700 mg, 2.45 mmol) in SOCl₂ (10 mL) was stirred at 100° C. for 2 hrs. The reaction mixture was cooled to room temperature and distilled-off the excess of SOCl₂. The residue was basified with saturated Na₂CO₃ solution (100 mL) and extracted with EtOAc (80 mL×3). The combined organic layers were washed with brine (80 mL), dried over Na₂SO₄, and filtered. The filtrate was concentrated under reduced pressure to afford the title compound (700 mg, 2.3 mmol, 93.9% yield) as a white solid.

LCMS m/e: 304.6, 306.6 (MH⁺).

### Step 4. Synthesis of (R)-6-bromo-2-(ethylthio)-N-(1-(2-methyl-3-(trifluoromethyl) phenyl)ethyl)-pyrido[3,4-d]pyrimidin-4-amine

To a solution of 6-bromo-4-chloro-2-(ethylthio)pyrido[3,4-d]pyrimidine (700 mg, 2.3 mmol) in THF (20 mL) was added (R)-1-(2-methyl-3-(trifluoromethyl)phenyl)ethan-1-amine (467 mg, 2.3 mmol) and Et₃N (466 mg, 4.6 mmol). The mixture was stirred at room temperature for 0.5 hrs. The resulting mixture was diluted with water (100 mL) and extracted with EtOAc (100 mL×3). The combined organic layers were washed with brine (100 mL), dried over Na₂SO₄, and filtered. The filtrate was concentrated under reduced pressure and purified by silica gel column chromatography (gradient, PE to PE:EA=3:1) to afford the title compound (800 mg, 1.7 mmol, 74.0% yield) as a white solid.

LCMS m/e: 471.3, 473.3 (MH⁺).

### Step 5. Synthesis of (R)-6-bromo-2-(ethylsulfonyl)-N-(1-(2-methyl-3-(trifluoromethyl)phenyl)-ethyl)pyrido[3,4-d]pyrimidin-4-amine

To a solution of (R)-6-bromo-2-(ethylthio)-N-(1-(2-methyl-3-(trifluoromethyl)phenyl)-ethyl)pyrido[3,4-d]pyrimidin-4-amine (800 mg, 1.7 mmol) in DCM (25 mL) was added m-CPBA (1.03 g, 5.95 mmol) and stirred at room temperature for 2 hrs. The reaction mixture was diluted with a saturated NaHCO₃ solution (60 mL) and extracted with EtOAc (60 mL×3). The combined organic layers were washed with brine (60 mL), dried over anhydrous Na₂SO₄, and filtered. The filtrate was concentrated under reduced pressure and purified by silica gel column chromatography (gradient, PE to PE:EA=2:1) to afford the title compound (750 mg, 1.49 mmol, 87.6% yield) as a white solid.

LCMS m/e: 503.4, 505.4 (MH⁺).

### Step 6. Synthesis of (R)-2-((6-bromo-4-((1-(2-methyl-3-(trifluoromethyl)phenyl)ethyl)-amino)pyrido[3,4-d]pyrimidin-2-yl)oxy)ethan-1-ol

To a solution of (R)-6-bromo-2-(ethylsulfonyl)-N-(1-(2-methyl-3-(trifluoromethyl)-phenyl)ethyl)pyrido[3,4-d]pyrimidin-4-amine (750 mg, 1.49 mmol) in DMF (20 mL) was added NaH (119 mg, 2.98 mmol, 60% suspended in mineral oil) and 2-((tert-butyldimethylsilyl)oxy)-ethan-1-ol (526 mg, 2.98 mmol). The reaction mixture was stirred at 80° C. for 2 hrs. The resulting mixture was cooled to room temperature, quenched with ice water (60 mL), and extracted with EtOAc (60 mL×3). The combined organic layers were washed with brine (60 mL), dried over anhydrous Na₂SO₄, and filtered. The filtrate was concentrated under reduced pressure and purified by silica gel column chromatography (gradient, PE to PE:EA=1:1) to afford the title compound (500 mg, 1.1 mmol, 71.2% yield) as a white solid.

LCMS m/e: 471.3, 473.1 (MH⁺).

### Step 7. Synthesis of (R)-6-bromo-2-(2-chloroethoxy)-N-(1-(2-methyl-3-(trifluoromethyl)-phenyl)ethyl)pyrido[3,4-d]pyrimidin-4-amine

To a solution of (R)-2-((6-bromo-4-((1-(2-methyl-3-(trifluoromethyl)phenyl)ethyl)-amino)pyrido[3,4-d]pyrimidin-2-yl)oxy)ethan-1-ol (500 mg, 1.1 mmol) in CHCl₃ (20 mL) was added SOCl₂ (1.32 g, 11 mmol). The mixture was stirred at 70° C. for 2 hrs. The reaction mixture was cooled to room temperature and concentrated under reduced pressure to afford the title compound (400 mg, 0.82 mmol, 74.3% yield) as a colorless oil.

LCMS m/e: 489.7, 491.7 (MH⁺).

### Step 8. Synthesis of (R,Z)-3-bromo-N-(1-(2-methyl-3-(trifluoromethyl)phenyl)ethyl)-8,9-dihydro-5H-oxazolo[3,2-a]pyrido[4,3-e]pyrimidin-5-imine

To a solution of (R)-6-bromo-2-(2-chloroethoxy)-N-(1-(2-methyl-3-(trifluoromethyl)-phenyl)ethyl)pyrido[3,4-d]pyrimidin-4-amine (400 mg, 0.82 mmol) in DMF (20 mL) was added K₂CO₃ (1.13 g, 8.2 mmol) and stirred at 100° C. for 4 hrs. The mixture was cooled to room temperature, diluted with water (60 mL), and extracted with EtOAc (60 mL×3). The combined organic layers were washed with brine (60 mL), dried over anhydrous Na₂SO₄, and filtered. The filtrate was concentrated under reduced pressure and purified by silica gel column chromatography (gradient, DCM to DCM:MeOH=30:1) to afford the title compound (300 mg, 0.66 mmol, 80.7% yield) as a white solid.

LCMS m/e: 453.2, 455.2 (MH⁺).

### Step 9. Synthesis of (R,Z)-1-(4-(5-((1-(2-methyl-3-(trifluoromethyl)phenyl)ethyl)imino)-8,9-di-hydro-5H-oxazolo[3,2-a]pyrido[4,3-e]pyrimidin-3-yl)-3,6-dihydropyridin-1(2H)-yl)ethan-1-one (Compound 32)

To a solution of (R,Z)-3-bromo-N-(1-(2-methyl-3-(trifluoromethyl)phenyl)ethyl)-8,9-dihydro-5H-oxazolo[3,2-a]pyrido[4,3-e]pyrimidin-5-imine (300 mg, 0.66 mmol) in dioxane (25 mL) and water (5 mL) was added 1-(4-(4,4,5,5-tetramethyl-1,3,2-dioxaborolan-2-yl)-3,6-dihydropyridin-1(2H)-yl)ethan-1-one (166 mg, 0.66 mmol), Pd(dppf)Cl₂ (51 mg, 0.07 mmol) and K₂CO₃ (268 mg, 1.98 mmol). The reaction mixture was stirred at 120° C. for 2 hrs. The resulting mixture was cooled to room temperature, diluted with water (60 mL), and extracted with EtOAc (60 mL×3). The combined organic layers were washed with brine (60 mL), dried over anhydrous Na₂SO₄, and filtered. The filtrate was concentrated under reduced pressure and purified by silica gel column chromatography (gradient, DCM to DCM:MeOH=15:1) to afford the title compound (200 mg, 0.4 mmol, 60.9% yield) as a brown solid.

¹H NMR (400 MHz, DMSO-d₆) δ 8.50 (s, 1H), 8.05 (d, J=9.0 Hz, 1H), 8.00 (d, J=7.9 Hz, 1H), 7.52 (d, J=7.7 Hz, 1H), 7.37 (t, J=7.8 Hz, 1H), 6.67 (d, J=17.5 Hz, 1H), 5.57 (q, J=6.5 Hz, 1H), 4.73 (t, J=8.6 Hz, 2H), 4.25 (dd, J=14.7, 7.9 Hz, 2H), 4.18 (d, J=19.7 Hz, 2H), 3.70-3.61 (m, 2H), 2.68-2.63 (m, 1H), 2.59-2.52 (m, 1H), 2.47 (s, 3H), 2.07 (d, J=15.1 Hz, 3H), 1.35 (d, J=6.5 Hz, 3H).

LCMS m/e: 498.6 (MH⁺).

### Step 10. Synthesis of (R,Z)-1-(4-hydroxy-4-(5-((1-(2-methyl-3-(trifluoromethyl)phenyl)ethyl)-imino)-8,9-dihydro-5H-oxazolo[3,2-a]pyrido[4,3-e]pyrimidin-3-yl)piperidin-1-yl)ethan-1-one

To a solution of (R,Z)-1-(4-(5-((1-(2-methyl-3-(trifluoromethyl)phenyl)ethyl)imino)-8,9-dihydro-5H-oxazolo[3,2-a]pyrido[4,3-e]pyrimidin-3-yl)-3,6-dihydropyridin-1(2H)-yl)ethan-1-one (200 mg, 0.4 mmol) and Mn(TMHD)₃ (73 mg, 0.12 mmol) in i-PrOH (18 mL) and DCM (2 mL) was added PhSiH₃ (56 mg, 0.52 mmol) at 0° C. under O₂. The mixture was stirred at room temperature for 2 hrs, diluted with water (60 mL), and extracted with EtOAc (60 mL×3). The combined organic layers were washed with brine (60 mL), dried over anhydrous Na₂SO₄, and filtered. The filtrate was concentrated under reduced pressure and purified by silica gel column chromatography (gradient, DCM to DCM:MeOH=10:1) to afford the title compound (150 mg, 0.29 mmol, 72.7% yield) as a brown solid.

LCMS m/e: 516.5 (MH⁺).

### Step 11. Synthesis of (R,Z)-1-(4-fluoro-4-(5-((1-(2-methyl-3-(trifluoromethyl)phenyl)ethyl)-imino)-8,9-dihydro-5H-oxazolo[3,2-a]pyrido[4,3-e]pyrimidin-3-yl)piperidin-1-yl)ethan-1-one (Compound 33)

To a solution of (R,Z)-1-(4-hydroxy-4-(5-((1-(2-methyl-3-(trifluoromethyl)phenyl)ethyl)-imino)-8,9-dihydro-5H-oxazolo[3,2-a]pyrido[4,3-e]pyrimidin-3-yl)piperidin-1-yl)ethan-1-one (150 mg, 0.29 mmol) in DCM (10 mL) was added DAST (234 mg, 1.45 mmol) at −20° C. under N₂ and stirred at −20° C. for 2 hrs. The reaction mixture was basified with saturated NaHCO₃ solution (60 mL) and extracted with EtOAc (60 mL×3). The combined organic layers were washed with brine (60 mL), dried over anhydrous Na₂SO₄, and filtered. The filtrate was concentrated under reduced pressure and purified by prep-HPLC to afford the title compound (43.3 mg, 0.084 mmol, 28.9% yield) as a white solid.

¹H NMR (400 MHz, DMSO-d₆) δ 8.51 (s, 1H), 8.13 (s, 1H), 7.97 (d, J=7.7 Hz, 1H), 7.52 (d, J=7.5 Hz, 1H), 7.38 (t, J=7.4 Hz, 1H), 5.56 (dd, J=12.2, 6.0 Hz, 1H), 4.73 (t, J=8.0 Hz, 2H), 4.46-4.38 (m, 1H), 4.25 (t, J=13.9, 7.4 Hz, 2H), 3.91-3.83 (m, 1H), 3.43-3.37 (m, 1H), 2.92-2.86 (m, 1H), 2.47 (s, 3H), 2.34-2.26 (m, 1H), 2.22-2.14 (m, 1H), 2.07 (s, 3H), 1.98-1.92 (m, 2H), 1.34 (d, J=6.3 Hz, 3H); LCMS m/e: 518.6 (MH⁺).

Following a similar procedure described above, the following compounds 34-41 were synthesized.

Compound 34: ¹H NMR (400 MHz, DMSO-d₆) δ 8.49 (s, 1H), 8.02 (t, J=9.3 Hz, 2H), 7.60 (d, J=7.7 Hz, 1H), 7.37 (t, J=7.7 Hz, 1H), 6.67 (d, J=18.7 Hz, 1H), 5.47 (q, J=6.5 Hz, 1H), 4.73 (t, J=8.7 Hz, 2H), 4.25 (t, J=14.7, 7.7 Hz, 2H), 4.18 (d, J=20.0 Hz, 2H), 3.69-3.60 (m, 2H), 2.69-2.58 (m, 2H), 2.57 (s, 3H), 2.07 (d, J=15.1 Hz, 3H), 1.34 (d, J=6.6 Hz, 3H); LCMS m/e: 455.2 (MH⁺).

Compound 35: ¹H NMR (400 MHz, DMSO-d₆) δ 8.53 (s, 1H), 8.14 (s, 1H), 7.97 (d, J=7.7 Hz, 1H), 7.61 (d, J=7.5 Hz, 1H), 7.38 (t, J=7.8 Hz, 1H), 5.47 (q, J=11.7, 5.3 Hz, 1H), 4.75 (t, J=8.5 Hz, 2H), 4.42 (dd, J=16.5, 7.6 Hz, 1H), 4.30-4.20 (m, 2H), 3.86 (dd, J=14.2, 6.3 Hz, 1H), 3.45-3.35 (m, 1H), 2.91-2.87 (m, 1H), 2.56 (s, 3H), 2.31-2.26 (m, 1H), 2.19-2.14 (m, 1H), 2.07 (s, 3H), 1.99-1.94 (m, 2H), 1.35 (d, J=6.1 Hz, 3H); LCMS m/e: 475.2 (MH⁺).

Compound 36: ¹HNMR (400 MHz, DMSO-d₆) δ 8.61 (s, 1H), 8.05 (d, J=7.6 Hz, 1H), 7.91 (d, J=7.8 Hz, 1H), 7.87-7.78 (m, 1H), 7.57 (d, J=7.7 Hz, 1H), 7.39 (t, J=7.9 Hz, 1H), 6.54 (s, 1H), 5.94-5.78 (m, 1H), 4.33-4.28 (m, 2H), 3.89 (t, J=5.3 Hz, 2H), 3.78 (s, 3H), 2.64-2.60 (m, 2H), 2.59 (s, 3H), 2.53 (s, 3H), 1.58 (d, J=6.6 Hz, 3H); LCMS m/e: 442 (MW).

Compound 37: ¹HNMR (400 MHz, DMSO-d₆) δ 8.52 (s, 1H), 7.98 (d, J=8.0 Hz, 1H), 7.89 (d, J=7.8 Hz, 1H), 7.81-7.72 (m, 1H), 7.56 (d, J=7.7 Hz, 1H), 7.39 (t, J=7.9 Hz, 1H), 6.40 (s, 1H), 5.90-5.80 (m, 1H), 4.19 (d, J=18.8 Hz, 2H), 3.74 (s, 3H), 3.72-3.68 (m, 2H), 2.71-2.69 (m, 1H), 2.65-2.59 (m, 1H), 2.57 (s, 3H), 2.54 (s, 3H), 2.11 (d, J=16.4 Hz, 3H), 1.55 (d, J=6.0 Hz, 3H); LCMS m/e: 483.3 (MH⁺).

Compound 38: ¹HNMR (400 MHz, DMSO-d₆) δ 8.41 (s, 1H), 7.94-7.90 (m, 2H), 7.71-7.60 (m, 2H), 7.38 (t, J=7.7 Hz, 1H), 6.45 (s, 1H), 5.75 (q, J=6.7 Hz, 1H), 4.29 (d, J=2.5 Hz, 2H), 3.88 (t, J=5.4 Hz, 2H), 3.68 (s, 3H), 2.65 (s, 3H), 2.56-2.51 (m, 5H), 1.48 (d, J=6.5 Hz, 3H); LCMS m/e: 399.3 (MH⁺).

Compound 39: ¹HNMR (400 MHz, DMSO-d₆) δ 8.88 (s, 1H), 8.12 (s, 1H), 7.94 (d, J=7.9 Hz, 1H), 7.52 (d, J=7.9 Hz, 1H), 7.36 (t, J=7.6 Hz, 1H), 6.81 (s, 1H), 5.78 (q, J=6.7 Hz, 1H), 4.30 (d, J=2.4 Hz, 2H), 3.86 (t, J=5.5 Hz, 2H), 3.65 (s, 3H), 2.57 (s, 2H), 2.52 (s, 3H), 2.47 (s, 3H), 1.42 (d, J=6.6 Hz, 3H); LCMS m/e: 443.2 (MH⁺).

Compound 40: ¹H NMR (400 MHz, DMSO-d₆) δ 8.80 (s, 1H), 8.08 (s, 1H), 7.97 (d, J=7.3 Hz, 1H), 7.59 (d, J=6.8 Hz, 1H), 7.36 (t, J=7.7 Hz, 1H), 6.58 (s, 1H), 5.72-5.64 (m, 1H), 3.60 (s, 3H), 2.86-2.74 (m, 4H), 2.60 (s, 3H), 2.43 (s, 3H), 2.27-2.13 (m, 2H), 1.37 (d, J=6.6 Hz, 3H); LCMS m/e: 434.4 (MH⁺).

Compound 41: ¹HNMR (400 MHz, DMSO-d₆) δ 8.85 (s, 1H), 8.08 (s, 1H), 7.96 (d, J=7.9 Hz, 1H), 7.60 (d, J=7.5 Hz, 1H), 7.36 (t, J=7.7 Hz, 1H), 6.80 (s, 1H), 5.69 (q, J=6.4 Hz, 1H), 4.32-4.28 m, 2H), 3.85 (t, J=5.5 Hz, 2H), 3.64 (s, 3H), 2.61 (s, 3H), 2.59-2.53 (m, 2H), 2.46 (s, 3H), 1.39 (d, J=6.6 Hz, 3H); LCMS m/e: 400 (MH⁺).

### Example 42

### Synthesis of (R,Z)-1,2-dimethyl-N-(1-(2-methyl-3-(trifluoromethyl)phenyl)ethyl)-6-(1-(methylsulfonyl)-1,2,3,6-tetrahydropyridin-4-yl)pyrido[3,4-d]pyrimidin-4(1H)-imine (Compound 42)

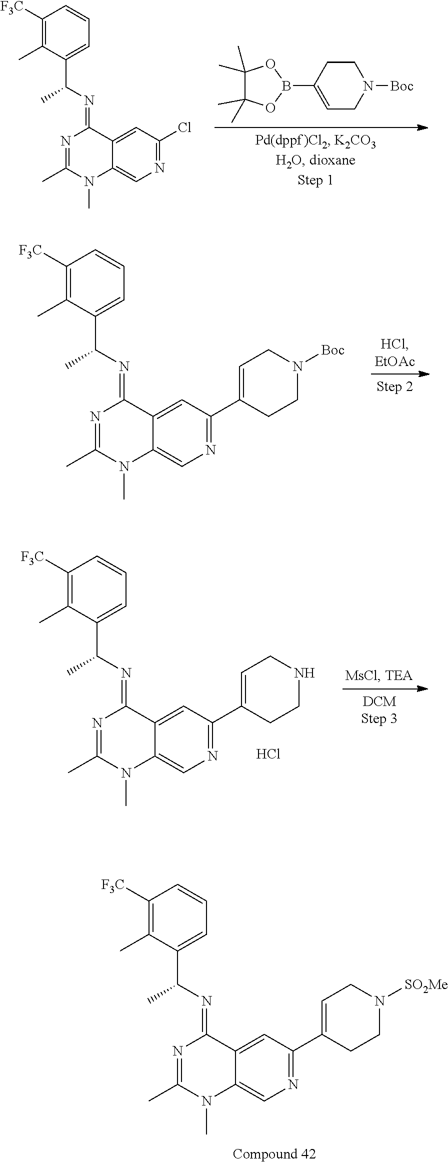

### Step 1. Synthesis of tert-butyl (R,Z)-4-(1,2-dimethyl-4-((1-(2-methyl-3-(trifluoromethyl)phenyl)ethyl)imino)-1,4-dihydropyrido[3,4-d]pyrimidin-6-yl)-3,6-dihydropyridine-1(2H)-carboxylate

To a solution of (R,Z)-6-chloro-1,2-dimethyl-N-(1-(2-methyl-3-(trifluoromethyl)phenyl)-ethyl)pyrido[3,4-d]pyrimidin-4(1H)-imine (300 mg, 0.76 mmol) in 1,4-dioxane (10 mL) and H₂O (2 mL) was added K₂CO₃ (315 mg, 2.28 mmol), tert-butyl 4-(4,4,5,5-tetramethyl-1,3,2-dioxaborolan-2-yl)-3,6-dihydropyridine-1(2H)-carboxylate (282 mg, 0.912 mmol), and Pd(dppf)Cl₂ (56 mg, 0.076 mmol) and stirred at 100° C. under N₂ for 5 hrs. The reaction mixture was cooled to room temperature, diluted with water (20 mL), and extracted with EtOAc (30 mL×3). The combined organic layers were washed with brine (50 mL), dried over Na₂SO₄, filtered, and concentrated to dryness. The residue was purified by silica gel column chromatography (gradient, DCM to DCM:MeOH=10:1) to afford the title compound (300 mg, 0.554 mmol, 72.9% yield) as a yellow solid.

LC-MS m/e: 542 (MH⁺)

### Step 2. Synthesis of (R,Z)-1,2-dimethyl-N-(1-(2-methyl-3-(trifluoromethyl)phenyl)ethyl)-6-(1,2,3,6-tetrahydropyridin-4-yl)pyrido[3,4-d]pyrimidin-4(1H)-imine hydrochloride

To a solution of tert-butyl (R,Z)-4-(1,2-dimethyl-4-((1-(2-methyl-3-(trifluoromethyl)-phenyl)ethyl)imino)-1,4-dihydropyrido[3,4-d]pyrimidin-6-yl)-3,6-dihydropyridine-1(2H)-carboxylate (300 mg, 0.554 mmol) in EtOAc (10 mL) was added HCl solution (5 mL, 4 M in EtOAc) and stirred at room temperature for 0.5 hrs. The resulting mixture was concentrated to afford the title compound (200 mg, 0.419 mmol, 75.7% yield) as a light-yellow solid.

LC-MS m/e: 442 (MH⁺)

### Step 3. Synthesis of (R,Z)-1,2-dimethyl-N-(1-(2-methyl-3-(trifluoromethyl)phenyl)ethyl)-6-(1-(methylsulfonyl)-1,2,3,6-tetrahydropyridin-4-yl)pyrido[3,4-d]pyrimidin-4(1H)-imine. (Compound 42)

To a solution of (R,Z)-1,2-dimethyl-N-(1-(2-methyl-3-(trifluoromethyl)phenyl)ethyl)-6-(1,2,3,6-tetrahydropyridin-4-yl)pyrido[3,4-d]pyrimidin-4(1H)-imine hydrochloride (70 mg, 0.147 mmol) and Et₃N (0.088 mL, 0.634 mmol) in DCM (5 mL) was added MsCl (0.025 mL, 0.317 mmol) at 0° C. and stirred at 0° C. for 2 hrs. The reaction mixture was poured into ice water (10 mL) and extracted with DCM (20 mL×3), the combined organic layers were washed with brine (20 mL), dried over anhydrous Na₂SO₄, filtered, and concentrated to dryness. The residue was purified by prep-HPLC to afford the title compound (25 mg, 0.048 mmol, 30.4% yield) as a yellow solid.

¹HNMR (400 MHz, DMSO-d₆) δ 8.86 (s, 1H), 8.14 (s, 1H), 7.96 (d, J=7.8 Hz, 1H), 7.52 (d, J=7.7 Hz, 1H), 7.36 (t, J=7.8 Hz, 1H), 6.78 (s, 1H), 5.78 (q, J=6.5 Hz, 1H), 3.95 (d, J=2.7 Hz, 2H), 3.64 (s, 3H), 3.42 (t, J=4.0 Hz, 2H), 2.95 (s, 3H), 2.73 (s, 2H), 2.52 (s, 3H), 2.46 (s, 3H), 1.41 (d, J=6.6 Hz, 3H).

LC-MS m/e: 520 (MH⁺).

### Example 43

### Synthesis of. (R,Z)-4-(1,2-dimethyl-4-((1-(2-methyl-3-(trifluoromethyl)phenyl)ethyl)imino)-1,4-dihydropyrido[3,4-d]pyrimidin-6-yl)-3,6-dihydropyridine-1(2H)-carboxamide (Compound 43)

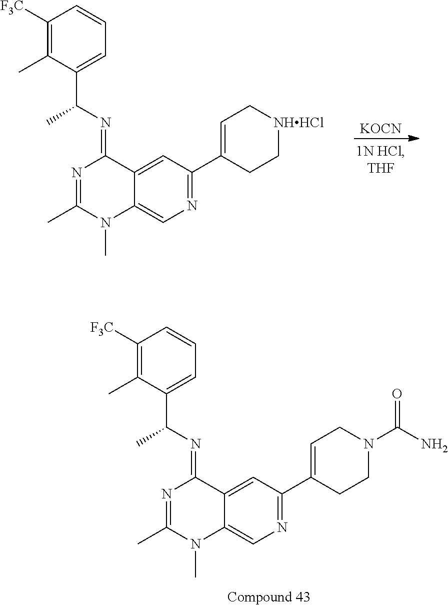

To a solution of (R,Z)-1,2-dimethyl-N-(1-(2-methyl-3-(trifluoromethyl)phenyl)ethyl)-6-(1,2,3,6-tetrahydropyridin-4-yl)pyrido[3,4-d]pyrimidin-4(1H)-imine hydrochloride (100 mg, 0.21 mmol) in THF (5 mL) and 1N HCl (2 mL) was added KOCN (85 mg, 1.05 mmol) and stirred at 25° C. for 12 hrs. The reaction mixture was basified with saturated NaHCO₃ solution (40 mL) and extracted with EtOAc (10 mL×3). The combined organic layers were washed with brine (10 mL), dried over anhydrous Na₂SO₄, filtered, and concentrated to dryness. The residue was purified by prep-HPLC to afford the title compound (21 mg, 0.043 mmol, 20.5% yield) as a white solid.

¹HNMR (400 MHz, DMSO-d₆) δ 8.97 (s, 1H), 8.27 (s, 1H), 7.96 (d, J=7.8 Hz, 1H), 7.53 (d, J=7.5 Hz, 1H), 7.37 (t, J=7.8 Hz, 1H), 6.80 (s, 1H), 6.04 (s, 2H), 5.80 (q, J=6.6 Hz, 1H), 4.07 (d, J=2.6 Hz, 2H), 3.70 (s, 3H), 3.56-3.56 (m, 2H), 2.60 (s, 2H), 2.54 (s, 3H), 2.50 (d, J=1.7 Hz, 3H), 1.47 (d, J=6.7 Hz, 3H).

LC-MS m/e: 485 (MH⁺).

### Examples 44 and 45

### Synthesis of 1-((R)-4-((Z)-1,2-dimethyl-4-(((R)-1-(2-methyl-3-(trifluoromethyl)phenyl)-ethyl)imino)-1,4-dihydropyrido[3,4-d]pyrimidin-6-yl)-6-methyl-3,6-dihydropyridin-1(2H)-yl)ethan-1-one and 1-((R)-4-((Z)-1,2-dimethyl-4-(((R)-1-(2-methyl-3-(trifluoromethyl)-phenyl)ethyl)imino)-1,4-dihydropyrido[3,4-d]pyrimidin-6-yl)-2-methyl-3,6-dihydropyridin-1(2H)-yl)ethan-1-one (Compound 44)

### 1-((2R)-4-((Z)-1,2-dimethyl-4-(((R)-1-(2-methyl-3-(trifluoromethyl)phenyl)ethyl)imino)-1,4-dihydropyrido[3,4-d]pyrimidin-6-yl)-4-fluoro-2-methylpiperidin-1-yl)ethan-1-one (Compound 45)

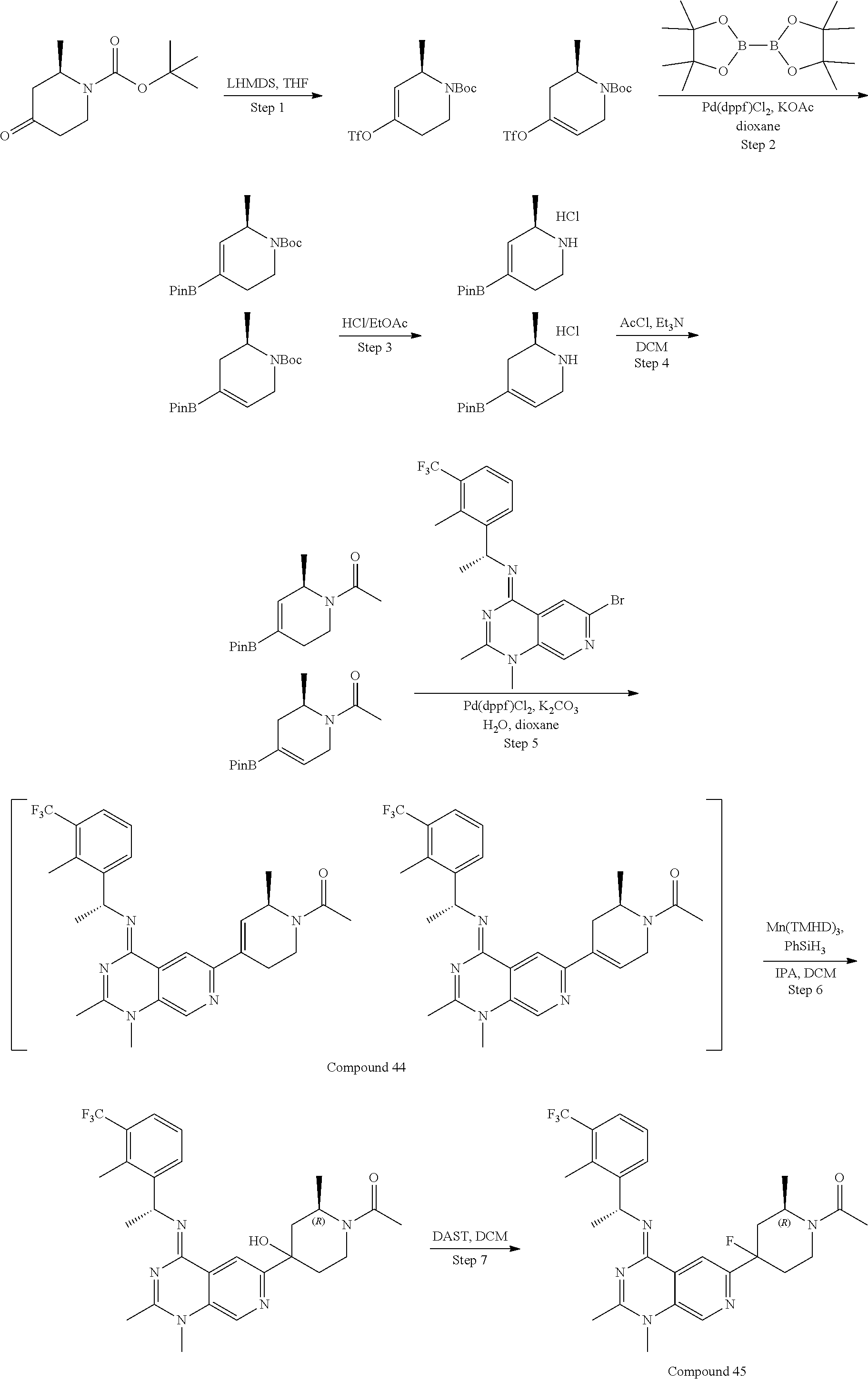

### Step 1. Synthesis of tert-butyl (R)-6-methyl-4-(((trifluoromethyl)sulfonyl)oxy)-3,6-dihydro-pyridine-1(2H)-carboxylate and tert-butyl (R)-2-methyl-4-(((trifluoromethyl)sulfonyl)oxy)-3,6-dihydropyridine-1(2H)-carboxylate

To a solution of tert-butyl (R)-2-methyl-4-oxopiperidine-1-carboxylate (1 g, 4.68 mmol) in THF (20 mL) was added dropwise LiHMDS (4.7 mL, 6.09 mmol, 1.3 M in THF) at −65° C. under N₂ and stirred for 0.5 hrs followed by addition of N-(5-chloro-pyridin-2-yl)-1,1,1-trifluoro-N-((trifluoromethyl)sulfonyl)methanesulfonamide (2.39 g, 6.09 mmol) in portions at −65° C. The reaction mixture was stirred at room temperature for 18 hrs, diluted with water (50 mL), and extracted with EtOAc (50 mL×3). The combined organic layers were washed with brine (50 mL), dried over anhydrous Na₂SO₄, filtered, and concentrated to dryness. The residue was purified by flash chromatography on silica gel (gradient, PE to PE:EtOAc=20:1) to afford the title compound (0.75 g, 2.17 mmol, 46.3% yield) as a yellow oil.

LCMS m/e: 346 (MH⁺).

### Step 2. Synthesis of tert-butyl (R)-6-methyl-4-(4,4,5,5-tetramethyl-1,3,2-dioxaborolan-2-yl)-3,6-dihydropyridine-1(2H)-carboxylate- and tert-butyl (R)-2-methyl-4-(4,4,5,5-tetramethyl-1,3,2-dioxaborolan-2-yl)-3,6-dihydropyridine-1(2H)-carboxylate

To a mixture of tert-butyl (R)-6-methyl-4-(((trifluoromethyl)sulfonyl)oxy)-3,6-dihydropyridine-1(2H)-carboxylate and tert-butyl (R)-2-methyl-4-(((trifluoromethyl)-sulfonyl)oxy)-3,6-dihydropyridine-1(2H)-carboxylate (0.75 g, 2.17 mmol) in dioxane (20 mL) was added bis(pinacolato)diboron (0.61 g, 2.38 mmol), KOAc (0.64 g, 6.51 mmol) and Pd(dppf)Cl₂ (160 mg, 0.217 mmol). The mixture was stirred at 100° C. under N₂ for 1 hr, cooled to room temperature, diluted with water (20 mL), and extracted with EtOAc (50 mL×3). The combined organic layers were washed with brine (50 mL), dried over anhydrous Na₂SO₄, filtered, and concentrated to dryness. The residue was purified by flash chromatography on silica gel (gradient, PE to PE:EtOAc=20:1) to afford the title compound (0.65 g, 2.01 mmol, 92.5% yield) as a yellow oil.

LCMS m/e: 324 (MH⁺).

### Step 3. Synthesis of (R)-6-methyl-4-(4,4,5,5-tetramethyl-1,3,2-dioxaborolan-2-yl)-1,2,3,6-tetrahydropyridine hydrochloride and (R)-2-methyl-4-(4,4,5,5-tetramethyl-1,3,2-dioxaborolan-2-yl)-1,2,3,6-tetrahydropyridine hydrochloride

To a mixture of tert-butyl (R)-6-methyl-4-(4,4,5,5-tetramethyl-1,3,2-dioxaborolan-2-yl)-3,6-dihydropyridine-1(2H)-carboxylate and tert-butyl (R)-2-methyl-4-(4,4,5,5-tetramethyl-1,3,2-dioxaborolan-2-yl)-3,6-dihydropyridine-1(2H)-carboxylate (325 mg, 1 mmol) in EtOAc (10 mL) was added dropwise HCl (2.5 mL, 10 mmol, 4 N in EtOAc). The mixture was stirred at room temperature for 1 hr and evaporated to afford the crude title compound (250 mg, 0.963 mmol, 95.7% yield) as a yellow solid.

LCMS m/e: 224 (MH⁺).

### Step 4. Synthesis of (R)-1-(6-methyl-4-(4,4,5,5-tetramethyl-1,3,2-dioxaborolan-2-yl)-3,6-dihydropyridin-1(2H)-yl)ethan-1-one and (R)-1-(2-methyl-4-(4,4,5,5-tetramethyl-1,3,2-dioxaborolan-2-yl)-3,6-dihydropyridin-1(2H)-yl)ethan-1-one

To a mixture of (R)-6-methyl-4-(4,4,5,5-tetramethyl-1,3,2-dioxaborolan-2-yl)-1,2,3,6-tetrahydropyridine hydrochloride, (R)-2-methyl-4-(4,4,5,5-tetramethyl-1,3,2-dioxaborolan-2-yl)-1,2,3,6-tetrahydropyridine hydrochloride (250 mg, 0.963 mmol) and Et₃N (291 mg, 2.88 mmol) in DCM (5 mL) was added dropwise AcCl (75 mg, 0.963 mmol) at 0° C. and stirred for 1 hr. The resulting mixture was diluted with water (10 mL) and extracted with DCM (30 mL×2). The combined organic layers were washed with brine (30 mL), dried over anhydrous Na₂SO₄, filtered, and concentrated to afford the title compound (190 mg, 0.717 mmol, 74.4% yield) as a yellow solid.

LCMS m/e: 266 (MH⁺).

### Step 5. Synthesis of 1-((R)-4-((Z)-1,2-dimethyl-4-(((R)-1-(2-methyl-3-(trifluoromethyl)-phenyl)ethyl)imino)-1,4-dihydropyrido[3,4-d]pyrimidin-6-yl)-6-methyl-3,6-dihydropyridin-1(2H)-yl)ethan-1-one and 1-((R)-4-((Z)-1,2-dimethyl-4-(((R)-1-(2-methyl-3-(trifluoromethyl)-phenyl)ethyl)imino)-1,4-dihydropyrido[3,4-d]pyrimidin-6-yl)-2-methyl-3,6-dihydropyridin-1(2H)-yl)ethan-1-one (Compound 44)

To a mixture of (R)-1-(6-methyl-4-(4,4,5,5-tetramethyl-1,3,2-dioxaborolan-2-yl)-3,6-dihydropyridin-1(2H)-yl)ethan-1-one and (R)-1-(2-methyl-4-(4,4,5,5-tetramethyl-1,3,2-dioxaborolan-2-yl)-3,6-dihydropyridin-1(2H)-yl)ethan-1-one (174 mg, 0.659 mmol), (R,Z)-6-chloro-1,2-dimethyl-N-(1-(2-methyl-3-(trifluoromethyl)phenyl)ethyl)pyrido[2,3-d]pyrimidin-4(1H)-imine (200 mg, 0.507 mmol) and Pd(dppf)Cl₂ (37 mg, 0.051 mmol) in dioxane (5 mL) and water (0.5 mL) was added K₂CO₃ (210 mg, 1.52 mmol). The mixture was stirred at 100° C. under N₂ for 2 hrs, cooled to room temperature, and concentrated to dryness. The residue was purified by flash chromatography on silica gel (gradient, DCM to DCM:MeOH=10:1) and reverse phase chromatography (SilaSep™ C18 silica flash cartridge, 0%-40% MeCN in H₂O with 0.1% formic acid) to afford the title mixture compound (110 mg, 0.221 mmol, 43.6% yield) as a yellow solid.

¹HNMR (400 MHz, DMSO-d₆) δ 8.86 (s, 1H), 8.17-8.06 (m, 1H), 7.91-7.95 (m, 1H), 7.52 (d, J=7.8 Hz, 1H), 7.36 (t, J=7.6 Hz, 1H), 6.73 (d, J=21.8 Hz, 1H), 5.84-5.73 (m, 1H), 5.02-4.56 (m, 1H), 4.47-3.92 (m, 1H), 3.64 (s, 3H), 3.59-3.47 (m, 1H), 3.22 (s, 3H), 2.64-2.80 (m, 2H), 2.46 (s, 3H), 2.14-2.01 (m, 3H), 1.42 (d, J=6.4 Hz, 3H), 1.36-1.11 (m, 3H).

LCMS m/e: 498 (MH⁺).

### Step 6. Synthesis of 1-((2R)-4-((Z)-1,2-dimethyl-4-(((R)-1-(2-methyl-3-(trifluoromethyl)phenyl)ethyl)imino)-1,4-dihydropyrido[3,4-d]pyrimidin-6-yl)-4-hydroxy-2-methylpiperidin-1-yl)ethan-1-one

To a solution of Mn(TMHD)₃ (103 mg, 0.171 mmol), 1-((R)-4-((Z)-1,2-dimethyl-4-(((R)-1-(2-methyl-3-(trifluoromethyl)phenyl)ethyl)imino)-1,4-dihydropyrido[3,4-d]pyrimidin-6-yl)-6-methyl-3,6-dihydropyridin-1(2H)-yl)ethan-1-one and 1-((R)-4-((Z)-1,2-dimethyl-4-(((R)-1-(2-methyl-3-(trifluoromethyl)phenyl)ethyl)imino)-1,4-dihydropyrido[3,4-d]pyrimidin-6-yl)-2-methyl-3,6-dihydropyridin-1(2H)-yl)ethan-1-one (170 mg, 0.342 mmol) in DCM (3 mL) and i-PrOH (8 mL) was added dropwise PhSiH₃ (55 mg, 0.512 mmol) at 0° C. under O₂. The mixture was stirred at 0° C. for 1 hr, diluted with water (10 mL), and extracted with DCM (20 mL×2). The combined organic layers were washed with brine (20 mL), dried over anhydrous Na₂SO₄, filtered, and concentrated to dryness. The residue was purified by neutral Al₂O₃ column chromatography (gradient, 100% PE to PE:DCM:MeOH=25:4:1) to give the title compound (50 mg, 0.097 mmol, 28.3% yield) as a yellow solid.

LCMS m/e: 516 (MH⁺).

### Step 7. Synthesis of 1-((2R)-4-((Z)-1,2-dimethyl-4-(((R)-1-(2-methyl-3-(trifluoromethyl)-phenyl)ethyl)imino)-1,4-dihydropyrido[3,4-d]pyrimidin-6-yl)-4-fluoro-2-methylpiperidin-1-yl)ethan-1-one (Compound 45)

To a solution of 1-((2R)-4-((Z)-1,2-dimethyl-4-(((R)-1-(2-methyl-3-(trifluoromethyl)-phenyl)ethyl)imino)-1,4-dihydropyrido[3,4-d]pyrimidin-6-yl)-4-hydroxy-2-methylpiperidin-1-yl)ethan-1-one (50 mg, 0.097 mmol) in DCM (3 mL) was added dropwise DAST (62 mg, 0.38 mmol) at −30° C. and stirred at 0° C. for 1 hr. The reaction was quenched with saturated NaHCO₃ solution (20 mL) and extracted with DCM (20 mL×2). The combined organic layers were dried over Na₂SO₄, filtered, concentrated, and purified by reverse phase column (SilaSep™ C18 silica flash cartridge, 0%-40% MeCN in H₂O with 0.1% TFA) to afford trifluoroacetate salt of the title compound (9 mg).

¹HNMR (400 MHz, DMSO-d₆) δ 10.88 (d, J=6.1 Hz, 1H), 9.50 (s, 1H), 8.85 (s, 1H), 7.82 (d, J=7.8 Hz, 1H), 7.63 (d, J=7.8 Hz, 1H), 7.45 (t, J=7.8 Hz, 1H), 5.92 (p, J=7.3 Hz, 1H), 4.98-4.90 (m, 0.5H), 4.56-4.49 (m, 0.5H), 4.34-4.38 (m, 0.5H), 4.02 (s, 3H), 3.83-3.86 (m, 0.5H), 3.58-3.49 (m, 1H), 3.05 (t, J=13.3 Hz, 0.5H), 2.77 (s, 3H), 2.60 (s, 3H), 2.16-2.40 (m, 1.5H), 2.10 (d, J=2.6 Hz, 3H), 2.06-1.89 (m, 2H), 1.68 (d, J=6.9 Hz, 3H), 1.36 (dd, J=50.2, 6.9 Hz, 3H).

LCMS m/e: 518 (MH⁺).

### Example 46

### Synthesis of 1-(4-((Z)-1,2-dimethyl-4-(((R)-1-(2-methyl-3-(trifluoromethyl)phenyl)ethyl)-imino)-1,4-dihydropyrido[3,4-d]pyrimidin-6-yl)-4-fluoro-2,2-dimethylpiperidin-1-yl)ethan-1-one (Compound 46)

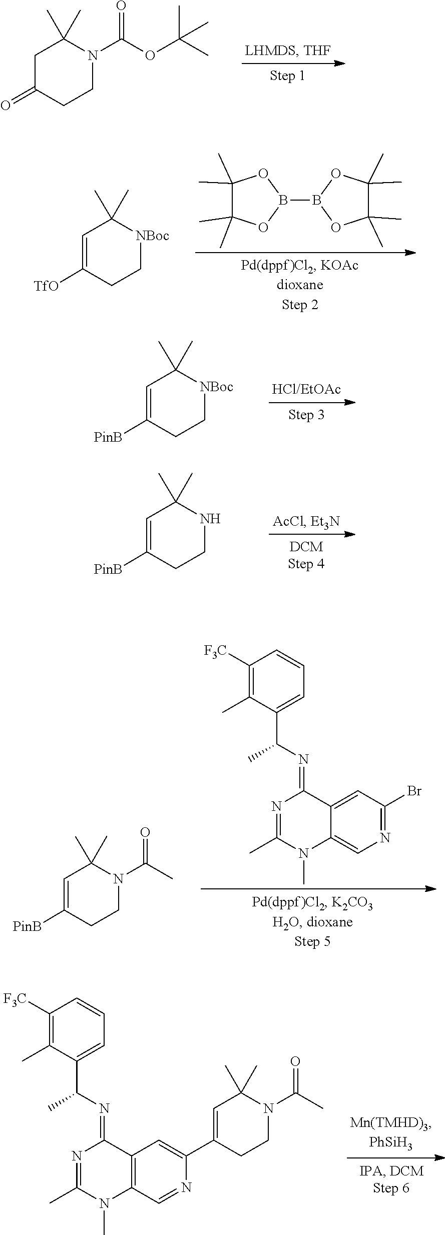

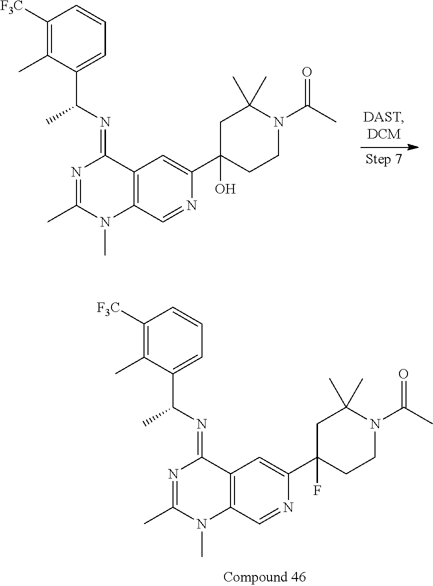

### Step 1. Synthesis of tert-butyl 6,6-dimethyl-4-(((trifluoromethyl)sulfonyl)oxy)-3,6-dihydropyridine-1(2H)-carboxylate

To a solution of tert-butyl 2,2-dimethyl-4-oxopiperidine-1-carboxylate (2 g, 8.8 mmol) in THF (50 mL) was added LiHMDS (1.3 M, 13.5 mL) at −50° C. After stirring at −50° C. for 0.5 hrs, 1,1,1-trifluoro-N-phenyl-N-((trifluoromethyl)sulfonyl)methanesulfonamide (3.8 g, 10.56 mmol) was added and the mixture was stirred at room temperature for 2 hrs. The resulting mixture was quenched with saturated NH₄Cl solution (40 mL), diluted with water (40 mL), and extracted with EtOAc (100 mL×3). The combined organic layers were washed with brine (80 mL), dried over anhydrous Na₂SO₄, and filtered. The filtrate was concentrated under reduced pressure and purified by silica gel column chromatography (gradient, PE to PE:EtOAc=20:1) to afford the title compound (2 g, 5.56 mmol, 63.2% yield) as a colorless oil.

LCMS m/e: 360.3 (MH⁺).

### Step 2. Synthesis of tert-butyl 6,6-dimethyl-4-(4,4,5,5-tetramethyl-1,3,2-dioxaborolan-2-yl)-3,6-dihydropyridine-1(2H)-carboxylate

To a solution of tert-butyl 6,6-dimethyl-4-(((trifluoromethyl)sulfonyl)oxy)-3,6-dihydro-pyridine-1(2H)-carboxylate (2 g, 5.56 mmol) in dioxane (50 mL) was added 4,4,4′,4′,5,5,5′,5′-octamethyl-2,2′-bi(1,3,2-dioxaborolane) (2.8 g, 11.1 mmol), Pd(dppf)Cl₂ (410 mg, 0.56 mmol) and KOAc (1.6 g, 16.7 mmol). The mixture was stirred at 120° C. under N₂ for 2 hrs, cooled to room temperature, diluted with water (100 mL), and extracted with EtOAc (100 mL×3). The combined organic layers were washed with brine (100 mL), dried over anhydrous Na₂SO₄, and filtered. The filtrate was concentrated under reduced pressure and purified by silica gel column chromatography (gradient, PE to PE:EtOAc=10:1) to afford the title compound (400 mg, 1.19 mmol, 21.3% yield) as a colorless oil.

LCMS m/e: 338.3 (MH⁺).

### Step 3. Synthesis of 6,6-dimethyl-4-(4,4,5,5-tetramethyl-1,3,2-dioxaborolan-2-yl)-1,2,3,6-tetrahydropyridine

To a solution of tert-butyl 6,6-dimethyl-4-(4,4,5,5-tetramethyl-1,3,2-dioxaborolan-2-yl)-3,6-dihydropyridine-1(2H)-carboxylate (400 mg, 1.19 mmol) in EtOAc (5 mL) was added HCl solution (5 mL, 4 M in EtOAc). The mixture was stirred at room temperature for 5 hrs. The reaction mixture was concentrated to dryness, basified with saturated NaHCO₃ solution (40 mL), and extracted with EtOAc (50 mL×3). The combined organic layers were washed with brine (80 mL), dried over Na₂SO₄, and filtered. The filtrate was concentrated under reduced pressure and purified by silica gel column chromatography (gradient, PE to PE:EtOAc=2:1) to give the title compound (250 mg, 1.05 mmol, 88.2% yield) as a white solid.

LCMS m/e: 238.3 (MH⁺).

### Step 4. Synthesis of 1-(6,6-dimethyl-4-(4,4,5,5-tetramethyl-1,3,2-dioxaborolan-2-yl)-3,6-dihydropyridin-1(2H)-yl)ethan-1-one

To a solution of 6,6-dimethyl-4-(4,4,5,5-tetramethyl-1,3,2-dioxaborolan-2-yl)-1,2,3,6-tetrahydropyridine (250 mg, 1.05 mmol) and Et₃N (319 mg, 3.15 mmol) in DCM (20 mL) was added AcCl (82 mg, 1.05 mmol). The mixture was stirred at room temperature for 2 hrs, diluted with water (20 mL), and extracted with DCM (20 mL×3). The combined organic layers were washed with brine (80 mL), dried over Na₂SO₄, and filtered. The filtrate was concentrated under reduced pressure and purified by silica gel column chromatography (gradient, PE to PE:EtOAc=5:1) to afford the title compound (150 mg, 0.54 mmol, 51.2% yield) as a white solid.

LCMS m/e: 280.2 (MH⁺).

### Step 5. Synthesis of (R,Z)-1-(4-(1,2-dimethyl-4-((1-(2-methyl-3-(trifluoromethyl)phenyl)-ethyl)imino)-1,4-dihydropyrido[3,4-d]pyrimidin-6-yl)-6,6-dimethyl-3,6-dihydropyridin-1(2H)-yl)ethan-1-one

To a solution of 1-(6,6-dimethyl-4-(4,4,5,5-tetramethyl-1,3,2-dioxaborolan-2-yl)-3,6-dihydropyridin-1(2H)-yl)ethan-1-one (150 mg, 0.54 mmol) in dioxane (20 mL) and water (5 mL) was added (R,Z)-6-bromo-1,2-dimethyl-N-(1-(2-methyl-3-(trifluoromethyl)phenyl)ethyl)pyrido-[3,4-d]pyrimidin-4(1H)-imine (285 mg, 0.65 mmol), Pd(dppf)Cl₂ (37 mg, 0.05 mmol) and K₂CO₃ (224 mg, 1.62 mmol). The mixture was stirred at 120° C. under N₂ for 2 hrs. The mixture was cooled to room temperature, diluted with water (60 mL), and extracted with EtOAc (60 mL×3). The combined organic layers were washed with brine (60 mL), dried over anhydrous Na₂SO₄, and filtered. The filtrate was concentrated under reduced pressure and purified by silica gel column chromatography (gradient, DCM to DCM:MeOH=15:1) to afford the title compound (80 mg, 0.16 mmol, 29.0% yield) as a brown solid.

LCMS m/e: 512.6 (MH⁺).

### Step 6. Synthesis of 1-(4-((Z)-1,2-dimethyl-4-(((R)-1-(2-methyl-3-(trifluoromethyl)phenyl)ethyl)imino)-1,4-dihydropyrido[3,4-d]pyrimidin-6-yl)-4-hydroxy-2,2-dimethylpiperidin-1-yl)ethan-1-one

To a solution of (R,Z)-1-(4-(1,2-dimethyl-4-((1-(2-methyl-3-(trifluoromethyl)phenyl)-ethyl)imino)-1,4-dihydropyrido[3,4-d]pyrimidin-6-yl)-6,6-dimethyl-3,6-dihydropyridin-1(2H)-yl)ethan-1-one (80 mg, 0.16 mmol) and Mn(TMHD)₃ (29 mg, 0.048 mmol) in i-PrOH (9 mL) and DCM (1 mL) was added PhSiH₃ (23 mg, 0.21 mmol) at 0° C. under O₂. The mixture was stirred at room temperature for 2 hrs, diluted with water (60 mL), and then extracted with EtOAc (60 mL×3). The combined organic layers were washed with brine (60 mL), dried over anhydrous Na₂SO₄, and filtered. The filtrate was concentrated under reduced pressure and purified by neutral Al₂O₃ column chromatography (gradient, 100% PE to PE:DCM:MeOH=25:4:1) to afford the title compound (60 mg, 0.11 mmol, 70.8% yield) as a brown solid.

LCMS m/e: 530.6 (MH⁺).

### Step 7. Synthesis of 1-(4-((Z)-1,2-dimethyl-4-(((R)-1-(2-methyl-3-(trifluoromethyl)phenyl)-ethyl)imino)-1,4-dihydropyrido[3,4-d]pyrimidin-6-yl)-4-fluoro-2,2-dimethylpiperidin-1-yl)ethan-1-one (Compound 46)

To a solution of 1-(4-((Z)-1,2-dimethyl-4-(((R)-1-(2-methyl-3-(trifluoromethyl)-phenyl)ethyl)imino)-1,4-dihydropyrido[3,4-d]pyrimidin-6-yl)-4-hydroxy-2,2-dimethylpiperidin-1-yl)ethan-1-one (60 mg, 0.11 mmol) in DCM (15 mL) was added DAST (89 mg, 0.55 mmol) at −20° C. and stirred for 2 hrs. The mixture was basified with saturated NaHCO₃ solution (50 mL), and the aqueous layer was extracted with DCM (60 mL×3). The combined organic layers were washed with brine (60 mL), dried over anhydrous Na₂SO₄, and filtered. The filtrate was concentrated under reduced pressure and purified by prep-HPLC to afford the title compound (22 mg, 0.04 mmol, 37.6% yield) as a brown solid.

¹H NMR (400 MHz, DMSO-d₆) δ 8.80 (s, 1H), 8.13 (s, 1H), 7.93 (d, J=7.8 Hz, 1H), 7.51 (d, J=7.7 Hz, 1H), 7.36 (t, J=7.7 Hz, 1H), 5.76 (q, 1H), 3.68-3.62 (m, 1H), 3.59 (s, 3H), 3.55-3.51 (m, 1H), 2.53 (s, 3H), 2.43 (s, 3H), 2.41-2.18 (m, 2H), 2.17-2.08 (m, 1H), 2.06 (s, 3H), 1.98-1.88 (m, 1H), 1.50 (t, J=4.9 Hz, 6H), 1.38 (d, J=6.6 Hz, 3H)

LCMS m/e: 532.4 (MH⁺).

### Example 47

### Synthesis of (R,Z)-1,2-dimethyl-N-(1-(2-methyl-3-(trifluoromethyl)phenyl)ethyl)-6-(3-(trifluoromethyl)imidazo[1,5-a]pyridin-7-yl)pyrido[3,4-d]pyrimidin-4(1H)-imine (Compound 47)

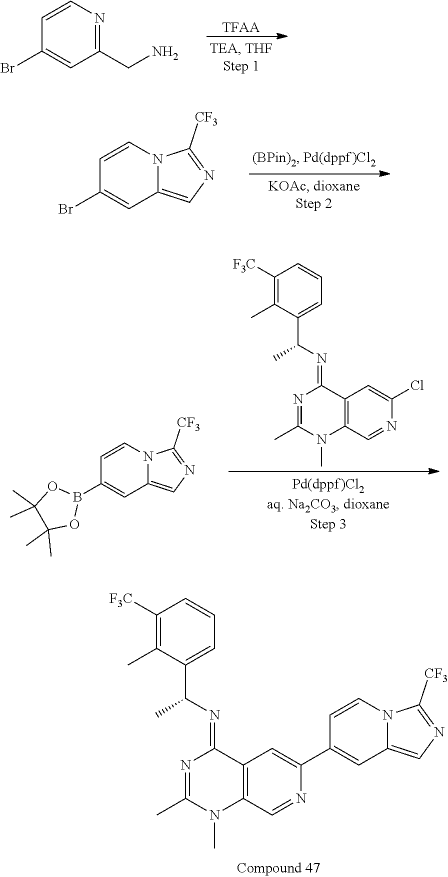 [SMILES: NCc1cc(Br)ccn1 ; "TFAA" ; FC(F)(F)c1ncc2cc(Br)ccn12 ; CC1(C)OB(c2ccn3c(C(F)(F)F)ncc3c2)OC1(C)C ; Cc1c([C@@H](C)/N=c2\nc(C)n(C)c3cnc(Cl)cc23)cccc1C(F)(F)F ; Cc1c([C@@H](C)/N=c2\nc(C)n(C)c3cnc(-c4ccn5c(C(F)(F)F)ncc5c4)cc23)cccc1C(F)(F)F]

### Step 1. Synthesis of 7-bromo-3-(trifluoromethyl)imidazo[1,5-a]pyridine

To a solution of (4-bromopyridin-2-yl)methanamine (600 mg, 3.2 mmol) in THF (12 mL) and TEA (811 mg, 8.02 mmol) was added dropwise TFAA (1.55 g, 7.37 mmol) at 0° C. and stirred at room temperature for 18 hrs. The mixture was diluted with MeOH (7 mL) and purified by flash chromatography on silica gel (gradient, PE to PE:EtOAc=10:1) to afford the title compound (700 mg, 2.64 mmol, 82.3% yield) as a yellow solid.

LCMS m/e: 265, 267 (MH⁺).

### Step 2. Synthesis of 7-(4,4,5,5-tetramethyl-1,3,2-dioxaborolan-2-yl)-3-(trifluoromethyl)-imidazo[1,5-a]pyridine

To a solution of 7-bromo-3-(trifluoromethyl)imidazo[1,5-a]pyridine (350 mg, 1.321 mmol) and bis(pinacolato)diboron (352 mg, 1.38 mmol) in dioxane (6 mL) was added KOAc (388 mg, 3.962 mmol) and Pd(dppf)Cl₂ (96 mg, 0.132 mmol). The mixture was stirred at 100° C. for 1 hr under N₂, cooled to room temperature, and concentrated to dryness. The residue was purified by flash chromatography on silica gel (gradient, PE to PE:EtOAc=10:1) to afford the title compound (400 mg, 1.28 mmol, 97.0% yield) as a yellow solid.

LCMS m/e: 313 (MH⁺).

### Step 3. Synthesis of (R,Z)-1,2-dimethyl-N-(1-(2-methyl-3-(trifluoromethyl)phenyl)ethyl)-6-(3-(trifluoromethyl)imidazo[1,5-a]pyridin-7-yl)pyrido[3,4-d]pyrimidin-4(1H)-imine (Compound 47)

To a solution of 7-(4,4,5,5-tetramethyl-1,3,2-dioxaborolan-2-yl)-3-(trifluoromethyl)-imidazo[1,5-a]pyridine (100 mg, 0.32 mmol) and (R,Z)-6-chloro-1,2-dimethyl-N-(1-(2-methyl-3-(trifluoromethyl)phenyl)ethyl)pyrido[3,4-d]pyrimidin-4(1H)-imine (126 mg, 0.32 mmol) in dioxane (4 mL) and water (0.4 mL) were added Na₂CO₃ (101 mg, 0.961 mmol) and Pd(dppf)Cl₂ (23 mg, 0.032 mmol). The mixture was stirred at 100° C. for 1 hr under N₂, cooled to room temperature, and concentrated to dryness. The residue was purified by reverse phase chromatography (SilaSep™ C18 silica flash cartridge, 0%-40% MeCN in H₂O with 0.1% formic acid) to give the title compound (10 mg, 0.018 mmol, 5.7% yield) as a pale-yellow solid.

¹HNMR (400 MHz, DMSO-d₆) δ 8.93 (s, 1H), 8.57 (s, 1H), 8.54 (s, 1H), 8.51 (d, J=7.2 Hz, 1H), 8.07 (d, J=8.0 Hz, 1H), 7.80 (s, 1H), 7.76 (dd, J=7.6, 1.6 Hz, 1H), 7.53 (d, J=7.6 Hz, 1H), 7.38 (t, J=8.0 Hz, 1H), 5.81 (q, J=6.6 Hz, 1H), 3.63 (s, 3H), 2.53 (s, 3H), 2.43 (s, 3H), 1.43 (d, J=6.6 Hz, 3H).

LCMS m/e: 545 (MH⁺).

### Examples 48-53

### Synthesis of (R,Z)-6-(4,4-difluoropiperidin-1-yl)-1,2-dimethyl-N-(1-(2-methyl-3-(trifluoromethyl)phenyl)ethyl)pyrido[3,4-d]pyrimidin-4(1H)-imine (Compound 48)

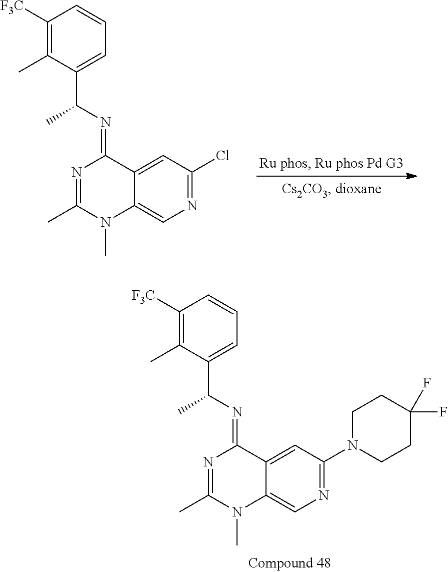 [SMILES: Cc1c([C@@H](C)/N=c2\nc(C)n(C)c3cnc(Cl)cc23)cccc1C(F)(F)F ; Cc1c([C@@H](C)/N=c2\nc(C)n(C)c3cnc(N4CCC(F)(F)CC4)cc23)cccc1C(F)(F)F]

A mixture of (R,Z)-6-chloro-1,2-dimethyl-N-(1-(2-methyl-3-(trifluoromethyl)phenyl)-ethyl)pyrido[3,4-d]pyrimidin-4(1H)-imine (124 mg, 1.024 mmol), RuPhos (32 mg, 0.068 mmol), RuPhos Pd G3 (50 mg, 0.068 mmol) and Cs₂CO₃ (333 mg, 1.02 mmol) in dioxane (15 mL) was stirred at 100° C. under N₂ overnight. The mixture was cooled to room temperature, diluted with water (20 mL), and extracted with EtOAc (30 mL×2). The combined organic layers were dried over anhydrous Na₂SO₄, filtered, concentrated, and purified by preparative HPLC to afford the title compound (7.2 mg, 0.015 mmol, 4.40% yield) as a yellow solid.

¹HNMR (400 MHz, DMSO-d₆) δ 8.59 (s, 1H), 7.92 (d, J=7.7 Hz, 1H), 7.52 (d, J=8.0 Hz, 2H), 7.36 (t, J=7.8 Hz, 1H), 5.78 (q, J=6.7 Hz, 1H), 3.77-3.68 (m, 4H), 3.62 (s, 3H), 2.52 (s, 3H), 2.44 (s, 3H), 2.10-2.01 (m, 4H), 1.43 (d, J=6.6 Hz, 3H).

LC-MS m/e: 480.3 (MH⁺).

Applying a procedure similar to that described above, the following compounds (Compound 49-53) were prepared.

Compound 49: ¹HNMR (400 MHz, DMSO-d₆) δ 8.75 (s, 1H), 7.90 (d, J=7.9 Hz, 1H), 7.61-7.52 (m, 2H), 7.38 (t, J=7.7 Hz, 1H), 5.81 (q, J=6.6 Hz, 1H), 3.71 (s, 3H), 3.64-3.55 (m, 8H), 2.55 (s, 3H), 2.50 (s, 3H), 2.07 (s, 3H), 1.51 (d, J=5.0 Hz, 3H); LC-MS m/e: 487.3 (MH⁺).

Compound 50: ¹HNMR (400 MHz, DMSO-d₆) δ 8.66 (s, 1H), 7.88 (d, J=7.7 Hz, 1H), 7.53 (d, J=7.5 Hz, 1H), 7.36 (t, J=7.7 Hz, 1H), 7.14 (s, 1H), 5.80 (q, J=6.9 Hz, 1H), 3.68 (s, 3H), 3.64 (t, J=6.8 Hz, 2H), 3.39 (s, 2H), 2.54 (s, 3H), 2.51 (s, 3H), 1.95 (t, J=6.7 Hz, 2H), 1.48 (d, J=6.6 Hz, 3H), 0.71-0.61 (m, 4H); LC-MS m/e: 456.3 (MH⁺).

Compound 51: ¹HNMR (400 MHz, DMSO-d₆) δ 8.60 (s, 1H), 7.89 (d, J=7.9 Hz, 1H), 7.52 (d, J=7.8 Hz, 1H), 7.45 (s, 1H), 7.36 (t, J=7.9 Hz, 1H), 5.78 (q, J=6.7 Hz, 1H), 3.67-3.60 (m, 7H), 2.53 (s, 3H), 2.45 (s, 3H), 1.48-1.39 (m, 7H), 0.37 (s, 4H); LC-MS m/e: 470.3 (MH⁺).

Compound 52: ¹HNMR (400 MHz, DMSO-d₆) δ 8.70 (s, 1H), 7.90 (d, J=7.6 Hz, 1H), 7.54 (d, J=7.7 Hz, 2H), 7.37 (t, J=7.9 Hz, 1H), 5.80 (q, J=6.6 Hz, 1H), 4.17 (d, J=12.4 Hz, 1H), 4.06 (d, J=13.0 Hz, 1H), 4.01-3.95 (m, 1H), 3.69 (s, 3H), 3.60 (dd, J=11.6, 8.8 Hz, 2H), 2.94-2.86 (m, 1H), 2.54 (s, 3H), 2.50 (s, 3H), 2.52-2.50 (m, 1H), 1.49 (d, J=6.7 Hz, 3H), 1.20 (d, J=6.2 Hz, 3H); LC-MS m/e: 460.6 (MH⁺).

Formate salt of Compound 53: ¹H NMR (400 MHz, DMSO-d₆) δ 8.74 (s, 1H), 8.46 (s, 1H), 8.36 (s, 1H), 7.95 (d, J=7.6 Hz, 1H), 7.55 (d, J=7.7 Hz, 1H), 7.37 (t, J=7.6 Hz, 1H), 5.84 (q, J=6.3 Hz, 1H), 3.81 (s, 3H), 3.64 (s, 4H), 3.41 (s, 2H), 3.34 (s, 2H), 2.58 (s, 6H), 2.07 (s, 3H), 1.55 (d, J=6.6 Hz, 3H); LC-MS m/e: 487 (MH⁺).

### Examples 54-79

### Synthesis of (R,Z)-3-(1-((1,2-dimethyl-6-morpholinopyrido[3,4-d]pyrimidin-4(1H)-ylidene)amino)ethyl)-2-methylbenzonitrile (Compound 54)

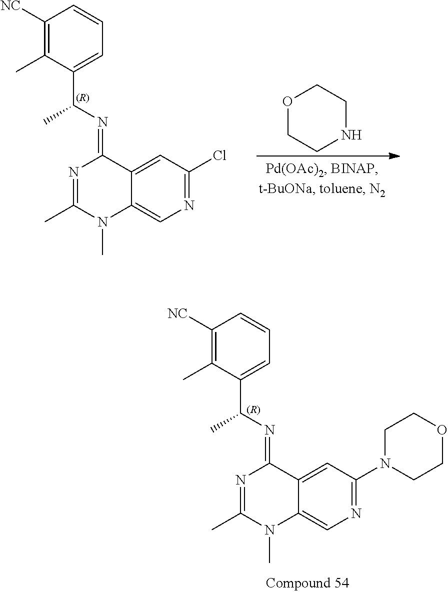

To a solution of (R,Z)-3-(1-((6-chloro-1,2-dimethylpyrido[3,4-d]pyrimidin-4(1H)-ylidene)amino)ethyl)-2-methylbenzonitrile (150 mg, 0.426 mmol) and morpholine (74 mg, 0.852 mmol) in toluene (10 mL) was added t-BuONa (123 mg, 1.28 mmol), BINAP (53 mg, 0.085 mmol), and Pd(OAc)₂ (19 mg, 0.085 mmol) at 25° C. After stirring the mixture at 100° C. under N₂ for 2 hrs, the reaction mixture was cooled to room temperature, poured into ice water (20 mL) and extracted with DCM (50 mL×3). The combined organic layers were dried over anhydrous Na₂SO₄, filtered, concentrated, and purified by pre-HPLC to give the title compound (74.6 mg, 0.185 mmol, 43.5% yield) as a yellow solid.

¹HNMR (400 MHz, DMSO-d₆) δ 8.69 (s, 1H), 8.32 (s, 1H), 7.93 (d, J=7.7 Hz, 1H), 7.62 (d, J=7.5 Hz, 1H), 7.52 (s, 1H), 7.37 (t, J=7.7 Hz, 1H), 5.72 (q, J=6.6 Hz, 1H), 3.77-3.72 (m, 4H), 3.68 (s, 3H), 3.55-3.46 (m, 4H), 2.63 (s, 3H), 2.50 (s, 3H), 1.46 (d, J=6.7 Hz, 3H).

LCMS m/e: 403.4 (MH⁺).

Applying a procedure similar to that described above, the following compounds 55-79 were prepared.

Compound 55: ¹H NMR (400 MHz, DMSO-d₆) δ 8.63 (s, 1H), 7.91 (d, J=7.6 Hz, 1H), 7.53 (d, J=8.0 Hz, 1H), 7.44 (s, 1H), 7.36 (t, J=7.8 Hz, 1H), 5.84-5.75 (m, 1H), 3.78-3.73 (m, 4H), 3.64 (s, 3H), 3.52-3.47 (m, 4H), 2.53 (s, 3H), 2.46 (s, 3H), 1.44 (d, J=6.4 Hz, 3H); LCMS m/e: 446.2 (MH⁺).

Formate salt of Compound 56: ¹HNMR (400 MHz, DMSO-d₆) δ 8.67 (s, 1H), 8.30 (s, 1H), 7.93 (d, J=7.7 Hz, 1H), 7.63 (d, J=7.6 Hz, 1H), 7.50 (s, 1H), 7.37 (t, J=7.7 Hz, 1H), 5.71 (q, J=6.8 Hz, 1H), 3.67 (s, 3H), 3.63-3.57 (m, 8H), 3.56-3.51 (m, 3H), 2.63 (s, 3H), 2.06 (s, 3H), 1.45 (d, J=6.6 Hz, 3H); LCMS m/e: 444.3 (MH⁺).

Formate salt of Compound 57: ¹HNMR (400 MHz, DMSO-d₆) δ 8.71 (s, 1H), 8.33 (s, 1H), 7.91 (d, J=7.8 Hz, 1H), 7.63 (d, J=7.6 Hz, 1H), 7.37 (t, J=7.8 Hz, 1H), 7.21 (s, 1H), 5.73 (q, J=6.5 Hz, 1H), 3.71 (s, 3H), 3.50-3.42 (m, 4H), 2.64 (s, 3H), 2.52 (s, 3H), 2.03-1.94 (s, 4H), 1.49 (d, J=6.7 Hz, 3H); LCMS m/e: 387.2 (MH⁺).

Compound 58: ¹HNMR (400 MHz, DMSO-d₆) δ 8.63 (s, 1H), 7.90 (d, J=7.8 Hz, 1H), 7.62 (d, J=7.5 Hz, 1H), 7.48 (s, 1H), 7.37 (t, J=7.8 Hz, 1H), 5.70 (q, J=6.7 Hz, 1H), 4.02-3.93 (m, 2H), 3.66 (s, 3H), 3.25-3.20 (m, 3H), 2.63 (s, 3H), 2.48 (s, 3H), 1.93 (dd, J=10.1, 3.4 Hz, 2H), 1.45 (d, J=6.7 Hz, 5H); LCMS m/e: 434.5 (MH⁺).

Formate salt of Compound 59: ¹HNMR (400 MHz, DMSO-d₆) δ 8.72-8.66 (m, 1H), 8.34-8.28 (m, 1H), 7.93 (d, J=6.0 Hz, 1H), 7.66-7.56 (m, 2H), 7.37 (t, J=7.7 Hz, 1H), 5.74-5.68 (m, 1H), 3.75 (s, 4H), 3.69-3.66 (m, 3H), 2.63 (s, 3H), 2.50 (s, 3H), 2.12-2.00 (m, 4H), 1.49-1.43 (m, 3H); LCMS m/e: 437.4 (MH⁺).

Formate salt of Compound 60: ¹HNMR (400 MHz, DMSO-d₆) δ 8.66 (s, 1H), 8.35 (s, 1H), 7.92 (d, J=7.8 Hz, 1H), 7.62 (d, J=7.6 Hz, 1H), 7.52 (s, 1H), 7.37 (t, J=7.7 Hz, 2H), 5.71 (q, J=6.2 Hz, 1H), 3.68 (s, 3H), 3.66-3.62 (m, 4H), 2.63 (s, 3H), 2.50 (s, 3H), 1.50-1.39 (m, 6H), 0.37 (s, 4H); LCMS m/e: 427.3 (MH⁺).

Formate salt of Compound 61: ¹HNMR (400 MHz, DMSO-d₆) δ 8.73-8.66 (m, 1H), 8.31-8.26 (m, 1H), 7.92 (d, J=7.8 Hz, 1H), 7.63 (d, J=7.6 Hz, 1H), 7.56-7.47 (m, 1H), 7.37 (t, J=7.7 Hz, 1H), 5.72 (q, J=5.7 Hz, 1H), 4.17 (d, J=12.6 Hz, 1H), 4.06 (d, J=11.1 Hz, 1H), 4.00-3.94 (m, 1H), 3.71-3.68 (m, 3H), 3.62-3.59 (m, 2H), 2.89 (t, J=12.0 Hz, 1H), 2.63 (s, 3H), 2.56 (t, J=11.0 Hz, 1H), 2.51 (s, 3H), 1.50-1.45 (m, 3H), 1.20 (d, J=6.2 Hz, 3H); LCMS m/e: 417.3 (MH⁺).

Compound 62: ¹HNMR (400 MHz, DMSO-d₆) δ 8.63 (s, 1H), 7.93 (d, J=7.8 Hz, 1H), 7.52 (d, J=7.6 Hz, 1H), 7.36 (t, J=7.8 Hz, 1H), 7.23 (s, 1H), 5.79 (q, J=6.7 Hz, 1H), 3.91 (t, J=13.2 Hz, 2H), 3.70 (t, J=7.3 Hz, 2H), 3.64 (s, 3H), 2.64-2.55 (m, 2H), 2.53 (s, 3H), 2.46 (s, 3H), 1.45 (d, J=6.7 Hz, 3H); LCMS m/e: 466 (MH⁺).

Formate salt of Compound 63: ¹H NMR (400 MHz, DMSO-d₆) δ 8.96 (s, 1H), 8.23 (brs, 1H), 7.88 (d, J=7.7 Hz, 1H), 7.77 (s, 1H), 7.59 (d, J=7.8 Hz, 1H), 7.41 (t, J=7.7 Hz, 1H), 5.85 (q, J=6.8 Hz, 1H), 3.85 (s, 3H), 3.64 (t, 4H), 2.76-2.71 (m, 1H), 2.61 (s, 3H), 2.60 (t, 3H), 2.58 (s, 4H), 1.62 (d, J=6.8 Hz, 3H), 1.02 (d, J=6.5 Hz, 6H); LCMS m/e: 487.3 (MH⁺).

Compound 64: ¹HNMR (400 MHz, DMSO-d₆) δ 8.58 (s, 1H), 7.93 (d, J=7.4 Hz, 1H), 7.61 (d, J=6.9 Hz, 1H), 7.42 (s, 1H), 7.36 (t, J=7.7 Hz, 1H), 5.70 (q, J=6.6 Hz, 1H), 3.63 (s, 3H), 3.58-3.54 (m, 4H), 2.62 (s, 3H), 2.46 (s, 3H), 1.65-1.54 (m, 6H), 1.43 (d, J=6.7 Hz, 3H); LCMS m/e: 401.4 (MH⁺).

Compound 65: ¹H NMR (400 MHz, DMSO-d₆) δ 8.54 (s, 1H), 7.90 (d, J=7.3 Hz, 1H), 7.60 (d, J=7.5 Hz, 1H), 7.35 (t, J=7.8 Hz, 1H), 7.02 (s, 1H), 5.70 (q, J=6.7 Hz, 1H), 3.63-3.60 (m, 4H), 3.36 (s, 3H), 2.61 (s, 3H), 2.44 (s, 3H), 1.93 (t, J=6.8 Hz, 2H), 1.41 (d, J=6.7 Hz, 3H), 069-0.61 (m, 4H); LCMS m/e: 413.3 (MH⁺).

Formate salt of Compound 66: ¹H NMR (400 MHz, DMSO-d₆) δ 8.64-8.58 (m, 1H), 8.30 (dd, J=7.5, 3.7 Hz, 1H), 7.94 (d, J=7.9 Hz, 1H), 7.61 (d, J=7.6 Hz, 1H), 7.36 (t, J=9.4 Hz, 1H), 7.18 (dd, J=14.6, 4.3 Hz, 1H), 5.70 (q, J=6.3 Hz, 1H), 3.91 (t, J=13.2 Hz, 2H), 3.69 (t, J=7.2 Hz, 2H), 3.64 (s, 3H), 2.62 (s, 3H), 2.60-2.53 (m, 2H), 2.47 (s, 3H), 1.42 (s, 3H); LCMS m/e: 423.2 (MH⁺).

Formate salt of Compound 67: ¹HNMR (400 MHz, DMSO-d₆) δ 8.67 (s, 1H), 8.37 (s, 1H), 7.93 (d, J=7.8 Hz, 1H), 7.62 (d, J=7.6 Hz, 1H), 7.53 (s, 1H), 7.37 (t, J=7.8 Hz, 1H), 5.71 (q, J=6.7 Hz, 1H), 3.93-3.87 (m, 2H), 3.69 (s, 3H), 3.45-3.32 (m, 2H), 3.11 (m, 1H), 2.64 (s, 3H), 2.53-2.50 (m, 3H), 1.64-1.41 (m, 7H), 1.16 (s, 3H); LCMS m/e: 431.3 (MH⁺).

Compound 68: ¹H NMR (400 MHz, DMSO-d₆) δ 8.71 (s, 1H), 8.45 (d, J=7.7 Hz, 1H), 7.89 (d, J=7.8 Hz, 1H), 7.51 (d, J=7.6 Hz, 1H), 7.35 (t, J=7.7 Hz, 1H), 5.77 (q, J=6.3 Hz, 1H), 4.36 (s, 1H), 4.25 (s, 1H), 4.10-4.05 (m, 1H), 3.98-3.92 (m, 1H), 3.83-3.81 (m, 1H), 3.79-3.75 (m, 1H), 3.61 (s, 3H), 2.51 (s, 3H), 2.45 (s, 3H), 2.07 (d, J=5.6 Hz, 3H), 1.38 (d, J=6.6 Hz, 3H); LCMS m/e: 501.2 (MH⁺).

Formate salt of Compound 69: ¹H NMR (400 MHz, DMSO-d₆) δ 8.61 (s, 1H), 8.30 (s, 1H), 7.90 (d, J=7.7 Hz, 1H), 7.62 (d, J=7.6 Hz, 1H), 7.36 (t, J=7.8 Hz, 1H), 7.17-7.11 (m, 1H), 5.73-5.66 (m, 1H), 5.64-5.43 (m, 1H), 4.41-4.31 (m, 2H), 4.13-4.03 (m, 2H), 3.64 (s, 3H), 2.62 (s, 3H), 2.47 (s, 3H), 1.44 (d, J=6.7 Hz, 3H); LCMS m/e: 391.3 (MH⁺).

Compound 70: ¹H NMR (400 MHz, CD₃OD) δ 9.03 (s, 1H), 7.75 (d, J=7.8 Hz, 1H), 7.61 (d, J=7.6 Hz, 1H), 7.49-7.43 (m, 1H), 7.37 (t, J=7.8 Hz, 1H), 5.74-5.65 (m, 1H), 4.53 (t, J=12.0 Hz, 4H), 3.99 (s, 3H), 3.31 (d, 3H), 2.73 (s, 3H), 1.71 (d, J=7.0 Hz, 3H); LCMS m/e: 409.2 (MH⁺).

Compound 71: ¹H NMR (400 MHz, DMSO-d₆) δ 8.41 (s, 1H), 7.90 (d, J=7.8 Hz, 1H), 7.58 (d, J=7.6 Hz, 1H), 7.34 (t, J=7.7 Hz, 1H), 6.91 (s, 1H), 5.70-5.62 (m, 1H), 3.97 (t, J=6.9 Hz, 4H), 3.54 (s, 3H), 2.60 (s, 3H), 2.39 (s, 3H), 2.37-2.29 (m, 2H), 1.37 (d, J=6.3 Hz, 3H); LCMS m/e: 373.3 (MH⁺).

Trifluoroacetate salt of Compound 72: ¹H NMR (400 MHz, DMSO-d₆) δ 10.84 (s, 1H), 9.05 (s, 1H), 7.98 (d, J=7.0 Hz, 1H), 7.69 (d, J=7.7 Hz, 2H), 7.42 (t, J=7.8 Hz, 1H), 5.82 (p, J=6.6 Hz, 1H), 3.91 (s, 3H), 3.84 (dd, J=15.0, 10.9 Hz, 2H), 3.51 (t, J=9.0 Hz, 2H), 2.68 (d, J=7.6 Hz, 6H), 1.83-1.75 (m, 2H), 1.69 (d, J=7.0 Hz, 3H), 0.86-0.76 (m, 1H), 0.20 (dd, J=8.3, 4.2 Hz, 1H); LCMS m/e: 399 (MH⁺).

Formate salt of Compound 73: ¹H NMR (400 MHz, DMSO-d₆) δ 8.63 (s, 1H), 8.32 (d, J=5.9 Hz, 1H), 7.91 (d, J=6.3 Hz, 1H), 7.76-7.55 (m, 1H), 7.37 (dd, J=13.4, 7.2 Hz, 1H), 7.14 (d, J=7.9 Hz, 1H), 5.81-5.64 (m, 1H), 3.91 (s, 2H), 3.86 (s, 2H), 3.72 (s, 1H), 3.67 (s, 3H), 2.63 (d, J=3.5 Hz, 3H), 2.55 (s, 3H), 1.47 (d, J=2.7 Hz, 6H); LCMS m/e: 403 (MH⁺).

Compound 74: ¹H NMR (400 MHz, CD₃OD) δ 9.04 (d, J=4.8 Hz, 1H), 7.73 (d, J=7.6 Hz, 1H), 7.61 (d, J=7.6 Hz, 1H), 7.50 (d, J=4.5 Hz, 1H), 7.36 (t, J=7.8 Hz, 1H), 5.98-5.85 (m, 1H), 4.60-4.49 (m, 1H), 4.10-4.02 (m, 2H), 4.00 (d, J=2.5 Hz, 3H), 3.86 (d, J=11.5 Hz, 1H), 3.79-3.74 (m, 1H), 3.68-3.56 (m, 1H), 3.38-3.33 (m, 1H), 3.31 (s, 3H), 2.73 (s, 3H), 1.71 (d, J=7.0 Hz, 3H), 1.29 (d, J=6.7 Hz, 3H); LCMS m/e: 417.4 (MH⁺).

Formate salt of Compound 75: ¹HNMR (400 MHz, CD₃OD) δ 9.05 (s, 1H), 8.49 (s, 1H), 7.73 (d, J=7.9 Hz, 1H), 7.61 (d, J=7.4 Hz, 1H), 7.52 (s, 1H), 7.37 (t, J=7.8 Hz, 1H), 5.91 (q, J=6.9 Hz, 1H), 4.56 (d, J=4.0 Hz, 1H), 4.06 (dd, J=11.1, 3.9 Hz, 2H), 4.01 (s, 3H), 3.86 (d, J=11.5 Hz, 1H), 3.77 (dd, J=11.5, 3.0 Hz, 1H), 3.63 (td, J=11.9, 3.0 Hz, 1H), 3.36 (dd, J=12.9, 4.0 Hz, 1H), 2.74 (s, 6H), 1.72 (d, J=7.0 Hz, 3H), 1.29 (d, J=6.7 Hz, 3H); LCMS m/e: 417 (MH⁺).

Formate salt of Compound 76: ¹H NMR (400 MHz, DMSO-d₆) δ 8.69 (s, 1H), 8.29 (d, J=9.1 Hz, 1H), 7.90 (d, J=7.8 Hz, 1H), 7.63 (d, J=7.5 Hz, 1H), 7.37 (t, J=7.8 Hz, 1H), 7.21 (d, J=16.4 Hz, 1H), 5.72 (q, J=6.7 Hz, 1H), 4.37 (dt, J=6.1, 5.1 Hz, 1H), 4.27-4.22 (m, 2H), 3.86-3.82 (m, 2H), 3.70 (s, 3H), 2.63 (s, 3H), 2.51 (s, 3H), 2.50 (s, 3H), 1.49 (d, J=3.9 Hz, 3H); LCMS m/e: 403 (MH⁺).

Formate salt of Compound 77: ¹H NMR (400 MHz, CDCl₃) δ 8.91 (s, 1H), 8.41-8.32 (m, 1H), 8.30 (s, 1H), 8.06 (d, J=7.5 Hz, 1H), 7.49 (d, J=7.7 Hz, 1H), 7.30 (d, J=7.9 Hz, 1H), 5.84 (q, J=6.8 Hz, 1H), 4.36-4.18 (m, 2H), 3.88-3.78 (m, 4H), 3.76-3.63 (m, 4H), 3.19-2.99 (m, 2H), 2.60 (s, 3H), 2.43 (dd, J=15.2, 8.2 Hz, 2H), 1.71 (d, J=6.8 Hz, 3H). LC-MS m/e: (MH⁺); LCMS m/e: 458 (MH⁺).

Formate salt of Compound 78: ¹H NMR (400 MHz, DMSO-d₆) δ 8.86 (s, 1H), 8.35 (s, 1H), 7.93 (d, J=7.7 Hz, 1H), 7.59 (s, 1H), 7.54 (d, J=7.7 Hz, 1H), 7.38 (t, J=7.8 Hz, 1H), 5.79 (q, J=6.7 Hz, 1H), 5.02-4.82 (m, 1H), 3.75 (s, 4H), 3.53 (s, 4H), 2.56 (d, J=6.8 Hz, 6H), 1.59 (t, J=7.4 Hz, 6H), 1.48 (d, J=6.6 Hz, 3H); LCMS m/e: 476 (MH⁺).

Compound 79: ¹H NMR (400 MHz, CD₃OD) δ 9.22 (s, 1H), 7.70 (d, J=7.8 Hz, 1H), 7.58 (d, J=7.8 Hz, 1H), 7.51 (s, 1H), 7.35 (t, J=7.8 Hz, 1H), 5.97 (q, J=6.8 Hz, 1H), 3.89-3.77 (m, 4H), 3.73-3.64 (m, 4H), 3.31 (s, 3H), 2.80-2.74 (m, 1H), 2.62 (s, 3H), 1.69 (d, J=6.8 Hz, 3H), 1.49 (d, J=7.0 Hz, 2H), 1.13 (s, 2H); LCMS m/e: 472 (MH⁺).

### Example 80

### Synthesis of (R,Z)-6-(3-(difluoromethyl)-5,6-dihydroimidazo[1,2-a]pyrazin-7(8H)-yl)-1,2-dimethyl-N-(1-(2-methyl-3-(trifluoromethyl)phenyl)ethyl)pyrido[3,4-d]pyrimidin-4(1H)-imine (Compound 80)

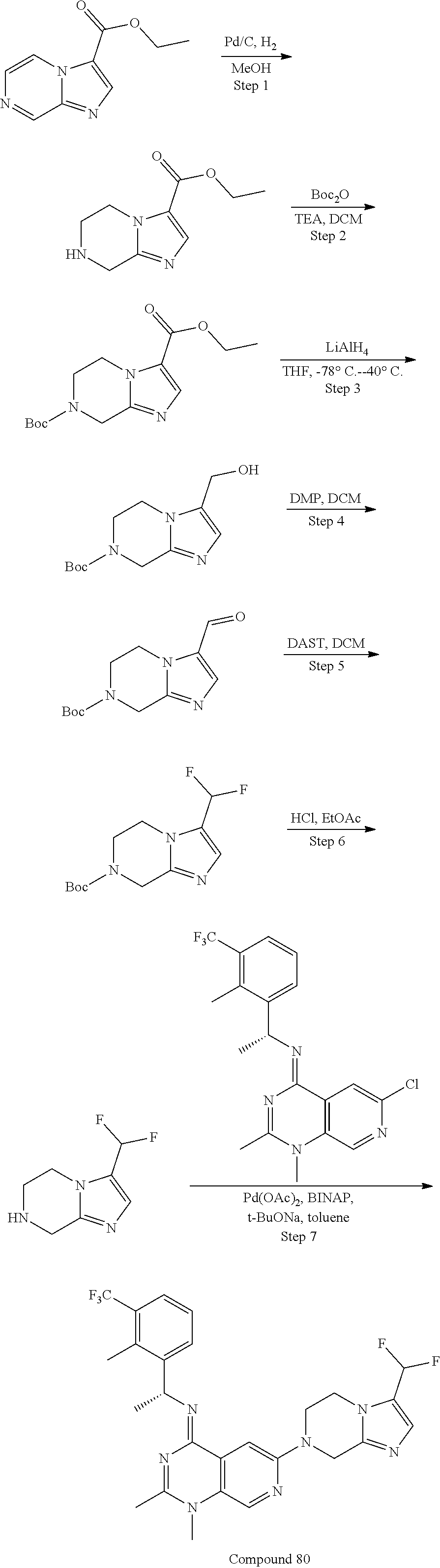

### Step 1. Synthesis of ethyl 5,6,7,8-tetrahydroimidazo[1,2-a]pyrazine-3-carboxylate

To a solution of ethyl imidazo[1,2-a]pyrazine-3-carboxylate (5 g, 26.2 mmol) in MeOH (300 mL) was added Pd/C (1 g, 10% and 55% wet) at 25° C. under H₂ balloon. After stirring for 16 hrs, the reaction mixture was filtered through a pad of Celite®. The filtrate was concentrated to afford the title compound (5 g, 25.6 mmol, 97.9% yield) as a white solid.

LC-MS m/e: 196.2 (MH⁺).

### Step 2. Synthesis of 7-(tert-butyl) 3-ethyl 5,6-dihydroimidazo[1,2-a]pyrazine-3,7(8H)-dicarboxylate

To a solution of ethyl 5,6,7,8-tetrahydroimidazo[1,2-a]pyrazine-3-carboxylate (2.4 g, 12.3 mmol) and di-tert-butyl dicarbonate (3.16 mL, 14.5 mmol) in DCM (50 mL) was added Et₃N (3.76 mL, 27.1 mmol) at 25° C. and stirred for 3 hrs. The reaction mixture was poured into ice water (20 mL) and the aqueous layer was extracted with DCM (50 mL×3). The combined organic layers were washed with brine (50 mL), dried over anhydrous Na₂SO₄, filtered, and concentrated to dryness. The residue was purified by flash column on silica gel (gradient, PE to PE:EtOAc=3:1) to afford the title compound (3 g, 10.2 mmol, 82.6% yield) as a yellow solid.

LCMS m/e: 296.2 (MH⁺).

### Step 3. Synthesis of tert-butyl 3-(hydroxymethyl)-5,6-dihydroimidazo[1,2-a]pyrazine-7(8H)-carboxylate

To a solution of 2-methylpropan-2-yl 3-(ethoxycarbonyl)-5,6,7,8-tetrahydroimidazo[1,2-a]pyrazine-7-carboxylate (2.4 g, 8.13 mmol) in THF (50 mL) was added LiAlH₄ (0.46 g, 12.1 mmol) at −78° C. After stirring the mixture at −40° C. for 2 hrs, THF (20 mL) was added, followed by dropwise addition of water (3 mL), 15% NaOH aqueous solution (3 mL), and water (9 mL). Anhydrous MgSO₄ was added. The mixture was stirred at room temperature for 0.5 hrs. The resulting mixture was filtered through a pad of Celite® and washed with EtOAc (100 mL). The filtrate was concentrated under reduced pressure to afford the title compound (1.5 g, 5.92 mmol, 72.9% yield) as a white solid.

LCMS m/e: 254.3 (MH⁺).

### Step 4. Synthesis of tert-butyl 3-formyl-5,6-dihydroimidazo[1,2-a]pyrazine-7(8H)-carboxylate

To a solution of tert-butyl 3-(hydroxymethyl)-5,6-dihydroimidazo[1,2-a]pyrazine-7(8H)-carboxylate (1 g, 3.95 mmol) in DCM (20 mL) was added Dess-Martin periodinane (1.67 g, 3.95 mmol) at 0° C. and stirred at 25° C. for 12 hrs. The reaction solution was poured into a mixture of saturated Na₂S₂O₃ solution (50 mL) and saturated NaHCO₃ solution (50 mL) and stirred for 0.5 hrs. The aqueous layer was extracted with DCM (20 mL×3). The combined organic layers were washed with brine (30 mL), dried over anhydrous Na₂SO₄, filtered, and concentrated to dryness. The residue was purified by flash column on silica gel (gradient, PE to PE:EtOAc=1:1) to afford the title compound (600 mg, 2.39 mmol, 60.5% yield) as a yellow solid.

LCMS m/e: 252.26 (MH⁺).

### Step 5. Synthesis of tert-butyl 3-(difluoromethyl)-5,6-dihydroimidazo[1,2-a]pyrazine-7(8H)-carboxylate

To a solution of tert-butyl 3-formyl-5,6-dihydroimidazo[1,2-a]pyrazine-7(8H)-carboxylate (200 mg, 0.732 mmol) in DCM (5 mL) was added DAST (1 mL, 7.96 mmol) at 0° C. and stirred at 0° C. for 4 hrs. The reaction mixture was basified by adding cold saturated NaHCO₃ solution (20 mL) and the aqueous layer was extracted with DCM (30 mL×3). The combined organic layers were washed with brine (50 mL), dried over anhydrous Na₂SO₄, filtered, and concentrated to dryness. The residue was purified by flash column on silica gel (gradient, PE to PE:EtOAc=3:1) to afford the title compound (150 mg, 0.549 mmol, 69% yield) as a yellow oil. LCMS m/e: 274.3 (MH⁺).

### Step 6. Synthesis of 3-(difluoromethyl)-5,6,7,8-tetrahydroimidazo[1,2-a]pyrazine

To a solution of tert-butyl 3-(difluoromethyl)-5,6-dihydroimidazo[1,2-a]pyrazine-7(8H)-carboxylate (150 mg, 0.55 mmol) in DCM (5 mL) was added TFA (1 mL) at 0° C. and stirred at room temperature for 12 hrs. The reaction mixture was concentrated to dryness. The residue was diluted with DCM (20 mL) and adjusted to pH 8 with saturated NaHCO₃ solution (20 mL). The organic layer was washed with brine (10 mL), dried over anhydrous Na₂SO₄, filtered, and concentrated to dryness. The residue was purified by flash column on silica gel (gradient, DCM to DCM:MeOH=20:1) to afford the title compound (70 mg, 0.404 mmol, 73.6% yield) as a yellow oil.

LCMS m/e: 174.3 (MH⁺).

### Step 7. Synthesis of (R,Z)-6-(3-(difluoromethyl)-5,6-dihydroimidazo[1,2-a]pyrazin-7(8H)-yl)-1,2-dimethyl-N-(1-(2-methyl-3-(trifluoromethyl)phenyl)ethyl)pyrido[3,4-d]pyrimidin-4(1H)-imine (Compound 80)

To a solution of (R,Z)-6-chloro-1,2-dimethyl-N-(1-(2-methyl-3-(trifluoromethyl)phenyl)-ethyl)pyrido[3,4-d]pyrimidin-4(1H)-imine (100 mg, 0.25 mmol) and 3-(difluoromethyl)-5,6,7,8-tetrahydroimidazo[3,2-a]pyrazine (53 mg, 0.304 mmol) in toluene (10 mL) was added t-BuONa (73 mg, 0.76 mmol), BINAP (32 mg, 0.051 mmol) and Pd(OAc)₂ (12 mg, 0.05 mmol) at 25° C. The mixture was stirred at 100° C. for 2 hrs under N₂. The reaction mixture was cooled to room temperature, poured into water (20 mL) and extracted with DCM (20 mL×3). The combined organic layers were washed with brine (30 mL), dried over anhydrous Na₂SO₄, filtered, and concentrated to dryness. The residue was purified by prep-HPLC to afford formate salt of the desired compound (15 mg) as a yellow solid.

¹HNMR (400 MHz, DMSO-d₆) δ 8.69 (s, 1H), 8.34 (s, 1H), 7.96 (d, J=7.7 Hz, 1H), 7.64 (s, 1H), 7.53 (d, J=7.6 Hz, 1H), 7.37 (t, J=7.8 Hz, 1H), 7.33-7.05 (m, 2H), 5.80 (q, J=6.7 Hz, 1H), 4.81 (s, 2H), 4.17 (dd, J=13.7, 4.2 Hz, 4H), 3.66 (s, 3H), 2.54 (s, 3H), 2.47 (s, 3H), 1.47 (d, J=6.7 Hz, 3H).

LCMS m/e: 532.2 (MH⁺).

### Example 81

### Synthesis of (R,Z)-6-(1-acetyl-4-hydroxypiperidin-4-yl)-1,7-dimethyl-4-((1-(2-methyl-3-(trifluoromethyl)phenyl)ethyl)imino)-1,7-dihydropyrido[3,4-d]pyrimidin-8(4H)-one (Compound 81)

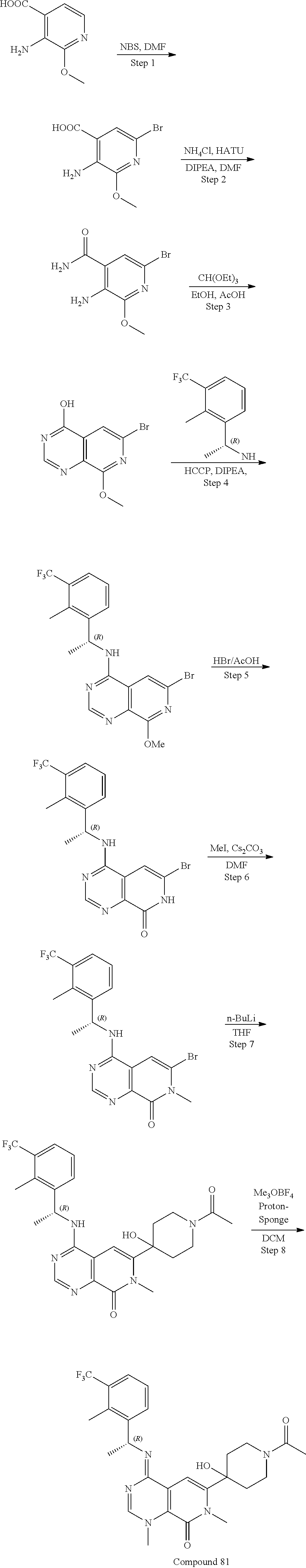

### Step 1. Synthesis of 3-amino-6-bromo-2-methoxyisonicotinic Acid

To a solution of 3-amino-2-methoxyisonicotinic acid (5 g, 27.3 mmol) in DMF (30 mL) was added NBS (5.11 g, 28.7 mmol) in portions, the mixture was stirred for 1 hr at 20° C., diluted with water (40 mL) and extracted with EtOAc (100 mL×2). The combined organic layers were washed with brine, dried over anhydrous Na₂SO₄, filtered, and concentrated to dryness. The residue was purified by flash column chromatography on silica gel (gradient, PE to PE:EtOAc=3:1) to afford the title compound (4.2 g, 17 mmol, 62.1% yield) as a brown solid.

LC-MS m/e: 247, 249 (MH⁺).

### Step 2. Synthesis of 3-amino-6-bromo-2-methoxyisonicotinamide

To a solution of 3-amino-6-bromo-2-methoxyisonicotinic acid (4.2 g, 17 mmol) in DMF (20 mL) was added DIPEA (4.39 g, 34 mmol), NH₄Cl (1.36 g, 25.5 mmol) and HATU (7.75 g, 20.4 mmol) and stirred for 1 hr at 20° C. The reaction mixture was diluted with water (40 mL) and extracted with EtOAc (100 mL×2). The combined organic layers were washed with brine (100 mL), dried over anhydrous Na₂SO₄, filtered, and concentrated to dryness. The residue was purified by flash column chromatography on silica gel (gradient, PE to PE:EtOAc=1:1) to afford the title compound (4 g, 16.2 mmol, 95.6% yield) as a yellow solid.

LC-MS m/e: 246, 248 (MH⁺).

### Step 3. Synthesis of 6-bromo-8-methoxypyrido[3,4-d]pyrimidin-4-ol

To a solution of 3-amino-6-bromo-2-methoxyisonicotinamide (4 g, 16.2 mmol) in EtOH (80 mL) was added AcOH (13.9 mL, 243 mmol) and CH(OEt)₃ (36 g, 243 mmol) and stirred at 120° C. for 3 hrs. The resulting mixture was cooled to room temperature, concentrated under reduced pressure and the residue was triturated with EtOAc (50 mL) to give the title compound (3.4 g, 13.2 mmol, 81.6% yield) as a yellow solid.

LC-MS m/e: 256, 258 (MH⁺).

### Step 4. Synthesis of (R)-6-bromo-8-methoxy-N-(1-(2-methyl-3-(trifluoromethyl)phenyl)ethyl)-pyrido[3,4-d]pyrimidin-4-amine

To a solution of 6-bromo-8-methoxypyrido[3,4-d]pyrimidin-4-ol (3.4 g, 13.2 mmol) in MeCN (30 mL) was added DIPEA (6.85 g, 53.1 mmol) and HCCP (5.08 g, 14.6 mmol). After stirring for 1 hr at 25° C., (1R)-1-[2-methyl-3-(trifluoromethyl)phenyl]ethan-1-amine hydrochloride (3.46 g, 14.6 mmol) was added. After stirring for 1 hr at 25° C., the mixture was diluted with water (30 mL) and extracted with EtOAc (50 mL×2). The combined organic layers were washed with brine (50 mL), dried over anhydrous Na₂SO₄, filtered, and concentrated to dryness. The residue was purified by flash column chromatography on silica gel (gradient, DCM to DCM:MeOH=30:1) to afford the title compound (4.5 g, 10.1 mmol, 76.8% yield) as a yellow solid.

LC-MS m/e: 441, 443 (MH⁺).

### Step 5. Synthesis of (R)-6-bromo-4-((1-(2-methyl-3-(trifluoromethyl)phenyl)ethyl)amino)-pyrido[3,4-d]pyrimidin-8(7H)-one

A suspension of (R)-6-bromo-8-methoxy-N-(1-(2-methyl-3-(trifluoromethyl)phenyl)-ethyl)pyrido[3,4-d]pyrimidin-4-amine (1.2 g, 2.72 mmol) in HBr (10 mL, 48% in water) was stirred at 100° C. for 2 hrs. The mixture was cooled to room temperature and basified with 20% NaOH to ˜pH 7 at 10° C. The solid obtained was collected by filtration and dried under vacuum to give the title compound (600 mg, 1.4 mmol, 51.6% yield) as a yellow solid.

LC-MS m/e: 427, 429 (MH⁺).

### Step 6. Synthesis of (R)-6-bromo-7-methyl-4-((1-(2-methyl-3-(trifluoromethyl)phenyl)ethyl)-amino)pyrido[3,4-d]pyrimidin-8(7H)-one

To a solution of (R)-6-bromo-4-((1-(2-methyl-3-(trifluoromethyl)phenyl)ethyl)-amino)pyrido[3,4-d]pyrimidin-8(7H)-one (600 mg, 1.4 mmol) in DMF (5 mL) was added Cs₂CO₃ (686 mg, 2.1 mmol) and stirred at 20° C. for 0.5 hrs. MeI (199 mg, 1.4 mmol) was added at 0° C. and the mixture was stirred at 20° C. for 1 hr. The resulting mixture was poured into ice water (50 mL) and extracted with EtOAc (50 mL×2). The combined organic layers were washed with brine (50 mL×2), dried over anhydrous Na₂SO₄, filtered, and concentrated to dryness. The residue was purified by silica gel chromatography (gradient, DCM to DCM:MeOH=10:1) to afford the title compound (250 mg, 0.567 mmol, 40.3% yield) as a yellow solid.

LC-MS m/e: 441, 443 (MH⁺).

### Step 7. Synthesis of (R)-6-(1-acetyl-4-hydroxypiperidin-4-yl)-7-methyl-4-((1-(2-methyl-3-(trifluoromethyl)phenyl)ethyl)amino)pyrido[3,4-d]pyrimidin-8(7H)-one

To a solution of (R)-6-bromo-7-methyl-4-((1-(2-methyl-3-(trifluoromethyl)phenyl)ethyl)-amino)pyrido[3,4-d]pyrimidin-8(7H)-one (230 mg, 0.521 mmol) in THF (10 mL) was added dropwise n-BuLi (1.04 mL, 2.6 mmol, 2.5M in Hexane) at −70° C. and stirred for 0.5 hrs followed by addition of 1-acetylpiperidin-4-one (154 mg, 0.782 mmol). The resulting mixture was stirred at −70° C. for 0.5 hrs, quenched with water (10 mL), and extracted with EtOAc (30 mL×2). The combined organic layers were washed with brine (10 mL), dried over anhydrous Na₂SO₄, and filtered. The filtrate was concentrated under reduced pressure and purified by silica gel flash column chromatography (gradient, DCM to DCM:MeOH=10:1) to give the title compound (50 mg, 0.099 mmol, 19.0% yield) as a yellow solid.

LC-MS m/e: 504 (MH⁺).

### Step 8. Synthesis of (R,Z)-6-(1-acetyl-4-hydroxypiperidin-4-yl)-1,7-dimethyl-4-((1-(2-methyl-3-(trifluoromethyl)phenyl)ethyl)imino)-1,7-dihydropyrido[3,4-d]pyrimidin-8(4H)-one (Compound 81)

To a solution of (R)-6-(1-acetyl-4-hydroxypiperidin-4-yl)-7-methyl-4-((1-(2-methyl-3-(trifluoromethyl)phenyl)ethyl)amino)pyrido[3,4-d]pyrimidin-8(7H)-one (50 mg, 0.099 mmol) in DCM (5 mL) was added proton sponge (21 mg, 0.099 mmol) and trimethyl-oxonium tetrafluoroborate (15 mg, 0.1 mmol) and stirred at 20° C. for 0.5 hrs. The resulting mixture was quenched with water (5 mL) and extracted with DCM (10 mL×2). The combined organic layers were washed with brine (10 mL), dried over anhydrous Na₂SO₄, filtered, and concentrated to dryness. The residue was purified by prep-HPLC (SilaSep™ C18 silica flash cartridge, 0%-40% MeCN in H₂O with 0.1% FA) to afford the title compound (13 mg) as a white solid.

¹H NMR (400 MHz, DMSO-d₆) δ 8.06 (s, 1H), 7.90 (d, J=8.0 Hz, 1H), 7.53 (d, J=8.0 Hz, 1H), 7.38 (t, J=8.0 Hz, 1H), 6.99 (s, 1H), 5.82 (s, 1H), 5.67 (q, J=6.8 Hz, 1H), 4.32 (d, J=12.8 Hz, 1H), 3.89 (s, 3H), 3.79 (s, 3H), 3.74 (s, 1H), 3.47 (d, J=3.1 Hz, 1H), 2.98 (t, J=13.1 Hz, 1H), 2.47 (s, 3H), 2.18-2.06 (m, 2H), 2.04 (s, 3H), 1.97-1.74 (m, 2H), 1.43-1.35 (m, 3H).

LCMS m/e: 518 (MH⁺).

### Example 82

### Synthesis of (R,Z)-6-(1-acetyl-1,2,3,6-tetrahydropyridin-4-yl)-1,7-dimethyl-4-((1-(2-methyl-3-(trifluoromethyl)phenyl)ethyl)imino)-1,7-dihydropyrido[3,4-d]pyrimidin-8(4H)-one (Compound 82)

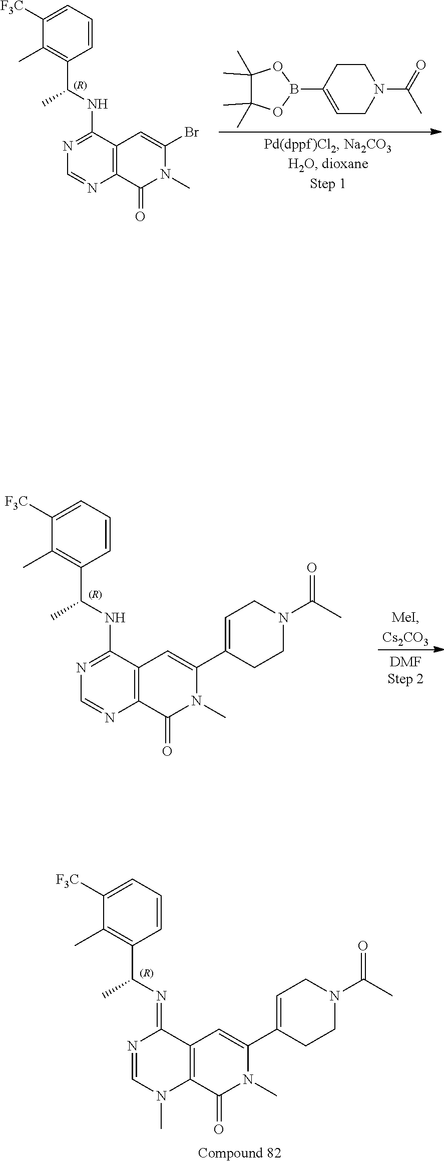

### Step 1. Synthesis of (R)-6-(1-acetyl-1,2,3,6-tetrahydropyridin-4-yl)-7-methyl-4-((1-(2-methyl-3-(trifluoromethyl)phenyl)ethyl)amino)pyrido[3,4-d]pyrimidin-8(7H)-one

To a solution of (R)-6-bromo-7-methyl-4-((1-(2-methyl-3-(trifluoromethyl)phenyl)ethyl)-amino)pyrido[3,4-d]pyrimidin-8(7H)-one (300 mg, 0.680 mmol) and 1-(4-(4,4,5,5-tetramethyl-1,3,2-dioxaborolan-2-yl)-3,6-dihydropyridin-1(2H)-yl)ethan-1-one (256 mg, 1.02 mmol) in dioxane (10 mL) and water (2 mL) were added Na₂CO₃ (216 mg, 2.04 mmol) and Pd(dppf)Cl₂ (49 mg, 0.068 mmol), and the mixture was stirred at 100° C. for 18 hrs under N₂. The solution was cooled and then purified by flash chromatography on silica gel (gradient, DCM to DCM: MeOH=10:1) to afford the title compound (250 mg, 0.515 mmol, 75.7% yield) as a pale brown solid. LCMS m/e: 486 (MH⁺).

### Step 2. Synthesis of (R,Z)-6-(1-acetyl-1,2,3,6-tetrahydropyridin-4-yl)-1,7-dimethyl-4-((1-(2-methyl-3-(trifluoromethyl)phenyl)ethyl)imino)-1,7-dihydropyrido[3,4-d]pyrimidin-8(4H)-one (Compound 82)

To a solution of (R)-6-(1-acetyl-1,2,3,6-tetrahydropyridin-4-yl)-7-methyl-4-((1-(2-methyl-3-(trifluoromethyl)phenyl)ethyl)amino)pyrido[3,4-d]pyrimidin-8(7H)-one (150 mg, 0.309 mmol) in DMF (2 mL) was added Cs₂CO₃ (151 mg, 0.463 mmol). The mixture was stirred at room temperature for 30 mins and then iodomethane (65 mg, 0.463 mmol) is added at 0° C. The resulting mixture was stirred at room temperature for 30 mins, poured into water (15 mL) and extracted with EtOAc (20 mL×2). The combined organic layers were washed with brine (15 mL×2), dried over anhydrous Na₂SO₄, filtered and concentrated to dryness. The residue was purified by reverse phase chromatography (SilaSep™ C18 silica flash cartridge, 0%-40% MeCN in H₂O with 0.1% formic acid) to afford a formate salt of the title compound (80 mg) as a yellow solid.

¹H NMR (400 MHz, DMSO-d₆) δ 8.21 (s, 1H), 8.15 (s, 1H), 7.85 (d, J=8.4 Hz, 1H), 7.54 (d, J=7.6 Hz, 1H), 7.38 (t, J=7.8 Hz, 1H), 6.85 (s, 1H), 6.03 (s, 1H), 5.72 (q, J=6.8 Hz, 1H), 4.20-4.12 (m, 2H), 3.98 (s, 3H), 3.73-3.64 (m, 2H), 3.40 (s, 3H), 2.49 (s, 3H), 2.45-2.37 (m, 1H), 2.35-2.29 (m, 1H), 2.08 (s, 3H), 1.45 (d, J=6.7 Hz, 3H). LCMS m/e: 500 (MH⁺).

### Example 83

### Synthesis of (R,Z)-6-(1-acetyl-1,2,3,6-tetrahydropyridin-4-yl)-1,8-dimethyl-4-((1-(2-methyl-3-(trifluoromethyl)phenyl)ethyl)imino)-4,8-dihydropyrido[2,3-d]pyrimidin-7(1H)-one (Compound 83)

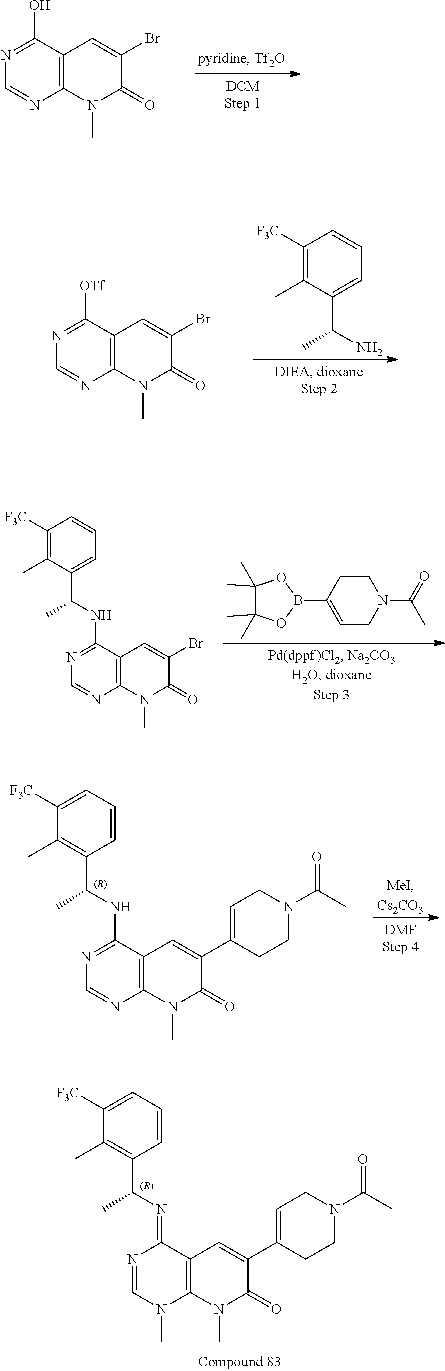

### Step 1. Synthesis of 6-bromo-8-methyl-7-oxo-7,8-dihydropyrido[2,3-d]pyrimidin-4-yl trifluoromethanesulfonate

To a solution of 6-bromo-4-hydroxy-8-methyl-7,8-dihydropyrido[2,3-d]pyrimidin-7-one (1 g, 3.91 mmol) in DCM (40 mL) was added pyridine (0.032 mL, 0.391 mmol) and Tf₂O (3.3 g, 11.8 mmol) at 0° C., and the mixture was stirred at room temperature for 3 hrs under N₂. The resulting mixture was partitioned between water (10 mL) and EtOAc (50 mL). The organic layer was washed with brine (20 mL), dried over anhydrous Na₂SO₄, filtered and concentrated to afford the title compound (595 mg, 1.53 mmol, 39.3% yield) as a yellow solid. LC-MS m/e: 388, 390 (MH⁺).

### Step 2. Synthesis of (R)-6-bromo-8-methyl-4-((1-(2-methyl-3-(trifluoromethyl)phenyl)-ethyl)amino)pyrido[2,3-d]pyrimidin-7(8H)-one

To a solution of 6-bromo-8-methyl-7-oxo-7,8-dihydropyrido[2,3-d]pyrimidin-4-yl trifluoromethanesulfonate (595 mg, 1.53 mmol) and (R)-1-(2-methyl-3-(trifluoromethyl)phenyl)-ethan-1-amine (312 mg, 1.53 mmol) in dioxane (5 mL) was added DIEA (1.27 mL, 7.67 mmol), and the mixture was stirred at 100° C. for 2 hrs under N₂. The resulting mixture was cooled to room temperature and concentrated to dryness. The residue was partitioned between water (10 mL) and EtOAc (30 mL). The organic layer was dried over anhydrous Na₂SO₄ and filtered. The filtrate was concentrated under reduced pressure and purified by flash chromatography on silica gel (gradient, PE to PE:EtOAc=10:1) to afford the title compound (503 mg, 1.18 mmol, 76.8% yield) as a yellow solid. LC-MS m/e: 441, 443 (MH⁺).

### Step 3. Synthesis of (R)-6-(1-acetyl-1,2,3,6-tetrahydropyridin-4-yl)-8-methyl-4-((1-(2-methyl-3-(trifluoromethyl)phenyl)ethyl)amino)pyrido[2,3-d]pyrimidin-7(8H)-one

To a solution of (R)-6-bromo-8-methyl-4-((1-(2-methyl-3-(trifluoromethyl)phenyl)-ethyl)amino)pyrido[2,3-d]pyrimidin-7(8H)-one (400 mg, 0.936 mmol), Pd(dppf)Cl₂ (67 mg, 0.091 mmol) and 1-[4-(4,4,5,5-tetramethyl-1,3,2-dioxaborolan-2-yl)-1,2,3,6-tetrahydropyridin-1-yl]ethan-1-one (342 mg, 1.36 mmol) in dioxane (15 mL) and H₂O (3 mL) was added Na₂CO₃ (289 mg, 2.73 mmol), and the mixture was stirred at 100° C. for 3 hrs under N₂. The resulting mixture was cooled to room temperature and purified by flash chromatography on silica gel (gradient, DCM to DCM:MeOH=10:1) to afford the title compound (422 mg, 0.869 mmol, 92.8% yield) as a yellow solid. LC-MS m/e: 486 (MH⁺).

### Step 4. Synthesis of (R,Z)-6-(1-acetyl-1,2,3,6-tetrahydropyridin-4-yl)-1,8-dimethyl-4-((1-(2-methyl-3-(trifluoromethyl)phenyl)ethyl)imino)-4,8-dihydropyrido[2,3-d]pyrimidin-7(1H)-one

A mixture of (R)-6-(1-acetyl-1,2,3,6-tetrahydropyridin-4-yl)-8-methyl-4-((1-(2-methyl-3-(trifluoromethyl)phenyl)ethyl)amino)pyrido[2,3-d]pyrimidin-7(8H)-one (200 mg, 0.412 mmol) and Cs₂CO₃ (201 mg, 0.618 mmol) in DMA (1 mL) was stirred at 15° C. for 10 mins under N₂, and CH₃I (88 mg, 0.618 mmol) is added at 0° C. The mixture was stirred at 15° C. for 12 hrs and then filtrated. The filtrate was purified by prep-HPLC (SilaSep™ C18 silica flash cartridge, 0%-40% MeCN in H₂O with 0.1% formic acid) to afford the title compound (24.6 mg, 0.049 mmol, 12.0% yield) as a white solid.

¹H NMR (400 MHz, DMSO-d₆) δ 8.49 (s, 1H), 7.82 (d, J=8.0 Hz, 1H), 7.75 (d, J=3.6 Hz, 1H), 7.68 (d, J=8.0 Hz, 1H), 7.48 (t, J=7.8 Hz, 1H), 6.49 (d, J=19.2 Hz, 1H), 6.16 (q, J=6.4 Hz, 1H), 4.05 (d, J=23.6 Hz, 2H), 3.61 (s, 3H), 3.58-3.46 (m, 2H), 2.94 (s, 3H), 2.50-2.23 (m, 2H), 2.20 (s, 3H), 2.00 (d, J=11.6 Hz, 3H), 1.61 (d, J=6.8 Hz, 3H). LC-MS m/e: 500 (MH⁺).

### Example 84

### Synthesis of (Z)-7-methoxy-1,2-dimethyl-N—((R)-1-(2-methyl-3-(trifluoromethyl)phenyl)-ethyl)-6-(((S)-tetrahydrofuran-3-yl)oxy)quinazolin-4(1H)-imine (Compound 84)

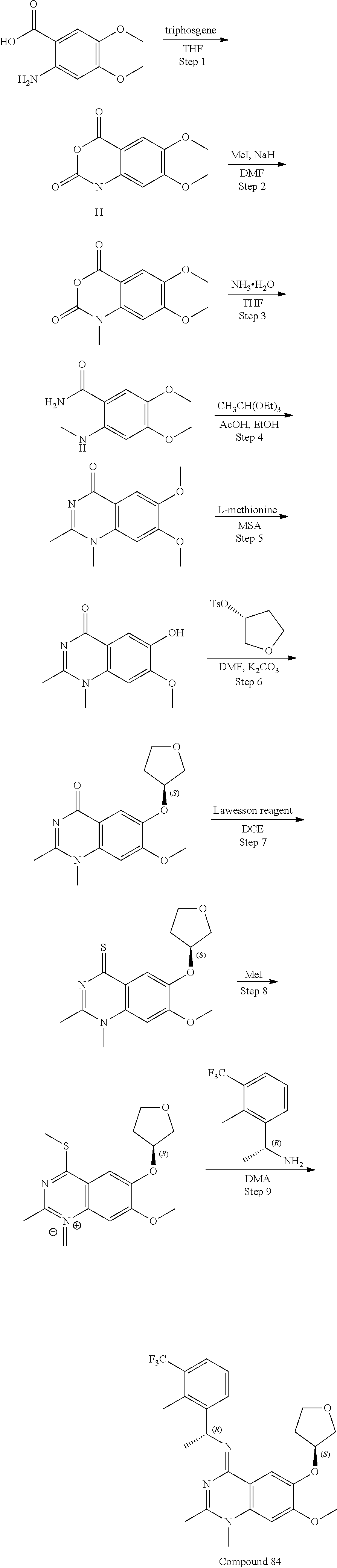

### Step 1. Synthesis of 6,7-dimethoxy-2H-benzo[d][1,3]oxazine-2,4(1H)-dione

To a solution of 2-amino-4,5-dimethoxybenzoic acid (10 g, 50.7 mmol) in THF (100 mL) at 0° C., triphosgene (4.51 g, 15.2 mmol) was added slowly and stirred at room temperature for 0.5 hrs. The solvent was removed under reduced pressure, petroleum ether (200 mL) was added and sonicated, the precipitated solid was filtered with suction. The filter cake was collected and dried under vacuum to afford the title compound (10 g, 44.8 mmol, 88.4% yield) as a gray solid.

LC-MS m/e: 224.1 (MH⁺).

### Step 2. Synthesis of 6,7-dimethoxy-1-methyl-2H-benzo[d][1,3]oxazine-2,4(1H)-dione

To a solution of 6,7-dimethoxy-2,4-dihydro-1H-benzo[2,1-d][1,3]oxazine-2,4-dione (5 g, 22.4 mmol) in DMF (50 mL) was added NaH (1.16 g, 29.0 mmol, 60% suspended in mineral oil) at 0° C. under N₂. The mixture was stirred at room temperature for 30 mins. After cooling to 0° C., MeI (2.1 mL, 33.6 mmol) was added and stirred at room temperature for 1 hr. The reaction mixture was diluted with ice water (300 mL) and the residue was filtrated, rinsed with water (20 mL) and ethyl ether (20 mL). The filter cake was collected and dried under vacuum to afford the title compound (4 g, 16.9 mmol, 75.3% yield) as a yellow solid. LC-MS m/e: 238.01 (MH⁺).

### Step 3. Synthesis of 4,5-dimethoxy-2-(methylamino)benzamide

To a solution of 6,7-dimethoxy-1-methyl-2,4-dihydro-1H-benzo[2,1-d][1,3]oxazine-2,4-dione (4 g, 16.9 mmol) in THF (60 mL) was added NH₄OH solution (30 mL, 30% in water) at 0° C. and stirred for 30 mins at 0° C. and then for 30 min at room temperature. THF was distilled under vacuum, the remaining suspension was neutralized with HCl solution (2M). The product was isolated by filtration. The filter cake was collected and dried under vacuum to afford the title compound (3.5 g, 16.6 mmol, 98.7% yield) as a yellow solid. LC-MS m/e: 211.1 (MH⁺).

### Step 4. Synthesis of 6,7-dimethoxy-1,2-dimethylquinazolin-4(1H)-one

To a solution of 4,5-dimethoxy-2-(methylamino)benzene-1-carboxamide (3.6 g, 17.1 mmol) in EtOH (75 mL) was added 1,1,1-triethoxyethane (47.1 mL, 257 mmol) and AcOH (14.7 mL, 257 mmol) at 25° C. The mixture was stirred at 120° C. in a sealed tube for 12 hrs. The reaction mixture was cooled to room temperature and filtered. The cake was washed with EtOAc (100 mL) and dried under vacuum to afford the title compound (3 g, 12.8 mmol, 74.8% yield) as a grey solid. LC-MS m/e: 235.1 (MH⁺).

### Step 5. Synthesis of 6-hydroxy-7-methoxy-1,2-dimethylquinazolin-4(1H)-one

To a solution of 6,7-dimethoxy-1,2-dimethylquinazolin-4(1H)-one (1 g, 4.27 mmol) in MSA (15 mL) was added L-methionine (0.96 g, 6.40 mmol) at 25° C. The mixture was stirred at 100° C. for 16 hrs. The reaction mixture was cooled to room temperature and basified to pH 5-6 with 40% sodium hydroxide solution under ice-water bath cooling. The solid was precipitated out, filtered, washed with water (30 mL×2) and dried under vacuum to afford the title compound (900 mg, 4.09 mmol, 95.7% yield) as a grey solid. LC-MS m/e: 221.10 (MH⁺).

### Step 6. Synthesis of (S)-7-methoxy-1,2-dimethyl-6-((tetrahydrofuran-3-yl)oxy)quinazolin-4(1H)-one

To a solution of 6-hydroxy-7-methoxy-1,2-dimethylquinazolin-4(1H)-one (200 mg, 0.908 mmol) and (3R)-tetrahydrofuran-3-yl 4-methylbenzenesulfonate (220 mg, 0.908 mmol) in DMF (10 mL) was added K₂CO₃ (377 mg, 2.72 mmol) at 25° C. The mixture was stirred at 100° C. for 16 hrs. The reaction mixture was cooled to room temperature, filtered and the filtrate was concentrated to dryness. The residue was purified by reverse phase column (C18, gradient, 0-30% CH₃CN in H₂O) to afford the title compound (100 mg, 0.344 mmol, 37.9% yield) as a yellow solid. LC-MS m/e: 291.3 (MH⁺).

### Step 7. Synthesis of (S)-7-methoxy-1,2-dimethyl-6-((tetrahydrofuran-3-yl)oxy)quinazoline-4(1H)-thione

To a solution of (S)-7-methoxy-1,2-dimethyl-6-((tetrahydrofuran-3-1)oxy)quinazolin-4(1H)-one (100 mg, 0.344 mmol) in dichloroethane (10 mL) was added Lawesson's reagent (84 mg, 0.207 mmol) at 25° C. The mixture was stirred at 80° C. for 12 hrs. The reaction mixture was cooled to room temperature and concentrated to dryness. The residue was purified by flash column (silica gel, gradient, 0-10% MeOH in DCM) to afford the title compound (44 mg, 0.144 mmol, 41.7% yield) as a yellow solid. LC-MS m/e: 307.2 (MH⁺).

### Step 8. Synthesis of (S)-7-methoxy-1,2-dimethyl-4-(methylthio)-6-((tetrahydrofuran-3-yl)oxy)quinazolin-1-ium iodide

A solution of (S)-7-methoxy-1,2-dimethyl-6-((tetrahydrofuran-3-yl)oxy)quinazoline-4(1H)-thione (44 mg, 0.144 mmol) in MeI (2 mL) is stirred at 25° C. for 2 hrs. The reaction mixture was concentrated to afford the title compound (44 mg, 0.136 mmol, 95% yield) as a red solid, which is used directly without further purification. LC-MS m/e: 322.4 (MH⁺).

### Step 9. Synthesis of (Z)-7-methoxy-1,2-dimethyl-N—((R)-1-(2-methyl-3-(trifluoromethyl)phenyl)ethyl)-6-(((S)-tetrahydrofuran-3-yl)oxy)quinazolin-4(1H)-imine (Compound 84)

To a solution of (S)-7-methoxy-1,2-dimethyl-4-(methylthio)-6-((tetrahydrofuran-3-yl)oxy)quinazolin-1-ium iodide (44 mg, 0.136 mmol) in DMA (2 mL) was added (1R)-1-[2-methyl-3-(trifluoromethyl)phenyl]ethan-1-amine (31 mg, 0.150 mmol) and stirred at 25° C. for 1 hr. The reaction mixture was purified by prep-HPLC to afford a formate salt of the title compound (20 mg) as a yellow solid.

¹H NMR (400 MHz, DMSO-d₆) δ 8.52 (s, 1H), 8.30 (d, J=24.6 Hz, 1H), 7.92 (t, J=8.5 Hz, 1H), 7.56 (d, J=7.4 Hz, 1H), 7.38 (t, J=7.6 Hz, 1H), 7.33-7.22 (m, 1H), 5.89-5.79 (m, 1H), 5.49-5.25 (m, 1H), 4.05-3.98 (m, 3H), 3.90-3.77 (m, 7H), 2.67 (s, 3H), 2.61 (s, 3H), 2.42-2.30 (m, 1H), 2.04-1.94 (m, 1H), 1.63 (d, J=6.7 Hz, 3H). LC-MS m/e: 476.3 (MH⁺).

### Example 85

### Synthesis of (R,Z)-3-(4-((1-(3-(difluoromethyl)-2-fluorophenyl)ethyl)imino)-1,2-dimethyl-1,4-dihydropyrido[3,4-d]pyrimidin-6-yl)prop-2-yn-1-amine (Compound 85)

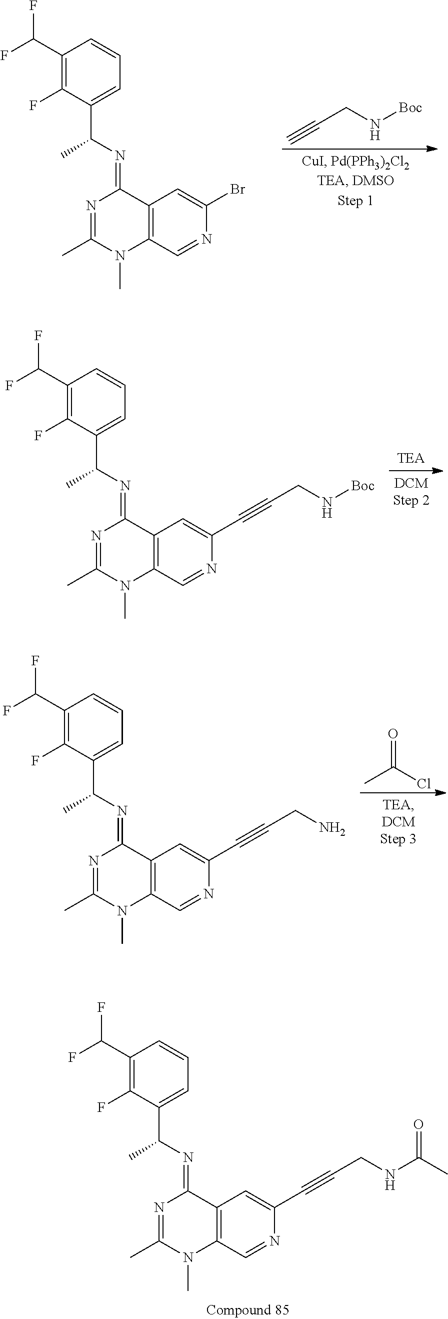

### Step 1. Synthesis of tert-butyl (R,Z)-(3-(4-((1-(3-(difluoromethyl)-2-fluorophenyl)ethyl)imino)-1,2-dimethyl-1,4-dihydropyrido[3,4-d]pyrimidin-6-yl)prop-2-yn-1-yl)carbamate

To a solution of (R,Z)-6-bromo-N-(1-(3-(difluoromethyl)-2-fluorophenyl)ethyl)-1,2-dimethylpyrido[3,4-d]pyrimidin-4(1H)-imine (300 mg, 0.705 mmol) and tert-butyl prop-2-yn-1-ylcarbamate (219 mg, 1.410 mmol) in DMSO (5 mL) and Et₃N (5 mL) were added CuI (27 mg, 0.141 mmol) and Pd(PPh₃)₂Cl₂ (99 mg, 0.141 mmol) and stirred at 50° C. for 16 hrs under N₂. The resulting solution was cooled to room temperature, poured into water (50 mL) and extracted with EtOAc (30 mL×2). The combined organic layers were washed with brine (30 mL×2), dried over anhydrous Na₂SO₄, filtered and concentrated. The residue was purified by an aluminum oxide column (gradient, PE to PE:DCM:MeOH=50:4:1) to afford the title compound (240 mg, 0.480 mmol, 68.1% yield) as light-yellow solid. LC-MS m/e: 500 (MH⁺).

### Step 2. Synthesis of (R,Z)-3-(4-((1-(3-(difluoromethyl)-2-fluorophenyl)ethyl)imino)-1,2-dimethyl-1,4-dihydropyrido[3,4-d]pyrimidin-6-yl)prop-2-yn-1-amine

To a solution of tert-butyl (R,Z)-(3-(4-((1-(3-(difluoromethyl)-2-fluorophenyl)ethyl)-imino)-1,2-dimethyl-1,4-dihydropyrido[3,4-d]pyrimidin-6-yl)prop-2-yn-1-yl)carbamate (240 mg, 0.48 mmol) in DCM (10 mL) was added TFA (2 mL) at 0° C. and stirred at room temperature for 1 hr. The resulting solution was quenched with saturated NaHCO₃ solution (40 mL) and extracted with DCM (20 mL×2). The combined organic layers were dried over anhydrous Na₂SO₄, filtered, concentrated and purified by an aluminum oxide column (gradient, DCM to DCM:MeOH=20:1) to afford the title compound (160 mg, 0.401 mmol, 83.5% yield) as a brown solid. LC-MS m/e: 400 (MH⁺).

### Step 3. Synthesis of (R,Z)—N-(3-(4-((1-(3-(difluoromethyl)-2-fluorophenyl)ethyl)imino)-1,2-dimethyl-1,4-dihydropyrido[3,4-d]pyrimidin-6-yl)prop-2-yn-1-yl)acetamide (Compound 85)

To a solution of (R,Z)-3-(4-((1-(3-(difluoromethyl)-2-fluorophenyl)ethyl)imino)-1,2-dimethyl-1,4-dihydropyrido[3,4-d]pyrimidin-6-yl)prop-2-yn-1-amine (60 mg, 0.150 mmol) in DCM (20 mL) was added Et₃N (0.021 mL, 0.150 mmol) followed by acetyl chloride (0.011 mL, 0.15 mmol) at 0° C. The mixture was stirred at 0° C. for 0.5 hrs. The resulting solution was poured into water (10 mL) and extracted with DCM (10 mL×2). The combined organic layers were washed with brine (10 mL), dried over anhydrous Na₂SO₄, and filtered. The filtrate was concentrated under reduced pressure and purified by prep-HPLC to afford a formate salt of the title compound (35 mg) as a white solid.

¹H NMR (400 MHz, DMSO-d₆) δ 8.81 (s, 1H), 8.44 (t, J=4.8 Hz, 1H), 8.19 (s, 1H), 8.05 (d, J=3.8 Hz, 1H), 7.93 (t, J=7.2 Hz, 1H), 7.47 (t, J=7.0 Hz, 1H), 7.35-7.04 (m, 2H), 5.77 (q, J=6.6 Hz, 1H), 4.15 (d, J=5.4 Hz, 2H), 3.61 (s, 3H), 2.45 (s, 3H), 1.87 (s, 3H), 1.38 (d, J=6.6 Hz, 3H). ¹⁹F NMR (377 MHz, DMSO) δ−112.76 (dd, J=13.4, 3.9 Hz), −125.65 (s).

LC-MS m/e: 442 (MH⁺).

### Synthesis of (R,Z)-3-(4-((1-(3-(difluoromethyl)-2-fluorophenyl)ethyl)imino)-1,2-dimethyl-1,4-dihydropyrido[3,4-d]pyrimidin-6-yl)-N-isopropylprop-2-yn-1-amine (Compound 86)

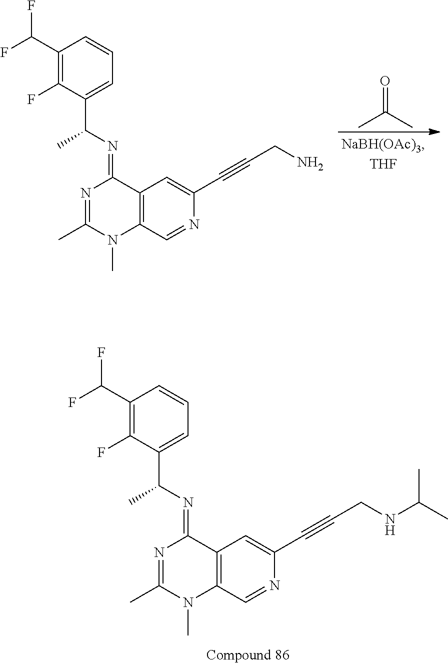

A solution of (R,Z)-3-(4-((1-(3-(difluoromethyl)-2-fluorophenyl)ethyl)imino)-1,2-dimethyl-1,4-dihydropyrido[3,4-d]pyrimidin-6-yl)prop-2-yn-1-amine (90 mg, 0.225 mmol) and propan-2-one (1.5 mL, 20 mmol) in THF (15 mL) was stirred at room temperature for 5 mins, then NaBH(OAc)₃ (96 mg, 0.451 mmol) was added and stirred for 0.5 hrs. The resulting solution was purified directly by prep-HPLC to give the title compound (5.6 mg, 0.013 mmol, 5.6% yield) as yellow solid.

¹H NMR (400 MHz, DMSO-d₆) δ 8.85 (s, 1H), 8.24 (s, 1H), 8.09 (s, 1H), 7.91 (t, J=7.0 Hz, 1H), 7.48 (t, J=6.8 Hz, 1H), 7.33 (t, J=9.8 Hz, 1H), 7.14 (t, J=54.4 Hz, 1H), 5.77 (q, J=6.4 Hz, 1H), 3.73 (s, 2H), 3.64 (s, 3H), 3.07 (s, 1H), 2.47 (s, 3H), 1.40 (d, J=6.6 Hz, 3H), 1.06 (d, J=6.0 Hz, 6H). LC-MS m/e: 442 (MH⁺).

### Synthesis of (R,Z)—N-(4-((1-(3-(difluoromethyl)-2-fluorophenyl)ethyl)imino)-1,2-dimethyl-1,4-dihydropyrido[3,4-d]pyrimidin-6-yl)propane-2-sulfonamide (Compound 87)

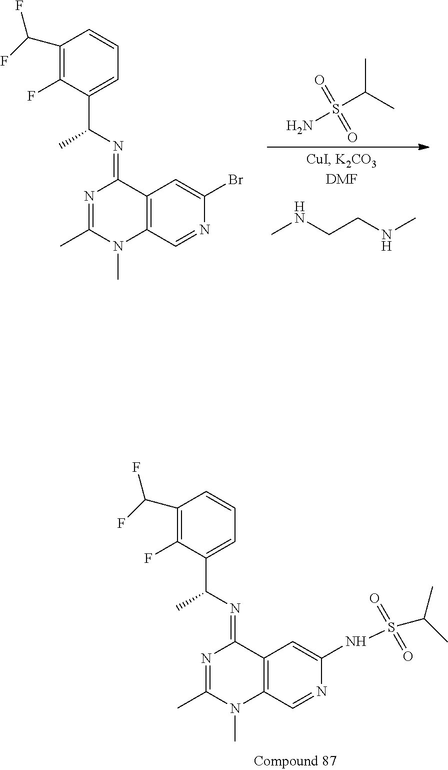

To a solution of (R,Z)-6-bromo-N-(1-(3-(difluoromethyl)-2-fluorophenyl)ethyl)-1,2-dimethylpyrido[3,4-d]pyrimidin-4(1H)-imine (50 mg, 0.12 mmol) and propane-2-sulfonamide (21.7 mg, 0.18 mmol) in DMF (5 mL) were added CuI (22.4 mg, 0.12 mmol), K₂CO₃ (49 mg, 0.35 mmol), and 2,5-diazahexane (21 mg, 0.24 mmol) at 25° C. The reaction mixture was stirred at 120° C. for 3 hrs under N₂. The resulting mixture was cooled to room temperature, poured into water (10 mL) and extracted with EtOAc (20 mL×3). The combined organic layers were washed with brine (10 mL), dried over anhydrous Na₂SO₄, and filtered. The filtrate was concentrated under vacuum and purified by prep-HPLC to afford a formate salt of the title compound (1.2 mg) as light-yellow solid.

¹HNMR (400 MHz, DMSO-d₆) δ 8.58 (s, 1H), 8.18 (s, 1H), 7.89 (t, J=7.2 Hz, 1H), 7.64 (s, 1H), 7.47 (t, J=7.1 Hz, 1H), 7.36-7.30 (m, 1H), 7.19 (t, J=43.7 Hz, 1H), 5.76 (q, J=6.7 Hz, 1H), 3.73 (dd, J=13.6, 6.7 Hz, 1H), 3.60 (s, 3H), 2.43 (s, 3H), 1.39 (d, J=6.6 Hz, 3H), 1.28 (d, J=5.8 Hz, 6H). LCMS m/e: 468.5 (MH⁺).

### Synthesis of (R,Z)—N-(4-((1-(3-(difluoromethyl)-2-fluorophenyl)ethyl)imino)-1,2-dimethyl-1,4-dihydropyrido[3,4-d]pyrimidin-6-yl)cyclopentanecarboxamide (Compound 88)

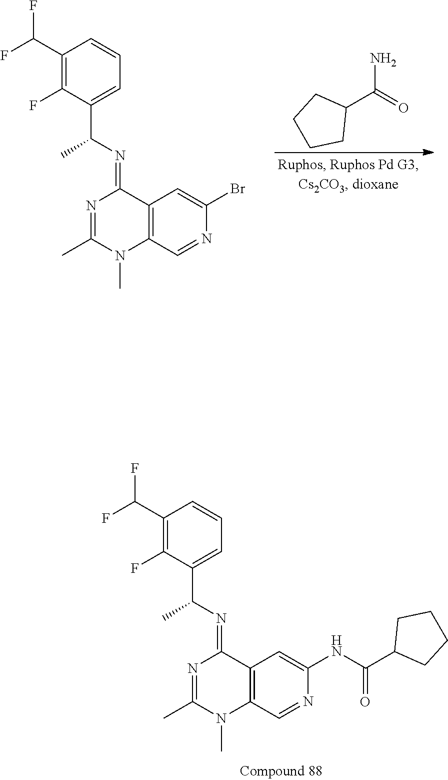 [SMILES: Cc1n/c(=N\[C@H](C)c2cccc(C(F)F)c2F)c2cc(Br)ncc2n1C ; NC(=O)C1CCCC1 ; Cc1n/c(=N\[C@H](C)c2cccc(C(F)F)c2F)c2cc(NC(=O)C3CCCC3)ncc2n1C]

To a solution of (R,Z)-6-bromo-N-(1-(3-(difluoromethyl)-2-fluorophenyl)ethyl)-1,2-dimethylpyrido[3,4-d]pyrimidin-4(1H)-imine (100 mg, 0.235 mmol) and cyclopentanecarboxamide (80 mg, 0.705 mmol) in dioxane (20 mL) were added RuPhos (44 mg, 0.094 mmol), Cs₂CO₃ (191 mg, 0.588 mmol) and RuPhos Pd G3 (39 mg, 0.047 mmol) and stirred at 100° C. for 16 hrs under N₂. The resulting solution was cooled to room temperature, poured into water (10 mL) and extracted with EtOAc (20 mL×2). The combined organic layers were washed with brine (10 mL), dried over anhydrous Na₂SO₄ and filtered. The filtrate was concentrated under vacuum and purified by prep-HPLC to afford a formate salt of the title compound (35 mg) as a light-yellow solid.

¹H NMR (400 MHz, DMSO-d₆) δ 10.60 (s, 1H), 8.75 (s, 1H), 8.64 (s, 1H), 8.19 (s, 1H), 7.87 (t, J=7.2 Hz, 1H), 7.47 (t, J=6.8 Hz, 1H), 7.33 (d, J=7.8 Hz, 1H), 7.19 (t, J=46.6 Hz, 1H), 5.82 (q, J=6.4 Hz, 1H), 3.63 (s, 3H), 2.95 (dd, J=15.4, 7.8 Hz, 1H), 2.45 (s, 3H), 1.93-1.82 (m, 2H), 1.78-1.64 (m, 4H), 1.61-1.50 (m, 2H), 1.41 (d, J=6.6 Hz, 3H). LC-MS m/e: 458 (MH⁺).

### Synthesis of (R,Z)-1-(4-(4-((1-(3-(difluoromethyl)-2-fluorophenyl)ethyl)imino)-1,2-dimethyl-1,4-dihydropyrido[3,4-d]pyrimidine-6-carbonyl)piperazin-1-yl)ethan-1-one (Compound 89)

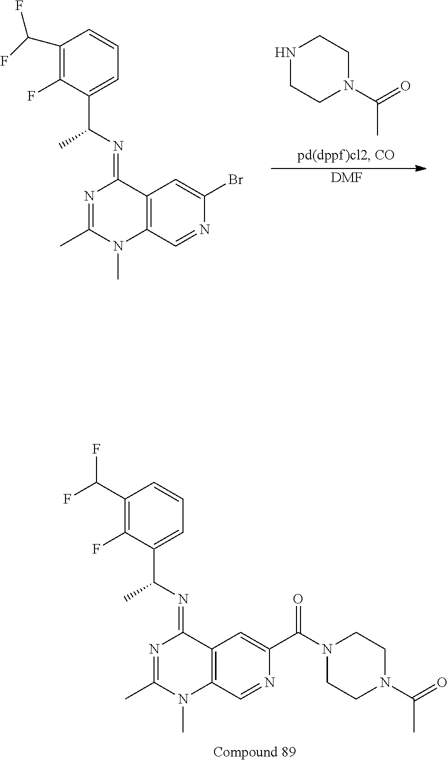

To a solution of (R,Z)-6-bromo-N-(1-(3-(difluoromethyl)-2-fluorophenyl)ethyl)-1,2-dimethylpyrido[3,4-d]pyrimidin-4(1H)-imine (100 mg, 0.24 mmol) and 1-(piperazin-1-yl)ethan-1-one (150.7 mg, 1.18 mmol) in DMF (5 mL) was added TEA (0.16 mL, 1.18 mmol), Pd(dppf)Cl₂ (34.4 mg, 0.05 mmol) at 25° C. The reaction mixture was stirred at 100° C. for 12 hrs under CO atmosphere. The reaction mixture was cooled to room temperature, poured into ice water (30 mL) and extracted with DCM (30 mL×3). The combined organic layers were washed with brine (30 mL*3), dried over NaSO₄, and filtered. The filtrate was concentrated under vacuum and purified by prep-HPLC to afford a formate salt of the title compound (6.1 mg) as brown solid.

¹H NMR (400 MHz, DMSO-d₆) δ 8.87 (d, J=4.0 Hz, 1H), 8.30 (s, 1H), 8.24 (s, 1H), 7.94 (t, J=6.0 Hz, 1H), 7.48 (t, J=6.0 Hz, 1H), 7.34 (d, J=8.0 Hz, 1H), 7.20 (t, J=46.0 Hz, 1H), 5.80 (q, J=8.0 Hz, 1H), 3.74-3.62 (m, 5H), 3.59-3.46 (m, 6H), 2.49 (s, 3H), 2.04 (d, J=20.0 Hz, 3H), 1.41 (d, J=4.0 Hz, 3H). LCMS m/e: 501.2 (MH⁺).

### Synthesis of (R,Z)-(4-((1-(3-(difluoromethyl)-2-fluorophenyl)ethyl)imino)-1-methyl-1,4,5,7-tetrahydro-6H-pyrrolo[3,4-d]pyrimidin-6-yl)(4-methoxytetrahydro-2H-pyran-4-yl)methanone (Compound 90)

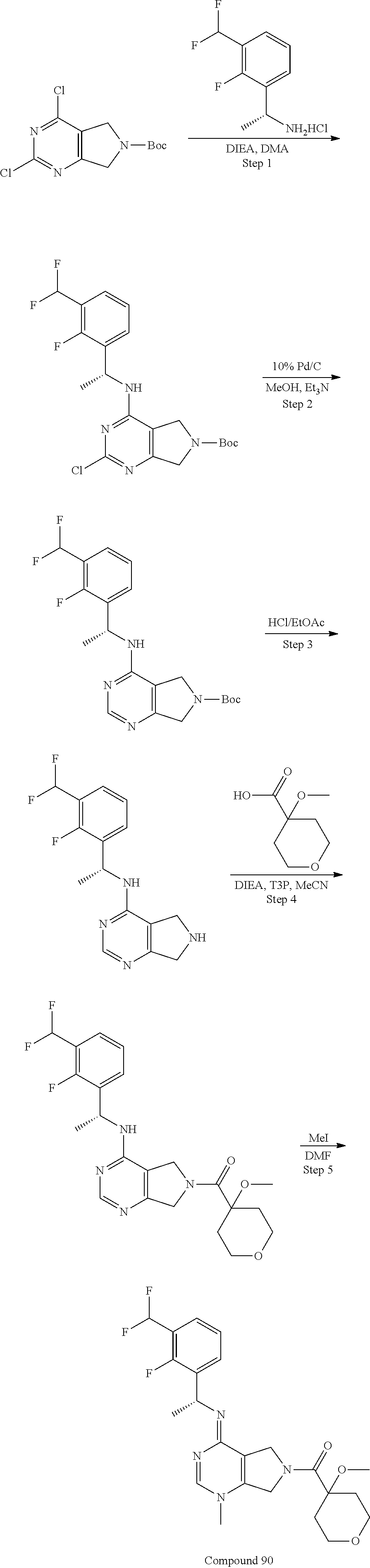

### Step 1. Synthesis of tert-butyl (R)-2-chloro-4-((1-(3-(difluoromethyl)-2-fluorophenyl)ethyl)amino)-5,7-dihydro-6H-pyrrolo[3,4-d]pyrimidine-6-carboxylate

To a solution of (R)-1-(3-(difluoromethyl)-2-fluorophenyl)ethan-1-amine hydrochloride (780 mg, 3.46 mmol) in DMA (3 mL) and DIPEA (1.11 g, 8.64 mmol) was added tert-butyl 2,4-dichloro-5,7-dihydro-6H-pyrrolo[3,4-d]pyrimidine-6-carboxylate (1.20 g, 4.15 mmol). The mixture was stirred at 20° C. for 2 hrs, then poured into ice-water (10 mL), and extracted with EtOAc (20 mL×3). The combined organic layers were washed with brine (20 mL) and dried over anhydrous Na₂SO₄, filtered and concentrated to dryness. The residue was purified by flash chromatography on silica gel (gradient, PE to PE:EtOAc=3:1) to afford the title compound (1.30 g, 2.94 mmol, 84.9% yield) as a yellow solid. LCMS m/e: 443, 445 (MH⁺).

### Step 2. Synthesis of tert-butyl (R)-4-((1-(3-(difluoromethyl)-2-fluorophenyl)ethyl)amino)-5,7-dihydro-6H-pyrrolo[3,4-d]pyrimidine-6-carboxylate

To a solution of tert-butyl (R)-2-chloro-4-((1-(3-(difluoromethyl)-2-fluorophenyl)-ethyl)amino)-5,7-dihydro-6H-pyrrolo[3,4-d]pyrimidine-6-carboxylate (1.2 g, 2.71 mmol) in MeOH (50 mL) were added Et₃N (5.0 mL, 36 mmol) and Pd/C (1 g, 2.71 mmol, 10% and contained 55% water), and the mixture was stirred at 30° C. under H₂ balloon for 2 hrs. The resulting mixture was filtered through a pad of Celite and washed with MeOH (50 mL). The filtrate was concentrated to afford the title compound (800 mg, 1.96 mmol, 72.3% yield) as a yellow solid. LCMS m/e: 409 (MH⁺).

### Step 3. Synthesis of (R)—N-(1-(3-(difluoromethyl)-2-fluorophenyl)ethyl)-6,7-dihydro-5H-pyrrolo[3,4-d]pyrimidin-4-amine hydrochloride

A solution of tert-butyl(R)-4-((1-(3-(difluoromethyl)-2-fluorophenyl)ethyl)amino)-5,7-dihydro-6H-pyrrolo[3,4-d]pyrimidine-6-carboxylate (0.8 g, 1.96 mmol) in HCl (50 mL, 100 mmol, 2 M in EtOAc) was stirred for 2 hrs at room temperature and then concentrated to afford the title compound (0.5 g, 1.45 mmol, 74.0% yield) as a yellow solid.

LCMS m/e: 309 (MH⁺).

### Step 4. Synthesis of (R)-(4-((1-(3-(difluoromethyl)-2-fluorophenyl)ethyl)amino)-5,7-dihydro-6H-pyrrolo[3,4-d]pyrimidin-6-yl)(4-methoxytetrahydro-2H-pyran-4-yl)methanone

To a solution of (R)—N-(1-(3-(difluoromethyl)-2-fluorophenyl)ethyl)-6,7-dihydro-5H-pyrrolo[3,4-d]pyrimidin-4-amine hydrochloride (500 mg, 1.45 mmol) and 4-methoxytetrahydro-2H-pyran-4-carboxylic acid (464 mg, 2.9 mmol) in MeCN (10 mL) and DIPEA (1.12 g, 8.7 mmol) was added dropwise T₃P (1.85 g, 2.9 mmol, 50% in EtOAc) at 0° C. The mixture was stirred at room temperature for 1 hr, diluted with sat. NaHCO₃ (10 mL) and extracted with EtOAc (20 mL×2). The combined organic layers were washed with brine (20 mL), dried over anhydrous Na₂SO₄, filtered and concentrated to dryness. The residue was purified by flash chromatography on silica gel (gradient, PE to PE:EtOAc=1:9) to afford the title compound (400 mg, 0.888 mmol, 61.2% yield) as a yellow solid. LCMS m/e: 451 (MH⁺).

### Step 5. Synthesis of (R,Z)-(4-((1-(3-(difluoromethyl)-2-fluorophenyl)ethyl)imino)-1-methyl-1,4,5,7-tetrahydro-6H-pyrrolo[3,4-d]pyrimidin-6-yl)(4-methoxytetrahydro-2H-pyran-4-yl)methanone (Compound 90)

To a solution of (R)-(4-((1-(3-(difluoromethyl)-2-fluorophenyl)ethyl)amino)-5,7-dihydro-6H-pyrrolo[3,4-d]pyrimidin-6-yl)(4-methoxytetrahydro-2H-pyran-4-yl)methanone (80 mg, 0.178 mmol) in DMF (1 mL) was added iodomethane (50 mg, 0.355 mmol). The mixture was stirred for 18 hrs at room temperature, poured into ice-water (10 mL) and extracted with EtOAc (20 mL×2). The combined organic layers were washed with brine (20 mL) and dried over anhydrous Na₂SO₄, filtered and concentrated to dryness. The residue was purified by reverse phase chromatography (SilaSep™ C18 silica flash cartridge, 0%-40% MeCN in H₂O with 0.1% formic acid) to afford the title compound (20.0 mg, 0.043 mmol, 24.3% yield) as a yellow solid.

¹H NMR (400 MHz, DMSO-d₆) δ 8.77 (d, J=12.4 Hz, 1H), 7.76 (dd, J=18.4, 7.6 Hz, 1H), 7.55 (t, J=6.8 Hz, 1H), 7.37-7.09 (m, 2H), 5.70 (q, J=6.8 Hz, 1H), 5.15 (s, 1H), 5.05 (d, J=7.6 Hz, 1H), 4.90 (d, J=8.4 Hz, 1H), 4.76 (d, J=12.4 Hz, 1H), 3.78 (d, J=5.6 Hz, 3H), 3.74-3.57 (m, 4H), 3.19 (d, J=6.4 Hz, 3H), 2.02-1.83 (m, 4H), 1.58 (d, J=6.8 Hz, 3H).

LCMS m/e: 465 (MH⁺).

### Synthesis of (R,Z)-1-(4-(4-((1-(3-(difluoromethyl)-2-fluorophenyl)ethyl)imino)-1,2-dimethyl-4,5,6,7-tetrahydro-1H-pyrrolo[3,4-d]pyrimidine-6-carbonyl)-4-fluoropiperidin-1-yl)ethan-1-one (compound 91)

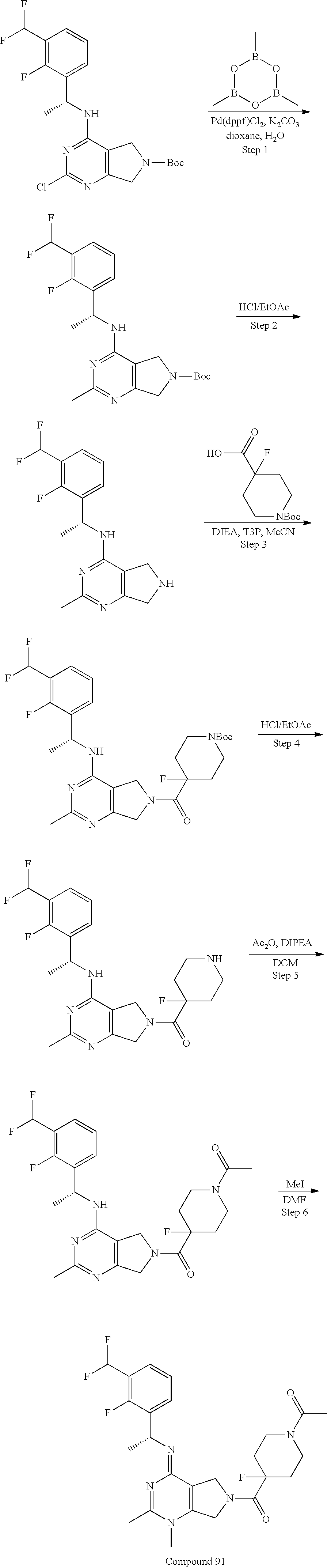

### Step 1. Synthesis of tert-butyl (R)-4-((1-(3-(difluoromethyl)-2-fluorophenyl)ethyl)amino)-2-methyl-5,7-dihydro-6H-pyrrolo[3,4-d]pyrimidine-6-carboxylate

To a solution of tert-butyl (R)-2-chloro-4-((1-(3-(difluoromethyl)-2-fluorophenyl)ethyl)-amino)-5,7-dihydro-6H-pyrrolo[3,4-d]pyrimidine-6-carboxylate (600 mg, 1.36 mmol) and 2,4,6-trimethyl-1,3,5,2,4,6-trioxatriborinane (1.94 mL, 6.77 mmol, 3M in THF) in dioxane (10 mL) and water (2 mL) were added K₂CO₃ (562 mg, 4 mmol) and Pd(dppf)Cl₂ (99 mg, 0.135 mmol). The mixture was stirred at 100° C. for 2 hrs under N₂. The resulting mixture was cooled to room temperature, concentrated and purified by flash chromatography on silica gel (gradient, PE to PE:EtOAc=4:1) to afford the title compound (350 mg, 0.829 mmol, 61.2% yield) as a yellow solid. LCMS m/e: 423 (MH⁺).

### Step 2. Synthesis of (R)—N-(1-(3-(difluoromethyl)-2-fluorophenyl)ethyl)-2-methyl-6,7-dihydro-5H-pyrrolo[3,4-d]pyrimidin-4-amine hydrochloride

A solution of tert-butyl (R)-4-((1-(3-(difluoromethyl)-2-fluorophenyl)ethyl)amino)-2-methyl-5,7-dihydro-6H-pyrrolo[3,4-d]pyrimidine-6-carboxylate (350 mg, 0.829 mmol) in HCl solution (10 mL, 40.0 mmol, 4 M in dioxane) is stirred at room temperature for 2 hrs and then concentrated to afford the title compound (290 mg, 0.808 mmol, 97.6% yield) as a yellow solid.

LCMS m/e: 323 (MH⁺).

### Step 3. Synthesis of tert-butyl (R)-4-(4-((1-(3-(difluoromethyl)-2-fluorophenyl)ethyl)amino)-2-methyl-6,7-dihydro-5H-pyrrolo[3,4-d]pyrimidine-6-carbonyl)-4-fluoropiperidine-1-carboxylate

To a solution of (R)—N-(1-(3-(difluoromethyl)-2-fluorophenyl)ethyl)-2-methyl-6,7-dihydro-5H-pyrrolo[3,4-d]pyrimidin-4-amine hydrochloride (290 mg, 0.836 mmol) and 1-(tert-butoxycarbonyl)-4-fluoropiperidine-4-carboxylic acid (414 mg, 1.67 mmol) in ACN (10 mL) were added DIEA (648 mg, 5.02 mmol) and T₃P (532 mg, 1.67 mmol, 50% in EtOAc). The mixture was stirred for 1 hr at room temperature under N₂, diluted with sat. NaHCO₃ (10 mL) and extracted with EtOAc (20 mL×2). The combined organic layers were washed with brine (20 mL), dried over anhydrous Na₂SO₄, filtered and concentrated to dryness. The residue was purified by flash chromatography on silica gel (gradient, PE to PE:EtOAc=1:9) to afford the title compound (380 mg, 0.689 mmol, 76.6% yield) as a yellow solid. LCMS m/e: 552 (MH⁺).

### Step 4. Synthesis of (R)-(4-((1-(3-(difluoromethyl)-2-fluorophenyl)ethyl)amino)-2-methyl-5,7-dihydro-6H-pyrrolo[3,4-d]pyrimidin-6-yl)(4-fluoropiperidin-4-yl)methanone

A solution of tert-butyl (R)-4-(4-((1-(3-(difluoromethyl)-2-fluorophenyl)ethyl)amino)-2-methyl-6,7-dihydro-5H-pyrrolo[3,4-d]pyrimidine-6-carbonyl)-4-fluoropiperidine-1-carboxylate (380 mg, 0.689 mmol) in HCl solution (10 mL, 2 M in EtOAc) was stirred for 1 hr at room temperature and then concentrated to afford the title compound (310 mg, 0.687 mmol, 99.7% yield) as a white solid. LCMS m/e: 452 (MH⁺).

### Step 5. Synthesis of (R)-1-(4-(4-((1-(3-(difluoromethyl)-2-fluorophenyl)ethyl)amino)-2-methyl-6,7-dihydro-5H-pyrrolo[3,4-d]pyrimidine-6-carbonyl)-4-fluoropiperidin-1-yl)ethan-1-one

To a solution of (R)-(4-((1-(3-(difluoromethyl)-2-fluorophenyl)ethyl)amino)-2-methyl-5,7-dihydro-6H-pyrrolo[3,4-d]pyrimidin-6-yl)(4-fluoropiperidin-4-yl)methanone (310 mg, 0.687 mmol) in DCM (8 mL) were added DIPEA (444 mg, 3.43 mmol) and Ac₂O (140 mg, 1.37 mmol). The mixture was stirred for 2 hrs at room temperature under N₂, diluted with ice-water (10 mL) and extracted with DCM (20 mL×3). The combined organic layers were washed with saturated NaHCO₃ solution (20 mL), brine (20 mL), dried over anhydrous Na₂SO₄, filtered and concentrated to dryness. The residue was purified by flash chromatography on silica gel (gradient, DCM to DCM:MeOH=5:1) to afford the title compound (230 mg, 0.466 mmol, 67.9% yield) as a white solid. LCMS m/e: 494 (MH⁺).

### Step 6. Synthesis of (R,Z)-1-(4-(4-((1-(3-(difluoromethyl)-2-fluorophenyl)ethyl)imino)-1,2-dimethyl-4,5,6,7-tetrahydro-1H-pyrrolo[3,4-d]pyrimidine-6-carbonyl)-4-fluoropiperidin-1-yl)ethan-1-one TFA salt (Compound 91)

To a solution of (R)-1-(4-(4-((1-(3-(difluoromethyl)-2-fluorophenyl)ethyl)amino)-2-methyl-6,7-dihydro-5H-pyrrolo[3,4-d]pyrimidine-6-carbonyl)-4-fluoropiperidin-1-yl)ethan-1-one (90 mg, 0.182 mmol) in DMF (1.5 mL) was added iodomethane (310 mg, 2.18 mmol) and stirred for 18 hrs at room temperature under N₂. The mixture was diluted with water (10 mL) at room temperature and extracted with EtOAc (30 mL×3). The combined organic layers were washed with saturated NaHCO₃ solution (20 mL), brine (20 mL), dried over anhydrous Na₂SO₄, filtered and concentrated to dryness. The residue was purified by prep-HPLC to afford a TFA salt of the title compound (14 mg) as a yellow solid.

¹H NMR (400 MHz, DMSO-d₆) δ 9.71 (t, J=7.2 Hz, 1H), 7.71-7.67 (m, 1H), 7.59-7.52 (m, 1H), 7.37-7.10 (m, 2H), 5.75-5.65 (m, 1H), 5.22 (s, 1H), 5.03 (s, 1H), 4.91 (d, J=8.0 Hz, 1H), 4.70 (s, 1H), 4.34 (d, J=12.0 Hz, 1H), 3.87-3.77 (m, 1H), 3.69 (d, J=7.2 Hz, 3H), 3.30 (t, J=12.4 Hz, 1H), 2.91-2.77 (m, 1H), 2.62 (d, J=2.4 Hz, 3H), 2.21-1.87 (m, 7H), 1.59 (dd, J=6.4, 4.4 Hz, 3H). LCMS m/e: 508 (MH⁺).

### Synthesis of 1-((R,Z)-11-(((R)-1-(3-(difluoromethyl)-2-fluorophenyl)ethyl)imino)-8-methyl-1,2,4a,5,8,11-hexahydropyrazino[1′,2′:4,5][1,4]oxazino[3,2-g]quinazolin-3(4H)-yl)ethan-1-one (Compound 92)

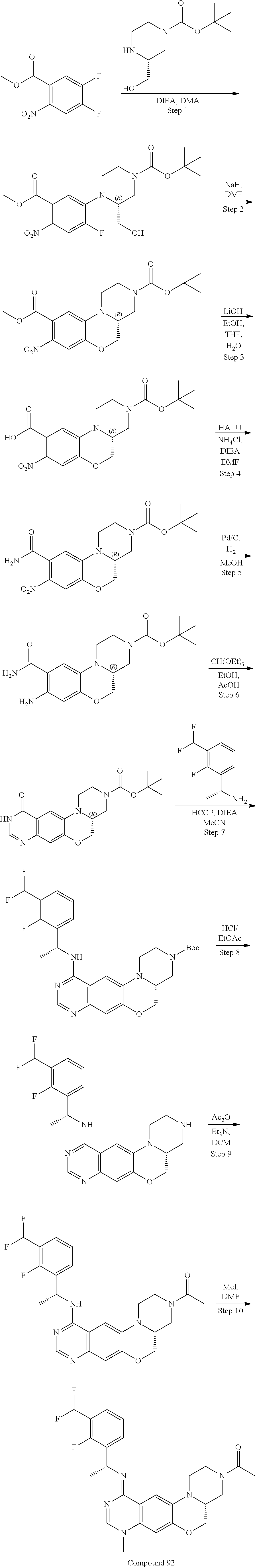

### Step 1. Synthesis of tert-butyl (R)-4-(2-fluoro-5-(methoxycarbonyl)-4-nitrophenyl)-3-(hydroxymethyl)piperazine-1-carboxylate

To a solution of methyl 4,5-difluoro-2-nitrobenzoate (10 g, 46 mmol) in DMF (100 mL) were added tert-butyl (R)-3-(hydroxymethyl)piperazine-1-carboxylate (12 g, 55.3 mmol) and DIEA (23.8 g, 184 mmol). The mixture was stirred at 90° C. for 15 hr under N₂. The reaction mixture was cooled to room temperature, poured into water (200 mL) and extracted with EtOAc (300 mL×3). The combined organic layers were washed with brine (200 mL×2), dried over anhydrous Na₂SO₄, filtered and concentrated to dryness. The residue was purified by column chromatography on silica gel (gradient, PE to PE:EtOAc=3:1) to afford the title compound (7 g, 16.9 mmol, 36.8% yield) as a yellow solid. LC-MS m/e: 414 (MH⁺).

### Step 2. Synthesis of 3-(tert-butyl) 9-methyl (R)-8-nitro-1,2,4a,5-tetrahydrobenzo[b]pyrazino[1,2-d][1,4]oxazine-3,9(4H)-dicarboxylate

To a solution of tert-butyl (R)-4-(2-fluoro-5-(methoxycarbonyl)-4-nitrophenyl)-3-(hydroxymethyl)piperazine-1-carboxylate (7 g, 16.9 mmol) in DMF (100 mL) was added NaH (0.68 g, 17.09 mmol) at 0° C. The mixture is stirred at room temperature for 2 hrs. The reaction mixture was poured into ice water (200 mL) and extracted with EtOAc (300 mL×3). The combined organic layers were washed with brine (200 mL×2), dried over anhydrous Na₂SO₄, filtered and concentrated to dryness. The residue was purified by column chromatography on silica gel (gradient, PE to PE:EtOAc=3:1) to afford the title compound (6 g, 15.3 mmol, 90.1% yield) as a yellow solid. LC-MS m/e: 394 (MH⁺).

### Step 3. Synthesis of (R)-3-(tert-butoxycarbonyl)-8-nitro-1,2,3,4,4a,5-hexahydrobenzo[b]-pyrazino[1,2-d][1,4]oxazine-9-carboxylic Acid

To a solution of 3-(tert-butyl) 9-methyl (R)-8-nitro-1,2,4a,5-tetrahydrobenzo[b]pyrazino-[1,2-d][1,4]oxazine-3,9(4H)-dicarboxylate (4 g, 10.2 mmol) in EtOH (10 mL), THF (10 mL) and H₂O (10 mL) was added LiOH monohydrate (4.27 g, 102 mmol). The mixture was stirred at 35° C. for 4 hrs. The reaction mixture was cooled to room temperature, acidified by HCl solution (1 M in water) to pH 4.0 and extracted with EtOAc (100 mL×3). The combined organic layers were washed with brine (100 mL), dried over anhydrous Na₂SO₄, filtered and concentrated to afford the title compound (3.5 g, 9.23 mmol, 90.7% yield).

LC-MS m/e: 380 (MH⁺)

### Step 4. Synthesis of tert-butyl (R)-9-carbamoyl-8-nitro-1,2,4a,5-tetrahydrobenzo[b]pyrazino[1,2-d][1,4]oxazine-3(4H)-carboxylate

To a solution of (R)-3-(tert-butoxycarbonyl)-8-nitro-1,2,3,4,4a,5-hexahydrobenzo[b]-pyrazino[1,2-d][1,4]oxazine-9-carboxylic acid (3.5 g, 9.23 mmol) in DMF (40 mL) were added HATU (5.26 g, 13.8 mmol), NH₄Cl (1.00 g, 18.5 mmol) and DIEA (3.57 g, 27.7 mmol). The mixture was stirred at room temperature for 4 hrs. The reaction mixture was poured into water (100 mL) and extracted with EtOAc (100 mL×3). The combined organic layers were washed with brine (200 mL×2), dried over anhydrous Na₂SO₄, filtered and concentrated to dryness. The residue was purified by column chromatography on silica gel (gradient, PE to PE:EtOAc=1:1) to afford the title compound (3.4 g, 8.99 mmol, 97.4% yield) as a white solid.

LC-MS m/e: 379 (MH⁺)

### Step 5. Synthesis of tert-butyl (R)-8-amino-9-carbamoyl-1,2,4a,5-tetrahydrobenzo[b]-pyrazino[1,2-d][1,4]oxazine-3(4H)-carboxylate

To a solution of tert-butyl (R)-9-carbamoyl-8-nitro-1,2,4a,5-tetrahydrobenzo[b]-pyrazino[1,2-d][1,4]oxazine-3(4H)-carboxylate (3.4 g, 9 mmol) in MeOH (50 mL) was added Pd/C (700 mg, 10% and 55% weight in water). The mixture was stirred at room temperature for 3 hrs under H₂ balloon. The reaction mixture was filtered through a pad of Celite® and the filter cake was washed with MeOH (50 mL). The filtrate was concentrated to afford the title compound (2.4 g, 6.89 mmol, 76.7% yield) as a green solid.

LC-MS m/e: 349 (MH⁺).

### Step 6. Synthesis of tert-butyl (R)-11-oxo-1,2,4a,5,10,11-hexahydropyrazino[1′,2′:4,5][1,4]-oxazino[3,2-g]quinazoline-3(4H)-carboxylate

To a solution of tert-butyl (R)-8-amino-9-carbamoyl-1,2,4a,5-tetrahydrobenzo[b]-pyrazino[1,2-d][1,4]oxazine-3(4H)-carboxylate (1.2 g, 3.44 mmol) in EtOH (20 mL) were added triethoxymethane (7.65 g, 51.7 mmol) and AcOH (3.1 g, 51.7 mmol). The mixture was stirred at 120° C. in a sealed tube for 15 hrs. The reaction mixture was cooled to room temperature, concentrated and purified by column chromatography (gradient, DCM to DCM:MeOH=20:1) to afford the title compound (500 mg, 1.4 mmol, 40.5% yield) as a grey solid.

LC-MS m/e: 359 (MH⁺).

### Step 7. Synthesis of tert-butyl (R)-11-(((R)-1-(3-(difluoromethyl)-2-fluorophenyl)ethyl)amino)-1,2,4a,5-tetrahydropyrazino[1′,2′:4,5][1,4]oxazino[3,2-g]quinazoline-3(4H)-carboxylate

To a solution of tert-butyl (R)-11-oxo-1,2,4a,5,10,11-hexahydropyrazino[1′,2′:4,5][1,4]-oxazino[3,2-g]quinazoline-3(4H)-carboxylate (450 mg, 1.256 mmol) in ACN (5 mL) were added HCCP (524 mg, 1.51 mmol) and DIEA (649 mg, 5.03 mmol). The mixture was stirred at room temperature for 2 hrs under N₂. (R)-1-(3-(difluoromethyl)-2-fluorophenyl)ethan-1-amine (356 mg, 1.88 mmol) was added and the reaction mixture was stirred at 100° C. for 2 hrs under N₂. The reaction mixture was cooled to room temperature, poured into water (20 mL) and extracted with EtOAc (20 mL×3). The combined organic layers were washed with brine (20 mL), dried over anhydrous Na₂SO₄, filtered and concentrated to dryness. The residue was purified by column chromatography on silica gel (gradient, DCM to DCM:MeOH=20:1) to afford the title compound (300 mg, 0.567 mmol, 45.1% yield) as a yellow solid.

LC-MS m/e: 530 (MH⁺).

### Step 8. Synthesis of (R)—N—((R)-1-(3-(difluoromethyl)-2-fluorophenyl)ethyl)-1,2,3,4,4a,5-hexahydropyrazino[1′,2′:4,5][1,4]oxazino[3,2-g]quinazolin-11-amine

To a solution of tert-butyl (R)-11-(((R)-1-(3-(difluoromethyl)-2-fluorophenyl)ethyl)-amino)-1,2,4a,5-tetrahydropyrazino[1′,2′:4,5][1,4]oxazino[3,2-g]quinazoline-3(4H)-carboxylate (300 mg, 0.567 mmol) in EtOAc (5 mL) was added HCl solution (5 mL, 4 M in EtOAc) and stirred at room temperature for 2 hrs. The reaction mixture was poured into water (10 mL), basified with NaOH solution (12 mL, 2 M) and extracted with DCM (50 mL×3). The combined organic layers were washed with brine (20 mL), dried over anhydrous Na₂SO₄, filtered and concentrated to dryness. The residue was purified by column chromatography on silica gel (gradient, DCM to DCM:MeOH=10:1) to afford the title compound (200 mg, 0.466 mmol, 82.2% yield).

LC-MS m/e: 430 (MH⁺).

### Step 9. Synthesis of 1-((R)-11-(((R)-1-(3-(difluoromethyl)-2-fluorophenyl)ethyl)amino)-1,2,4a,5-tetrahydropyrazino[1′,2′:4,5][1,4]oxazino[3,2-g]quinazolin-3(4H)-yl)ethan-1-one

To a solution of (R)—N—((R)-1-(3-(difluoromethyl)-2-fluorophenyl)ethyl)-1,2,3,4,4a,5-hexahydropyrazino[1′,2′:4,5][1,4]oxazino[3,2-g]quinazolin-11-amine (200 mg, 0.466 mmol) in DCM (10 mL) were added Et₃N (71 mg, 0.703 mmol) and Ac₂O (71 mg, 0.699 mmol). The mixture was stirred at room temperature for 2 hrs. The reaction mixture was concentrated to dryness. The residue was purified by column chromatography (gradient, DCM to DCM:MeOH=20:1) to afford the title compound (200 mg, 0.424 mmol, 91.1% yield).

¹H NMR (400 MHz, DMSO-d₆) δ 8.15 (d, J=2.0 Hz, 2H), 8.09 (d, J=5.9 Hz, 1H), 7.70 (s, 1H), 7.61 (t, J=7.2 Hz, 1H), 7.50 (t, J=6.9 Hz, 1H), 7.26 (dt, J=62.8, 42.5 Hz, 2H), 6.93 (d, J=3.1 Hz, 1H), 5.78 (dd, J=13.4, 6.8 Hz, 1H), 4.62-4.42 (m, 2H), 4.05 (dt, J=25.4, 13.9 Hz, 3H), 3.13-2.66 (m, 3H), 2.48-2.37 (m, 1H), 2.09 (t, J=7.5 Hz, 3H), 1.60 (d, J=6.9 Hz, 3H). LC-MS m/e: 472 (MH⁺).

### Step 10. Synthesis of 1-((R,Z)-11-(((R)-1-(3-(difluoromethyl)-2-fluorophenyl)ethyl)imino)-8-methyl-1,2,4a,5,8,11-hexahydropyrazino[1′,2′:4,5][1,4]oxazino[3,2-g]quinazolin-3(4H)-yl)ethan-1-one (Compound 92)

To a solution of 1-((R)-11-(((R)-1-(3-(difluoromethyl)-2-fluorophenyl)ethyl)amino)-1,2,4a,5-tetrahydropyrazino[1′,2′:4,5][1,4]oxazino[3,2-g]quinazolin-3(4H)-yl)ethan-1-one (60 mg, 0.127 mmol) in DMF (1 mL) was added MeI (27 mg, 0.191 mmol) and stirred at room temperature for 12 hrs. The reaction mixture was extracted with EtoAc. The combined organic layers were washed with brine, dried over Na₂SO₄, filtered and concentrated to dryness. The residue is purified by prep-HPLC to afford the title compound (7 mg, 0.014 mmol, 11.3% yield) as a white solid.

¹H NMR (400 MHz, DMSO-d₆) δ 8.46 (s, 1H), 7.92 (s, 1H), 7.83 (s, 1H), 7.52 (s, 1H), 7.39-7.21 (m, 2H), 7.09 (s, 1H), 5.86 (d, J=7.1 Hz, 1H), 4.53 (t, J=17.1 Hz, 2H), 4.14-3.98 (m, 3H), 3.73 (s, 3H), 3.17-3.08 (m, 1H), 2.99-2.78 (m, 2H), 2.33 (s, 1H), 2.09 (d, J=6.7 Hz, 3H), 1.57 (s, 3H). LC-MS m/e: 486 (MH⁺).

### Synthesis of (R,Z)-3-(1-((6-(1-acetyl-4-fluoropiperidin-4-yl)-1,2-dimethylpyrido[3,4-d]pyrimidin-4(1H)-ylidene)amino)ethyl)-2-methylbenzonitrile (Compound 93)

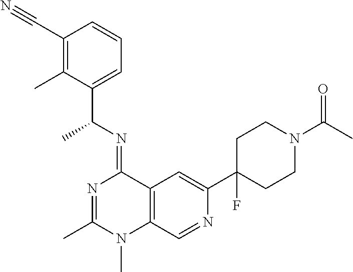 [SMILES: CC(=O)N1CCC(F)(c2cc3/c(=N/[C@H](C)c4cccc(C#N)c4C)nc(C)n(C)c3cn2)CC1]

The formate salt of the title compound was synthesized following a procedure similar to Compound 1

¹HNMR (400 MHz, DMSO-d₆): δ 8.94 (s, 1H), 8.30 (s, 1H), 8.24 (s, 1H), 7.94 (d, J=7.8 Hz, 1H), 7.62 (d, J=7.6 Hz, 1H), 7.39 (t, J=7.7 Hz, 1H), 5.70 (q, J=6.4 Hz, 1H), 4.44 (d, J=12.4 Hz, 1H), 3.88 (d, J=14.0 Hz, 1H), 3.68 (s, 3H), 3.40 (t, J=12.9 Hz, 1H), 2.90 (t, J=12.9 Hz, 1H), 2.62 (s, 3H), 2.51 (s, 3H), 2.37-2.12 (m, 2H), 2.08 (s, 3H), 2.02-1.90 (m, 2H), 1.43 (d, J=6.5 Hz, 3H). LCMS m/e: 461.4 (MH⁺).

### Synthesis of (R,Z)—N-(1-(3-(difluoromethyl)-2-fluorophenyl)ethyl)-6-(1-(difluoromethyl)cyclopropyl)-1,2-dimethylpyrido[3,4-d]pyrimidin-4(1H)-imine (Compound 94)

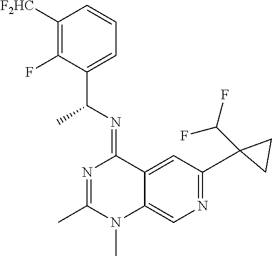 [SMILES: Cc1n/c(=N\[C@H](C)c2cccc(C(F)F)c2F)c2cc(C3(C(F)F)CC3)ncc2n1C]

The formate salt of the title compound was synthesized following a similar procedure to Compound 18.

¹H NMR (400 MHz, DMSO-d₆) δ 8.83 (s, 1H), 8.21 (s, 1H, HCOOH), 8.16 (s, 1H), 7.92 (t, J=7.2 Hz, 1H), 7.47 (t, J=7.0 Hz, 1H), 7.33 (d, J=8.4 Hz, 1H), 7.19 (t, J=54.4 Hz, 1H), 6.39 (t, J=56.0 Hz, 1H), 5.79 (q, J=6.6 Hz, 1H), 3.63 (s, 3H), 2.46 (s, 3H), 1.41 (d, J=6.4 Hz, 3H), 1.33-1.21 (m, 4H). LCMS m/e: 437.4 (MH⁺).

### Synthesis of (R,Z)-1-(4-(5-((1-(3-(difluoromethyl)-2-fluorophenyl)ethyl)imino)-5,7,8,9-tetrahydropyrido[4,3-e]pyrrolo[1,2-a]pyrimidin-3-yl)-4-hydroxypiperidin-1-yl)ethan-1-one (Compound 95)

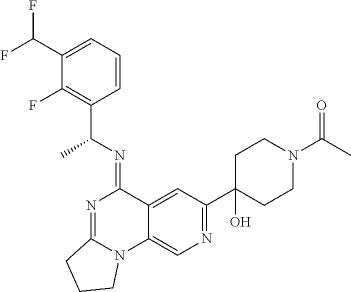 [SMILES: CC(=O)N1CCC(O)(c2cc3/c(=N/[C@H](C)c4cccc(C(F)F)c4F)nc4n(c3cn2)CCC4)CC1]

The title compound was synthesized following a procedure similar to Compound 26.

¹HNMR (400 MHz, CDCl₃) δ 9.63 (d, J=8.8 Hz, 1H), 8.84 (d, J=3.2 Hz, 1H), 8.54 (s, 1H), 7.96 (t, J=7.0 Hz, 1H), 7.53-7.47 (m, 1H), 7.26-7.22 (m, 1H), 6.89 (t, J=55.0 Hz, 1H), 6.11-6.08 (m, 1H), 4.62-4.49 (m, 3H), 3.68 (d, J=6.8 Hz, 2H), 3.42-3.26 (m, 2H), 3.13 (t, J=12.2 Hz, 1H), 2.62-2.55 (m, 2H), 2.25-1.99 (m, 5H), 1.85 (d, J=7.2 Hz, 3H), 1.77-1.61 (m, 2H). LCMS m/e: 500.2 (MH⁺).

### Synthesis of (R,Z)-1-(4-(5-((1-(3-(difluoromethyl)-2-fluorophenyl)ethyl)imino)-5,7,8,9-tetrahydropyrido[4,3-e]pyrrolo[1,2-a]pyrimidin-3-yl)-4-fluoropiperidin-1-yl)ethan-1-one (Compound 96)

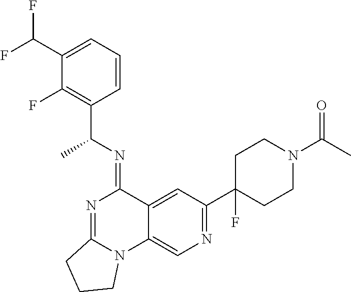 [SMILES: CC(=O)N1CCC(F)(c2cc3/c(=N/[C@H](C)c4cccc(C(F)F)c4F)nc4n(c3cn2)CCC4)CC1]

The formate salt of the title compound was synthesized following a procedure similar to Compound 27.

¹H NMR (400 MHz, CDCl₃) δ 8.98 (s, 1H), 8.65 (s, 1H), 8.44 (s, 1H), 7.96 (t, J=7.4 Hz, 1H), 7.46 (t, J=6.8 Hz, 1H), 7.25-7.18 (m, 1H), 6.90 (t, J=55.1 Hz, 1H), 5.95-6.01 (m, 1H), 4.66 (d, J=10.3 Hz, 1H), 4.30-4.41 (m, 2H), 3.83 (d, J=11.2 Hz, 1H), 3.57 (t, J=13.1 Hz, 1H), 3.31-3.10 (m, 2H), 3.04 (t, J=13.1 Hz, 1H), 2.46-2.51 (m, 2H), 2.36-2.17 (m, 2H), 2.15 (s, 3H), 2.12-1.96 (m, 2H), 1.70 (d, J=6.8 Hz, 3H). LCMS m/e: 502.3 (MH⁺).

### Synthesis of (R,Z)—N-(1-(3-(difluoromethyl)-2-fluorophenyl)ethyl)-1,2-dimethyl-6-(1-(methylsulfonyl)-1,2,3,6-tetrahydropyridin-4-yl)pyrido[3,4-d]pyrimidin-4(1H)-imine (Compound 97)

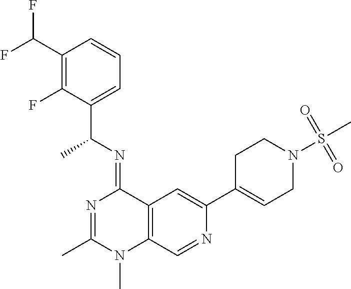 [SMILES: Cc1n/c(=N\[C@H](C)c2cccc(C(F)F)c2F)c2cc(C3=CCN(S(C)(=O)=O)CC3)ncc2n1C]

The trifluoroacetate salt of the title compound was synthesized following a procedure similar to Compound 42.

¹HNMR (400 MHz, DMSO-d₆) δ 10.72 (d, J=6.9 Hz, 1H), 9.51 (s, 1H), 8.63 (s, 1H), 7.80 (t, J=7.3 Hz, 1H), 7.59 (t, J=6.9 Hz, 1H), 7.38 (t, J=6.9 Hz, 1H) 7.11 (t, J=52 Hz, 1H), 7.04-6.98 (m, 1H), 5.99-5.86 (m, 1H), 4.04 (s, 5H), 3.47 (t, J=5.9 Hz, 2H), 2.99 (s, 3H), 2.84-2.73 (m, 5H), 1.74 (d, J=7.0 Hz, 3H). LCMS m/e: 506.5 (MH⁺).

### Synthesis of N-(3-((Z)-4-(((R)-1-(3-(difluoromethyl)-2-fluorophenyl)ethyl)imino)-1,2-dimethyl-1,4-dihydropyrido[3,4-d]pyrimidin-6-yl)allyl)acetamide (Compound 98)

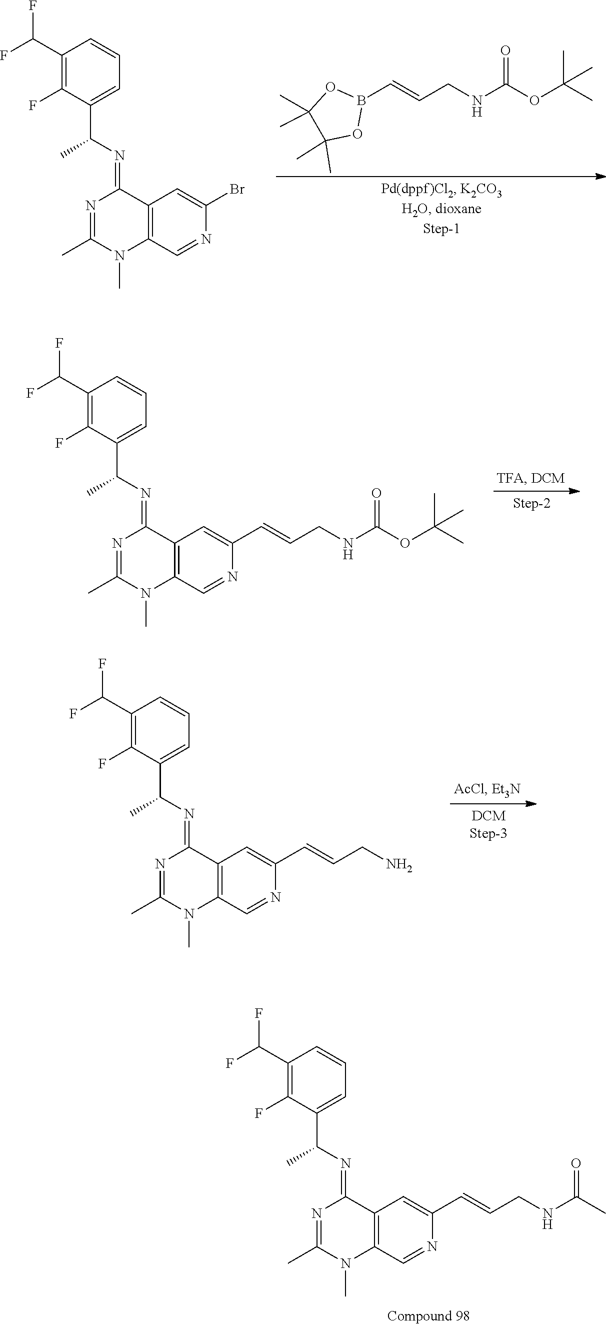 [SMILES: Cc1n/c(=N\[C@H](C)c2cccc(C(F)F)c2F)c2cc(Br)ncc2n1C ; CC(C)(C)OC(=O)NC/C=C/B1OC(C)(C)C(C)(C)O1 ; Cc1n/c(=N\[C@H](C)c2cccc(C(F)F)c2F)c2cc(/C=C/CNC(=O)OC(C)(C)C)ncc2n1C ; Cc1n/c(=N\[C@H](C)c2cccc(C(F)F)c2F)c2cc(/C=C/CN)ncc2n1C ; CC(=O)NC/C=C/c1cc2/c(=N/[C@H](C)c3cccc(C(F)F)c3F)nc(C)n(C)c2cn1]

### Step 1. Synthesis of tert-butyl ((E)-3-((Z)-4-(((R)-1-(3-(difluoromethyl)-2-fluorophenyl)ethyl)imino)-1,2-dimethyl-1,4-dihydropyrido[3,4-d]pyrimidin-6-yl)allyl)carbamate

To a solution of tert-butyl (E)-(3-(4,4,5,5-tetramethyl-1,3,2-dioxaborolan-2-yl)allyl)-carbamate (200 mg, 0.7 mmol) in H₂O (1 mL) and dioxane (5 mL) were added K₂CO₃ (292 mg, 2.118 mmol), Pd(dppf)Cl₂ (51.66 mg, 0.071 mmol) and (R,Z)-6-bromo-N-(1-(3-(difluoro-methyl)-2-fluorophenyl)ethyl)-1,2-dimethylpyrido[3,4-d]pyrimidin-4(1H)-imine (360.27 mg, 0.847 mmol). The mixture was stirred at 110° C. for 15 h. Water (30 mL) was added, the mixture was extracted with EtOAc, the combined organic layers were washed with brine, dried over NaSO₄, filtered and concentrated to dryness. The residue was purified by column chromatography (PE:EA=3:1) to give the tittle compound (150 mg, 0.299 mmol, 42.35%).

LC-MS m/e: 502 (MH⁺).

### Step 2. Synthesis of (E)-3-((Z)-4-(((R)-1-(3-(difluoromethyl)-2-fluorophenyl)ethyl)imino)-1,2-dimethyl-1,4-dihydropyrido[3,4-d]pyrimidin-6-yl)prop-2-en-1-amine

To a solution of tert-butyl ((E)-3-((Z)-4-(((R)-1-(3-(difluoromethyl)-2-fluorophenyl)-ethyl)imino)-1,2-dimethyl-1,4-dihydropyrido[3,4-d]pyrimidin-6-yl)allyl)carbamate (150 mg, 0.299 mmol) in DCM (5 mL) was added TFA (2 mL, 26.118 mmol). The mixture was stirred at rt for 1 h and concentrated to give the desired product (110 mg, 0.274 mmol, 91.67%).

LC-MS m/e: 402 (MH⁺).

### Step 3. Synthesis of N-((E)-3-((Z)-4-(((R)-1-(3-(difluoromethyl)-2-fluorophenyl)ethyl)imino)-1,2-dimethyl-1,4-dihydropyrido[3,4-d]pyrimidin-6-yl)allyl)acetamide (Compound 98)

To a solution of (E)-3-((Z)-4-(((R)-1-(3-(difluoromethyl)-2-fluorophenyl)ethyl)imino)-1,2-dimethyl-1,4-dihydropyrido[3,4-d]pyrimidin-6-yl)prop-2-en-1-amine (110 mg, 0.274 mmol) in DCM (2 mL) were added acetylchloride (0.029 mL, 0.4 mmol) and TEA (0.114 mL, 0.8 mmol). The mixture was stirred at 0° C. for 0.5 h. Water (30 mL) was added, the mixture was extracted with EtOAc, the combined organic layers were washed with brine, dried over Na₂SO₄, filtered and concentrated to dryness. The residue was purified by column chromatography (PE:EA=3:1) to give the title compound (27 mg, 0.061 mmol, 22.2%).

¹H NMR (400 MHz, DMSO-d₆) δ 8.82 (s, 1H), 8.19 (s, 1H), 8.14 (t, J=4.0 Hz, 1H), 8.00 (s, 1H), 7.96 (t, J=6.0 Hz, 1H), 7.47 (t, J=8.0 Hz, 1H), 7.34-7.07 (m, 2H), 6.72-6.64 (m, 2H), 5.79 (q, J=8.0 Hz, 1H), 3.93-3.88 (m, 2H), 3.63 (s, 3H), 2.45 (s, 3H), 1.88 (s, 3H), 1.40 (d, J=8.0 Hz, 3H). LC-MS m/e: 444.1 (MH⁺).

Compounds 99-113 can be readily prepared following general Scheme XII and the procedure described above for compound 92.

### HTRF Based Protein-Protein Interaction Assay

A KRAS-G12D and SOS1 binding/interaction assay is designed to measure the interaction between KRAS-G12D and SOS1 proteins. Utilizing HTRF (Homogeneous Time-Resolved Fluorescence) technology, the assay enables simple and rapid characterization of compound and protein interaction in a high throughput format. This assay was used to examine the potency of compounds to inhibit the protein-protein interaction between SOS1 and KRAS-G12D, demonstrating the molecular mode of action of compounds. Low IC₅₀ values are indicative of high potency of a SOS1 inhibitor to disrupt SOS1 and Kras-G12D interaction.

### Protein Expression and Purification

The corresponding gene sequence to human SOS1 (residues 564-1049, UniProt ID: Q07889) was synthesized and fused in frame with GST expression vector. The sequence was confirmed and then plasmid containing desired sequence was transformed into the E. coli strain BL21 (DE3). Bacteria were growing at 37° C. in LB media containing 50 μg/ml Kanamycin to OD₆₀₀ of 0.6-0.8. Then 0.2 mM IPTG was added to induce protein expression for 16 h at 17° C. Bacteria were harvested by centrifugation and stored at −80° C. Bacteria pellets were resuspended in lysis buffer (25 mM Tris-HCl, 500 mM NaCl, 2 mM DTT, 2.3% sucrose, 0.3% dextran-10, 1 mM PMSF, pH 7.5) and lysed using High-pressure homogenizer. The lysate was cleared by centrifugation for 30 min. (12000 rpm at 4° C.). The supernatant containing GST-SOS1 fragment was purified sequentially through Glutathione column and gel filtration (Hiload 16/600 Superdex® 200 μg column, Cytiva). The purified GST-SOS1 fragment was confirmed by SDS-PAGE and stored in 25 mM Tris-HCl, 100 mM NaCl, 1 mM DTT, 2.3% sucrose, 0.3% dextran-10, pH 7.5 at −80° C.

### Kras-G12D Protein Purification

The sequence corresponding to human KRAS-G12D (residues 1-169, UniProt P01116-2) was synthesized and fused in frame with His-AVI-TEV vector. The plasmid was transformed into the E. coli strain BL21 (containing a plasmid that can generate BirA enzyme). Bacteria were growing at 37° C. in TB media containing 100 ug/ml ampicillin and 50 ug/ml Kanamycin to OD₆₀₀ of 0.6-0.8. The 0.5 mM IPTG and 50 mg/l biotin were added to induce protein expression for 16 h at 25° C. Bacteria were harvested by centrifugation and stored at −80° C. Bacteria pellets were resuspended in lysis buffer (20 mM Tris-HCl, 500 mM NaCl, 5 mM MgCl₂, 2 mM 3-ME, 5% glycerol, pH 8) and lysed using High-pressure homogenizer. The lysate was cleared by centrifugation (12000 rpm at 4° C.) for 30 min. The supernatant containing HIS-AVI-TEV-KRAS-G12D fragment was purified sequentially through Ni-NTA column (SMART), Streptactin® column (SMART), and gel filtration (Hiload 16/600 Superdex® 75 μg, GE). The purified HIS-AVI-TEV-KRAS-G12D fragment was confirmed by SDS-PAGE and stored in 50 mM HEPES-NaOH, 100 mM NaCl, 1 mM DTT, 5 mM MgCl₂, pH 7.5 at −80° C.

### Protein-Protein Interaction Assay

An assay buffer containing 50 mM HEPES, pH 7.5, 50 mM NaCl, 0.01% Brij-35, 1 mM TCEP, and 0.1% BSA was prepared. Concentration series of test compounds were generated spanning 0.5 nM to 10 μM over 10 3-fold serial dilutions in a 384-well assay plate at a volume of 20 pL. The purified GST-SOS1 catalytic domain (residues 564-1049) was first diluted in assay buffer and 5 μl of SOS1 (final concentration 2.5 nM in assay mixture) was directly dispensed into compound plates. After a spin down at 1000 RPM for 30 seconds the SOS1/compound mixture was incubated at 25° C. for 15 min to allow the reaction between SOS1 and compound. A KRAS-G12D mixture was prepared by incubation of avi-tagged Kras-G12D (residue 1-169) and GDP in assay buffer containing 10 mM MgCl₂ at room temperature for 10 min. KRAS-G12D and GDP mixture (5 μL) was added to the assay plate with a final KRAS-G12D concentration at 100 nM and GDP at 10 μM). The plate was centrifuged at 1000 rpm for 30 sec and incubated at 25° C. for 60 min. A monoclonal antibody to GST-conjugated with Tb cryptate and Streptavidin-XL665 in 1× assay buffer was prepared and 10 μl of the detection mixture was added to each well. The plate was incubated at 25° C. for 5 hours. A reading in HTRF mode with PerkinElmer Envision™ plate reader was taken at the end of incubation. An assay buffer with KRAS-G12D plus DMSO and mixture of SOS1 plus kRAS-G12D plus DMSO were used as negative controls (minimum signal, column 1 and 2) and positive controls (maximum signal, column 23 and 24), respectively. Percent (%) inhibition for each dilution of compound was calculated as follows: [percent (%) inhibition=(1−(sample signal-negative control)/(positive control-negative control))*100]. Two to three separate experiments were performed for each compound and the data were analyzed using a four-parameter logistic fit.

A group of exemplified compounds were evaluated in the above assay. These data demonstrate compounds of the invention are potent inhibitors of the protein-protein interaction between SOS1 and KRAS-G12D.

pERK Potency Assay

The assay measures the ability of a compound to inhibit SOS1 function in cells. SOS1 activates RAS proteins by catalyzing the conversion of RAS GDP to RAS GTP in response to receptor tyrosine kinase activation. Activation of RAS induces a sequence of cellular signaling events that result in increased phosphorylation of ERK at Threonine 202 and Tyrosine 204 (pERK). The procedure (i.e., In Cell ELISA as described below) measures the level of cellular pERK in response to a test compound in NCI-H1975 cells (EGFR/L858R-T790M).

First, NCI-H1975 cells were grown and maintained using media and procedures recommended by the ATCC. A day before compound addition, cells were plated in poly-D-lysine coated 96-well cell culture plates (Corning® BioCoat® Cat #356640) at 25000 cells/well/100 μL and grown overnight in an incubator at 37° C. with 5% CO₂. A solution of a test compound was prepared with 3-fold serial dilutions in DMSO having a top concentration of 6 mM. On the day of the assay, 50 μL of a test compound dilution in media was added to each well of cell culture plate with a final compound concentration in the range of 0.001 μM to 20 μM. After addition of the compound, cells were incubated for 1 h at 37° C. with 5% CO₂, after which the culture medium was removed. Cells were fixed with 4% formaldehyde in phosphate-buffered saline (PBS) and incubated at RT for 20 min. The plate was washed three times with PBST (PBS with 0.05% Tween®-20), followed by incubation with 100 μL of pre-cooled methanol at −20° C. for 20 min. After incubation, methanol was removed. The plate was again washed with PBST. Cells were then permeabilized with 100 μL/well of 0.1% Triton X-100 in PBS at RT for 20 min and then quenched with a quenching buffer (PBST containing 1% H₂O₂ and 0.1% sodium azide) for 20 min at RT with gentle shaking. After washing once with PBST, a blocking buffer (250 uL/well, Pierce™ Protein-Free-PBS Blocking Buffer, Cat #37572) was then added to the cells for 1-h incubation at RT, followed by an anti-pERK antibody (cell signaling, 1:1000 dilution in 5% BSA in PBST) incubation overnight at 4° C. and a secondary antibody-HRP (Jackson immunoresearch, 1:3000 dilution in 5% BSA in PBST) incubation at RT for 1 h. PBST wash was carried out after each step three times. Before the addition of a substrate (Advansta ELISABright™), the plate was washed with PBS twice to remove detergent residues. The Cellular pERK level was determined using the microplate reader (Biotek Synergy™ H1) to detect the chemiluminescence signal. IC₅₀ was determined by fitting a 4-parameter sigmoidal concentration-response model.

Compounds 1-98 were evaluated using the pERK potency assay described above. Compounds 1-62, 64-69, 71-82, 84, 85, 89, and 92-98 each showed an IC₅₀ below 100 nM.

### In Vivo Efficacy Study

Anti-tumor activity of the SOS1 inhibitor in the invention was evaluated in mice using the subcutaneous xenograft human tumor models which were conducted in accordance with Animal Use Protocols approved by Local Animal Welfare Committee. Animals were housed in barrier facilities fully accredited by the Association for Assessment and Accreditation International. Six to eight-week-old female mice (balb/c nude mice or NOD SCID mice) were injected subcutaneously with cell line derived tumors (NCI-H1975, Miapaca-2, Kyse-410, NCI-H358) or inoculated with patient derived tumors (LU11692 and CR6256). When tumors reached a volume of approximately 150-200 mm³ mice were randomized into groups and dosed via oral gavage with either vehicle control, SOS1 inhibitor (3, 10, 30 mg/kg), osimertinib (1 mg/kg), sotorasib (5, 10, 50 mg/kg), or combination studies with SOS1 inhibitor plus osimertinib or SOS1 inhibitor plus sotorasib depending on tumor models. Compounds were given twice a day (SOS1 inhibitor) or once a day (osimertinib and sotorasib) for 21 to 28 days. Body weight and tumor volume were measured twice per week. Tumor volume was calculated as mean and standard error of the mean for each treatment group.

Compounds of this invention as tested demonstrated good tumor regression in the in vivo efficacy study.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their efficacy in treating a condition that relates to SOS1. Thus, other embodiments were also within the claims.

What is claimed is:

1. A compound of formula I: 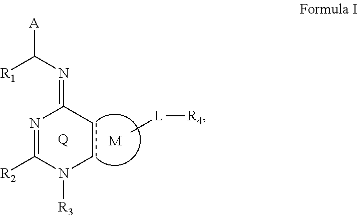
or a pharmaceutical acceptable salt, solvate, isomer, prodrug, or tautomer thereof, wherein:
A is C₃-C₆ cycloalkyl, 4-10 membered heterocycloalkyl, aryl, or 5-10 membered heteroaryl;
L is a single bond, —O—, —C(O)—, —C(O)O—, —O—CO—, —C(O)NR′—, —NR′—, —NR′CO—, —NR′SO₂—, —SO₂NR′—, —S—, —SO—, —S(O)₂—, C₁-C₃ alkylene, C₁-C₃ haloalkylene, C₂-C₃ alkenylene, or C₂-C₃ alkynylene, in which R′ is H, C₁-C₆ alkyl, C₁-C₆ haloalkyl, C₃-C₆ cycloalkyl, 3-6 membered heterocycloalkyl, 5-10 membered heteroaryl, or 6-10 membered aryl;
M is a C₅-C₁₀ cycloalkyl, 5-15 membered heterocyclyl, 6-10 membered aryl, or 5-10 membered heteroaryl;
Q indicates the left ring of Formula I to which M is fused;
R₁ is CN, C₁-C₃ alkyl, C₂-C₃ alkenyl, or C₂-C₃ alkynyl;
R₂ is H, CN, halogen, C₁-C₃ alkyl, C₁-C₃ alkoxy, C₂-C₃ alkynyl, or C₃-C₆ cycloalkyl;
R₃ is C₁-C₆ alkyl, C₃-C₆ cycloalkyl, 4-6 membered heterocycloalkyl, 5-6 membered heteroaryl, or phenyl; or R₂ and R₃, together with the carbon atom to which R₂ bonds and the nitrogen atom to which R₃ bonds, form a 4-7 membered heterocycloalkyl;
R₄ is H, C₁-C₁₀ alkyl, C₁-C₁₀ alkylamino, C₁-C₁₀ alkylamido, C₂-C₁₀ alkylcarbonyl, C₁-C₁₀ alkylsulfonyl, C₁-C₁₀ alkysulfonamido, C₃-C₆ cycloalkyl, C₃-C₆ cycloalkylcarbonyl, C₃-C₆ cycloalkylamido, 3-10 membered heterocycloalkyl, 6-10 membered aryl, or 5-10 membered heteroaryl; [symbol]
is a single or double bond; and
each of alkyl, alkylene, alkenyl, alkynyl, alkoxy, haloalkylene, alkylamino, alkylamido, alkylcarbonyl, alkylsulfonyl, alkysulfonamido, alkynylene, cycloalkyl, cycloalkylcarbonyl, cycloalkylamido, heterocycloalkyl, heterocyclyl, phenyl, aryl, and heteroaryl is unsubstituted or substituted with one or more groups selected from deuterium, halogen, CN, oxo, C₁-C₆ alkyl, C₃-C₆ cycloalkyl, C₁-C₆ haloalkyl, C₂-C₆ alkenyl, C₂-C₆ alkynyl, —OR′, —C(O)R′, —C(O)OR′, —O—COR′, —C(O)NR′R″, —NR′R″, —SR′, —SOR′, —S(O)₂R′, and —POR′R″, in which each of R′ and R″, independently, is H, halogen, C₁-C₆ alkyl, C₁-C₆ haloalkyl, C₃-C₆ cycloalkyl, 3-6 membered heterocycloalkyl, 5-10 membered heteroaryl, or 6-10 membered aryl.

2. The compound of claim 1, wherein
M is a C₅-C₁₀ cycloalkyl, 5-12 membered heterocycloalkyl, 6-10 membered aryl, or 5-10 membered heteroaryl;
R₁ is CN, C₁-C₃ alkyl, ethenyl, or ethynyl;
R₂ is H, CN, halogen, C₁-C₃ alkyl, C₁-C₃ alkoxy, ethenyl, or cyclopropyl; R₃ is C₁-C₆ alkyl, C₃-C₆ cycloalkyl, 4-6 membered heterocycloalkyl, 5-6 membered heteroaryl, or phenyl; or R₂ and R₃, together with the carbon atom to which R₂ bonds and the nitrogen atom to which R₃ bonds, form a 4-7 membered heterocycloalkyl;
R₄ is H, C₁-C₁₀ alkyl, C₃-C₆ cycloalkyl, 3-10 membered heterocycloalkyl, 6-10 membered aryl, or 5-10 membered heteroaryl; and
each of alkyl, cycloalkyl, heterocycloalkyl, phenyl, aryl, and heteroaryl is unsubstituted or substituted with one or more groups selected from deuterium, halogen, CN, oxo, C₁-C₆ alkyl, C₃-C₆ cycloalkyl, C₁-C₆ haloalkyl, C₂-C₆ alkenyl, C₂-C₆ alkynyl, —OR′, —C(O)R′, —C(O)OR′, —O—COR′, —C(O)NR′R″, —NR′R″, —SR′, —SOR′, —S(O)₂R′, or —POR′R″, in which each of R′ and R″, independently, is H, halogen, C₁-C₆ alkyl, C₁-C₆ haloalkyl, C₃-C₆ cycloalkyl, 3-6 membered heterocycloalkyl, 5-10 membered heteroaryl, or 6-10 membered aryl.

3. The compound of claim 1, wherein the compound is of formula II: 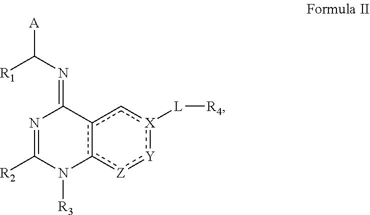
in which each of [symbol] is a single or double bond and the number of double bonds represented by [symbol] in formula II is 2 or 3; X is C or N; and each of Y and Z, independently, is C═O, C—Rx, or N—Ry, provided that when X is N, then Y is C═O and Z is C—Rx; Rx is H, CN, halogen, C₁-C₃ alkyl, C₁-C₃ alkoxy, C₁-C₃ haloalkyl, cyclopropyl, four to six membered heterocycloalkoxy, or NR′R″; and Ry is absent, H, C₁-C₃ alkyl, or cyclopropyl.

4. The compound of claim 3, wherein the compound is of one of formula II-A to formular II-F.

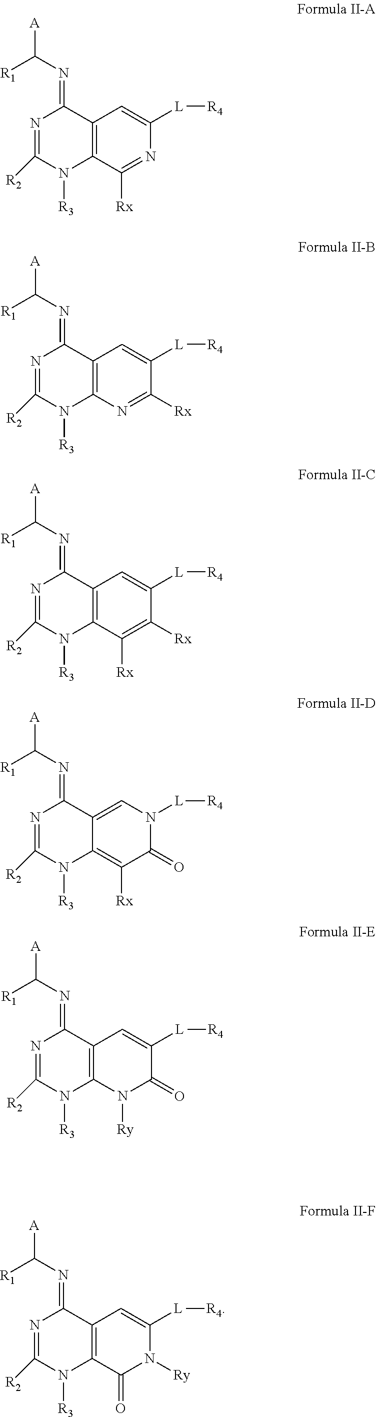

5. The compound of claim 3, wherein the compound is of formula II-G or formula II-H: 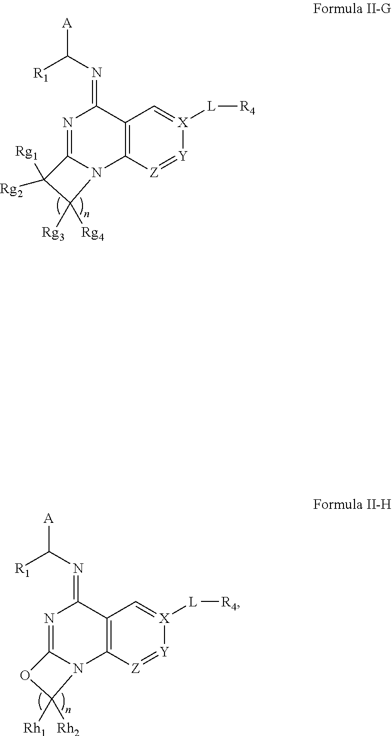
in which n is 2, 3, or 4, preferably 2; and each of Rg₁, Rg₂, Rg₃, Rg₄, Rh₁, and Rh₂, independently in each occurrence, is H, halogen, or methyl.

6. The compound of claim 1, wherein

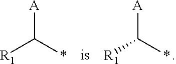

7. The compound of claim 3, wherein the compound is of formula III: 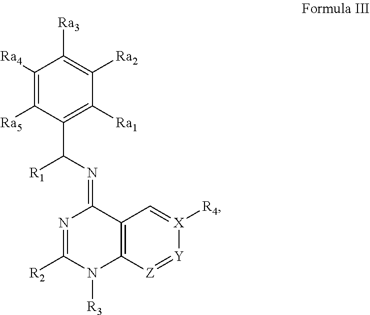
in which each of Ra₁, Ra₂, Ra₃, Ra₄, and Ra₅, independently, is H, halogen, amino, CN, C₁-C₆ alkyl, C₂-C₆ alkenyl, C₁-C₆ haloalkyl, C₃-C₆ cycloalkyl, or 4-6 membered heterocycloalkyl.

8. The compound of claim 7, wherein the compound is of formula IIIa:

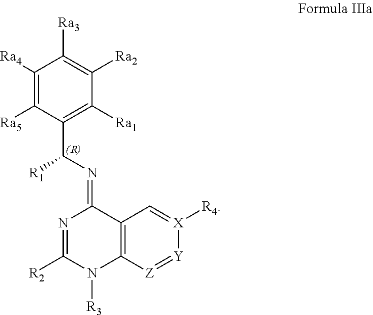

9. The compound of claim 1, wherein R₁ is methyl or ethynyl.

10. The compound of claim 1, wherein R₂ is H, C₁-C₃ alkyl, or cyclopropyl.

11. The compound of claim 1, wherein R₃ is C₁-C₄ alkyl, C₃-C₆ cycloalkyl, or 4-6 membered heterocycloalkyl, preferably, R₃ is C₁-C₄ alkyl or cyclopropyl.

12. The compound of claim 1, wherein Rx is H, halogen, C₁-C₃ alkyl, cyclopropyl, C₁-C₃ alkoxy, NH₂, C₁-C₃ alkylamino; and Ry is H, C₁-C₃ alkyl, or cyclopropyl.

13. The compound of claim 1, wherein A is phenyl, 2,3-dihydro-1H-indene, or benzofuran, and A is unsubstituted or substituted with one or more groups selected from deuterium, halogen, NH₂, CN, C₁-C₄ alkyl, C₂-C₄ alkenyl, C₁-C₄ haloalkyl, or C₃-C₆ cycloalkyl; preferably A is phenyl substituted with one or more groups selected from F, Cl, Br, NH₂, CN, C₁-C₃ alkyl, and C₁-C₃ haloalkyl.

14. The compound of claim 1, wherein A is

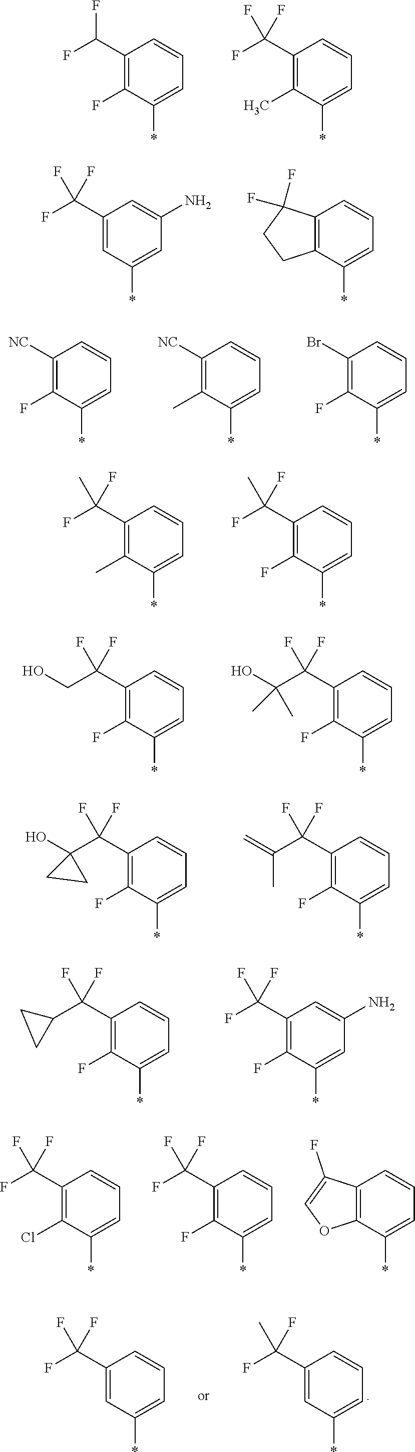

15. The compound of claim 14, wherein A is

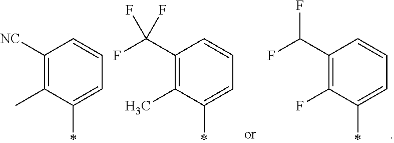

16. The compound of claim 1, wherein L is a single bond, —O—, —C(O)—, —NHC(O)—, —NHS(O)₂—, C₂-C₃ alkenylene, or C₂-C₃ alkynylene.

17. The compound of claim 1, wherein L is a single bond.

18. The compound of claim 1, wherein R₄ is selected from the group consisting of:

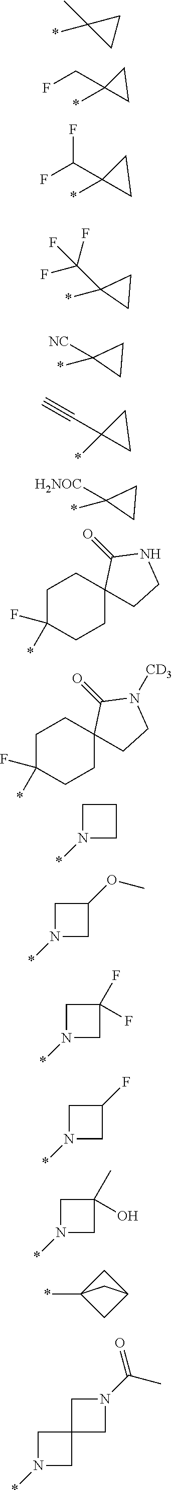

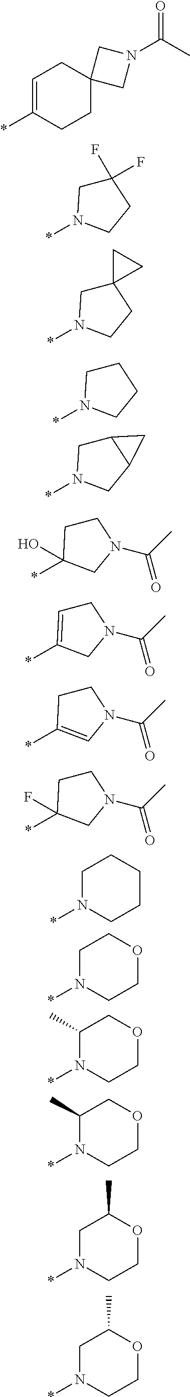

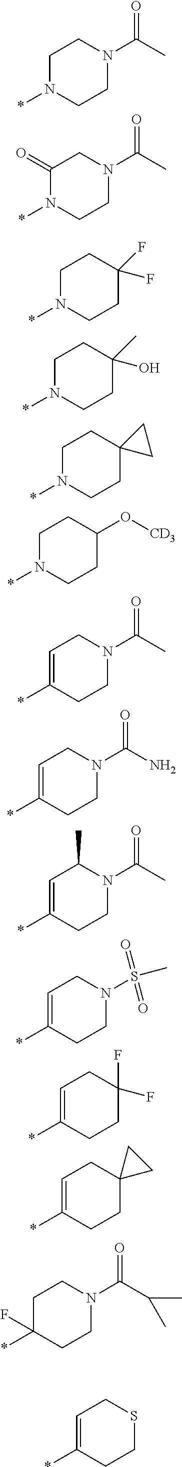

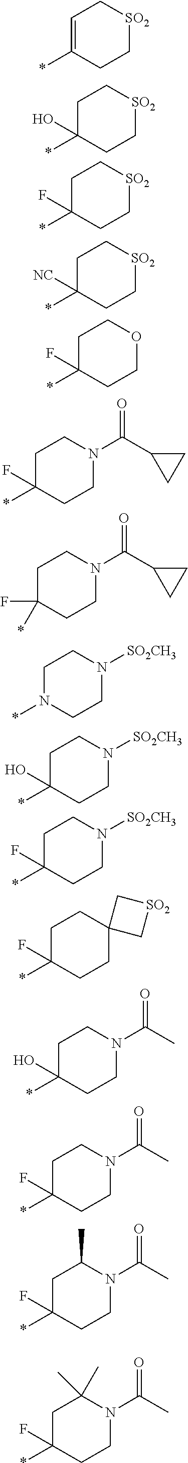

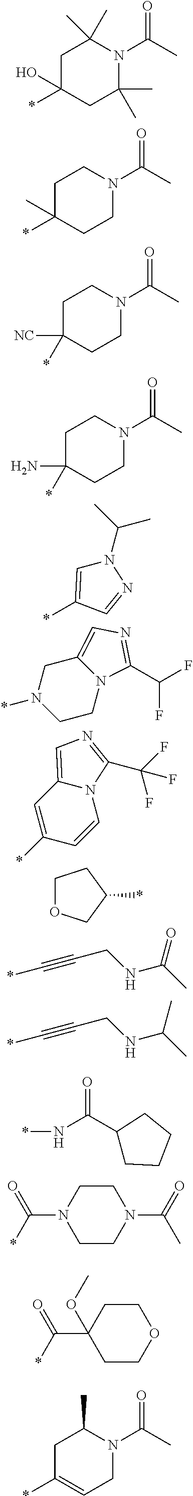

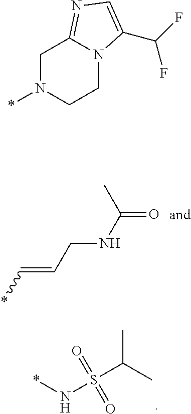

19. The compound of claim 18, wherein R₄ is selected from the group consisting of

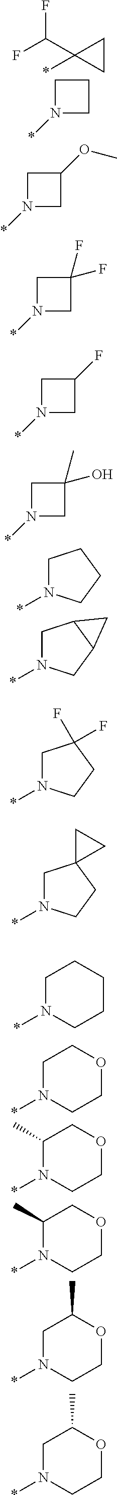

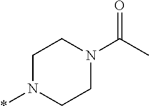 [SMILES: *N1CCN(C(C)=O)CC1]

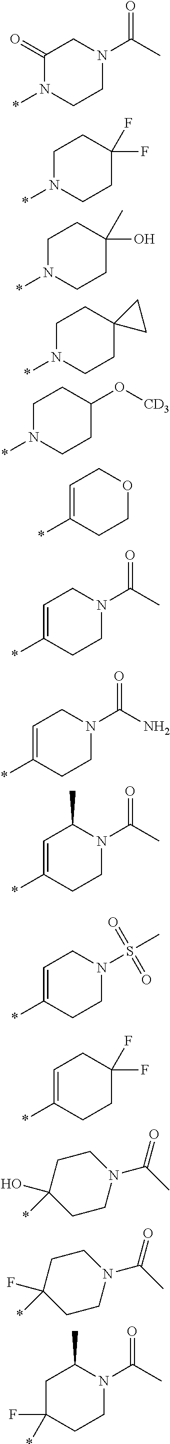

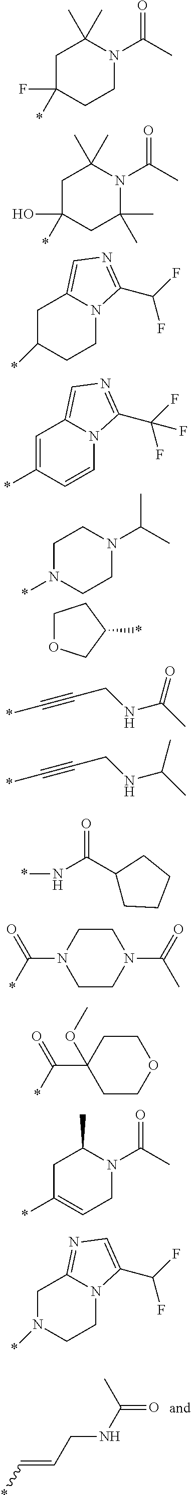

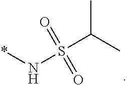 [SMILES: *NS(=O)(=O)C(C)C]

20. The compound of claim 1, wherein the compound is of formula IV, V, or VI, 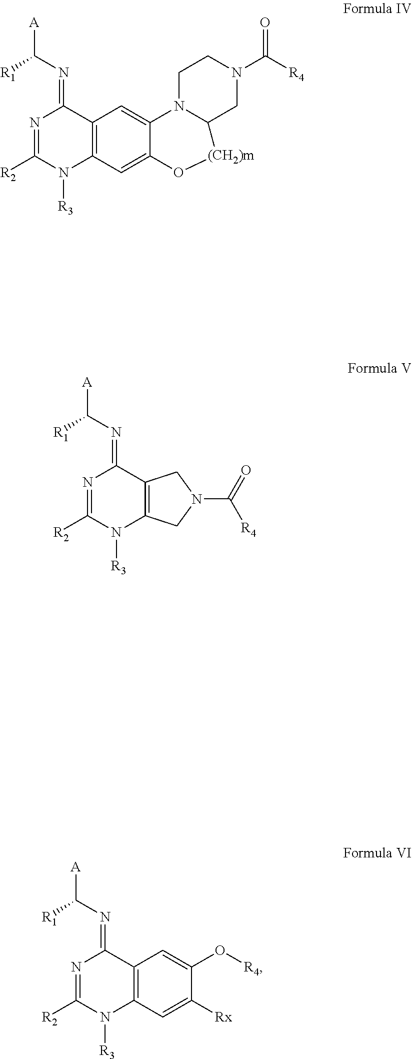
in which m is 0, 1, or 2, and Rx is H or C₁-C₁₀ alkoxy.

21. The compound of claim 20, wherein
R₁ is methyl or ethynyl,
R₃ is C₁-C₄ alkyl, C₃-C₆ cycloalkyl, or 4 to 6-membered heterocycloalkyl;
R₄ is H, C₁-C₆ alkyl, C₁-C₆ haloalkyl, C₃-C₆ cycloalkyl, or 4 to 6-membered heterocycloalkyl;
A is 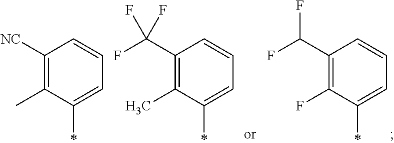
and
m is 1 or 2.

22. The compound of claim 20, wherein R₃ is C₁-C₄ alkyl or cyclopropyl.

23. The compound of claim 1, wherein the compound is of formula IV, 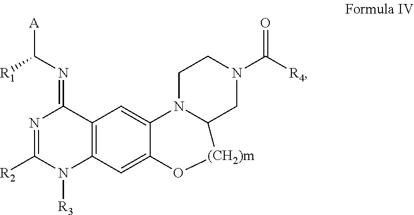
in which R₁ is methyl; R₂ is H or methyl, R₃ is methyl, or R₂ and R₃, together with the carbon atom to which R₂ bonds and the nitrogen atom to which R₃ bonds, form a 5 or 6-membered heterocycloalkyl; R₄ is methyl or cyclopropyl; and m is 1 or 2.

24. The compound of claim 1, wherein the compound is one of Compounds 1-113.

25. A pharmaceutical composition comprising a compound of claim 1 and a pharmaceutically acceptable carrier thereof.

26. A method of treating cancer comprising administering to a subject in need thereof an effective amount of a compound of claim 1.

27. A method of inhibiting SOS1 comprising administering to a subject in need thereof an effective amount of a compound of claim 1.
